(19) United States
(12) Patent Application Publication
(10) Pub. No.: US 2023/0178239 A1
(43) Pub. Date: Jun. 8, 2023

(54) METHODS OF IDENTIFYING FEATURES ASSOCIATED WITH CLINICAL RESPONSE AND USES THEREOF

(71) Applicant: Juno Therapeutics, Inc., Seattle, WA (US)

(72) Inventors: Ronald James HAUSE, JR., Seattle, WA (US); Yue JIANG, Seattle, WA (US)

(73) Assignee: Juno Therapeutics, Inc., Seattle, WA (US)

(21) Appl. No.: 17/924,652

(22) PCT Filed: May 12, 2021
(86) PCT No.: PCT/US2021/032104

§ 371 Date: Nov. 10, 2022

Related U.S. Application Data
(60) Provisional application No. 63/037,592, filed on Jun. 10, 2020.
(60) Provisional application No. 63/024,494, filed on May 13, 2020.

(51) Int. Cl. G16H 50/20; A61K 35/17; G06N 20/20

(52) U.S. Cl. CPC G16H 50/20; A61K 35/17; G06N 20/20

(57) ABSTRACT

The present disclosure relates to methods for identifying features, such as attributes of subjects, therapeutic cell compositions, and input compositions used to produce therapeutic cell compositions, associated with clinical responses of subjects, e.g., patients, following treatment with the therapeutic cell composition in connection with a cell therapy. The cells of the therapeutic cell composition express recombinant receptors such as chimeric receptors, e.g., chimeric antigen receptors (CARs) or other transgenic receptors such as T cell receptors (TCRs). The methods provide for the identification of features associated with clinical responses. In some embodiments, the methods can be used to determine (e.g., predict) a subject's response to treatment with the therapeutic cell composition.

### CROSS-REFERENCE TO RELATED APPLICATIONS

This application claims priority from U.S. provisional application No. 63/024,494, filed May 13, 2020, entitled “METHODS OF IDENTIFYING FEATURES ASSOCIATED WITH CLINICAL RESPONSE AND USES THEREOF,” and U.S. provisional application No. 63/037,592, filed Jun. 10, 2020, entitled “METHODS OF IDENTIFYING FEATURES ASSOCIATED WITH CLINICAL RESPONSE AND USES THEREOF,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042023740SeqList.TXT, created May 8, 2021, which is 51,373 bytes in size. The information in the electronic format of the Sequence Listing is incorporated by reference in its entirety.

### FIELD

The present disclosure relates to methods for identifying features, such as attributes of subjects, therapeutic cell compositions, and input compositions used to produce therapeutic cell compositions, associated with clinical responses of subjects, e.g., patients, following treatment with the therapeutic cell composition in connection with a cell therapy. The cells of the therapeutic cell composition express recombinant receptors such as chimeric receptors, e.g., chimeric antigen receptors (CARs) or other transgenic receptors such as T cell receptors (TCRs). The methods provide for the identification of features associated with clinical responses. In some embodiments, the methods can be used to determine (e.g., predict) a subject's response to treatment with the therapeutic cell composition.

### BACKGROUND

Various immunotherapy and/or cell therapy methods are available for treating diseases and conditions. For example, adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be beneficial in the treatment of cancer or other diseases or disorders. Improved approaches are needed for determining whether a treatment will result in a beneficial clinical response. Provided herein are methods that address such needs.

### SUMMARY

Provided herein are methods of identifying features associated with a clinical response, the method comprising: (a) receiving features comprising: (i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a CAR; and (iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects; (b) preprocessing the features to identify informative features, the informative features comprising a subset of the features comprising one or more of the subject features, one or more the input composition features, and one or more of the therapeutic cell composition features; (c) obtaining clinical responses from each of the plurality of subjects following treatment with one of the plurality of therapeutic compositions; (d) applying the informative features and the obtained clinical responses from the plurality of subjects as input to train a random forests model using supervised learning; and (e) identifying from the trained random forests model the informative features associated with the clinical responses.

Provided herein are methods of identifying features associated with a clinical response, the method comprising: (a) receiving features comprising: (i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and (iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects; (b) preprocessing the features to identify informative features, the informative features comprising a subset of the features comprising one or more of the subject features, one or more of the input composition features, and one or more of the therapeutic cell composition features; (c) obtaining clinical responses over time from each of the plurality of subjects following treatment with one of the plurality of therapeutic compositions; (d) applying the informative features and clinical responses from the plurality of subjects as input to train a random survival forests model using supervised learning; and (e) identifying from the trained random survival forests model the informative features associated with the clinical responses.

In some embodiments of any of the methods provided herein, the identifying the informative features associated with the clinical responses comprises determining an importance measure for each of the informative features. In some embodiments, the importance measure comprises a permutation importance measure, a mean minimal depth, and/or a total number of trees from the random forests, e.g., the trained random forests model, wherein the informative feature splits a root node. In some embodiments, the importance measure comprises a permutation importance measure, a mean minimal depth, and/or a total number of trees from the random survival forests, e.g., the trained random survival forests model, wherein the informative feature splits a root node. In some embodiments, the importance measure is the permutation importance measure. In some embodiments, the importance measure is the mean minimal depth. In some embodiments, the importance measure is the total number of trees from the random forests, e.g., the trained random forests model, wherein the informative feature splits a root node. In some embodiments, the importance measure is the total number of trees from the random survival forests, e.g., the trained random survival forests model, wherein the informative feature splits a root node. In some embodiments, the informative features associated with the clinical responses are the first 10, 9, 8, 7, 6, 5, 4, 3, 2, or 1 informative features identified by rank ordering, e.g., rank ordering values, of the importance measure for each of the informative features, wherein the importance measure is the same for each informative feature. In some embodiments, the informative features associated with the clinical responses are the first 5 informative features identified by rank ordering values of the importance measure for each of the informative features, wherein the importance measure is the same for each informative feature. In some embodiments, the informative features associated with the clinical responses is the first informative feature identified by rank ordering values of the importance measure for each of the informative features, wherein the importance measure is the same for each informative feature.

Provided herein are methods of determining, e.g., predicting, a clinical response, the method comprising: (a) receiving features comprising: (i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from an input composition, wherein the input composition comprises T cells selected from a sample from the subject, wherein the T cells are used for producing the therapeutic cell composition comprising T cells comprising a chimeric antigen receptor (CAR); and (iii) therapeutic cell composition features determined from the therapeutic cell composition, wherein the therapeutic cell composition is produced from the input composition and expresses the CAR, wherein the therapeutic composition is to be administered to the subject; and (b) applying the features as input to a random forests model trained to determine, e.g., predict, based on informative features identified by preprocessing, clinical responses of the subject to treatment with the therapeutic cell composition prior to treating the subject with the therapeutic cell composition, wherein the features applied as input are the same informative features as those used to train the random forests model.

Provided herein are methods of determining, e.g., predicting, a clinical response, the method comprising: (a) receiving features comprising: (i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from an input composition, wherein the input composition comprises T cells selected from a sample from the subject, wherein the T cells are used for producing the therapeutic cell composition comprising T cells comprising a chimeric antigen receptor (CAR); and (iii) therapeutic cell composition features determined from the therapeutic cell composition, wherein the therapeutic cell composition is produced from the input composition and expresses the CAR, wherein the therapeutic composition is to be administered to the subject; and (b) applying the features as input to a random survival forests model trained to determine, e.g., predict, based on informative features identified by preprocessing, clinical responses of the subject to treatment with the therapeutic cell composition prior to treating the subject with the therapeutic cell composition, wherein the features applied as input are the same informative features as those used to train the random survival forests model.

Provided herein are methods of treating a subject, the method comprising: (a) selecting T cells from a sample from a subject to produce an input composition comprising T cells; (b) determining features comprising: (i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from an input composition, wherein the input composition comprises T cells selected from a sample from the subject, wherein the T cells are used for producing the therapeutic cell composition comprising T cells comprising a chimeric antigen receptor (CAR); and (iii) therapeutic cell composition features determined from the therapeutic cell composition, wherein the therapeutic cell composition is produced from the input composition and expresses the CAR, wherein the therapeutic composition is to be administered to the subject; and (c) applying the features as input to a random forests model trained to determine, e.g., predict, based on informative features identified by preprocessing, clinical responses of the subject to treatment with the therapeutic cell composition prior to treating the subject with the therapeutic cell composition, wherein the features applied as input are the same informative features as those used to train the random forests model; and administering a treatment to the subject wherein: (1) if the subject is determined, e.g., predicted, to have a clinical response selected from the group consisting of a complete response (CR), a partial response (PR), a durable response of greater than 3 months, progression free survival (PFS) for more than 3 months, overall response rate (ORR), objective response (OR), a desired pharmacokinetic response that is or is greater than a target pharmacokinetic response, and no or a mild toxicity response (optionally wherein the toxicity is grade 2 or less CRS or grade 2 or less neurotoxicity), a predetermined treatment regimen comprising the therapeutic cell composition is administered; or (2) if the subject is determined, e.g., predicted, to have a clinical response selected from the group consisting of a toxicity response (optionally wherein the toxicity response is a severe cytokine release syndrome or severe neurotoxicity), a reduced pharmacokinetics response compared to a target pharmacokinetic response, progressive disease (PD), a durable response of less than 3 months, and PFS of less than 3 months, administering to the subject a treatment regimen comprising the therapeutic cell composition that is altered compared to the predetermined treatment regimen comprising the therapeutic cell composition.

Provided herein are methods of treating a subject, the method comprising: (a) selecting T cells from a sample from a subject to produce an input composition comprising T cells; (b) determining features comprising: (i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from an input composition, wherein the input composition comprises T cells selected from a sample from the subject, wherein the T cells are used for producing the therapeutic cell composition comprising T cells comprising a chimeric antigen receptor (CAR); and (iii) therapeutic cell composition features determined from the therapeutic cell composition, wherein the therapeutic cell composition is produced from the input composition and expresses the CAR, wherein the therapeutic composition is to be administered to the subject; and (c) applying the features as input to a random survival forests model trained to determine, e.g., predict, based on informative features identified by preprocessing, clinical responses in the subject to be treated with the therapeutic cell composition prior to treating the subject with the therapeutic cell composition, wherein the features applied as input are the same informative features as those used to train the random survival forests model; and administering a treatment to the subject wherein: (1) if the subject is determined, e.g., predicted, to have a clinical response selected from the group consisting of a complete response (CR), a partial response (PR), a durable response of greater than 3 months, progression free survival (PFS) for more than 3 months, overall response rate (ORR), objective response (OR), a desired pharmacokinetic response that is or is greater than a target pharmacokinetic response, and no or a mild toxicity response (optionally wherein the toxicity is grade 2 or less CRS or grade 2 or less neurotoxicity), a predetermined treatment regimen comprising the therapeutic cell composition is administered; or (2) if the subject is determined, e.g., predicted, to have a clinical response selected from the group consisting of a toxicity response (optionally wherein the toxicity response is a severe cytokine release syndrome or severe neurotoxicity), a reduced pharmacokinetics response compared to a target pharmacokinetic response, progressive disease (PD), a durable response of less than 3 months, and PFS of less than 3 months, administering to the subject a treatment regimen comprising the therapeutic cell composition that is altered compared to the predetermined treatment regimen comprising the therapeutic cell composition.

In some of any embodiments, the method further comprises generating the therapeutic cell composition.

Provided herein in some embodiments is a method of treating a subject, the method comprising: (a) selecting T cells from a sample from a subject to produce an input composition comprising T cells; (b) generating a therapeutic cell composition comprising T cells comprising a chimeric antigen receptor (CAR) that binds to an antigen associated with a disease or condition, wherein the therapeutic cell composition (i) is for treating the disease or condition, (ii) is produced from the input composition, and (iii) is to be administered to the subject; (c) determining features comprising: (i) subject features determined from the subject prior to the subject being treated with the therapeutic cell composition; (ii) input composition features determined from the input composition; and (iii) therapeutic cell composition features determined from the therapeutic cell composition; (c) applying the features as input to a random forests model trained to determine, based on informative features identified by preprocessing, a clinical response in the subject to be treated with the therapeutic cell composition prior to treating the subject with the therapeutic cell composition, wherein the features applied as input are the same informative features as those used to train the random forests model; and (d) administering a treatment to the subject wherein: (1) if the subject is determined to have a clinical response selected from the group consisting of a complete response (CR), a partial response (PR), a durable response of greater than 3 months, progression free survival (PFS) for more than 3 months, objective response (OR), a desired pharmacokinetic response that is or is greater than a target pharmacokinetic response, and no or a mild toxicity response (optionally wherein the mild toxicity response is grade 2 or less cytokine release syndrome (CRS) or grade 2 or less neurotoxicity), a predetermined treatment regimen comprising the therapeutic cell composition is administered; or (2) if the subject is determined to have a clinical response selected from the group consisting of a toxicity response (optionally wherein the toxicity response is a severe cytokine release syndrome (CRS) or severe neurotoxicity), a reduced pharmacokinetics response compared to a target pharmacokinetic response, progressive disease (PD), a durable response of less than 3 months, and PFS of less than 3 months, administering to the subject a treatment regimen comprising the therapeutic cell composition that is altered compared to the predetermined treatment regimen comprising the therapeutic cell composition.

Provided herein in some embodiments is a method of treating a subject, the method comprising: (a) selecting T cells from a sample from a subject to produce an input composition comprising T cells; (b) generating a therapeutic cell composition comprising T cells comprising a chimeric antigen receptor (CAR) that binds to an antigen associated with a disease or condition, wherein the therapeutic cell composition (i) is for treating the disease or condition, (ii) is produced from the input composition, and (iii) is to be administered to the subject; (c) determining features comprising: (i) subject features determined from the subject prior to the subject being treated with the therapeutic cell composition; (ii) input composition features determined from the input composition; and (iii) therapeutic cell composition features determined from the therapeutic cell composition; (c) applying the features as input to a random survival forests model trained to determine based on informative features identified by preprocessing, a clinical response in the subject to be treated with the therapeutic cell composition prior to treating the subject with the therapeutic cell composition, wherein the features applied as input are the same informative features as those used to train the random survival forests model; and (d) administering a treatment to the subject wherein: (1) if the subject is determined to have a clinical response selected from the group consisting of a complete response (CR), a partial response (PR), a durable response of greater than 3 months, progression free survival (PFS) for more than 3 months, objective response (OR), a desired pharmacokinetic response that is or is greater than a target pharmacokinetic response, and no or a mild toxicity response (optionally wherein the mild toxicity response is grade 2 or less cytokine release syndrome (CRS) or grade 2 or less neurotoxicity), a predetermined treatment regimen comprising the therapeutic cell composition is administered; or (2) if the subject is determined to have a clinical response selected from the group consisting of a toxicity response (optionally wherein the toxicity response is a severe cytokine release syndrome (CRS) or severe neurotoxicity), a reduced pharmacokinetics response compared to a target pharmacokinetic response, progressive disease (PD), a durable response of less than 3 months, and PFS of less than 3 months, administering to the subject a treatment regimen comprising the therapeutic cell composition that is altered compared to the predetermined treatment regimen comprising the therapeutic cell composition.

In some embodiments, the random forests model is trained to determine if the subject will have a complete response (CR). In some embodiments, (1) the subject is administered the predetermined treatment regimen if the subject is determined to have a complete response (CR), or (2) the subject is administered the altered treatment regimen if the subject is determined to have progressive disease (PD).

In some embodiments, the random forests model is trained to determine if the subject will have a partial response (PR). In some embodiments, (1) the subject is administered the predetermined treatment regimen if the subject is determined to have a partial response (PR), or (2) the subject is administered the altered treatment regimen if the subject is determined to have progressive disease (PD).

In some embodiments, the random forests model is trained to determine if the subject will have a durable response of greater than 3 months. In some embodiments, the random forests survival model is trained to determine if the subject will have a durable response of greater than 3 months. In some embodiments, (1) the subject is administered the predetermined treatment regimen if the subject is determined to have a durable response of greater than three months; or (2) the subject is administered the altered treatment regimen if the subject is determined to have a durable response of less than three months.

In some embodiments, the random forests model is trained to determine if the subject will have progression free survival (PFS) for more than 3 months. In some embodiments, the random survival forests model is trained to determine if the subject will have progression free survival (PFS) for more than 3 months. In some embodiments, (1) the subject is administered the predetermined treatment regimen if the subject is determined to have progression free survival (PFS) for more than three months, or (2) the subject is administered the altered treatment regimen if the subject is determined to have progression free survival (PFS) of less than three months.

In some embodiments, the random forests model is trained to determine if the subject will have an objective response (OR). In some embodiments, (1) the subject is administered the predetermined treatment regimen if the subject is determined to have an objective response (OR), or (2) the subject is administered the altered treatment regimen if the subject is determined to have progressive disease (PD).

In some embodiments, the random forests model is trained to determine a pharmacokinetic response of the subject. In some embodiments, (1) the subject is administered the predetermined treatment regimen if the subject is determined to have a desired pharmacokinetic response that is or is greater than a target pharmacokinetic response, or (2) the subject is administered the altered treatment regimen if the subject is determined to have a reduced pharmacokinetic response compared to the target pharmacokinetic response.

In some embodiments, the random forests model is trained to determine if the subject will have a toxicity response. In some embodiments, (1) the subject is administered the predetermined treatment regimen if the subject is determined to have no or a mild toxicity response, or (2) the subject is administered the altered treatment regimen if the subject is determined to have a toxicity response. In some embodiments, the toxicity response is severe CRS. In some embodiments, the toxicity response is severe neurotoxicity.

In some embodiments, the random forests model is trained using supervised training, the supervised training comprising: (a) receiving features comprising: (i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and (iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects; (b) preprocessing the features to identify informative features, the informative features comprising a subset of the features comprising one or more subject features, one or more input composition features, and one or more therapeutic cell composition features; (c) obtaining clinical responses from each of the plurality of subjects following treatment with one of the plurality of therapeutic compositions; (d) applying the informative features from a plurality of subjects and the obtained clinical responses as input to train a random forests model.

In some embodiments, the random survival forests model is trained using supervised training, the supervised training comprising: (a) receiving features comprising: (i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and (iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects; (b) preprocessing the features to identify informative features, the informative features comprising a subset of the features comprising one or more subject features, one or more input composition features, and one or more therapeutic cell composition features; (c) obtaining clinical responses over time from each of the plurality of subjects following treatment with one of the plurality of therapeutic compositions; (d) applying the informative features and clinical responses from the plurality of subjects as input to train a random survival forests model using supervised learning.

Provided herein are methods of developing a random forests model comprising: (a) receiving features comprising: (i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and (iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects; (b) preprocessing the features to identify informative features, the informative features comprising a subset of the features comprising one or more subject features, one or more input composition features, and one or more therapeutic cell composition features; (c) obtaining clinical responses from each of the plurality of subjects following treatment with one of the plurality of therapeutic compositions; (d) applying the informative features from a plurality of subjects and the obtained clinical responses as input to train a random forests model.

Provided herein are methods of developing a random survival forests model comprising: (a) receiving features comprising: (i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition; (ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and (iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects; (b) preprocessing the features to identify informative features, the informative features comprising a subset of the features comprising one or more subject features, one or more input composition features, and one or more therapeutic cell composition features; (c) obtaining clinical responses over time from each of the plurality of subjects following treatment with one of the plurality of therapeutic compositions; (d) applying the informative features and clinical responses from the plurality of subjects as input to train a random survival forests model using supervised learning.

In some embodiments, each of the plurality of subjects is administered one of the plurality of therapeutic cell compositions, wherein the one therapeutic cell composition administered to the subject is the therapeutic cell composition produced from the input composition of the sample from the subject.

In some embodiments, the preprocessing to identify informative features comprises one or more of: a) removing subject features, input composition features, and therapeutic cell composition features having greater than, than about, or 50% of the data missing; b) removing subject features, input composition features, and therapeutic cell composition features having zero variance or greater than, than about or 95% of data values equal to a single value and/or fewer than 0.1n unique values, wherein n=number of samples; c) imputing missing data for subject features, input composition features, and therapeutic cell composition features by multivariate imputation by chained equations; d) identifying covariate clusters, the covariate clusters comprising sets of subject features, input composition features, and therapeutic cell composition features and combinations thereof, with correlation coefficients of greater than, about, or equal to 0.5, and iteratively selecting subject features, input composition features, and therapeutic cell composition features from the covariate cluster, wherein the selected subject features, input composition features, and therapeutic cell composition features have the lowest mean absolute correlation with all remaining subject features, input composition features, and therapeutic cell composition features. In some embodiments, the preprocessing to identify informative features comprises or is removing subject features, input composition features, and therapeutic cell composition features having greater than, about, or 50% of the data is missing. In some embodiments, the preprocessing to identify informative features comprises or is removing subject features, input composition features, and therapeutic cell composition features having zero variance or greater than, about or 95% of data values equal to a single value and fewer than 0.1n unique values, wherein n=number of samples. In some embodiments, the preprocessing to identify informative features comprises or is imputing missing data for subject features, input composition features, and therapeutic cell composition features by multivariate imputation by chained equations. In some embodiments, the preprocessing to identify informative features comprises or is identifying covariate clusters, the covariate clusters comprising sets of subject features, input composition features, and therapeutic cell composition features and combinations thereof with correlation coefficients of greater than, about, or equal to 0.5, and iteratively selecting subject features, input composition features, and therapeutic cell composition features from the covariate cluster, wherein the selected subject features, input composition features, and therapeutic cell composition features have the lowest mean absolute correlation with all remaining subject features, input composition features, and therapeutic cell composition features.

In some embodiments, the random forests model is evaluated using cross validation. In some embodiments, the random survival forests model is evaluated using cross validation. In some embodiments, the cross validation is or is at least 10-fold cross validation. In some embodiments, the cross validation is nested cross validation.

In some embodiments, the plurality of subjects is or is about 500, 400, 300, 200, 150, 100, 50, 25, 15, or 10 subjects, or is any number between any of the foregoing. In some embodiments, the plurality of subjects is, is about, or is greater than 10 subjects and less than 250 subjects. In some embodiments, the plurality of subjects is, is about, or is greater than 20 subject and less than 200 subjects. In some embodiments, the plurality of subjects is, is about, or is greater than 20 and less than 150 subjects. In some embodiments, the plurality of subjects is, is about, or is greater than 20 subjects and less than 150 subjects. In some embodiments, the plurality of subjects is, is about, or is greater than 20 subjects and less than 100 subjects. In some embodiments, the plurality of subjects are participating in a clinical trial.

In some embodiments, the subject features comprise one or more of subject attributes and clinical attributes. In some embodiments, the subject attributes comprise one or more of age, weight, height, ethnicity, race, sex, and body mass index. In some embodiments, the clinical attributes comprise one or more of biomarkers, disease diagnosis, disease burden, disease duration, disease grade, and treatment history. In some embodiments, the input composition features comprise cell phenotypes. In some embodiments, the therapeutic cell composition features comprise one or more of a cell phenotype, a recombinant receptor-dependent activity, and a dose. In some embodiments, the clinical responses comprise one or more of a complete response (CR), a partial response (PR), a durable response, progression free survival (PFS), overall response rate (ORR), objective response (OR), a pharmacokinetic response that is or is greater than a target pharmacokinetic response, no or a mild toxicity response, a toxicity response, a reduced pharmacokinetics response compared to a target response, or a lack of CR, PR, durable response, ORR, OR, or PFS.

In some embodiments, the clinical response is or comprises a complete response (CR), a partial response (PR), a durable response, progression free survival (PFS), objective response (OR), a pharmacokinetic response that is or is greater than a target pharmacokinetic response, no or a mild toxicity response, a toxicity response, a reduced pharmacokinetics response compared to a target response, or a lack of CR, PR, durable response, or objective response (OR).

In some embodiments, the clinical response is a complete response (CR). In some embodiments, the clinical response is a lack of complete response (CR). In some embodiments, the clinical response is a partial response (PR). In some embodiments, the clinical response is a lack of partial response (PR). In some embodiments, the clinical response is an objective response (OR). In some embodiments, the clinical response is a lack of objective response (OR). In some embodiments, the clinical response is a toxicity response. In some embodiments, the clinical response is a lack of toxicity response. In some embodiments, the toxicity response is a mild toxicity response. In some embodiments, the toxicity response is a severe toxicity response. In some embodiments, the toxicity response is severe CRS. In some embodiments, the toxicity response is severe neurotoxicity. In some embodiments, the clinical response is a durable response. In some embodiments, the clinical response is a lack of durable response. In some embodiments, the clinical response is the duration of response (DOR). In some embodiments, the clinical response is a duration of response (DOR) of at least or at least about three months. In some embodiments, the clinical response is progression free survival (PFS). In some embodiments, the clinical response is progression free survival (PFS) of at least or at least about three months. In some embodiments, the clinical response is a pharmacokinetic response that is or is greater than a target pharmacokinetic response. In some embodiments, the pharmacokinetic response is a measure of expansion of CAR T cells of the therapeutic cell composition following treatment of the subject with the therapeutic cell composition. In some embodiments, the pharmacokinetic response is a measure of maximum CAR T cell concentration in the subject following treatment of the subject with the therapeutic cell composition. In some embodiments, the pharmacokinetic response is a measure of a timepoint at which CAR T cell concentration is maximal in the subject following treatment of the subject with the therapeutic cell composition. In some embodiments, the pharmacokinetic response is a measure of exposure of the subject to CAR T cells of the therapeutic cell composition following treatment of the subject with the therapeutic cell composition.

In some embodiments, the sample comprises a whole blood sample, a buffy coat sample, a peripheral blood mononuclear cell (PBMC) sample, an unfractionated T cell sample, a lymphocyte sample, a white blood cell sample, an apheresis product, or a leukapheresis product. In some embodiments, the sample is an apheresis product or leukapheresis product. In some embodiments, the apheresis product or leukapheresis product has been previously cryopreserved. In some embodiments, the T cells comprise primary cells obtained from the subject. In some embodiments, the T cells comprise CD3+, CD4+, and/or CD8+.

In some embodiments, the input composition comprises CD4+, CD8+, or CD4+ and CD8+ T cells and the therapeutic cell composition comprises CD4+, CD8+, or CD4+ and CD8+ T cells expressing a recombinant receptor and is produced from the input composition, wherein the input composition features comprise input composition features from the CD4+, CD8+, or CD4+ and CD8+ T cell compositions of the input composition, and the therapeutic cell composition features comprise therapeutic cell composition features from the CD4+, CD8+, or CD4+ and CD8+ T cells of the therapeutic composition.

In some embodiments, the input composition comprises separate compositions of CD4+ and CD8+ T cells and the therapeutic cell composition comprises separate compositions of CD4+ and CD8+ T cells expressing a recombinant receptor, and is produced from the respective CD4+ or CD8+ T cell composition of the input composition, wherein the input composition features comprise input composition features from the CD4+ and CD8+ T cell compositions of the input composition, and the therapeutic cell composition features comprise therapeutic cell composition features from the CD4+ and CD8+ T cells of each of the separate compositions of the therapeutic composition.

In some embodiments, the input composition comprises separate compositions of CD4+ and CD8+ T cells and the therapeutic cell composition comprises a mixed composition of CD4+ and CD8+ T cells expressing a recombinant receptor, and is produced from the separate CD4+ and CD8+ T cell compositions of the input composition, wherein the input composition features comprise input composition features from the separate CD4+ and CD8+ T cell compositions of the input composition, and the therapeutic cell composition features comprise therapeutic cell composition features from the mixed composition of CD4+ and CD8+ cells of the therapeutic composition.

In some embodiments, the recombinant receptor is a chimeric antigen receptor (CAR).

In some embodiments, the predetermined treatment regimen comprises or is a single treatment comprising administering: a) 25×10⁶ CD8+CAR+ T cells and 25×10⁶ CD4+CAR+ T cells separately to the subject; b) 50×10⁶ CD8+CAR+ T cells and 50×10⁶ CD4+CAR+ T cells separately to the subject; or c) 75×10⁶ CD8+CAR+ T cells and 75×10⁶ CD4+CAR+ T cells separately to the subject. In some embodiments, altering the predetermined treatment regimen, e.g., the altered treatment regimen, comprises or is a single treatment comprising administering: 50×10⁶ CD8+CAR+ T cells and 50×10⁶ CD4+CAR+ T cells separately to the subject when the predetermined treatment regimen comprises or is a single treatment comprising administering 25×10⁶ CD8+CAR+ T cells and 25×10⁶ CD4+CAR+ T cells separately to the subject; 75×10⁶ CD8+CAR+ T cells and 75×10⁶ CD4+CAR+ T cells separately to the subject when the predetermined treatment regimen comprises or is a single treatment comprising administering 50×10⁶ CD8+CAR+ T cells and 50×10⁶ CD4+CAR+ T cells separately to the subject; or 75×10⁶ CD8+CAR+ T cells and 75×10⁶ CD4+CAR+ T cells separately to the subject when the predetermined treatment regimen comprises or is a single treatment comprising administering 25×10⁶ CD8+CAR+ T cells and 25×10⁶ CD4+CAR+ T cells separately to the subject. In some embodiments, altering the predetermined treatment regimen, e.g., the altered treatment regimen, comprises or is a single treatment comprising administering: 25×10⁶ CD8+CAR+ T cells and 25×10⁶ CD4+CAR+ T cells separately to the subject when the predetermined treatment regimen comprises or is a single treatment comprising administering 50×10⁶ CD8+CAR+ T cells and 50×10⁶ CD4+CAR+ T cells separately to the subject; 50×10⁶ CD8+CAR+ T cells and 50×10⁶ CD4+CAR+ T cells separately to the subject when the predetermined treatment regimen comprises or is a single treatment comprising administering 75×10⁶ CD8+CAR+ T cells and 75×10⁶ CD4+CAR+ T cells separately to the subject; or 25×10⁶ CD8+CAR+ T cells and 25×10⁶ CD4+CAR+ T cells separately to the subject when the predetermined treatment regimen comprises or is a single treatment comprising administering 75×10⁶ CD8+CAR+ T cells and 75×10⁶ CD4+CAR+ T cells separately to the subject. In some embodiments, wherein altering the predetermined treatment regimen, e.g., the altered treatment regimen, comprises administering the therapeutic cell composition in combination with a second therapeutic agent.

### BRIEF DESCRIPTION OF THE DRAWINGS

FIGS. 1A and 1B show exemplary decisions trees contained in a random forests model.

FIG. 2A shows exemplary significant feature clusters identified using random forests and survival forests as important for correlating with log₁₀AUC (AUC₀₋₂₈, area under the concentration-time curve through 28 days after infusion) following treatment with the therapeutic cell composition. Arrows indicate directionality of the correlation. FIG. 2B shows the correlation between log₁₀AUC and patient age. FIG. 2C shows the accumulated local effect of patient age on log₁₀AUC, independent of all other features. FIG. 2D shows the correlation between log₁₀AUC and the total number of prior treatments a patient received. FIG. 2E shows the accumulated local effect of the number of prior treatments a patient received on log₁₀AUC, independent of all other features. FIG. 2F shows the correlation between log₁₀AUC and effector cytokine secretion of CD8+ T cells of the therapeutic cell composition. FIG. 2G shows the accumulated local effect of effector cytokine secretion of CD8+ T cells of the therapeutic cell composition on log₁₀AUC, independent of all other features.

FIG. 3A shows exemplary significant feature clusters identified using random forests and survival forests as important for correlating with progression free survival (PFS) following treatment with the therapeutic cell composition. Arrows indicate directionality of the correlation. FIG. 3B shows the accumulated local effect of antigen-specific cytokine production of CD4+ T cells in the therapeutic cell composition on PFS, independent of all other features. FIG. 3C shows the accumulated local effect of lactate dehydrogenase (LDH) levels before treatment with lymphodepleting chemotherapy (pre-LDC) on PFS, independent of all other features.

FIG. 4A shows exemplary significant feature clusters identified using random forests and survival forests as important for correlating with complete response (CR) following treatment with the therapeutic cell composition. Arrows indicate directionality of the correlation. FIG. 4B shows the accumulated local effect of antigen-specific cytokine production of CD4+ T cells of the therapeutic cell composition on CR, independent of all other features. FIG. 4C shows the accumulated local effect of antigen-specific cytokine production of CD8+ T cells of the therapeutic cell composition on CR, independent of all other features. FIG. 4D shows the accumulated local effect of tumor burden, measured by sum of products by diameters (SPD) before treatment with lymphodepleting chemotherapy (pre-LDC), on CR, independent of all other features. FIG. 4E shows the accumulated local effect of tumor burden, measured by LDH before treatment with lymphodepleting chemotherapy (pre-LDC), on CR, independent of all other features.

FIG. 5A shows exemplary significant feature clusters identified using random forests and survival forests as important for correlating with neurological events (NE) following treatment with the therapeutic cell composition. Arrows indicate directionality of the correlation. FIG. 5B shows significant feature clusters identified as important for correlating with cytokine release syndrome (CRS) following treatment with the therapeutic cell composition. Arrows indicate directionality of the correlation. FIG. 5C shows the accumulated local effect of tumor burden measured by LDH before treatment with lymphodepleting chemotherapy (pre-LDC) on neurologic events (NE), independent of all other features. FIG. 5D shows the accumulated local effect of tumor burden measured by LDH before treatment with lymphodepleting chemotherapy (pre-LDC) on CRS, independent of all other features. FIG. 5E shows the accumulated local effect of bridging therapy on CRS, independent of all other features.

### DETAILED DESCRIPTION

Provided herein are methods for identifying features associated with clinical responses in subjects following treatment with a cell therapy, such as an engineered T cell therapy (e.g., therapeutic cell composition) for the treatment of diseases and conditions, including various cancers. In some embodiments, the methods determine, e.g., predict, a subject's clinical response to treatment with a cell therapy (e.g., therapeutic cell composition) prior to the subject being treated. Aspects of the methods provided herein, including embodiments thereof, relate to determining effective dosing and administration of a cell therapy, e.g., a therapeutic cell composition.

Numerous subject (patient) attributes, attributes of starting materials (e.g., input composition characteristics) for producing a drug product, and drug product (e.g., therapeutic cell composition) attributes have demonstrated nominally significant, univariate relationships with clinical endpoints, e.g., responses, in cell therapy trials. However, clinical responses to cell therapy may depend upon many factors, including, but not limited to, the features of the subject, the features of the therapeutic cell composition, and the features of the input composition from which the therapeutic cell composition is produced. Quantifying the multifactorial contributions of subject features, starting material (e.g., input composition) features, and drug product (e.g., therapeutic cell composition) features on efficacy, safety, and pharmacokinetic (PK) responses is a challenge in the field of cell therapy.

The methods provided herein address the challenge of multivariate feature assessment through the use of supervised machine learning. For example, machine learning models provided herein are capable of assessing how a plurality of diverse features can contribute to (e.g., determine or predict) clinical responses. The models can be queried or interrogated to identify features, e.g., groups of features, which correlate with clinical response. In some cases, according to the methods herein, it is also possible to rank the importance of each feature of a set of features in determining a clinical response. In some cases, this information is useful for optimizing clinical experience across heterogeneous patient populations. In some cases, this information is useful for optimizing drug product production and manufacturing.

The methods provided herein include machine learning models trained to determine (e.g., predict) a subject's clinical response to a cell therapy, e.g., therapeutic composition, such as a complete response (CR), a partial response (PR), a durable response (e.g., durability of response, DOR), toxicity response, and/or a pharmacokinetic response, based on features, such as attributes of subjects (e.g., subject features), therapeutic cell compositions (e.g., therapeutic cell composition features), and input compositions used to produce therapeutic cell compositions (e.g., input composition features). In some embodiments, subject features include subject attributes such as age and weight, and clinical attributes, such as expression of biomarkers and combinations of biomarkers, disease burden (e.g., a measurement of tumor burden), treatment history, and combinations thereof. In some embodiments, the therapeutic cell composition features include, but are not limited, to cell phenotypes, such as cell health (e.g., viable cell count, number of dead cells), the presence and/or expression of a surface marker, the absence or lack of expression of a surface marker, the presence and/or expression of a cytokine, the absence or lack of expression of a cytokine, recombinant receptor expression (e.g., CAR+), recombinant receptor-dependent activity (e.g., cytolytic activity, cytokine production), and combinations thereof. In some embodiments, the input composition features include, but are not limited, cell phenotypes, such as cell health (e.g., viable cell concentration, number of dead cells), the presence and/or expression of a surface marker, the absence or lack of expression of a surface marker, and combinations thereof.

In some aspects, the methods provided herein include machine learning models trained to determine (e.g., predict) a subject's clinical response to a cell therapy, e.g., therapeutic composition, based on attributes of subjects (e.g., subject features), therapeutic cell compositions (e.g., therapeutic cell composition features), and input compositions used to produce therapeutic cell compositions (e.g., input composition features). In some aspects, the therapeutic cell composition is generated using a input composition as a starting material. In some aspects, training the machine learning models using subject features, therapeutic cell composition features, and input composition features affords certain advantages over training with only a subset of these feature sets. These advantages include the ability to more accurately predict a subject's clinical response or to better identify informative features associated with clinical response. In some aspects, the provided methods are based on the appreciation that even when an input composition is used as starting material for production of a therapeutic cell composition, features of the input composition prior to manufacturing can contain information associated with clinical response to the therapeutic cell composition that is not included in or accounted for by features of the therapeutic cell composition following manufacturing thereof. Thus, in some aspects the inclusion of input composition features in model training can improve model performance or identification of informative features, relative to that achieved when training with subject features and/or therapeutic cell composition features alone.

In some aspects, the machine learning models contemplated for use according to the methods provided herein are transparent machine learning models. Use of a transparent machine learning model is particularly advantageous because it allows for features associated with clinical responses in subjects to be identified. In some embodiments, features identified as associated with a clinical response can be assessed in a subject prior to treating the subject with a cell therapy to determine, e.g., predict, whether a subject will have desirable or advantageous clinical responses to treatment.

In some cases, models, including traditional “black box” models, may be considered transparent if they can be queried or interrogated in such a way that an understanding of how the model arrived at a particular decision can be appreciated. In some embodiments, an understanding of how the model arrived at a particular decision is or includes identifying a feature or group of features, e.g., variable(s), which contribute to the decision. For example, in view of the methods provided herein, in some embodiments, a model is considered transparent if it can be interrogated or queried to identify features associated with clinical responses (e.g., determine feature importance for clinical response). In some embodiments, the contribution of each feature to the arrival at a particular decision is quantified. In some cases, identification and/or quantification of a feature is determined by manipulating the model, e.g., systematically or under controlled and known conditions, and assessing the accuracy of the model (e.g., prediction accuracy), and change thereof, across the manipulations.

In some embodiments, the machine learning models are random forests models. In some embodiments, the machine learning models are random survival forests models. As described above, an advantage of using random forests and random survival forests is their transparency. For example, the random forests and random survival forests models can be interrogated such that features used to predict a subject's clinical response to a cell therapy, e.g., therapeutic cell composition, can be identified. In some embodiments, the features used to determine, e.g., predict, the clinical response are considered associated with the clinical response. In some embodiments, identifying features used to determine, e.g., predict, a subject's clinical response comprises assessing feature importance, for example as described herein (see, Sections I.B.1a and I.B.2a).

In some embodiments, the random forests models provided herein are interrogated to identify features associated with clinical responses. In some embodiments, the random forests models provided herein are used to determine (e.g., classify or predict) which clinical responses a subject who has not yet been treated with a cell therapy (e.g., therapeutic cell composition) will have. In some embodiments, determining, e.g., predicting, which clinical responses a subject will have prior to treatment can result in the subject being treated according to a predetermined treatment regimen or according to a treatment regimen different (e.g., altered) from the predetermined treatment regimen. In some embodiments, altering the predetermined treatment regimen in view of the determined, e.g., predicted, clinical responses can result in an improved or advantageous clinical response, or increase the probability or likelihood of the subject having an improved or advantageous clinical response.

Random survival forests models are capable of dealing with right-censored survival data, and circumventing restrictive assumptions, such as proportional hazards or parametric assumptions. In some embodiments, the random survival forests model is capable of handling nonlinear effects and interactions between multiple variables. These characteristics are advantageous for building a risk prediction model, e.g., a risk prediction model of clinical responses. In some embodiments, the random survival forests models provided herein are interrogated to identify features associated with clinical responses. For example, features associated with a probability of having a clinical response within a given amount of time can be identified. In some embodiments, the random survival forests models provided herein are used to determine the probability of a subject having clinical responses following treatment with a cell therapy, e.g., therapeutic cell composition, prior to the subject being treated. In some embodiments, the random survival forests models provided herein are used to determine a clinical response function and cumulative hazards function for a subject. In some embodiments, the random survival forests model can estimate the risk of a subject having clinical responses. In some embodiments, the random survival forests model can estimate the risk of a subject not having clinical responses. In some embodiments, the determination (e.g., estimation or prediction) of a subject having or not having clinical responses following treatment prior to treatment can result in the subject being treated according to a predetermined treatment regimen or according to a treatment regimen different (e.g., altered) from the predetermined treatment regimen. In some embodiments, altering the predetermined treatment regimen may result in an improved or advantageous clinical response, or increase the probability or likelihood of the subject having an improved or advantageous clinical response.

The machine learning models, e.g., random forests and random survival forests, provided herein are trained to predict clinical responses based on various features associated with the subjects to be treated (e.g., patients), the therapeutic cell compositions to be administered to the subject, and the input composition (e.g., starting materials derived from the subject) for the production of the therapeutic cell composition. In some embodiments, the machine learning models provided herein are trained using features associated with the subjects to be treated (e.g., subject features, prior to treatment), the therapeutic cell compositions to be administered to the subject (e.g., therapeutic cell composition features), and the input composition (e.g., starting materials derived from the subject) for the production of the therapeutic cell composition (e.g., input composition features).

In some embodiments, the training is supervised learning. In cases where the models are trained using supervised learning, clinical responses from a subject who has been treated with a therapeutic cell composition, and for which features, e.g., subject features, therapeutic cell composition features, and input compositions features have been obtained, are determined, obtained, or otherwise received. As described above, in some embodiments, the clinical responses include, but are not limited to, an efficacy outcome, such as an overall response; a complete response (CR); a partial response (PR); a durable response (e.g., durability of response, DOR), such a response that is durable for at least 3 months, 6 months, or more; a safety outcome, such as a development of a toxicity, for example, neurotoxicity or CRS; and a pharmacokinetic response, such as maximum serum concentration of cell (Cₘₐₓ) and exposure (e.g., area under the curve (AUC)).

By determining, obtaining, or receiving features, e.g., subject features prior to treatment, therapeutic cell composition features, and input composition features, in addition to clinical responses following treatment with the therapeutic cell composition, the models may be trained using labeled data. The models can be tested using test data to determine the prediction accuracy of the trained model. It should be appreciated that for training of random survival forests models, time and censor components, e.g., time to event, will accompany the clinical response.

In some embodiments, the subject features, therapeutic cell composition features, and input composition features are preprocessed. Data preprocessing, in some aspects, avoids creating a model that produces misleading or inaccurate results. In some embodiments, preprocessing prevents out-of-range values, missing values, impossible data combinations, highly correlated features, and other confounding features from being incorporated into (e.g., learned by) the model. In provided embodiments, it is found that the provided preprocessing steps are particularly advantageous for training data from a small data cohort, as may be present from data related to clinical trials of a therapeutic drug, including those involving T cell therapies (e.g., CAR T cells).

In some cases, for example during clinical trials of a therapeutic drug, different dose levels may be used to treat different numbers of subjects. For example, a cohort of 100 subjects may receive a particular dose, while a different cohort of 50 subjects may receive a markedly difference dose. In some cases, this can lead to an imbalanced dataset. In some cases, the difference in sample size may also pose an issue for training a model. In some aspects, the imbalance is remedied by including the dose as a feature on which the model is trained.

In some cases, preprocessing leads to the identification of informative features. For example, preprocessing may be used to remove features with little or no variance, features that are highly correlated, or features with missing values, or to replace missing values, such that the remaining features are informative, discriminating, and independent (e.g., informative features). In some embodiments, the machine learning models, e.g., random forests and random survival forests, are trained on the informative features identified by preprocessing. In some embodiments, the machine learning models, e.g., random forests and random survival forests, are trained using supervised learning on the informative features identified by preprocessing. In some embodiments, the features, e.g., subject features, therapeutic cell composition feature, and input features, used as input to a model to determine clinical responses are informative features that are the same informative features used to train the model.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS FOR IDENTIFYING FEATURES ASSOCIATED WITH CLINICAL OUTCOME AND DETERMINING CLINICAL OUTCOME

The methods provided herein allow for the identification of features, such as subject features, therapeutic cell composition features, and input composition features, associated with clinical responses in a subject following treatment with a therapeutic cell composition. In some embodiments, the methods allow for determining clinical responses in subjects to be treated with a therapeutic cell composition prior to treatment with the therapeutic cell composition based on the features, e.g., subject features, the therapeutic cell composition features, and the input composition features. Having this type of information at an early stage, e.g., prior to treatment, allows for the development of treatment strategies (e.g., combination treatment, dosing) prior to treating the subject, thereby increasing the probability of a subject having a positive or advantageous clinical response (e.g., durable response, progression free survival).

The methods provided herein include generating therapeutic cell compositions that include engineered CD3+, CD4+, CD8+, or CD4+ and CD8+ cells (e.g., therapeutic T cell compositions), where the therapeutic cell compositions are produced from input compositions that include CD3+, CD4+, CD8+, or CD4+ and CD8+ T cells. In some embodiments, the input composition comprises two separate compositions, e.g., a CD4+ composition and a CD8+ composition. In some embodiments, the input composition contains a single composition including CD4+ and CD8+ cells. In some embodiments, the methods provided herein for generating therapeutic cell compositions include generating both CD4+ and CD8+ engineered cells for therapeutic cell compositions. In some embodiments, the CD4+ and CD8+ cells are engineered separately, for example to produce separate therapeutic cell compositions. In some embodiments, the CD4+ and CD8+ cells are engineered separately to produce separate therapeutic cell compositions from the separate CD4+ and CD8+ input compositions. In some embodiments, the therapeutic cell compositions contain mixed CD4+ and CD8+ engineered cells. In some embodiments, the separate CD4+ and CD8+ engineered cells of the separate therapeutic cell compositions are combined to produce the mixed CD4+ and CD8+ engineered cell therapeutic cell composition. In some embodiments, the mixed CD4+ and CD8+ engineered cell therapeutic cell composition is produced from a single input composition containing mixed CD4+ and CD8+. The features of the input composition and the therapeutic cell composition can be determined, received, or obtained from mixed or separate compositions.

A. Features and Clinical Responses

It is contemplated that clinical responses of a subject to treatment with a therapeutic cell composition depend upon many factors, including, but not limited to, the features of the subject, the features of the therapeutic cell composition, and the features of the input composition from which the therapeutic cell composition is produced. Thus, the methods provided herein are directed to assessing the relationship between features associated with a subject to be treated with a therapeutic cell composition, features associated with the therapeutic cell composition, and features of an input composition from which the therapeutic cell composition is produced and clinical responses in the subject following treatment with the therapeutic cell composition using machine learning models.

In some embodiments, the features associated with the subject used in the methods provided herein include subject attributes, such as age and weight, clinical attributes, such as expression of biomarkers and combinations of biomarkers, disease burden (e.g., a measurement of tumor burden), treatment history, and combinations thereof.

In some embodiments, the features associated with the therapeutic cell composition and the input composition include cell phenotypes. In some embodiments, cell phenotype is determined by assessing the presence or absence of one or more specific molecules, including surface molecules and/or molecules that may accumulate or be produced by the cells or a subpopulation of cells within an input composition or therapeutic cell composition. In some embodiments, cell phenotype may include cell activity, such as production of a factor (e.g., cytokine) in response to a stimulus. In some embodiments, the production of a factor (e.g., cytokine) is in response to recombinant receptor-dependent activation. In some embodiments, recombinant receptor-dependent activity of cells of a therapeutic cell composition is determined by assessing one or more specific molecules (e.g., cytokines) that may accumulate or be produced by the cells or a subpopulation of cells within a therapeutic cell composition. In some embodiments, recombinant receptor-dependent activity is assessed by determining the cytolytic activity of the cells of the therapeutic cell composition.

In some embodiments, features of the input composition and/or therapeutic cell composition include a determination, detection, quantification, or other assessment of a phenotype of the cell composition (e.g., surface molecule, cytokine, recombinant receptor). In particular embodiments, features of the composition (e.g., input composition, therapeutic cell composition) include a determination, detection, quantification, or other assessment of the presence, absence, degree of expression or level of a specific molecule (e.g., surface molecule, cytokine, recombinant receptor). In some embodiments, the percentage, number, ratio, and/or proportion of cells having an attribute is determined. In some embodiments, the percentage, number, ratio, and/or proportion of cells having an attribute is a therapeutic cell composition feature or an input composition feature which can be used as input for a machine learning algorithm provided herein.

In some embodiments, the therapeutic cell composition feature or the input composition feature is a phenotype, e.g., cell phenotype. In some embodiments, the therapeutic cell composition feature or the input composition feature is a phenotype indicative of viability of a cell. In some embodiments, the phenotype is indicative of absence of apoptosis, absence of early stages of apoptosis or absence of late stages of apoptosis. In some embodiments, the phenotype is the absence of a factor indicative of absence of apoptosis, early apoptosis or late stages of apoptosis. In some embodiments, the phenotype is a phenotype of a sub-population or subset of T cells, such as recombinant receptor-expressing T cells (e.g., CAR⁺ T cells), CD8⁺ T cells, or CD4⁺ T cells in the therapeutic cell composition. In some embodiments, the phenotype is a phenotype of cells that are not activated and/or that lack or are reduced for or low for expression of one or more activation marker. In some embodiments, the phenotype is a phenotype of cells that are not exhausted and/or that lack or are reduced for or low for expression of one or more exhaustion markers.

In some embodiments, the phenotype is the production of one or more cytokines. In some embodiments, for example when the cytokine is produced and/or secreted by an engineered cell of a therapeutic cell composition in response to engagement of a recombinant receptor expressed by the cell with its antigen, this activity is referred to as recombinant receptor-dependent activity. In some embodiments, the therapeutic cell composition feature is recombinant receptor-dependent activity.

In some embodiments, the production of one or more cytokines is measured, detected, and/or quantified by intracellular cytokine staining. In particular embodiments, the phenotype is the lack of the production of the cytokine. In particular embodiments, the phenotype is positive for or is a high level of production of a cytokine. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. ICS can also be used in combination with other flow cytometry protocols for immunephenotyping using cell surface markers or with MHC multimers to access cytokine production in a particular subgroup of cells, making it an extremely flexible and versatile method. Other single-cell techniques for measuring or detecting cytokine production include, but are not limited to ELISPOT, limiting dilution, and T cell cloning.

In particular embodiments, for example in the therapeutic cell composition, the features include recombinant receptor-dependent activity. In some embodiments, the activity is a recombinant receptor, e.g., a CAR, dependent activity that is or includes the production and/or secretion of a soluble factor. In certain embodiments, the soluble factor is a cytokine or a chemokine.

Suitable techniques for the measurement of the production or secretion of a soluble factor are known in the art. Production and/or secretion of a soluble factor can be measured by determining the concentration or amount of the extracellular amount of the factor, or determining the amount of transcriptional activity of the gene that encodes the factor. Suitable techniques include, but are not limited to assays such as an immunoassay, an aptamer-based assay, a histological or cytological assay, an mRNA expression level assay, an enzyme linked immunosorbent assay (ELISA), alphalisa assay, immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay, protein microarrays, high-performance liquid chromatography (HPLC), Meso Scale Discovery (MSD) electrochemiluminescence and bead based multiplex immunoassays (MIA). In some embodiments, the suitable technique may employ a detectable binding reagent that specifically binds the soluble factor.

In some embodiments, the phenotype is indicated by the presence, absence, or level of expression in a cell of one or more specific molecules, such as certain surface markers indicative of the phenotype, e.g., surface proteins, intracellular markers indicative of the phenotype, or nucleic acids indicative of the phenotype or other molecules or factors indicative of the phenotype. In some embodiments, the phenotype is or comprises a positive or negative expression of the one or more of specific molecules. In some embodiments, the specific molecules include, but are not limited to, a surface marker, e.g., a membrane glycoprotein or a receptor; a marker associated with apoptosis or viability; or a specific molecule that indicates the status of an immune cells, e.g., a marker associated with activation, exhaustion, or a mature or naïve phenotype. In some embodiments, any known method for assessing or measuring, counting, and/or quantifying cells based on specific molecules can be used to determine the number of cells of the phenotype in the composition (e.g., input composition, therapeutic cell composition).

In some embodiments, a phenotype is or includes a positive or negative expression of one or more specific molecules in a cell. In some embodiments, the positive expression is indicated by a detectable amount of the specific molecule in the cell. In certain embodiments, the detectable amount is any detected amount of the specific molecule in the cell. In particular embodiments, the detectable amount is an amount greater than a background, e.g., background staining, signal, etc., in the cell. In certain embodiments, the positive expression is an amount of the specific molecule that is greater than a threshold, e.g., a predetermined threshold. Likewise, in particular embodiments, a cell with negative expression of a specific molecule may be any cell not determined to have positive expression, or is a cell that lacks a detectable amount of the specific molecule or a detectable amount of the specific molecule above background. In some embodiments, the cell has negative expression of a specific molecule if the amount of the specific molecule is below a threshold. One of skill in the art will understand how to define a threshold to define positive and/or negative expression for a specific molecule as a matter of routine skill, and that the thresholds may be defined according to specific parameters of, for example, but not limited to, the assay or method of detection, the identity of the specific molecule, reagents used for detection, and instrumentation.

Examples of methods that can be used to detect a specific molecule and/or analyze a phenotype of the cells include, but are not limited to, biochemical analysis; immunochemical analysis; image analysis; cytomorphological analysis; molecule analysis such as PCR, sequencing, high-throughput sequencing, determination of DNA methylation; proteomics analysis such as determination of protein glycosylation and/or phosphorylation pattern; genomics analysis; epigenomics analysis (e.g., ChIP-seq or ATAC-seq); transcriptomics analysis (e.g., RNA-seq); and any combination thereof. In some embodiments, the methods can include assessment of immune receptor repertoire, e.g., repertoire of T cell receptors (TCRs). In some aspects, determination of any of the phenotypes can be assessed in high-throughput, automated and/or by single-cell-based methods. In some aspects, large-scale or genome-wide methods, can be used to identify one or more molecular signatures. In some aspects, one or more molecular signatures, e.g., expression of specific RNA or proteins in the cell, can be determined. In some embodiments, molecular features of the phenotype analyzed by image analysis, PCR (including the standard and all variants of PCR), microarray (including, but not limited to DNA microarray, MMchips for microRNA, protein microarray, cellular microarray, antibody microarray, and carbohydrate array), sequencing, biomarker detection, or methods for determining DNA methylation or protein glycosylation pattern. In particular embodiments, the specific molecule is a polypeptide, i.e. a protein. In some embodiments, the specific molecule is a polynucleotide.

In some embodiments, positive or negative expression of a specific molecule is determined by incubating cells with one or more antibodies or other binding agents that specifically bind to one or more surface markers expressed or expressed (marker⁺) at a relatively higher level (marker^{high}) on the positively or negatively selected cells, respectively. In particular embodiments, the positive or negative expression is determined by flow cytometry, immunohistochemistry, or any other suitable method for detecting specific markers.

In particular embodiments, expression of a specific molecule is assessed with flow cytometry. Flow cytometry is a laser- or impedance-based, biophysical technology employed in cell counting, cell sorting, biomarker detection and protein engineering, by suspending cells in a stream of fluid and passing them by an electronic detection apparatus. It allows simultaneous multiparametric analysis of the physical and chemical characteristics of up to thousands of particles per second.

The data generated by flow-cytometers can be plotted in a single dimension, to produce a histogram, or in two-dimensional dot plots or even in three dimensions. The regions on these plots can be sequentially separated, based on fluorescence intensity, by creating a series of subset extractions, termed “gates.” Specific gating protocols exist for diagnostic and clinical purposes especially in relation to immunology. Plots are often made on logarithmic scales. Because different fluorescent dyes' emission spectra overlap, signals at the detectors have to be compensated electronically as well as computationally. Data accumulated using the flow cytometer can be analyzed using software, e.g., JMP (statistical software), WinMDI, Flowing Software, and web-based Cytobank), Cellcion, FCS Express, FlowJo, FACSDiva, CytoPaint (aka Paint-A-Gate), VenturiOne, CellQuest Pro, Infinicyt or Cytospec.

Flow Cytometry is a standard technique in the art and one of skill would readily understand how to design or tailor protocols to detect one or more specific molecules and analyze the data to determine the expression of one or more specific molecules in a population of cells. Standard protocols and techniques for flow cytometry are found in Loyd “Flow Cytometry in Microbiology; Practical Flow Cytometry by Howard M. Shapiro; Flow Cytometry for Biotechnology by Larry A. Sklar, Handbook of Flow Cytometry Methods by J. Paul Robinson, et al., Current Protocols in Cytometry, Wiley-Liss Pub, Flow Cytometry in Clinical Diagnosis, v4, (Carey, McCoy, and Keren, eds), ASCP Press, 2007, Ormerod, M. G. (ed.) (2000) Flow Cytometry—A practical approach. 3rd edition. Oxford University Press, Oxford, UK, Ormerod, M. G. (1999) Flow Cytometry. 2nd edition. BIOS Scientific Publishers, Oxford., and Flow Cytometry—A basic introduction. Michael G. Ormerod, 2008.

In some embodiments, cells are sorted by phenotype for further analysis. In some embodiments, cells of different phenotypes within the same cell composition are sorted by Fluorescence-activated cell sorting (FACS). FACS is a specialized type of flow cytometry that allows for sorting a heterogeneous mixture of cells into two or more containers, one cell at a time, based upon the specific light scattering and fluorescent characteristics of each cell. It is a useful scientific instrument as it provides fast, objective and quantitative recording of fluorescent signals from individual cells as well as physical separation of cells of particular interest.

In some embodiments, an input composition feature or therapeutic composition feature can include any one or more of the features of a cell composition, e.g., parameters or activities associated with an input cell composition or therapeutic T cell composition (e.g., CAR−T cells), respectively, described in published international applications WO 2019/032929, WO 2018/223101, WO 2019/089848, WO 2020/113194, WO 2019/090003, WO 2020/092848, WO 2019/113559, and WO 2018/157171, which are incorporated herein by reference in their entirety. In some embodiments, a subject feature can include any one or more of the features or characteristics of or associated with a subject (e.g., attributes of the subject or clinical attributes related to the subject in a clinical trial involving administration of the therapeutic T cell composition) described in published international applications WO 2019/032929, WO 2018/223101, WO 2019/089848, WO 2020/113194, WO 2019/090003, WO 2020/092848, WO 2019/113559, and WO 2018/157171, which are incorporated herein by reference in their entirety. In some embodiments, a clinical response to a therapeutic cell composition (e.g., CAR−T cells) can include any one or more clinical response to a therapeutic cell composition (e.g., CAR−T cells) described in published international applications WO 2019/032929, WO 2018/223101, WO 2019/089848, WO 2020/113194, WO 2019/090003, WO 2020/092848, WO 2019/113559, and WO 2018/157171, which are incorporated herein by reference in their entirety. Any one or more of such features can be used as data to determine (e.g., predict) any one or more clinical responses in accord with the provided methods.

Non-limiting examples of subject features, input composition features, therapeutic cell composition features used as data in the provided methods to determine (e.g., predict) one or more non-limiting clinical responses are described in the following subsections.

1. Subject Features

Various features associated with the subject to be treated with a therapeutic cell composition are contemplated for use according to the methods provided herein, e.g., machine learning methods. A subject to be treated with a therapeutic cell composition may also be referred to herein as a patient.

In some embodiments, the subject features include subject attributes, such as age and weight. In some embodiments, the subject feature is subject weight, e.g., body weight. In some embodiments, the subject weight is the weight of the subject at the time when the therapeutic cell composition is administered. In certain embodiments, weight is measured in lbs. or kg. In some embodiments, the subject feature is age, e.g., subject age at the initiation of administration of the therapeutic cell composition. Other exemplary subject features include, height, ethnicity, race, sex, gender, and body mass index.

In some embodiments, the features associated with the subject are clinical attributes. Exemplary clinical attributes include, but are not limited to, biomarkers and combinations of biomarkers, disease diagnosis, disease burden, disease duration, disease severity (e.g., disease grade), and treatment history.

In some embodiments, the clinical attribute associated with the subject (e.g., subject features) include an amount of prior therapies, e.g., one or more therapies prior to initiation of administration of the therapeutic T cell composition. In some embodiments, the prior therapies have been administered to treat the same disease and/or condition as the therapeutic cell composition.

In certain embodiments, the clinical attribute is platelet count.

In some embodiments, the clinical attribute is the recency of diagnosis with a disease. In some embodiments, the clinical attribute is the diagnosis the subject received.

In particular embodiments, the clinical attribute is having a leukemia. In some embodiments, the subject feature is having a B cell leukemia. In certain embodiments, the leukemia is acute lymphoblastic leukemia (ALL), non-Hodgkin's lymphoma (NHL), chronic lymphocytic leukemia (CLL), diffuse large B-cell lymphoma (DLBCL), or acute myeloid leukemia (AML). In certain embodiments, the clinical feature is having acute lymphocytic leukemia (ALL). In some embodiments, the clinical attribute is having lymphoma. In some embodiments, the clinical attribute is having a specific grade of lymphoma. In some embodiments, the clinical attribute is having DLBCL. In some embodiments, the clinical attribute is having follicular lymphoma. In some embodiments, the clinical attribute is having DLBCL transformed from follicular lymphoma. In some embodiments, the clinical attribute is the origin cell of DLBCL. For example, in some embodiments, the original cell is an activated B-cell, a non-germinal center B-cell, or a germinal center B-cell-like cell. In some embodiments, the clinical attribute is whether the disease is de novo or other. For example, in some embodiments, the clinical attribute is de novo DLBCL or DLBCL that is not de novo.

In some embodiments, the clinical attribute is a gene phenotype, such as identification of mutations in a gene known to correlate or be associated with a disease or condition. In some embodiments, the clinical attribute is whether a gene, e.g., gene correlated or associated with a disease or condition, has one or more mutations, e.g., deletions, insertions, substitutions, rearrangements, translocations. In some embodiments, the clinical attribute is the number of genes mutated (e.g., hits). In some embodiments, the clinical attribute is a gene double hit. For example, in some cases in lymphoma, two genes, e.g., MYC and BCL2, may be mutated. In some embodiments, the clinical attribute is a gene triple hit. For example, in some cases in lymphoma, three genes, e.g., MYC, BCL6, and BCL2, may be mutated. In some embodiments, the clinical attribute is a gene double or triple hit. In some embodiments, the clinical attribute is a gene double expressor. For example, in some cases in lymphoma, dual-expressor or double-expressor, refers to immunohistochemical detection of MYC and BCL2 overexpression. In some embodiments, double expressor refers to genes which are over-expressed, e.g., relative to a baseline.

In some aspects, the clinical attribute is whether the subject has relapsed or refractory disease. In some aspects, the clinical attribute is whether the subject has relapsed or has been refractory to the one or more prior therapies. In some embodiments, the clinical attribute is whether the subject is relapsed or refractory following chemotherapy treatment.

It is contemplated that subject to be treated with the therapeutic cell composition may have received prior treatment in an attempt to treat the disease or condition. Thus, in some embodiments, the clinical attribute is the number of prior lines of therapy the subject received prior to treatment with the therapeutic cell composition. In some embodiments, the clinical attribute is the number of prior lines of systemic therapy the subject received prior to treatment with the therapeutic cell composition. In some embodiments, the clinical attribute is whether the subject received allogenic hematopoietic stem cell transplantation prior to treatment with the therapeutic cell composition. In some embodiments, the clinical attribute is whether the subject received autologous hematopoietic stem cell transplantation prior to treatment with the therapeutic cell composition. In some embodiments, the clinical attribute is best overall response to prior treatment.

In some embodiments, the clinical attribute is the disease stage.

In some embodiments, the clinical attribute is disease burden. In particular embodiments, the clinical attribute is high disease burden, e.g., a high disease burden prior to initiation of administration of the therapeutic T cell composition. In certain embodiments, the clinical attribute is high disease burden immediately prior to, or within 1 week, 2 weeks, 3 weeks, 4 weeks, 5 weeks, 6 weeks, 7 weeks, 8 weeks, 9 weeks, one month, two months, three months, four months, five months, six months, or greater than six months prior to initiation of administration of the therapeutic cell composition. In some embodiments, disease burden is determined by lesion count. In some embodiments, the high disease burden is determined based on percent of bone marrow blasts. In certain embodiments, the subject feature is high disease burden, such as the sum of product diameter (SPD) or levels of lactate dehydrogenase (LDH).

In some embodiments, the clinical attribute is lesion count. In some embodiments, the clinical attribute is SPD. In some embodiments, the clinical attribute is LDH level. In some embodiments, the clinical attribute is a fold change in SPD. In some embodiments, the clinical attribute is a fold change LDH level. In some embodiments, the fold change is determined between the timing of an initial screen and the time at which a lymphodepleting therapy is delivered before administration of the therapeutic cell composition. In some embodiments, the fold change is determined between the timing of an initial screen and the time at which the therapeutic cell composition is administered. In some embodiments, the clinical attributes SPD, LDH, lesion count, and fold changes, differences, or other quantifications thereof are used to assess disease burden.

In some embodiments, the clinical attribute is disease burden, for example, as described by tumor burden. In some embodiments, the clinical attribute is high tumor burden, e.g., a high disease burden prior to initiation of administration of the therapeutic cell composition. In some embodiments, the tumor burden is determined by a volumetric measure of tumor(s). In some embodiments, the volumetric measure is a measure of the lesion(s), such as the tumor size, tumor diameter, tumor volume, tumor mass, tumor load or bulk, tumor-related edema, tumor-related necrosis, and/or number or extent of metastases. In addition, “bulky disease” can be used to describe large tumors in the chest. In some embodiments, the volumetric measure of tumor is a bidimensional measure. For example, in some embodiments, the area of lesion(s) is calculated as the product of the longest diameter and the longest perpendicular diameter of all measurable tumors. In some cases, the volumetric measure of tumor is a unidimensional measure. In some cases, the size of measurable lesions is assessed as the longest diameter. In some embodiments, tumor size is assessed as the longest diameter. In some embodiments, tumor size is assessed as the perpendicular diameter. In some embodiments, the sum of the products of diameters (SPD), longest tumor diameters (LD), sum of longest tumor diameters (SLD), necrosis, tumor volume, necrosis volume, necrosis-tumor ratio (NTR), peritumoral edema (PTE), and edema-tumor ratio (ETR) is measured. Exemplary methods for measuring and assessing tumor burden include those described in, e.g., Carceller et al., Pediatr Blood Cancer. (2016) 63(8):1400-1406 and Eisenhauer et al., Eur J Cancer. (2009) 45(2):228-247. In some embodiments, the volumetric measure is a sum of the products of diameters (SPD) measured by determining the sum of the products of the largest perpendicular diameters of all measurable tumors. In some aspects, the tumor or lesion are measured in one dimension with the longest diameter (LD) and/or by determining the sum of longest tumor diameters (SLD) of all measurable lesions. In some embodiments, the volumetric measure of tumor is a volumetric quantification of tumor necrosis, such as necrosis volume and/or necrosis-tumor ratio (NTR), see Monsky et al., Anticancer Res. (2012) 32(11): 4951-4961. In some aspects, the volumetric measure of tumor is a volumetric quantification of tumor-related edema, such as peritumoral edema (PTE) and/or edema-tumor ratio (ETR). In some embodiments, measuring can be performed using imaging techniques such as computed tomography (CT), positron emission tomography (PET), and/or magnetic resonance imaging (MRI) of the subject.

In some embodiments, the volumetric measure of tumor is determined at a screening session, such as a routine assessment or blood draw to confirm and/or identify the condition or disease in the subject. In some embodiments, the measure of the tumor burden, such as a volumetric measure (e.g., SPD) is measured prior to lymphodepleting chemotherapy (LDC). For example, in some embodiments, the measure of the tumor burden, such as a volumetric measure (e.g., SPD) is measured or assessed within one month, two weeks, or one week prior to LDC, such as within 7 days, 6 days, 5 days, 4 days, 3 days, 2 days or 1 day prior to LDC. In particular embodiments, the measure of the tumor burden, such as a volumetric measure (e.g., SPD) is measured prior to the infusion of the T cell therapy to tumor-bearing subjects.

In some embodiments, the subject feature is a categorical cut of whether the measure of tumor burden, e.g., a volumetric measure, is above or below a threshold level. For example, the feature is a categorical cut of a measure of tumor burden (e.g., volumetric measure), such as measured before infusion of a T cell therapy, e.g., before LDC, in which the feature is whether the subject has a measure of the tumor burden that is at or below the threshold level or is greater than the threshold level. In particular embodiments, the measure of tumor burden is SPD and the threshold level for SPD is at or at about 30 cm², 40 cm², 50 cm², 60 cm², 70 cm², 80 cm² or 90cm². For example, the feature is a categorical cut of SPD, such as measured before infusion of a T cell therapy, e.g., before LDC, in which the feature is whether the subject has an SPD that is at or below 50 cm² or greater than 50 cm².

In some embodiments, the factor indicative of tumor burden is assessed at two time points, and a fold change of the factor indicative of disease burden between two time points is determined. In some embodiments, the two time points comprises a first time point and a second time point, and wherein the fold change is a ratio of the factor indicative of disease burden at the first time point and the factor indicative of disease burden at the second time point. In some embodiments, the volumetric measure of tumor is determined at two time points prior to the administration of the therapy, e.g., cell therapy. In some embodiments, the volumetric measure of tumor is determined at a screening session, such as a routine assessment or blood draw to confirm and/or identify the condition or disease in the subject. In particular embodiments, the volumetric measurement of the tumor(s) is determined or measured in a subject who has been, who will be, or is a candidate to be administered a T cell therapy. In particular embodiments, the measurement is determined prior to treatment or administration with the therapy, e.g., the cell therapy. In some embodiments, the two time points are both no more than one month or two months prior to receiving the cell therapy. In some embodiments, the two time points are not less than one week, two weeks, three weeks, four weeks, or five weeks apart. In some embodiments, the two time points are not less than three weeks apart. In some embodiments, the two time points are not more than four weeks apart, five weeks, or six weeks apart. In some embodiments, the second time point is more than 1, 2, 3, 4, 5, 6, or 7 days before administration of the cell therapy.

In some embodiments, the clinical attribute is disease burden as determined by extranodal disease classification. For example, the clinical attribute may be whether the disease, e.g., lymphoma, has spread to an organ outside the lymph system. In some embodiments, the clinical attribute is the number of extranodal sites affected.

In some embodiments, the clinical attribute is whether the subject has a central nervous system (CNS) disease at the time of administering the therapeutic cell composition. In some embodiments, the subject does not have a CNS disease at the time of administering the therapeutic cell composition. In some embodiments, the CNS disease is a primary CNS lymphoma (PCNSL). In some embodiments, the PCNSL involves the central nervous system (CNS) without systemic lymphoma presence. In some embodiments, the PCNSL is confined to the brain, spine, cerebrospinal fluid (CSF), and eyes. In some embodiments, the PCNSL is a diffuse large B-cell lymphoma (DLBCL). In some embodiments, the PCNSL is a Burkitt, low-grade or T-cell lymphoma. In some embodiments, the PCNSL includes neurological signs. In some embodiments, the neurological signs include focal neurologic deficits, mental status and behavioral changes, symptoms of increased intracranial pressure, and/or seizures. In some embodiments, exemplary features associated with the disease or condition include those described in Grommes et al. (J. Clin Oncol 2017; 35(21):2410-18).

In some embodiments, the CNS disease is a secondary central nervous system lymphoma (SCNSL). In some embodiments, the SCNSL is in patients with systemic lymphoma. In some embodiments, the SCNSL is referred to as metastatic lymphoma. In some embodiments, the SCNSL is a DLBCL. In some embodiments, the SCNSL is an aggressive lymphoma that may involve the brain, meninges, spinal cord, and eyes. In some embodiments, the SCNSL includes leptomeningeal spread. In some embodiments, the SCNSL includes brain parenchymal disease. In some embodiments, exemplary features associated with the disease or condition include those described in Malikova et al. (Neurophychiatric Disease and Treatment 2018; 14:733-40. In some embodiments, the secondary CNS lymphoma involves the brain parenchyma and/or leptomeninges.

In some embodiments, the clinical attribute is a comorbidity. For example, in some cases, the comorbidity is creatinine clearance (CrCl) prior to the subject receiving lymphodepleting chemotherapy prior to administration of the therapeutic cell composition. In some embodiments, the comorbidity is left ventrical ejection fraction (LVEF).

In some embodiments, the clinical attribute is an Eastern Cooperative Oncology Group Performance Status (ECOG). In some embodiments, the subject's ECOG status is defined as: Grade 0—Fully active, able to carry on all pre-disease performance without restriction; Grade 1—Restricted in physically strenuous activity but ambulatory and able to carry out work of a light or sedentary nature; Grade 2—Ambulatory and capable of all selfcare but unable to carry out any work activities; up and about more than 50% of waking hours; Grade 3—Capable of only limited selfcare; confined to bed or chair more than 50% of waking hours; Grade 4—Completely disabled; cannot carry on any selfcare; totally confined to bed or chair; or Grade 5—Dead.

In some embodiments, the clinical attribute is an International Prognostic Index Score (IPI), e.g., a lymphoma IPI score. In some embodiments, the subject's IPI score is defined as: Low risk (0-1 points)—5-year survival of 73%; Low-intermediate risk (2 points)—5-year survival of 51%; High-intermediate risk (3 points)—5-year survival of 43%; or High risk (4-5 points)—5-year survival of 26%.

In some embodiments, the clinical attribute is a subject's temperature. In some embodiments, the clinical attribute is blood oxygenation level. In some embodiments, the clinical attribute is albumin level. In some embodiments, the clinical attribute is alkaline phosphatase level. In some embodiment, the clinical attribute is basophils count. In some embodiments, the clinical attribute is absolute basophils count. In some embodiments, the clinical attribute is direct bilirubin. In some embodiments, the clinical attribute is total bilirubin. In some embodiments, the clinical attribute is lymphocyte count or absolute count. In some embodiments, the lymphocyte count is a count prior to performing leukapheresis on a subject to obtain cells for generating a therapeutic cell composition. In some embodiments, the clinical attribute is a blood urea nitrogen level. In some embodiments, the clinical attribute is a calcium level. In some embodiments, the clinical attribute is a carbon dioxide level. In some embodiments, the clinical attribute is a chloride level. In some embodiments, the clinical attribute is creatinine level. In some embodiments, the clinical attribute is an eosinophils count or an absolute count. In some embodiments, the clinical attribute is a glucose level. In some embodiments, the clinical attribute is hematocrit level. In some embodiments, the clinical attribute is hemoglobin level. In some embodiments, the clinical attribute is a magnesium level. In some embodiments, the clinical attribute is monocyte count or absolute count. In some embodiments, the clinical attribute is neutrophil count or absolute count. In some embodiments, the clinical attribute is a platelet count. In some embodiments, the clinical attribute is potassium level. In some embodiments, the clinical attribute is a total protein level. In some embodiments, the clinical attribute is a red blood cell count. In some embodiments, the clinical attribute is a white blood cell count. In some embodiments, the clinical attribute is a uric acid level. In some embodiments, the clinical attribute is sodium level. In some embodiments, the clinical attribute is a triglyceride level. In some embodiments, the clinical attribute is an asparate amintransferase level. In some cases, asparate amintransferase level may be determined by a serum glutamic-oxaloacetic transaminase test. In some embodiments, the clinical attribute is an alanine aminotransferase level. In some cases, asparate amintransferase level may be determined by a serum glutamic-pyruvic transaminase test. Any suitable method for detecting levels or counts as described is contemplated.

In some embodiments, the clinical attribute is a level, amount, and/or a concentration of an inflammatory marker. In some embodiments, the inflammatory marker is or includes the level or presence of C-reactive protein (CRP), erythrocyte sedimentation rate (ESR), albumin, ferritin, β2 microglobulin (β2-M), or lactate dehydrogenase (LDH) is detected and assessed. In some embodiments, the inflammatory marker is assessed using an immune assay. For example, an enzyme-linked immunosorbent assay (ELISA), enzyme immunoassay (EIA), radioimmunoassay (RIA), surface plasmon resonance (SPR), Western Blot, Lateral flow assay, immunohistochemistry, protein array or immuno-PCR (iPCR) can be used to detect the inflammatory marker. In some embodiments, the presence, level, amount, and/or concentration of an inflammatory marker is indicative of tumor burden, e.g., a high tumor burden. In some cases, the assaying or assessing of an inflammatory marker is using flow cytometry. In some cases, the reagent is a soluble protein that binds the inflammatory marker. In some examples, the reagent is a protein that binds C-reactive protein (CRP), erythrocyte sedimentation rate (ESR), albumin, ferritin, β2 microglobulin (β2-M), or lactate dehydrogenase (LDH).

In some embodiments, the clinical attribute is a biomarker. In some embodiments, the biomarker is an inflammatory marker, such as C-reactive protein (CRP). In some embodiments, CRP is assessed using an in vitro enzyme-linked immunosorbent assay to obtain a quantitative measurement of human CRP from a sample such as serum, plasma, or blood. In some examples, CRP is detected using a human Enzyme-Linked Immunosorbent Assay (ELISA). In some embodiments, the biomarker is an inflammatory marker, such as an erythrocyte sedimentation rate (ESR). In some embodiments, ESR is assessed by measuring the distance (in millimeters per hour) that red cells have fallen after separating from the plasma in a vertical pipette or tube. In some embodiments the biomarker is or includes albumin. In some aspects, albumin is assessed using a colorimetric test or an in vitro enzyme-linked immunosorbent assay. In some examples, albumin is detected using a human Enzyme-Linked Immunosorbent Assay (ELISA). In some embodiments, the biomarker is an inflammatory marker, such as ferritin or β2 microglobulin. In some embodiments, ferritin or β2 microglobulin is assessed using an immunoassay or detected using an ELISA. In some aspects, the biomarker is an inflammatory marker, such as lactate dehydrogenase (LDH), and LDH is assessed using a colorimetric test or an in vitro enzyme-linked immunosorbent assay. In some embodiments, the clinical attribute is a level, concentration, and/or amount of LDH.

In some embodiments, the level, concentration and/or number of LDH is a surrogate for disease burden, e.g., for tumors or cancers.

In some embodiments, the clinical attribute is receiving a bridging chemotherapy prior to initiation of administration of the therapeutic T cell composition. In some embodiments, the bridging chemotherapy is a systemic treatment. In some embodiments, the clinical attribute is receiving a bridging chemotherapy and radiotherapy prior to initiation of administration of the therapeutic T cell composition. A treating physician can determine if bridging therapy is necessary, for example for disease control, during manufacturing of the provided compositions or cells.

In particular embodiments, the clinical attribute is preconditioning with a lymphodepleting therapy, e.g., prior to initiation of administration of the therapeutic T cell composition. In some embodiments, the lymphodepleting therapy is or includes the administration of a chemotherapy. In particular embodiments, the subject feature is preconditioning with fludarabine and/or cyclophosphamide prior to initiation of administration of the therapeutic cell composition. In certain embodiments, the subject feature is preconditioning with cyclophosphamide prior to initiation of administration of the therapeutic T cell composition. In some embodiments, the subject feature is preconditioning with fludarabine and cyclophosphamide prior to initiation of administration of the therapeutic cell composition.

In some embodiments, the clinical attribute is a level, amount or concentration of a cytokine in a blood, serum, or plasma sample prior to initiation of administration of the therapeutic T cell composition. In some embodiments, the cytokine is an interleukin, e.g., interleukin-15 (IL-15).

In some embodiments, the subject feature is the dosing arm of a study in which the subject is treated. In some cases, this feature may be particularly useful, for example, in some clinical studies where different dosing levels are used, to account for differences in dosing when assessing clinical response according to the methods provided herein.

In some embodiments, the subject features include any one or more subject feature, including clinical attributes and subject attributes, described herein.

In some embodiments, the subject feature is one or more or all of a dosing arm, bridging chemotherapy, bridging chemotherapy and radiotherapy, bridging chemotherapy systemic treatment, cell origin (e.g., ABC (activated B-cell-like, or non-GCB) or GCB (germinal center B-cell-like), relapsed or refractory following chemotherapy, type of diagnosis, disease cohort (e.g., DLBCL), disease burden, relapsed or refractory disease, disease origin (e.g., de novo DLBCL or other DLBCL), gender, therapeutic cell composition administration route (e.g., infusion), fold change in LDH, height, lesion count, oxygen saturation, temperature (° c), longest tumor diameter pre-treatment with therapeutic cell composition, fold change in SPD, SPD value pre-lymphodepleting chemotherapy (e.g., with a categorical threshold for groups with SPD<=50 cm\hat{ }2 or >50 cm\hat{ }2), BMI, weight, sex, ethnicity, race, age, IPI score, ECOG score, disease stage, disease burden based on pre-lymphodepleting chemotherapy LDH, disease burden based on pre-lymphodepleting chemotherapy SPD, subject having active CNS disease at time of treatment, disease burden based on extranodal disease classification, number of extranodal sites, disease burden based on bulky disease classification, disease histology, number of prior lines of therapy, number of prior lines of systemic therapy, prior allogenic hematopoietic stem cell transplantation (allo-HSCT), prior autologous hematopoietic stem cell transplantation (auto-HSCT), chemorefractory or chemosensitive disease type, bridging anticancer therapy for disease control, days from date of leukapheresis to first infusion, months from diagnosis to treatment with therapeutic cell composition, comorbidity (e.g., creatinine clearance (CrCl) prior to lymphodepleting, left ventrical ejection fraction (LVEF) at screening), baseline C Reactive Protein (CRP), pre-leukapheresis lymphocyte count (10\hat{ }9/L), gene double expressor, gene double hit, gene triple hit, gene double or triple hit, gene double or triple hit or double expressor, albumin level, alkaline phosphatase level, basophils count, absolute basophil count, direct bilirubin, total bilirubin, blood urea nitrogen level, calcium level, carbon dioxide level, chloride level, creatinine level, eosinophils count, eosinophils absolute count, glucose level, hematocrit level, hemoglobin level, LDH level, lesion count, lymphocyte count, lymphocyte absolute count, magnesium level, monocyte absolute count, monocyte count, neutrophil absolute count, neutrophil count, phosphate level, platelet count, potassium level, total protein, red blood cell count, aspartate aminotransferase level, alanine aminotransferase level, sodium level, sum of products of diameters, triglycerides, longest tumor diameter, perpendicular tumor diameter, uric acid level, and white blood cell count.

In some embodiments, the subject features include any one or more of the subject features shown in Table E4 below.

In some embodiments, the subject features are determined at an initial screening. For example, a screen that takes place prior to leukapheresis for generating an input composition for producing the therapeutic cell composition. In some embodiments, the subject features are determined before administration of a lymphodepleting therapy prior to administering the therapeutic cell composition. In some embodiments, the subject features are determined at the time the therapeutic cell composition is administered. In some cases, for example when the subject feature is or includes a change in a subject feature, the subject feature may be determined at two or more time points, for example at initial screen, prior to lymphodepleting therapy administration, prior to therapeutic cell composition administration, to determine a difference or change, e.g., percent change, fold change, in the subject feature.

2. Input Composition Features

In some embodiments, the input composition contains cells isolated from samples (e.g., biological samples), such as those obtained from or derived from a subject, such as a subject in need of a cell therapy or to which cell therapy will be administered. Methods for isolating cells from samples (e.g., biological samples) are described, for example, in Section II-A. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. In some embodiments, the input composition contains CD4+ and CD8+ T cells. In some embodiments, the input composition contains CD4+ or CD8+ T cells.

In some embodiments, the input composition feature includes cell phenotypes. In some embodiments, the phenotype is the number of total T cells. In some embodiments, the phenotype is the number of total CD3⁺ T cells. In some embodiments, the phenotype is or includes the identity of a T cell subtype. Different populations or subtypes of T cells include, but are not limited to effector T cells, helper T cells, memory T cell, Regulatory T cells, naïve T cells, CD4⁺ cells, and CD8⁺ T cells. In some embodiments, a T cell subtype may be identified by detecting the presence or absence of a specific molecule. In certain embodiments, the specific molecule is a surface marker that can be used to identify a T cell subtype.

In some embodiments, the phenotype is positive or high level expression of one or more specific molecule that are surface markers, e.g., CD3, CD4, CD8, CD28, CD62L, CCR7, CD27, CD127, CD4, CD8, CD45RA, and/or CD45RO. In certain embodiments, the phenotype is a surface marker of T cells or of a subpopulation or subset of T cells, such as based on positive surface marker expression of one or more surface markers, e.g., CD3⁺, CD4⁺, CD8⁺, CD28⁺, CD62L⁺, CCR7⁺, CD27⁺, CD127⁺, CD4⁺, CD8⁺, CD45RA⁺, and/or CD45RO⁺. In some embodiments, the phenotype is positive or high level expression of one or more specific molecule that are surface markers, e.g., C-C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin. In some embodiments, the phenotype is negative or the absence of expression of one or more specific molecules that are surface markers, e.g., CD3, CD4, CD8, CD28, CD62L, CCR7, CD27, CD127, CD4, CD8, CD45RA, and/or CD45RO. In certain embodiments, the phenotype is a surface marker of T cells or of a subpopulation or subset of T cells, such as based on the absence of surface marker expression of one or more surface markers, e.g., CD3⁻, CD4⁻, CD8−, CD28⁻, CD62L⁻, CCR7⁻, CD27⁻, CD127 , CD4⁻, CD8⁻, CD45RA⁻, and/or CD45RO⁻. In some embodiments, the phenotype is negative or the absence of expression of one or more specific molecule that are surface markers, e.g., C-C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin.

In certain embodiments, the phenotype is or includes positive or negative expression of CD27, CCR7 and/or CD45RA. In some embodiments, the phenotype is CCR7⁺. In some embodiments, the phenotype is CD27⁺. In some embodiments, the phenotype is CCR7⁻. In some embodiments, the phenotype is CD27⁻. In some embodiments, the phenotype is CCR7⁺/CD27⁺. In some embodiments, the phenotype is CCR7⁻/CD27⁺. In some embodiments, the phenotype is CCR7⁺/CD27−. In some embodiments, the phenotype is CCR7⁻/CD27⁻. In some embodiments, the phenotype is CD45RA⁻. In some embodiments, the phenotype is CD45RA⁺. In some embodiments, the phenotype is CCR7⁺/CD45RA⁻. In some embodiments, the phenotype is CD27⁻/CD45RA⁻. In some embodiments, the phenotype is CD27⁺/CD45RA⁺. In some embodiments, the phenotype is CD27⁻/CD45RA⁺. In some embodiments, the phenotype is CD27⁻/CD45RA⁻. In some embodiments, the phenotype is CCR7⁺/CD27⁺/CD45RA⁻. In some embodiments, the phenotype is CCR7⁺/CD27⁺/CD45RA+.

In some embodiments, the phenotype is viability. In certain embodiments, the phenotype is the positive expression of a marker that indicates that the cell undergoes normal functional cellular processes and/or has not undergone or is not under the process of undergoing necrosis or programmed cell death. In some embodiments, viability can be assessed by the redox potential of the cell, the integrity of the cell membrane, or the activity or function of mitochondria. In some embodiments, viability is the absence of a specific molecule associated with cell death, or the absence of the indication of cell death in an assay.

In some embodiments, the phenotype is or comprises cell viability. In certain embodiments, the viability of cells can be detected, measured, and/or assessed by a number of means that are routine in the art. Non-limiting examples of such viability assays include, but are not limited to, dye uptake assays (e.g., calcein AM assays), XTT cell viability assays, and dye exclusion assays (e.g., trypan blue, Eosin, or propidium dye exclusion assays). Viability assays are useful for determining the number or percentage (e.g., frequency) of viable cells in a cell dose, a cell composition, and/or a cell sample. In particular embodiments, the phenotype comprises cell viability along with other features, e.g., surface makers, molecules.

In certain embodiments, the phenotype is or includes cell viability, viable CD3⁺, viable CD4⁺, viable CD8⁺, viable CD4⁺/CCR7⁺, viable CD8⁺/CD27⁺, viable CD4⁺/CD27⁺, viable CD8⁺/CCR7⁺/CD27⁺, viable CD4⁺/CCR7⁺/CD27⁺, viable CD8⁺/CCR7⁺/CD45RA⁻ or viable CD4⁺/CCR7⁺/CD45RA⁻ or a combination thereof.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death. These changes include blebbing, cell shrinkage, nuclear fragmentation, chromatin condensation, chromosomal DNA fragmentation, and global mRNA decay. Apoptosis is a well characterized process, and specific molecules associated with various stages are well known in the art.

In some embodiments, the phenotype is the absence of an early stage of apoptosis, and/or an absence of an indicator and/or a specific molecule associated with an early stage of apoptosis. In the early stages of apoptosis, changes in the cellular and mitochondrial membrane become apparent. Biochemical changes are also apparent in the cytoplasm and nucleus of the cell. For example, the early stages of apoptosis can be indicated by activation of certain caspases, e.g., 2, 8, 9, and 10. In particular embodiments, the phenotype is the absence of a late stage of apoptosis, and/or an absence of an indicator and/or a specific molecule associated with a late stage of apoptosis. The middle to late stages of apoptosis are characterized by further loss of membrane integrity, chromatin condensation and DNA fragmentation, and include biochemical events such as activation of caspases 3, 6, and 7.

In certain embodiments, the phenotype is the negative expression of one or more factors associated with apoptosis, including pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In some embodiments, the phenotype is a negative or low amount of a marker of apoptosis. In certain embodiments, the phenotype is the negative expression of a marker of apoptosis. In certain embodiments, the phenotype is the absence of an indicator, e.g., an Annexin V molecule, which will preferentially bind to cells undergoing apoptosis when incubated with or contacted to a cell composition. In some embodiments, the phenotype is or includes the expression of one or more markers that are indicative of an apoptotic state in the cell.

In some embodiments, the phenotype is the negative (or low) expression of a specific molecule that is a marker for apoptosis. Various apoptosis markers are known to those of ordinary skill in the art and include, but are not limited to, an increase in activity of one or more caspases i.e. an activated caspase (e.g., an active caspase, CAS), an increase in PARP cleavage, activation and/or translocation of Bcl-2 family proteins, members of the cell death pathway, e.g., Fas and FADD, presence of nuclear shrinkage (e.g., monitored by microscope) and presence of chromosome DNA fragmentation (e.g., presence of chromosome DNA ladder) or with apoptosis assays that include TUNEL staining, and Annexin V staining.

Caspases are enzymes that cleave proteins after an aspartic acid residue, the term is derived from “cysteine-aspartic acid proteases.” Caspases are involved in apoptosis, thus activation of caspases, such as caspase-3 is indicative of an increase or revival of apoptosis. In some embodiments, activated caspase-3 is referred to herein as 3CAS. In certain embodiments, caspase activation can be detected by methods known to the person of ordinary skill. In some embodiments, an antibody that binds specifically to an activated caspase (i.e., binds specifically to the cleaved polypeptide) can be used to detect caspase activation. In another example, a fluorochrome inhibitor of caspase activity (FLICA) assay can be utilized to detect caspase-3 activation by detecting hydrolysis of acetyl Asp-Glu-Val-Asp 7-amido-4-methylcoumarin (Ac-DEVD-AMC) by caspase-3 (i.e., detecting release of the fluorescent 7-amino-4-methylcoumarin (AMC)). FLICA assays can be used to determine caspase activation by a detecting the product of a substrate processed by multiple caspases (e.g., FAM-VAD-FMK FLICA). Other techniques include The CASPASE-GLO® caspase assays (PROMEGA) that use luminogenic caspase-8 tetrapeptide substrate (Z-LETD-aminoluciferin), the caspase-9 tetrapeptide substrate (Z-LEHD-aminoluciferin), the caspase-3/7 substrate (Z-DEVD-aminoluciferin), the caspase-6 substrate (Z-VEID-aminoluciferin), or the caspase-2 substrate (Z-VDVAD-aminoluciferin).

In certain embodiments, the phenotype is or includes negative expression of activated caspase-1, activated caspase-2, activated caspase-3, activated caspase-7, activated caspase-8, activated caspase-9, activated caspase-10 and/or activated caspase-13 in a cell. In particular embodiments, the phenotype is or includes activated caspase In some embodiments, the proform (zymogen cleaved) form of a caspase, such as any above, also is a marker indicating the presence of apoptosis. In some embodiments, the phenotype is or includes the absence of or negative expression of a proform of a caspase, such as the proform of caspase-3.

In some embodiments, the marker of apoptosis is cleaved the Poly ADP-ribose polymerase 1 (PARP). PARP is cleaved by caspase during early stages of apoptosis. Thus, detection of a cleaved PARP peptide is a marker for apoptosis. In particular embodiments, the phenotype is or includes positive or negative expression of cleaved PARP.

In some embodiments, the marker of apoptosis is a reagent that detects a feature in a cell that is associated with apoptosis. In certain embodiments, the reagent is an annexin V molecule. During the early stages of apoptosis the lipid phosphatidylserine (PS) translocates from the inner to the outer leaflet of the plasma membrane. PS is normally restricted to the internal membrane in healthy and/or non-apoptotic cells. Annexin V is a protein that preferentially binds phosphatidylserine (PS) with high affinity. When conjugated to a fluorescent tag or other reporter, Annexin V can be used to rapidly detect this early cell surface indicator of apoptosis. In some embodiments, the presence of PS on the outer membrane will persist into the late stages of apoptosis. Thus in some embodiments, annexin V staining is an indication of both early and late stages of apoptosis. In certain embodiments, an Annexin, e.g., Annexin V, is tagged with a detectable label and incubated with, exposed to, and/or contacted with cells of a cell composition to detect cells that are undergoing apoptosis, for example by flow cytometry. In some embodiments, fluorescence tagged annexins, e.g., annexin V, are used to stain cells for flow cytometry analysis, for example with the annexin⁻V/7⁻AAD assay. Alternative protocols suitable for apoptosis detection with annexin include techniques and assays that utilize radiolabeled annexin V. In certain embodiments, the phenotype is or includes negative staining by annexin, e.g., annexin V⁻. In particular embodiments, the phenotype is or includes the absence of PS on the outer plasma membrane. In certain embodiments, the phenotype is or includes cells that are not bound by annexin e.g., annexin V. In certain embodiments, the cell that lacks detectable PS on the outer membrane is annexin V⁻. In particular embodiments, the cell that is not bound by annexin V⁻ in an assay, e.g., flow cytometry after incubation with labeled annexin V, is annexin V⁻.

In particular embodiments, the phenotype is annexin V⁻, annexin V⁻CD3⁺, annexin V⁻CD4⁺, annexin V⁻CD8⁺, annexin V⁻CD3⁺, annexin V⁻CD4⁺, annexin V⁻CD8⁺, activated caspase 3⁻, activated caspase 3⁻/CD3⁺, activated caspase 3⁻/CD4⁺, activated caspase 3⁻ /CD8⁺, activated caspase 3⁻/CD3⁺, activated caspase 3⁻/CD4⁺, activated caspase 3⁻/CD8⁺, annexin V⁻/CD4⁺/CCR7⁺, annexin V⁻/CD8⁺/CD27⁺, annexin V−/CD4⁺/CD27⁺, annexin V⁻/CD8⁺/CCR7⁺/CD27⁺, annexin V−/CD4⁺/CCR7⁺/CD27⁺, annexin V⁻/CD8⁺/CCR7⁺/CD45RA⁻ or annexin V−/CD4⁺/CCR7⁺/CD45RA⁻; activated caspase 3⁻/CD4⁺/CCR7⁺, activated caspase 3−/CD8⁺/CD27⁺, activated caspase 3⁻/CD4⁺/CD27⁺, activated caspase 3⁻/CD8⁺/CCR7⁺/CD27⁺, activated caspase 3⁻/CD4⁺/CCR7⁺/CD27⁺, activated caspase 3⁻/CD8⁺/CCR7⁺/CD45RA⁻ or activated caspase 3⁻/CD4⁺/CCR7⁺/CD45RA⁻ or a combination thereof. In some embodiments, the phenotype is 3CAS−/CCR7−/CD27−. In some embodiments, the phenotype is 3CAS−/CCR7−/CD27+. In some embodiments, the phenotype is 3CAS−/CCR7+. In some embodiments, the phenotype is 3CAS−/CCR7+/CD27−. In some embodiments, the phenotype is 3CAS−/CCR7+/CD27+. In some embodiments, the phenotype is 3CAS−/CD27+. In some embodiments, the phenotype is 3CAS−/CD28−/CD27−. In some embodiments, the phenotype is 3CAS−/CD28−/CD27+. In some embodiments, the phenotype is 3CAS−/CD28+. In some embodiments, the phenotype is 3CAS−/CD28+/CD27−. In some embodiments, the phenotype is 3CAS−/CD28+/CD27+. In some embodiments, the phenotype is 3CAS−/CCR7−/CD45RA−. In some embodiments, the phenotype is 3CAS−/CCR7−/CD45RA+. In some embodiments, the phenotype is 3CAS−/CCR7+/CD45RA−. In some embodiments, the phenotype is 3CAS−/CCR7+/CD45RA+. In some embodiments, the phenotype further is CD4+. In some embodiments, the phenotype further is CD8+.

Particular embodiments contemplate that cells positive for expression of a marker for apoptosis are undergoing programmed cell death, show reduced or no immune function, and have diminished capabilities if any to undergo activation, expansion, and/or bind to an antigen to initiate, perform, or contribute to an immune response or activity. In particular embodiments, the phenotype is defined by negative expression for an activated caspase and/or negative staining with annexin V.

In certain embodiments, the phenotype is or includes activated caspase 3 (caspase 3, 3CAS) and/or annexin V.

Among the phenotypes are the expression or surface expression of one or more markers generally associated with one or more sub-types or subpopulations of T cells, or phenotypes thereof. T cell subtypes and subpopulations may include CD4+ and/or of CD8+ T cells and subtypes thereof that may include naïve T (T_{N}) cells, naïve-like cells, effector T cells (T), memory T cells and sub-types thereof, such as stem cell memory T (T_{SCM}), central memory T (T_{CM}), effector memory T (T_{EM}), T_{EMRA} cells or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the input composition feature is the clonality of the cells of the input composition. In some embodiments, assessing the clonality of the population of T cells is an assessment of clonal diversity of the population of T cells. In some embodiments, the T cells are polyclonal or multiclonal. Clonality, such as polyclonality, of said input composition of T cells is a measure of the breadth of the response of the population to a given antigen. In some aspects, the input composition can be assessed by measuring the number of different epitopes recognized by antigen-specific cells. This can be carried out using standard techniques for generating and cloning antigen-specific T cells in vitro. In some embodiments, the T cells are polyclonal (or multiclonal) with no single clonotypic population predominating in the population of naïve-like T cells.

In the context of a population of T cells, such as of the input composition, in some aspects, the signature of polyclonality refers to a population of T cells that has multiple and broad antigen specificity. In some embodiments, polyclonality relates to a population of T cells that exhibits high diversity in the TCR repertoire. In some cases, diversity of the TCR repertoire is due to V(D)J recombination events that, in some respects, are triggered by selection events to self and foreign antigens. In some embodiments, a population of T cells that is diverse or polyclonal is a population of T cells in which analysis indicates the presence of a plurality of varied or different TCR transcripts or products present in the population. In some embodiments, a population of T cells that exhibits high or relatively high clonality is a population of T cells in which the TCR repertoire is less diverse. In some embodiments, T cells are oligoclonal if analysis indicates the presence of several, such as two or three, TCR transcripts or products in a population of T cells. In some embodiments, monoclonality refers to a population of T cells that is of low diversity. In some embodiments, T cells are monoclonal if analysis indicates the presence of a single TCR transcript or product in a population of T cells.

The clonality of the cells in the input composition, such as T cells is, in some examples, determined by clonal sequencing, such as next-generation sequencing, or spectratype analysis. In some aspects, next-generation sequencing methods can be employed, using genomic DNA or cDNA from T cells, to assess the TCR repertoire, including sequences encoding the complementarity-determining region 3 (CDR3). In some embodiments, whole transcriptome sequencing by RNA-seq can be employed. In some embodiments, single-cell sequencing methods can be used.

In some embodiments, clonality, such as polyclonality, can be assessed or determined by spectratype analysis (a measure of the TCR Vβ, Vα, Vγ, or Vδ chain hypervariable region repertoire). Spectratype analysis distinguishes rearranged variable genes of a particular size, not sequence. Thus, it is understood that a single peak could represent a population of T cells expressing any one of a limited number of rearranged TCR variable genes (Vβ, Vα, Vγ, or Vδ) comprising any one of the 4 potential nucleotides (adenine (a), guanine (g), cytosine (c), or thymine (t)) or a combination of the 4 nucleotides at the junctional region. A population of T cells is considered polyclonal when the Vβ spectratype profile for a given TCR Vβ, Vα, Vγ, or Vδ family has multiple peaks, typically 5 or more predominant peaks and in most cases with Gaussian distribution. Polyclonality can also be defined by generation and characterization of antigen-specific clones to an antigen of interest. In the context of a population of T cells, such as of the input composition, monoclonality refers to a population of T cells that has a single specificity as defined by spectratype analysis (a measure of the TCR Vβ, Vα, Vγ, or Vδ chain hypervariable region repertoire). A population of T cells is considered monoclonal (or mono-specific) when the Vβ, Vα, Vγ, and/or Vδ spectratype profile for a given TCR Vβ, Vα, Vγ, and/or Vδ family has a single predominant peak.

In some embodiments, the methods for assessing clonality can include various features of the methods as described in International Publication Nos. WO2012/048341, WO2014/144495, WO2017/053902, WO2016044227, WO2016176322 and WO2012048340 each incorporated by reference in their entirety. In some embodiments, such methods can be used to obtain sequence information about a target polynucleotide of interest within a cell, such as a TCR. The target genes can be obtained from genomic DNA or mRNA of a cell from a sample or population of cells. The sample or population of cells can include immune cells. For example, for target TCR molecules, the genes encoding chains of a TCR can be obtained from genomic DNA or mRNA of immune cells or T cells. In some embodiments, the starting material is RNA from T cells composed of genes that encode for a chain of a TCR.

In some embodiments, the Shannon index is applied to the clonality as a threshold to filter clones (“Shannon-adjusted clonality”), see, Chaara et al. (2018) Front Immunol 9:1038). In some embodiments, the input composition feature is the clonality of the CD4+ cells of the input composition. In some embodiments, the input composition feature is the clonality of the CD8+ cells of the input composition.

In some aspects, among the phenotypes include expression or markers or functions, e.g., antigen-specific functions such as cytokine secretion, that are associated with a less differentiated cell subset or a more differentiated subset. In some embodiments, the phenotypes are those associated with a less differentiated subset, such as one or more of CCR7⁺, CD27⁺ and interleukin-2 (IL-2) production. In some aspects, less differentiated cells, e.g., central memory cells, are longer lived and exhaust less rapidly, thereby increasing persistence and durability. In some embodiments, the phenotypes are those associated with a more differentiated subset, such as one or more of interferon-gamma (IFN-γ) or IL-13 production. In some aspects, more differentiated subsets can also be related to senescence and effector function.

In some embodiments, the phenotype is or includes a phenotype of a memory T cell or memory T cell subset exposed to their cognate antigen. In some embodiments the phenotype is or includes a phenotype of a memory T cell (or one or more markers associated therewith), such as a T_{CM} cell, a T_{EM} cell, or a T_{EMRA} cell, a T_{SCM} cell, or a combination thereof. In particular embodiments, the phenotype is or includes the expression of one or more specific molecules that is a marker for memory and/or memory T cells or subtypes thereof. In some aspects, exemplary phenotypes associated with T_{CM} cells can include one or more of CD45RA⁻, CD62L⁺, CCR7⁺, CD27+, CD28+ and CD95⁺. In some aspects, exemplary phenotypes associated with T_{EM} cells can include one or more of CD45RA⁻, CD62L⁻, CCR7⁻, CD27−, CD28−, and CD95⁺.

In particular embodiments, the phenotype is or includes the expression of one or more specific molecules that is a marker for naïve T cells.

In some embodiments, the phenotype is or includes a memory T cell or a naive T cell. In certain embodiments, the phenotype is the positive or negative expression of one or more specific molecules that are markers for memory. In some embodiments, the memory marker is a specific molecule that may be used to define a memory T cell population.

In some embodiments, the phenotype is or includes a phenotype of or one or more marker associated with a non-memory T cell or sub-type thereof; in some aspects, it is or includes a phenotype or marker(s) associated with a naïve cell. In some aspects, exemplary phenotypes associated with naïve T cells can include one or more of CCR7+, CD45RA+, CD27+, and CD28+. In some embodiments, the phenotype is CCR7⁺/CD27⁺/CD28⁺/CD45RA⁺. In certain embodiments, the phenotype is or includes CCR7⁺/CD45RA⁺. In certain embodiments, the phenotype is or includes CCR7⁺/CD27+. In certain embodiments, the phenotype is or includes CD27+/CD28+. In some embodiments, the phenotype is or includes a phenotype of a central memory T cell. In particular embodiments, the phenotype is or includes CCR7⁺/CD27⁺/CD28⁺/CD45RA⁻. In some embodiments, the phenotype is or includes CCR7⁻/CD27⁺/CD28⁺/CD45RA⁻. In some embodiments, the phenotype is or includes CCR7⁺/CD27⁺. In some embodiments, the phenotype is or includes CD27⁺/CD28⁺. In certain embodiments, the phenotype is or includes that of a T_{EMRA} cell or a T_{SCM} cell. In certain embodiments, the phenotype is or includes CD45RA⁺. In particular embodiments, the phenotype is or includes CCR7⁻/CD27⁻/CD28⁻/CD45RA⁺. In some embodiments, the phenotype is or includes one of CD27⁺/CD28⁺, CD27⁻/CD28⁺, CD27⁺/CD28⁻, or CD27⁻/CD28⁻. In some embodiments, the phenotype is CCR7⁺/CD27⁺/CD45RA⁺. In certain embodiments, the phenotype is or includes CCR7⁺/CD45RA⁺. In certain embodiments, the phenotype is or includes CD27−/CD28−. In particular embodiments, the phenotype is or includes CCR7⁺/CD27⁺/CD45RA⁻. In some embodiments, the phenotype is or includes CCR7⁻/CD27⁺/CD45RA⁻. In certain embodiments, the phenotype is or includes CD45RA⁺. In some embodiments, the phenotype is or includes CCR7⁻/CD27⁻/CD45RA⁺. In some embodiments, the phenotype is or includes CCR7⁺/CD27⁺/CD28⁺/CD45RA⁻; CCR7⁻/CD27⁺/CD28⁺/CD45RA⁻; CCR7⁻/CD27⁻/CD28⁻/CD45RA⁺; CD27⁺/CD28⁺; CD27⁻/CD28⁺; CD27⁺/CD28⁻; or CD27⁻/CD28⁻. In particular embodiments, the phenotype is or includes CCR7⁺/CD27⁺/CD45RA⁻; CCR7⁻/CD27⁺/CD45RA⁻; CCR7⁻/CD27⁻/CD28⁻/CD45RA⁺; CD27⁺; CD27⁻; CD27⁺/CD28⁻; or CD27⁻/CD28⁻.

In some embodiments, the phenotype is or includes a phenotype of or one or more marker associated with a naïve-like T cell. In some embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+/CCR7+, where the cells are CD45RA+ or CD45RA−. In certain embodiments, naïve-like T cells or the T cells that are surface positive for a marker expressed on naïve-like T cells are CD62L-−CCR7+.

In certain embodiments, the phenotype is or includes a phenotype of a T cell that is negative for a marker of apoptosis. In certain embodiments, the phenotype is or includes a naïve cell that is negative for a marker of apoptosis. In some embodiments, the marker of apoptosis is activated caspase 3 (3CAS). In some embodiments, the marker of apoptosis is positive staining by annexin V. In particular embodiments, the phenotype is or includes CD27⁺/CD28⁺, CD27⁻/CD28⁺, CD27⁺/CD28⁻, CD27⁻/CD28⁻, or a combination thereof.

In certain embodiments, the phenotype is or includes activated caspase 3⁻/CD27⁺/CD28⁺, activated caspase 3⁻/CD27⁻/CD28⁺, activated caspase 3⁻/CD27⁺/CD28⁻, activated caspase 3⁻/CD27⁻/CD28⁻, or a combination thereof. In particular embodiments, the phenotype is or includes annexin V⁻/CD27⁺/CD28⁺, annexin V⁻/CD27⁻/CD28⁺, annexin V⁻/CD27⁺/CD28⁻, annexin V⁻/CD27⁻/CD28⁻, or a combination thereof. In particular embodiments, the phenotype is or includes CD27⁺, CD27⁻, CD27⁺, CD27⁻, or a combination thereof. In some embodiments, the phenotype is or includes CD27⁺, CD27⁻, CD27⁺, CD27⁻, or a combination thereof. In certain embodiments, the phenotype is or includes activated caspase 3⁻/CD27⁺, activated caspase 3⁻/CD27⁻, activated caspase 3⁻/CD27⁺, activated caspase 3⁻/CD27⁻, or a combination thereof. In particular embodiments, the phenotype is or includes annexin V⁻/CD27⁺, annexin V⁻/CD27⁻, annexin V⁻/CD27⁺, annexin V⁻/CD27⁻, or a combination thereof.

In particular embodiments, the phenotype is or includes CCR7⁺/CD28⁺, CCR7⁻/CD28⁺, CCR7⁺/CD28⁻, CCR7⁻/CD28⁻, or a combination thereof. In some embodiments, the phenotype is or includes CCR7⁺/CD28⁺, CCR7⁻/CD28⁺, CCR7⁺/CD28⁻, CCR7⁻/CD28⁻, or a combination thereof. In certain embodiments, the phenotype is or includes activated caspase 3⁻/CCR7⁺/CD28⁺, activated caspase 3⁻/CCR7⁻/CD28⁺, activated caspase 3⁻/CCR7⁺/CD28⁻, activated caspase 3⁻/CCR7⁻/CD28⁻, or a combination thereof. In particular embodiments, the phenotype is or includes annexin V⁻/CCR7⁺/CD28⁺, annexin V⁻/CCR7⁻/CD28⁺, annexin V⁻/CCR7⁺/CD28⁻, annexin V⁻/CCR7⁻/CD28⁻, or a combination thereof. In particular embodiments, the phenotype is or includes CCR7⁺, CCR7⁻, CCR7⁺, CCR7⁻, or a combination thereof. In some embodiments, the phenotype is or includes CCR7⁺, CCR7⁻, CCR7⁺, CCR7⁻, or a combination thereof. In certain embodiments, the phenotype is or includes activated caspase 3⁻/CCR7⁺, activated caspase 3⁻/CCR7⁻, activated caspase 3⁻/CCR7⁺, activated caspase 3⁻/CCR7⁻, or a combination thereof. In particular embodiments, the phenotype is or includes annexin V⁻/CCR7⁺, annexin V⁻/CCR7⁻, annexin V⁻/CCR7⁺, annexin V⁻/CCR7⁻, or a combination thereof.

In some embodiments, the input composition features include any one or more or all input composition features, including phenotypes, described herein. In some embodiments, the input composition features include one or more of CAS3−/CCR7−/CD27−, CAS3−/CCR7−/CD27+, CAS3−/CCR7+, CAS3−/CCR7+/CD27−, CAS3−/CCR7+/CD27+, CAS3−/CD27+, CAS3−/CD28−/CD27−, CAS3−/CD28−/CD27+, CAS3−/CD28+, CAS3−/CD28+/CD27−, CAS3−/CD28+/CD27+, CAS3−/CCR7−/CD45RA−, CAS3−/CCR7−, CD45RA+, CAS3−/CCR7+/CD45RA−, CAS3−/CCR7+/CD45RA+, CAS+, CAS+/CD3+, and clonality. In some embodiments, the input composition features include one or more of CAS3−/CCR7−/CD27−/CD4+, CAS3−/CCR7−/CD27+/CD4+, CAS3−/CCR7+/CD4+, CAS3−/CCR7+/CD27−/CD4+, CAS3−/CCR7+/CD27+/CD4+, CAS3−/CD27+/CD4+, CAS3−/CD28−/CD27−/CD4+, CAS3−/CD28−/CD27+/CD4+, CAS3−/CD28+/CD4+, CAS3−/CD28+/CD27−/CD4+, CAS3−/CD28+/CD27+/CD4+, CAS3−/CCR7−/CD45RA−/CD4+, CAS3−/CCR7−/CD4+, CD45RA+/CD4+, CAS3−/CCR7+/CD45RA−/CD4+, CAS3−/CCR7+/CD45RA+/CD4+, CAS+/CD4+, CAS+/CD3+/CD4+, and CD4+ clonality. In some embodiments, the input composition features include one or more of CAS3−/CCR7−/CD27−/CD8+, CAS3−/CCR7−/CD27+/CD8+, CAS3−/CCR7+/CD8+, CAS3−/CCR7+/CD27−/CD8+, CAS3−/CCR7+/CD27+/CD8+, CAS3−/CD27+/CD8+, CAS3−/CD28−/CD27−/CD8+, CAS3−/CD28−/CD27+/CD8+, CAS3−/CD28+/CD8+, CAS3−/CD28+/CD27−/CD8+, CAS3−/CD28+/CD27+/CD8+, CAS3−/CCR7−/CD85RA−/CD8+, CAS3−/CCR7−/CD8+, CD85RA+/CD8+, CAS3−/CCR7+/CD85RA−/CD8+, CAS3−/CCR7+/CD85RA+/CD8+, CAS+/CD8+, CAS+/CD3+/CD8+, and CD8+ clonality. In some embodiments, the input composition features include one or more of CAS3−/CCR7−/CD27−/CD4+, CAS3−/CCR7−/CD27+/CD4+, CAS3−/CCR7+/CD4+, CAS3−/CCR7+/CD27−/CD4+, CAS3−/CCR7+/CD27+/CD4+, CAS3−/CD27+/CD4+, CAS3−/CD28−/CD27−/CD4+, CAS3−/CD28−/CD27+/CD4+, CAS3−/CD28+/CD4+, CAS3−/CD28+/CD27−/CD4+, CAS3−/CD28+/CD27+/CD4+, CAS3−/CCR7−/CD45RA−/CD4+, CAS3−/CCR7−/CD4+, CD45RA+/CD4+, CAS3−/CCR7+/CD45RA−/CD4+, CAS3−/CCR7+/CD45RA+/CD4+, CAS+/CD4+, CAS+/CD3+/CD4+, CD4+ clonality, CAS3−/CCR7−/CD27−/CD8+, CAS3−/CCR7−/CD27+/CD8+, CAS3−/CCR7+/CD8+, CAS3−/CCR7+/CD27−/CD8+, CAS3−/CCR7+/CD27+/CD8+, CAS3−/CD27+/CD8+, CAS3−/CD28−/CD27−/CD8+, CAS3−/CD28−/CD27+/CD8+, CAS3−/CD28+/CD8+, CAS3−/CD28+/CD27−/CD8+, CAS3−/CD28+/CD27+/CD8+, CAS3−/CCR7−/CD85RA−/CD8+, CAS3−/CCR7−/CD8+, CD85RA+/CD8+, CAS3−/CCR7+/CD85RA−/CD8+, CAS3−/CCR7+/CD85RA+/CD8+, CAS+/CD8+, CAS+/CD3+/CD8+, and CD8+ clonality.

In some embodiments, the input composition features include any one or more of the input composition features shown in Table E4 below. In some of any of the above embodiments, the percentage, number, and/or proportion of cells having a phenotype as described above, is determined, measured, obtained, detected, observed, and/or identified. In some embodiments, the number of cells of the phenotype is the total amount of cells of the phenotype of the input composition. In some embodiments, the number of the cells of the phenotype may be expressed as a frequency, ratio, and/or a percentage of cells of the phenotype present in the input composition. In some embodiments, the input composition feature is a frequency, ratio, and/or a percentage of cells having the phenotype described herein.

3. Therapeutic Cell Composition Features

In some embodiments, a therapeutic cell composition is generated (e.g., as described herein) from an input composition, for example as described above. In some embodiments, the therapeutic cell composition is a therapeutic T cell composition. In some embodiments, the therapeutic cell composition contains engineered CD4+ T cells. In some embodiments, the therapeutic cell composition contains engineered CD8+ T cells. In some embodiments, the therapeutic cell composition contains engineered CD4+ and CD8+ T cells. In some embodiments, the engineered T cells, e.g., CD4+ and/or CD8+ engineered T cells, of the therapeutic cell composition express recombinant receptors, such as recombinant T cell receptors (TCR) or chimeric antigen receptors (CAR). In some embodiments, the recombinant receptor, e.g., TCR or CAR, binds to an antigen associated with a disease or condition. For example, an antigen associated with a disease or condition may be an antigen expressed on a cell or tissue of a disease or condition. In some embodiments, the recombinant receptor specifically binds to an antigen associated with the disease or condition or expressed in cells of the environment of a lesion associated with the disease or condition. In some embodiments, the antigen is associated with and/or involved in the etiology of a disease condition or disorder, e.g., causes, exacerbates or otherwise is involved in such disease, condition, or disorder. Exemplary diseases and conditions can include diseases or conditions associated with malignancy or transformation of cells (e.g., cancer), autoimmune or inflammatory disease, or an infectious disease, e.g., caused by a bacterial, viral or other pathogen.

In some embodiments, the therapeutic cell composition, e.g., therapeutic T cell composition is for treating a disease or condition.

In some embodiments, therapeutic cell composition features include cell phenotypes. In some embodiments, the phenotype is the number of total T cells. In some embodiments, the phenotype is the number of total CD3⁺ T cells. In particular embodiments, phenotype includes cells that express a recombinant receptor or a CAR. In some embodiments, the recombinant or CAR binds to an antigen associated with a disease or condition. In some embodiments, the phenotype includes one or more different subtypes of T cells. In some embodiments, the one or more different subtypes further express a recombinant receptor or a CAR. In some embodiments, the phenotype is or includes the identity of a T cell subtype. Different populations or subtypes of T cells include, but are not limited to effector T cells, helper T cells, memory T cell, effector memory T cells, Regulatory T cells, naïve T cells, naïve-like T cells, CD4⁺ cells, and CD8⁺ T cells. In certain embodiments, a T cell sub-type may be identified by detecting the presence or absence of a specific molecule. In certain embodiments, the specific molecule is a surface marker that can be used to identify a T cell subtype.

In some embodiments, the phenotype is positive or high level expression of one or more specific molecule that are surface markers, e.g., CD3, CD4, CD8, CD28, CD62L, CCR7, CD27, CD127, CD4, CD8, CD45RA, and/or CD45RO. In certain embodiments, the phenotype is a surface marker of T cells or of a subpopulation or subset of T cells, such as based on positive surface marker expression of one or more surface markers, e.g., CD3⁺, CD4⁺, CD8⁺, CD28⁺, CD62L⁺, CCR7⁺, CD27⁺, CD127⁺, CD4⁺, CD8⁺, CD45RA⁺, and/or CD45RO⁺. In some embodiments, the phenotype is positive or high level expression of one or more specific molecules that are surface markers, e.g., C-C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin. In some embodiments, the phenotype is negative or the absence of expression of one or more specific molecule that are surface markers, e.g., CD3, CD4, CD8, CD28, CD62L, CCR7, CD27, CD127, CD45RA, and/or CD45RO. In certain embodiments, the phenotype is a surface marker of T cells or of a subpopulation or subset of T cells, such as based on the absence of surface marker expression of one or more surface markers, e.g., CD3⁻, CD4⁻, CD8−, CD28⁻, CD62L⁻, CCR7⁻, CD27⁻, CD127⁻, CD4⁻, CD8⁻, CD45RA⁻, and/or CD45RO⁻. In some embodiments, the phenotype is negative or the absence of expression of one or more specific molecule that are surface markers, e.g., C-C chemokine receptor type 7 (CCR7), Cluster of Differentiation 27 (CD27), Cluster of Differentiation 28 (CD28), and Cluster of Differentiation 45 RA (CD45RA). In certain embodiments, the phenotype markers include CCR7, CD27, CD28, CD44, CD45RA, CD62L, and L-selectin.

In certain embodiments, the phenotype is or includes positive or negative expression of CD27, CCR7 and/or CD45RA. In some embodiments, the phenotype is CCR7⁺. In some embodiments, the phenotype is CD27⁺. In some embodiments, the phenotype is CCR7⁻. In some embodiments, the phenotype is CD27⁻. In some embodiments, the phenotype is CCR7⁺/CD27⁺. In some embodiments, the phenotype is CCR7⁻/CD27⁺. In some embodiments, the phenotype is CCR7⁺/CD27−. In some embodiments, the phenotype is CCR7⁻/CD27⁻. In some embodiments, the phenotype is CD45RA⁻. In some embodiments, the phenotype is CD45RA⁺. In some embodiments, the phenotype is CCR7⁺/CD45RA⁻. In some embodiments, the phenotype is CD27⁺/CD45RA⁺. In some embodiments, the phenotype is CD27⁻/CD45RA⁺. In some embodiments, the phenotype is CD27⁻/CD45RA⁻. In some embodiments, the phenotype is CD27⁺/CD45RA⁻. In some embodiments, the phenotype is CCR7⁺/CD27⁺/CD45RA⁻. In some embodiments, the phenotype is CCR7⁺/CD27⁺/CD45RA+.

In certain embodiments, the surface marker indicates expression of a recombinant receptor, e.g., a CAR. In particular embodiments, the surface marker is expression of the recombinant receptor, e.g., CAR, which, in some aspects, can be determined using an antibody, such as an anti-idiotype antibody. In some embodiments, the surface marker that indicates expression of the recombinant receptor is a surrogate marker. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A (e.g., SEQ ID NOS: 1 and 4), a P2A (e.g., SEQ ID NOS: 5 and 6), a E2A (e.g., SEQ ID NO: 7) or a F2A (e.g., SEQ ID NO: 8). Extrinsic marker genes may in some cases be utilized in connection with engineered cells to permit detection or selection of cells and, in some cases, also to promote cell suicide.

Exemplary surrogate markers can include truncated cell surface polypeptides, such as a truncated human epidermal growth factor receptor 2 (tHER2), a truncated epidermal growth factor receptor (EGFRt, exemplary EGFRt sequence set forth in SEQ ID NO:2 or 3) or a prostate-specific membrane antigen (PSMA) or modified form thereof. EGFRt may contain an epitope recognized by the antibody cetuximab (Erbitux®) or other therapeutic anti-EGFR antibody or binding molecule, which can be used to identify or select cells that have been engineered with the EGFRt construct and a recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434). In some aspects, the marker, e.g., surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR, EGFRt) that is, optionally, linked to a linker sequence, such as a T2A cleavable linker sequence. An exemplary polypeptide for a truncated EGFR (e.g., tEGFR, EGFRt) comprises the sequence of amino acids set forth in SEQ ID NO: 2 or 3 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 2 or 3. In some embodiments, the phenotype is EGFRt+.

In some embodiments, the marker is or comprises a fluorescent protein, such as green fluorescent protein (GFP), enhanced green fluorescent protein (EGFP), such as super-fold GFP, red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT),β-glucuronidase (GUS) or variants thereof.

In certain embodiments, the phenotype comprises expression, e.g., surface expression, of one or more of the surface markers CD3, CD4, CD8, and/or a recombinant receptor (e.g., CAR) or its surrogate marker indicating or correlating to expression of a recombinant receptor (e.g., CAR). In some embodiments, the surrogate marker is EGFRt.

In particular embodiments, the phenotype is identified by the expression of one or more specific molecules that are surface markers. In certain embodiments, the phenotype is or includes positive or negative expression of CD3, CD4, CD8, and/or a recombinant receptor, e.g., a CAR. In certain embodiments, the recombinant receptor is a CAR. In particular embodiments the phenotype comprises CD3⁺/CAR⁺, CD4⁺/CAR⁺, and/or CD8⁺/CAR⁺.

In certain embodiments, the phenotype is or includes positive or negative expression of CD27, CCR7 and/or CD45RA, and/or a recombinant receptor, e.g., a CAR. In some embodiments, the phenotype is CCR7⁺/CAR⁺. In some embodiments, the phenotype is CD27⁺/CAR⁺. In some embodiments, the phenotype is CCR7⁺/CD27⁺/CAR⁺. In some embodiments, the phenotype is CD45RA⁻/CAR⁺. In some embodiments, the phenotype is CCR7⁺/CD45RA⁻/CAR⁺. In some embodiments, the phenotype is CD27⁺/CD45RA⁻/CAR⁺. In some embodiments, the phenotype is CCR7⁺/CD27⁺/CD45RA⁻/CAR⁺. In some embodiments, the phenotype is CCR7−/CD27−/CAR+. In some embodiments, the phenotype is CCR7−/CD27+/CAR+. In some embodiments, the phenotype is CCR7+/CD27−/CAR+. In some embodiments, the phenotype is CD28−/CD27−/CAR+. In some embodiments, the phenotype is CD28−/CD27+/CAR+. In some embodiments, the phenotype is CD28+/CAR+. In some embodiments, the phenotype is CD28+/CD27−/CAR+. In some embodiments, the phenotype is CD28+/CD27+/CAR+. In some embodiments, the phenotype is CCR7−/CD45RA−/CAR+. In some embodiments, the phenotype is CCR7−/CD45RA+/CAR+. In some embodiments, the phenotype is CCR7+/CD45RA−/CAR+. In some embodiments, the phenotype is CCR7+/CD45RA+/CAR+. In some embodiments, the phenotype further is CD4+. In some embodiments, the phenotype further is CD8+.

In some embodiments, the phenotype is viability. In certain embodiments, the phenotype is the positive expression of a marker that indicates that the cell undergoes normal functional cellular processes and/or has not undergone or is not under the process of undergoing necrosis or programmed cell death. In some embodiments, viability can be assessed by the redox potential of the cell, the integrity of the cell membrane, or the activity or function of mitochondria. In some embodiments, viability is the absence of a specific molecule associated with cell death, or the absence of the indication of cell death in an assay. In some embodiments, the phenotype is viable cell concentration.

In some embodiments, the phenotype is or comprises cell viability. In certain embodiments, the viability of cells can be detected, measured, and/or assessed by a number of means that are routine in the art. Non-limiting examples of such viability assays include, but are not limited to, dye uptake assays (e.g., calcein AM assays), XTT cell viability assays, and dye exclusion assays (e.g., trypan blue, Eosin, or propidium dye exclusion assays). Viability assays are useful for determining the number or percentage (e.g., frequency) of viable cells in a cell dose, a cell composition, and/or a cell sample. In particular embodiments, the phenotype comprises cell viability along with other features, e.g., recombinant receptor expression. In some embodiments, the phenotype is or includes soluble CD137 (sCD137, 4-IBB). In some embodiments, sCD137 indicates activation induced cell death. In some embodiments, sCD137 is detected in supernatant.

In certain embodiments, the phenotype is or includes cell viability, viable CD3⁺, viable CD4⁺, viable CD8⁺, viable CD3⁺/CAR⁺, viable CD4⁺/CAR⁺, viable CD8⁺/CAR⁺, viable CD4⁺/CCR7⁺/CAR⁺, viable CD8⁺/CD27⁺/CAR⁺, viable CD4⁺/CD27⁺/CAR⁺, viable CD8⁺/CCR7⁺/CD27⁺/CAR⁺, viable CD4⁺/CCR7⁺/CD27⁺/CAR⁺, viable CD8⁺/CCR7⁺/CD45RA⁻/CAR⁺ or viable CD4⁺/CCR7⁺/CD45RA⁻ or a combination thereof.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death. These changes include blebbing, cell shrinkage, nuclear fragmentation, chromatin condensation, chromosomal DNA fragmentation, and global mRNA decay. Apoptosis is a well characterized process, and specific molecules associated with various stages are well known in the art.

In some embodiments, the phenotype is the absence of an early stage of apoptosis, and/or an absence of an indicator and/or a specific molecule associated with an early stage of apoptosis. In the early stages of apoptosis, changes in the cellular and mitochondrial membrane become apparent. Biochemical changes are also apparent in the cytoplasm and nucleus of the cell. For example, the early stages of apoptosis can be indicated by activation of certain caspases, e.g., 2, 8, 9, and 10. In particular embodiments, the phenotype is the absence of a late stage of apoptosis, and/or an absence of an indicator and/or a specific molecule associated with a late stage of apoptosis. The middle to late stages of apoptosis are characterized by further loss of membrane integrity, chromatin condensation and DNA fragmentation, and include biochemical events such as activation of caspases 3, 6, and 7.

In certain embodiments, the phenotype is the negative expression of one or more factors associated with apoptosis, including pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In some embodiments, the phenotype is a negative or low amount of a marker of apoptosis. In certain embodiments, the phenotype is the negative expression of a marker of apoptosis. In certain embodiments, the phenotype is the absence of an indicator, e.g., an Annexin V molecule, which will preferentially bind to cells undergoing apoptosis when incubated with or contacted to a cell composition. In some embodiments, the phenotype is or includes the expression of one or more markers that are indicative of an apoptotic state in the cell.

In some embodiments, the phenotype is the negative (or low) expression of a specific molecule that is a marker for apoptosis. Various apoptosis markers are known to those of ordinary skill in the art and include, but are not limited to, an increase in activity of one or more caspases i.e. an activated caspase (e.g., an active caspase), an increase in PARP cleavage, activation and/or translocation of Bcl-2 family proteins, members of the cell death pathway, e.g., Fas and FADD, presence of nuclear shrinkage (e.g., monitored by microscope) and presence of chromosome DNA fragmentation (e.g., presence of chromosome DNA ladder) or with apoptosis assays that include TUNEL staining, and Annexin V staining.

Caspases are enzymes that cleave proteins after an aspartic acid residue, the term is derived from “cysteine-aspartic acid proteases.” Caspases are involved in apoptosis, thus activation of caspases, such as caspase-3 is indicative of an increase or revival of apoptosis. In certain embodiments, caspase activation can be detected by methods known to the person of ordinary skill. In some embodiments, an antibody that binds specifically to an activated caspase (i.e., binds specifically to the cleaved polypeptide) can be used to detect caspase activation. In another example, a fluorochrome inhibitor of caspase activity (FLICA) assay can be utilized to detect caspase-3 activation by detecting hydrolysis of acetyl Asp-Glu-Val-Asp 7-amido-4-methylcoumarin (Ac-DEVD-AMC) by caspase-3 (i.e., detecting release of the fluorescent 7-amino-4-methylcoumarin (AMC)). FLICA assays can be used to determine caspase activation by a detecting the product of a substrate processed by multiple caspases (e.g., FAM-VAD-FMK FLICA). Other techniques include The CASPASE-GLO® caspase assays (PROMEGA) that use luminogenic caspase-8 tetrapeptide substrate (Z-LETD-aminoluciferin), the caspase-9 tetrapeptide substrate (Z-LEHD-aminoluciferin), the caspase-3/7 substrate (Z-DEVD-aminoluciferin), the caspase-6 substrate (Z-VEID-aminoluciferin), or the caspase-2 substrate (Z-VDVAD-aminoluciferin).

In certain embodiments, the phenotype is or includes negative expression of activated caspase-1, activated caspase-2, activated caspase-3, activated caspase-7, activated caspase-8, activated caspase-9, activated caspase-10 and/or activated caspase-13 in a cell. In particular embodiments, the phenotype is or includes activated caspase 3⁻. In some embodiments, the proform (zymogen cleaved) form of a caspase, such as any above, also is a marker indicating the presence of apoptosis. In some embodiments, the phenotype is or includes the absence of or negative expression of a proform of a caspase, such as the proform of caspase-3.

In some embodiments, the marker of apoptosis is cleaved the Poly ADP-ribose polymerase 1 (PARP). PARP is cleaved by caspase during early stages of apoptosis. Thus, detection of a cleaved PARP peptide is a marker for apoptosis. In particular embodiments, the phenotype is or includes positive or negative expression of cleaved PARP.

In some embodiments, the marker of apoptosis is a reagent that detects a feature in a cell that is associated with apoptosis. In certain embodiments, the reagent is an annexin V molecule. During the early stages of apoptosis the lipid phosphatidylserine (PS) translocates from the inner to the outer leaflet of the plasma membrane. PS is normally restricted to the internal membrane in healthy and/or non-apoptotic cells. Annexin V is a protein that preferentially binds phosphatidylserine (PS) with high affinity. When conjugated to a fluorescent tag or other reporter, Annexin V can be used to rapidly detect this early cell surface indicator of apoptosis. In some embodiments, the presence of PS on the outer membrane will persist into the late stages of apoptosis. Thus in some embodiments, annexin V staining is an indication of both early and late stages of apoptosis. In certain embodiments, an Annexin, e.g., Annexin V, is tagged with a detectable label and incubated with, exposed to, and/or contacted with cells of a cell composition to detect cells that are undergoing apoptosis, for example by flow cytometry. In some embodiments, fluorescence tagged annexins, e.g., annexin V, are used to stain cells for flow cytometry analysis, for example with the annexin⁻V/7⁻AAD assay. Alternative protocols suitable for apoptosis detection with annexin include techniques and assays that utilize radiolabeled annexin V. In certain embodiments, the phenotype is or includes negative staining by annexin, e.g., annexin V⁻. In particular embodiments, the phenotype is or includes the absence of PS on the outer plasma membrane. In certain embodiments, the phenotype is or includes cells that are not bound by annexin e.g., annexin V. In certain embodiments, the cell that lacks detectable PS on the outer membrane is annexin V⁻. In particular embodiments, the cell that is not bound by annexin V⁻ in an assay, e.g., flow cytometry after incubation with labeled annexin V, is annexin V⁻.

In particular embodiments, the phenotype is annexin V⁻, annexin V⁻CD3⁺, annexin V⁻CD4⁺, annexin V⁻CD8⁺, annexin V⁻CD3⁺/CAR⁺, annexin V⁻CD4⁺/CAR⁺, annexin V⁻CD8⁺/CAR⁺, activated caspase 3⁻, activated caspase 3⁻/CD3⁺, activated caspase 3⁻/CD4⁺, activated caspase 3⁻ /CD8⁺, activated caspase 3⁻/CD3⁺/CAR⁺, activated caspase 3⁻/CD4⁺/CAR⁺, activated caspase 3⁻/CD8⁺/CAR⁺, annexin V⁻/CD4⁺/CCR7⁺/CAR⁺, annexin V⁻/CD8⁺/CD27⁺/CAR⁺, annexin V−/CD4⁺/CD27⁺/CAR⁺, annexin V⁻/CD8⁺/CCR7⁺/CD27⁺/CAR⁺, annexin V−/CD4⁺/CCR7⁺/CD27⁺/CAR⁺, annexin V⁻/CD8⁺/CCR7⁺/CD45RA⁻/CAR⁺ or annexin V−/CD4⁺/CCR7⁺/CD45RA⁻; activated caspase 3⁻/CD4⁺/CCR7⁺/CAR⁺, activated caspase 3−/CD8⁺/CD27⁺/CAR⁺, activated caspase 3⁻/CD4⁺/CD27⁺/CAR⁺, activated caspase 3⁻/CD8⁺/CCR7⁺/CD27⁺/CAR⁺, activated caspase 3⁻/CD4⁺/CCR7⁺/CD27⁺/CAR⁺, activated caspase 3⁻/CD8⁺/CCR7⁺/CD45RA⁻/CAR⁺ or activated caspase 3⁻/CD4⁺/CCR7⁺/CD45RA⁻ or a combination thereof. In some embodiments, the phenotype is 3CAS−/CCR7−/CD27−/CAR+. In some embodiments, the phenotype is 3CAS−/CCR7−/CD27+/CAR+. In some embodiments, the phenotype is 3CAS−/CCR7+/CAR+. In some embodiments, the phenotype is 3CAS−/CCR7+/CD27−/CAR+. In some embodiments, the phenotype is 3CAS−/CCR7+/CD27+/CAR+. In some embodiments, the phenotype is 3CAS−/CD27+/CAR+. In some embodiments, the phenotype is 3CAS−/CD28−/CD27−/CAR+. In some embodiments, the phenotype is 3CAS−/CD28−/CD27+/CAR+. In some embodiments, the phenotype is 3CAS−/CD28+/CAR+. In some embodiments, the phenotype is 3CAS−/CD28+/CD27−/CAR+. In some embodiments, the phenotype is 3CAS−/CD28+/CD27+/CAR+. In some embodiments, the phenotype is 3CAS−/CCR7−/CD45RA−/CAR+. In some embodiments, the phenotype is 3CAS−/CCR7−/CD45RA+/CAR+. In some embodiments, the phenotype is 3CAS−/CCR7+/CD45RA−/CAR+. In some embodiments, the phenotype is 3CAS−/CCR7+/CD45RA+/CAR+. In some embodiments, the phenotype further is CD4+. In some embodiments, the phenotype further is CD8+.

Particular embodiments contemplate that cells positive for expression of a marker for apoptosis are undergoing programmed cell death, show reduced or no immune function, and have diminished capabilities if any to undergo activation, expansion, and/or bind to an antigen to initiate, perform, or contribute to an immune response or activity. In particular embodiments, the phenotype is defined by negative expression for an activated caspase and/or negative staining with annexin V.

In certain embodiments, the phenotype is or includes activated caspase 3⁻ (3CAS−, caspase 3⁻)and/or annexin V⁻.

Among the phenotypes are the expression or surface expression of one or more markers generally associated with one or more sub-types or subpopulations of T cells, or phenotypes thereof. T cell subtypes and subpopulations may include CD4⁺ and/or of CD8⁺ T cells and subtypes thereof that may include naïve T (T_{N}) cells, naïve-like T cells, effector T cells (T_{EFF}), memory T cells and sub-types thereof, such as stem cell memory T (T_{SCM}), central memory T (T_{CM}), effector memory T (T_{EM}), T_{EMRA} cells or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some aspects, among the phenotypes include expression or markers or functions, e.g., antigen-specific functions such as cytokine secretion, that are associated with a less differentiated cell subset or a more differentiated subset. In some embodiments, the phenotypes are those associated with a less differentiated subset, such as one or more of CCR7⁺, CD27⁺ and interleukin-2 (IL-2) production. In some aspects, less differentiated subsets can also be related to therapeutic efficacy, self-renewal, survival functions or graft-versus-host disease. In some embodiments, the phenotypes are those associated with a more differentiated subset, such as one or more of interferon-gamma (IFN-γ) or IL-13 production. In some aspects, more differentiated subsets can also be related to senescence and effector function.

In some embodiments, the phenotype is or includes a phenotype of a memory T cell or memory T cell subset exposed to their cognate antigen. In some embodiments the phenotype is or includes a phenotype of a memory T cell (or one or more markers associated therewith), such as a T_{CM} cell, a T_{EM} cell, or a T_{EMRA} cell, a T_{SCM} cell, or a combination thereof. In particular embodiments, the phenotype is or includes the expression of one or more specific molecules that is a marker for memory and/or memory T cells or subtypes thereof. In some aspects, exemplary phenotypes associated with T_{CM} cells can include one or more of CD45RA⁻, CD62L⁺, CCR7⁺, CD27+, CD28+, and CD95⁺. In some aspects, exemplary phenotypes associated with T_{EM} cells can include one or more of CD45RA⁻, CD62L⁻, CCR7⁻, CD27−, CD28−, and CD95⁺.

In particular embodiments, the phenotype is or includes the expression of one or more specific molecules that is a marker for naïve T cells.

In some embodiments, the phenotype is or includes a memory T cell or a naive T cell. In certain embodiments, the phenotype is the positive or negative expression of one or more specific molecules that are markers for memory. In some embodiments, the memory marker is a specific molecule that may be used to define a memory T cell population.

In some embodiments, the phenotype is or includes a phenotype of or one or more marker associated with a naïve-like T cell. In certain embodiments, naïve-like T cells may include cells in various differentiation states and may be characterized by positive or high expression (e.g., surface expression or intracellular expression) of certain cell markers and/or negative or low expression (e.g., surface expression or intracellular expression) of other cell markers. In some aspects, naïve-like T cells are characterized by positive or high expression of CCR7, CD45RA, CD28, and/or CD27. In some aspects, naïve-like T cells are characterized by negative expression of CD25, CD45RO, CD56, CD62L, and/or KLRG1. In some aspects, naïve-like T cells are characterized by low expression of CD95. In certain embodiments, naïve-like T cells or the T cells that are surface positive for a marker expressed on naïve-like T cells are CCR7+CD45RA+, where the cells are CD27+ or CD27−. In certain embodiments, naïve-like T cells or the T cells that are surface positive for a marker expressed on naïve-like T cells are CD27+CCR7+, where the cells are CD45RA+ or CD45RA−. In certain embodiments, naïve-like T cells or the T cells that are surface positive for a marker expressed on naïve-like T cells are CD62L-−CCR7+.

In some embodiments, the phenotype is or includes a phenotype of or one or more marker associated with a non-memory T cell or sub-type thereof; in some aspects, it is or includes a phenotype or marker(s) associated with a naïve cell. In some aspects, exemplary phenotypes associated with naïve T cells can include one or more of CCR7+, CD45RA+, CD27+, and CD28+. In some embodiments, the phenotype is CCR7⁺/CD27⁺/CD28⁺/CD45RA⁺. In certain embodiments, the phenotype is or includes CCR7⁺/CD45RA⁺. In certain embodiments, the phenotype is or includes CCR7⁺/CD27+. In certain embodiments, the phenotype is or includes CD27+/CD28+. In some embodiments, the phenotype is or includes a phenotype of a central memory T cell. In particular embodiments, the phenotype is or includes CCR7⁺/CD27⁺/CD28⁺/CD45RA⁻. In some embodiments, the phenotype is or includes CCR7⁻/CD27⁺/CD28⁺/CD45RA⁻. In some embodiments, the phenotype is or includes CCR7⁺/CD27⁺. In some embodiments, the phenotype is or includes CD27⁺/CD28⁺. In certain embodiments, the phenotype is or includes that of a T_{EMRA} cell or a T_{SCM} cell. In certain embodiments, the phenotype is or includes CD45RA⁺. In particular embodiments, the phenotype is or includes CCR7⁻/CD27⁻/CD28⁻/CD45RA⁺. In some embodiments, the phenotype is or includes one of CD27⁺/CD28⁺, CD27⁻/CD28⁺, CD27⁺/CD28⁻, or CD27⁻/CD28⁻. In some embodiments, the phenotype is CCR7⁺/CD27⁺/CD45RA⁺. In certain embodiments, the phenotype is or includes CCR7⁺/CD45RA⁺. In certain embodiments, the phenotype is or includes CD27−/CD28−. In particular embodiments, the phenotype is or includes CCR7⁺/CD27⁺/CD45RA⁻. In some embodiments, the phenotype is or includes CCR7⁻/CD27⁺/CD45RA⁻. In certain embodiments, the phenotype is or includes CD45RA⁺. In particular embodiments, the phenotype is or includes CCR7⁻/CD27⁻/CD45RA⁺.

In some embodiments the phenotype is or includes any of the foregoing phenotypic properties and further includes the expression of a recombinant receptor, such as phenotype associated with a memory T cell or memory subtype and that expresses a CAR, or a phenotype associated with a naïve cell that expresses a CAR. In certain embodiments, the phenotype is or includes that of a central memory T cell or stem central memory T cell that expresses a CAR. In particular embodiments, the phenotype is or includes that of an effector memory cell that expresses a CAR. In some embodiments, the phenotype is or includes that of a T_{EMRA} cell that expresses a CAR. In particular embodiments, the phenotype is or includes CAR⁺/CCR7⁺/CD27⁺/CD28⁺/CD45RA⁻; CAR⁺/CCR7⁻/CD27⁺/CD28⁺/CD45RA⁻; CAR⁺/CCR7⁻/CD27⁻/CD28⁻/CD45RA⁺; CAR⁺/CD27⁺/CD28⁺; CAR⁺/CD27⁻/CD28⁺; CAR⁺/CD27⁺/CD28⁻; or CAR⁺/CD27⁻/CD28⁻. In particular embodiments, the phenotype is or includes CAR⁺/CCR7⁺/CD27⁺/CD45RA⁻; CAR⁺/CCR7⁻/CD27⁺/CD45RA⁻; CAR⁺/CCR7⁻/CD27⁻/CD28⁻/CD45RA⁺; CAR⁺/CD27⁺; CAR⁺/CD27⁻; CAR⁺/CD27⁺/CD28⁻; or CAR⁺/CD27⁻/CD28⁻.

In certain embodiments, the phenotype is or includes a phenotype of a T cell that is negative for a marker of apoptosis. In certain embodiments, the phenotype is or includes a naïve cell that is negative for a marker of apoptosis. In some embodiments, the marker of apoptosis is activated caspase 3 (3CAS). In some embodiments, the marker of apoptosis is positive staining by annexin V.

In particular embodiments, the phenotype is or includes that of a memory T cell or subtype thereof that is negative for a marker of apoptosis that expresses a CAR. In particular embodiments, the phenotype is or includes that of a memory T cell or particular subtype that is negative for a marker of apoptosis that expresses a CAR. In certain embodiments, the phenotype is or includes a naïve cell that is negative for a marker of apoptosis that expresses a CAR. In certain embodiments, the phenotype is or includes that of a central memory T cell or T_{SCM} cell or naïve cell that is negative for a marker of apoptosis that expresses a CAR. In particular embodiments, the phenotype is or includes that of an effector memory cell that is negative for a marker of apoptosis that expresses a CAR. In certain embodiments, the phenotype is or includes annexin V⁻/CAR⁺/CCR7⁺/CD27⁺/CD28⁺/CD45RA⁻; annexin V⁻/CAR⁺/CCR7⁻/CD27⁺/CD28⁺/CD45RA⁻; annexin V⁻/CAR⁺/CCR7⁻/CD27⁻/CD28⁻/CD45RA⁺; annexin V⁻/CAR⁺/CD27⁺/CD28⁺; annexin V⁻/CAR⁺/CD27⁻/CD28⁺; annexin V⁻/CAR⁺/CD27⁺/CD28⁻; or annexin V⁻/CAR⁺/CD27⁻/CD28⁻. In certain embodiments, the phenotype is or includes activated caspase 3⁻/CAR⁺/CCR7⁺/CD27⁺/CD28⁺/CD45RA⁻; activated caspase 3⁻/CAR⁺/CCR7⁻/CD27⁺/CD28⁺/CD45RA⁻; activated caspase 3⁻/CAR⁺/CCR7⁻/CD27⁻/CD28⁻/CD45RA⁺; activated caspase 3⁻/CAR⁺/CD27⁺/CD28⁺; activated caspase 3⁻/CAR⁺/CD27⁻/CD28⁺; activated caspase 3⁻/CAR⁺/CD27⁺/CD28⁻; or activated caspase 3⁻/CAR⁺/CD27⁻/CD28⁻. In certain embodiments, the phenotype is or includes annexin V⁻/CAR⁺/CCR7⁺/CD27⁺/CD45RA⁻; annexin V⁻/CAR⁺/CCR7⁻/CD27⁺/CD45RA⁻; annexin V⁻/CAR⁺/CCR7⁻/CD27⁻/CD45RA⁺; annexin V⁻/CAR⁺/CD27⁺/CD28⁺; annexin V⁻/CAR⁺/CD27⁻/CD28⁺; annexin V⁻/CAR⁺/CD27⁺; or annexin V⁻/CAR⁺/CD27⁻. In certain embodiments, the phenotype is or includes activated caspase 3⁻/CAR⁺/CCR7⁺/CD27⁺/CD45RA⁻; activated caspase 3⁻/CAR⁺/CCR7⁻/CD27⁺/CD45RA⁻; activated caspase 3⁻/CAR⁺/CCR7⁻/CD27⁻/CD45RA⁺; activated caspase 3⁻/CAR⁺/CD27⁺/CD28⁺; activated caspase 3⁻/CAR⁺/CD27⁻/CD28⁺; activated caspase 3⁻/CAR⁺/CD27⁺; or activated caspase 3⁻/CAR⁺/CD27⁻.

In particular embodiments, the phenotype is or includes CD27⁺/CD28⁺, CD27⁻/CD28⁺, CD27⁺/CD28⁻, CD27⁻/CD28⁻, or a combination thereof. In some embodiments, the phenotype is or includes CAR⁺/CD27⁺/CD28⁺, CAR⁺/CD27⁻/CD28⁺, CAR⁺/CD27⁺/CD28⁻, CAR⁺/CD27⁻/CD28⁻, or a combination thereof. In certain embodiments, the phenotype is or includes activated caspase 3⁻/CAR⁺/CD27⁺/CD28⁺, activated caspase 3⁻/CAR⁺/CD27⁻/CD28⁺, activated caspase 3⁻/CAR⁺/CD27⁺/CD28⁻, activated caspase 3⁻/CAR⁺/CD27⁻/CD28⁻, or a combination thereof. In particular embodiments, the phenotype is or includes annexin V⁻/CAR⁺/CD27⁺/CD28⁺, annexin V⁻/CAR⁺/CD27⁻/CD28⁺, annexin V⁻/CAR⁺/CD27⁺/CD28⁻, annexin V⁻/CAR⁺/CD27⁻/CD28⁻, or a combination thereof. In particular embodiments, the phenotype is or includes CD27⁺, CD27⁻, CD27⁺, CD27⁻, or a combination thereof. In some embodiments, the phenotype is or includes CAR⁺/CD27⁺, CAR⁺/CD27⁻, CAR⁺/CD27⁺, CAR⁺/CD27⁻, or a combination thereof. In certain embodiments, the phenotype is or includes activated caspase 3⁻/CAR⁺/CD27⁺, activated caspase 3⁻/CAR⁺/CD27⁻, activated caspase 3⁻/CAR⁺/CD27⁺, activated caspase 3⁻/CAR⁺/CD27⁻, or a combination thereof. In particular embodiments, the phenotype is or includes annexin V⁻/CAR⁺/CD27⁺, annexin V⁻/CAR⁺/CD27⁻, annexin V⁻/CAR⁺/CD27⁺, annexin V⁻/CAR⁺/CD27⁻, or a combination thereof.

In particular embodiments, the phenotype is or includes CCR7⁺/CD28⁺, CCR7⁻/CD28⁺, CCR7⁺/CD28⁻, CCR7⁻/CD28⁻, or a combination thereof. In some embodiments, the phenotype is or includes CAR⁺/CCR7⁺/CD28⁺, CAR⁺/CCR7⁻/CD28⁺, CAR⁺/CCR7⁺/CD28⁻, CAR⁺/CCR7⁻/CD28⁻, or a combination thereof. In certain embodiments, the phenotype is or includes activated caspase 3⁻/CAR⁺/CCR7⁺/CD28⁺, activated caspase 3⁻/CAR⁺/CCR7⁻/CD28⁺, activated caspase 3⁻/CAR⁺/CCR7⁺/CD28⁻, activated caspase 3⁻/CAR⁺/CCR7⁻/CD28⁻, or a combination thereof. In particular embodiments, the phenotype is or includes annexin V⁻/CAR⁺/CCR7⁺/CD28⁺, annexin V⁻/CAR⁺/CCR7⁻/CD28⁺, annexin V⁻/CAR⁺/CCR7⁺/CD28⁻, annexin V⁻/CAR⁺/CCR7⁻/CD28⁻, or a combination thereof. In particular embodiments, the phenotype is or includes CCR7⁺, CCR7⁻, CCR7⁺, CCR7⁻, or a combination thereof. In some embodiments, the phenotype is or includes CAR⁺/CCR7⁺, CAR⁺/CCR7⁻, CAR⁺/CCR7⁺, CAR⁺/CCR7⁻, or a combination thereof. In certain embodiments, the phenotype is or includes activated caspase 3⁻/CAR⁺/CCR7⁺, activated caspase 3⁻/CAR⁺/CCR7⁻, activated caspase 3⁻/CAR⁺/CCR7⁺, activated caspase 3⁻/CAR⁺/CCR7⁻, or a combination thereof. In particular embodiments, the phenotype is or includes annexin V⁻/CAR⁺/CCR7⁺, annexin V⁻/CAR⁺/CCR7⁻, annexin V⁻/CAR⁺/CCR7⁺, annexin V⁻/CAR⁺/CCR7⁻, or a combination thereof.

In some embodiments, the phenotype is assessed by a response to a stimulus, for example a stimulus that triggers, induces, stimulates, or prolongs an immune cell function. In certain embodiments, the cells are incubated in the presence of stimulating conditions or a stimulatory agent, the phenotype is or includes the response to the stimulation. In particular embodiments, the phenotype is or includes the production or secretion of a soluble factor in response to one or more stimulations. In some embodiments, the phenotype is or includes a lack or production or secretion of a soluble factor in response to one or more stimulations. In certain embodiments, the soluble factor is a cytokine. In some embodiments, the cytokine is IL-2. In some embodiments, the cytokine is TNFa. In some embodiments, the cytokine is IL-17. In some embodiments, the cytokine is IL-10. In some embodiments, the cytokine is IFNG. In some embodiments, the cytokine is IL-13. In some embodiments, the cytokine is IL-5. In some embodiments, the cytokine is GMSCF. In some embodiments, the cell does not produce cytokines (cyto−). In some embodiments, the cell phenotype is cytokine negative (Cyto−).

The conditions used for stimulating cells can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the cells are stimulated and the phenotype is determined by whether or not a soluble factor, e.g., a cytokine or a chemokine, is produced or secreted. In some embodiments, the stimulation is nonspecific, i.e., is not an antigen-specific stimulation. In some embodiments, the stimulation comprises PMA and ionomycin. In some embodiments, cells are incubated in the presence of stimulating conditions or a stimulatory agent for about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 48 hours, or for a duration of time between 1 hour and 4 hours, between 1 hour and 12 hours, between 12 hours and 24 hours, or for more than 24 hours.

In some embodiments, the therapeutic cell composition features include recombinant receptor-dependent activity. For example, in some embodiments, the cells of the therapeutic cell composition are stimulated with an agent that is an antigen or an epitope thereof that is specific to the recombinant receptor, or is an antibody or fragment thereof that binds to and/or recognizes the recombinant receptor, or a combination thereof. Particular embodiments contemplate that a recombinant receptor-dependent activity, e.g., a CAR dependent activity, is an activity that occurs in a cell that expresses a recombinant receptor which does not and/or cannot occur in a cell that does not express the recombinant receptor. In some embodiments, the recombinant receptor-dependent activity is an activity that depends on an activity or presence of the recombinant receptor. The recombinant receptor-dependent activity may be any cellular process that is directly or indirectly influenced by the expression and/or presence of the recombinant receptor or by a change in activity, such as receptor stimulation, of the recombinant receptor. In some embodiments, the recombinant receptor-dependent activity may include, but is not limited to cellular processes such as cell division, DNA replication, transcription, protein synthesis, membrane transport, protein translocation, and/or secretion, or it may be an immune cell function, e.g., a cytolytic activity. In certain embodiments, recombinant receptor-dependent activity may be measured by a change in the confirmation of the CAR receptor, the phosphorylation of an intracellular signaling molecule, degradation of a protein, transcription, translation, translocation of a protein, and/or production and secretion of a factor, such as a protein, or growth factor, cytokine.

In some embodiments, the recombinant receptor is a CAR, and the agent is an antigen or an epitope thereof that is specific to the CAR, or is an antibody or fragment thereof that binds to and/or recognizes the CAR, or a combination thereof. In particular embodiments, the cells are stimulated by incubating the cells in the presence of target cells with surface expression of the antigen that is recognized by the CAR. In certain embodiments, the recombinant receptor is a CAR, and the agent is an antibody or an active fragment, variant, or portion thereof that binds to the CAR. In certain embodiments, the antibody or the active fragment, variant, or portion thereof that binds to the CAR is an anti-idiotypic (anti-ID) antibody. In certain embodiments, the recombinant receptor specific agent is a cell, e.g., target cell, that expresses the antigen on its surface. In some embodiments, the recombinant receptor dependent activity is stimulated by an antigen or an epitope thereof that is bound by and/or recognized by (e.g., engages) the recombinant receptor.

In some embodiments, the stimulating conditions or agents include one or more agent, e.g., ligand, which is capable of stimulating or activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to a solid support such as a bead, and/or one or more cytokines. In some embodiments, the one or more agents are PMA and ionomycin.

In certain embodiments, the recombinant receptor-dependent activity, e.g., a CAR dependent activity is a measurement of a factor, e.g., an amount or concentration, or a change in the amount or concentration following stimulation of the cell composition. In certain embodiments, the factor may be a protein, a phosphorylated protein, a cleaved protein, a translocated protein, a protein in an active confirmation, a polynucleotide, an RNA polynucleotide, an mRNA, and/or an shRNA. In certain embodiments, the measurement may include, but is not limited to, an increase or decrease of kinase activity, protease activity, phosphatase activity, cAMP production, ATP metabolism, translocation, e.g., a nuclear localization of a protein, an increase in transcriptional activity, an increase in translational activity, production and/or secretion of a soluble factor, cellular uptake, ubiquitination, and/or protein degradation. In particular embodiments, the factor is a soluble factor that is secreted, such as a hormone, a growth factor, a chemokine, and/or a cytokine.

In some embodiments, the recombinant receptor-dependent activity, e.g., a CAR dependent activity is a response to stimulation. In certain embodiments, the cells are incubated in the presence of stimulating conditions or a stimulatory agent, and the activity is or includes at least one aspect of a response to the stimulation. A response may include, but is not limited to, an intracellular signaling event, such as an increased activity of a receptor molecule, an increased kinase activity of one or more kinases, an increase in the transcription of one or more genes, increased protein synthesis of one or more proteins, and/or an intracellular signaling molecule e.g., an increased kinase activity of a protein. In some embodiments, the response or activity is associated with an immune activity, and may include, but is not limited to, production and/or section of a soluble factor, e.g., a cytokine, an increase in antibody production, and/or an increase in cytolytic activity.

In particular embodiments, the response to a stimulation of a cell composition is assessed by measuring, detecting, or quantifying a response to a stimulus, i.e. at least one activity that is initiated, triggered, supported, prolonged, and/or caused by the stimulus. In certain embodiments, the cells are stimulated and the response to the stimulation is an activity that is specific to cells that express a recombinant receptor. In certain embodiments, the activity is a recombinant receptor specific activity and the activity occurs in cells that express the recombinant receptor, but does not occur, or only minimally occurs, in cells that do not express the receptor. In particular embodiments, the recombinant receptor is a CAR. In some embodiments, the activity is a CAR dependent activity.

The conditions used for stimulating cells, e.g., immune cells or T cells, can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the cells are stimulated and the activity is determined by whether or not a soluble factor, e.g., a cytokine or a chemokine, is produced or secreted.

In some embodiments, the activity is specific to cells that express a recombinant receptor. In some embodiments, an activity that is specific to cells that express a recombinant receptor does not occur in cells that lack expression of the recombinant receptor. In certain embodiments, the recombinant receptor is a CAR, and the activity is a CAR dependent activity. In particular embodiments, the activity is not present in cells that lack expression of the recombinant receptor under the same conditions where the activity is present in cells that express the recombinant receptor. In certain embodiments, the CAR dependent activity is about 10%, about 20%, about 30%, about 40%, about 50%, about 60% about 70%, about 75%, about 80%, about 85%, about 90%, about 95%, about 97%, about 98%, about 99%, or about 99% less than the CAR dependent activity in CAR− cells under the same conditions.

In some embodiments, the activity is specific to cells that express a recombinant receptor, e.g., a CAR, and the activity is produced by stimulation with an agent or under stimulatory conditions that are specific to cells that express the recombinant receptor. In some embodiments, the recombinant receptor is a CAR, and a CAR specific stimulation stimulates, triggers, initiates, and/or prolongs an activity in CAR+ cells, but does not stimulate, trigger, initiate, and/or prolong the activity in CAR− cells. In some embodiments, the CAR dependent activity is about 10%, about 20%, about 30%, about 40%, about 50%, about 60% about 70%, about 75%, about 80%, about 85%, about 90%, about 95%, about 97%, about 98%, about 99%, or about 99% less in CAR− cells than in the CAR+ cells following stimulation by the CAR specific stimulus.

In some embodiments, the activity is measured in the cell composition containing cells expressing a recombinant receptor, e.g., a CAR, and the measurement is compared to one or more controls. In certain embodiments, the control is a similar or identical composition of cells that was not stimulated. For example, in some embodiments, the activity is measured in a cell composition following or during incubation with an agent, and the resulting measurement is compared to a control measurement of the activity from the similar or identical cell composition that is not incubated with the agent. In some embodiments, the activity is a recombinant receptor-dependent activity, and both the cell composition and the control cell composition contain cells that express the recombinant receptor. In some embodiments, the activity is a recombinant receptor-dependent activity, and the control is taken from a similar cell composition that does not contain cells that express the recombinant receptor, e.g., CAR+ cells. Thus in some embodiments, a cell composition that contains recombinant receptor expressing cells and a control cell composition that does not contain recombinant receptor expressing cells are contacted with a recombinant receptor expressing specific agent. In certain embodiments, the control is a measurement from the same cell composition that expresses a recombinant receptor that is taken prior to any stimulation. In certain embodiments, a control measurement is obtained to determine a background signal, and control measurement is subtracted from the measurement of the activity. In some embodiments, the measurement of the activity in the cell composition is divided by the control measurement, to obtain a value that is a ratio of the activity over a control level.

In particular embodiments, the activity is or includes the production and/or secretion of a soluble factor. In some embodiments, the activity is a recombinant receptor, e.g., a CAR, dependent activity that is or includes the production and/or secretion of a soluble factor. In certain embodiments, the soluble factor is a cytokine or a chemokine.

In particular embodiments, the measurement of the soluble factor is measured by ELISA (enzyme-linked immunosorbent assay). ELISA is a plate-based assay technique designed for detecting and quantifying substances such as peptides, cytokines, antibodies and hormones. In an ELISA, the soluble factor must be immobilized to a solid surface and then complexed with an antibody that is linked to an enzyme. Detection is accomplished by assessing the conjugated enzyme activity via incubation with a substrate to produce a detectable signal. In some embodiments, the CAR dependent activity is measured with an ELISA assay.

In some embodiments, the recombinant receptor-dependent activity is a secretion or production of the soluble factor. In certain embodiments, production or secretion is stimulated in a cell composition that contains recombinant receptor expressing cells, e.g., CAR expressing cells, by a recombinant receptor specific agent, e.g., a CAR+ specific agent. In some embodiments, the recombinant receptor specific agent that is an antigen or an epitope thereof that is specific to the recombinant receptor; a cell, e.g., a target cell, that expresses the antigen; or an antibody or a portion or variant thereof that binds to and/or recognizes the recombinant receptor; or a combination thereof. In certain embodiments, the recombinant receptor specific agent is a recombinant protein that comprises the antigen or epitope thereof that is bound by or recognized by the recombinant receptor.

In certain embodiments, the recombinant receptor dependent soluble factor production and/or secretion is measured by incubating the cell composition that contains cells expressing the recombinant receptor, e.g., a CAR, with a recombinant receptor specific agent, e.g., CAR+ specific agent. In certain embodiments, the soluble factor is a cytokine or a chemokine. In some embodiments, cells of the cell composition that contain recombinant receptor expressing cells are incubated in the presence of recombinant receptor specific agent for an amount of time, and the production and/or secretion of the soluble factor is measured at one or more time points during the incubation. In some embodiments, the cells are incubated with the CAR specific agent for up to or about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 19 hours, about 20 hours, about 21 hours, about 22 hours, about 23 hours, about 24 hours, about 48 hours, or for a duration of time between 1 hour and 4 hours, between 1 hour and 12 hours, between 12 hours and 24 hours, each inclusive, or for more than 24 hours and the amount of a soluble factor, e.g., a cytokine is detected.

In some embodiments, the recombinant receptor specific agent is a target cell that expresses an antigen recognized by the recombinant receptor. In some embodiments, the recombinant receptor is a CAR, and the cells of the cell composition are incubated with the target cells at ratio of total cells, CAR+ cells, CAR+/CD8+ cells, or Annexin−/CAR+/CD8+ cells of the cell composition to target cells of about 10:1, about 5:1, about 4:1, about 3:1, about 2:1, about 1:1, about 1:2, about 1:3, about 1:4, about 1:5, about 1:6, about 1:7, about 1:8, about 1:9, or about 1:10, or a range between any of the foregoing, such as at a ratio between 10:1 and 1:1, 3:1 and 1:3, or 1:1 and 1:10, each inclusive.

In certain embodiments, the measurement of the recombinant receptor-dependent activity, e.g., the CAR+ specific activity, is the amount or concentration, or a relative amount or concentration, of the soluble factor in the T cell composition at a time point during or at the end of the incubation. In particular embodiments, the measurement is subtracted by or normalized to a control measurement. In some embodiments, the control measurement is a measurement from the same cell composition taken prior to the incubation. In particular embodiments the control measurement is a measurement taken from an identical control cell composition that was not incubated with the recombinant receptor specific stimulation agent. In certain embodiments, the control is a measurement taken at an identical time point during incubation with the recombinant receptor specific agent from a cell composition that does not contain recombinant receptor positive cells.

In some embodiments, the measurement is a normalized ratio of the amount or concentration as compared to the control. In particular embodiments, the measurement is the amount or concentration of the soluble factor per an amount of time, e.g., per minute or per hour. In some embodiments, the measurement is an amount or concentration of the soluble factor per cell or per a set or reference number of cells, e.g., per 100 cells, per 10³ cells, per 10⁴ cells, per 10⁵ cells, per 10⁶ cells, etc. In certain the measurement is the amount or concentration of the soluble factor per an amount of time, per cell or per reference number of cells. In some embodiments, the measurement is the amount or concentration of the soluble factor per cell that expresses the recombinant receptor, CAR+ cell, CAR+/CD8+ cell, Annexin−/CAR+/CD8+ cell, 3CAS−/CAR+/CD8+ cell, CAR+/CD4+ cell, Annexin−/CAR+/CD4+ cell, or 3CAS−/CAR+/CD4+ cell of the cell composition. In certain embodiments, the measurement is the amount or concentration of the soluble factor per amount of time (e.g., per minute or per hour) per cell that expresses the recombinant receptor, CAR+ cell, CAR+/CD8+ cell, Annexin−/CAR+/CD8+ cell, 3CAS−/CAR+/CD8+ cell, CAR+/CD4+ cell, Annexin−/CAR+/CD4+ cell, or 3CAS−/CAR+/CD4+ cell of the cell composition. In some embodiments, the measurement is the amount or concentration of the soluble factor per an amount of time per amount or concentration of the recombinant receptor or CAR+ specific agent. In some embodiments, the measurement is an amount or concentration of the soluble factor per cell or per a set or reference number of cells per amount or concentration of the CAR+ specific agent. In certain the measurement is the amount or concentration of the soluble factor per an amount of time, per amount or concentration of the recombinant receptor or CAR+ specific agent, per cell or per reference number of cells. In some embodiments, the measurement is the amount or concentration of the soluble factor per amount or concentration of the recombinant receptor or CAR+ specific agent, per cell that expresses the recombinant receptor, CAR+ cell, CAR+/CD8+ cell, Annexin−/CAR+/CD8+ cell, 3CAS−/CAR+/CD8+ cell, CAR+/CD4+ cell, Annexin−/CAR+/CD4+ cell, or 3CAS−/CAR+/CD4+ cell of the cell composition. In certain embodiments, the measurement is the amount or concentration of the soluble factor per amount of time, per amount or concentration of the recombinant receptor or CAR+ specific agent, per amount of CAR+ cell, CAR+/CD8+ cell, Annexin−/CAR+/CD8+ cell, 3CAS−/CAR+/CD8+ cell, CAR+/CD4+ cell, Annexin−/CAR+/CD4+ cell, or 3CAS−/CAR+/CD4+ cells of the cell composition.

In particular embodiments, the recombinant receptor or CAR dependent activity is the production or secretion of two or more soluble factors. In certain embodiments, the recombinant receptor or CAR dependent activity is the production or secretion of two, three, four, five, six, seven, eight, nine, ten, or more than ten soluble factors. In some embodiments, the measurements of the two, three, four, five, six, seven, eight, nine, ten, or more than ten soluble factors are combined into an arithmetic mean or a geometric mean. In certain measurements, measurement of the recombinant receptor activity is the secretion of are composites of two, three, four, five, six, seven, eight, nine, ten, or more than ten soluble factors.

In certain embodiments, the soluble factor is a cytokine. In particular embodiments, the recombinant receptor-dependent activity is or includes the production or secretion of a cytokine in response to one or more stimulations. Cytokines are a large group of small signaling molecules that function extensively in cellular communication. Cytokines are most often associated with various immune modulating molecules that include interleukins, chemokines, and interferons. Alternatively cytokines may be characterized by their structure, which are categorized in four families, the four alpha helix family that includes the IL-2 subfamily, and the IFN subfamily; the IL-1 family, the IL-17 family, the IL-10 family, and cysteine-knot cytokines that include members of the transforming growth factor beta family The production and/or the secretion of cytokines contributes to immune responses, and is involved in different processes including the induction of anti-viral proteins and the induction of T cell proliferation. Cytokines are not pre-formed factors but are rapidly produced and secreted in response to cellular activation. The production or secretion of cytokines may be measured, detected, and/or quantified by any suitable technique known in the art. In some embodiments, the recombinant receptor-dependent activity is the production or secretion of one or more soluble factors that include interleukins, interferons, and chemokines. In particular embodiments, the recombinant receptor-dependent activity is the production or secretion of one or more of an IL-2 family member, an IFN subfamily member, an IL-1 family member, IL-10 member, an IL-17 family member, a cysteine-knot cytokine, and/or a member of the transforming growth factor beta family

In certain embodiments, the phenotype is the production of one or more cytokines. In some embodiments, the production of two or more cytokines from the same cell can be indicative of polyfunctional features of such cells. In particular embodiments, the production of one or more cytokines is measured, detected, and/or quantified by intracellular cytokine staining. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. In some embodiments, as described above, the stimulation can be performed using nonspecific stimulation, e.g., is not an antigen-specific stimulation. For example, PMA/ionomycin can be used for nonspecific cell stimulation. In some embodiments, the stimulation can be performed by an agent that is an antigen or an epitope thereof that is specific to the recombinant receptor (e.g., CAR), or is an antibody or fragment thereof that binds to and/or recognizes the recombinant receptor, or a combination thereof. ICS can also be used in combination with other flow cytometry protocols for immunephenotyping using cell surface markers or with MHC multimers to access cytokine production in a particular subgroup of cells, making it an extremely flexible and versatile method. Other single-cell techniques for measuring or detecting cytokine production include, but are not limited to ELISPOT, limiting dilution, and T cell cloning.

In some embodiments, the phenotype is the production of a cytokine, such as following stimulation of the recombinant receptor with an antigen specific to and/or recognized by the recombinant receptor. In particular embodiments, the phenotype is the lack of the production of the cytokine, such as following stimulation of the recombinant receptor with an antigen specific to and/or recognized by the recombinant receptor. In particular embodiments, the phenotype is positive for or is a high level of production of a cytokine. In certain embodiments, the phenotype is negative for or is a low level of production of a cytokine. Cytokines may include, but are not limited to, interleukin-1 (IL-1), IL-1β, IL-2, sIL-2Ra, IL-3, IL-5, IL-6, IL-7, IL-8, IL-10, IL-12, IL-13, IL-17, IL 27, IL-33, IL-35, TNF, tumor necrosis factor alpha (TNFA), CXCL2, CCL2, CCL3, CCL5, CCL17, CCL24, PGD2, LTB4, interferon gamma (IFNG), granulocyte macrophage colony stimulating factor (GMCSF), macrophage inflammatory protein MIP1α, MIP1β, Flt-3L, fracktalkine, and/or IL-5. In some embodiments, the phenotype includes production of cytokines, e.g., cytokines associated with particular cell types, such as cytokines associated with Th1, Th2, Th17 and/or Treg subtypes. In some embodiments, exemplary Th1-related cytokines include IL-2, IFN-γ, and transforming growth factor beta (TGF-β), and in some cases are involved in cellular immune responses. In some embodiments, exemplary Th2-related cytokines include IL-4, IL-5, IL-6, IL-10, and IL-13, and in some cases are associated with humoral immunity and anti-inflammatory properties. In some embodiments, exemplary Th17-related cytokines include IL-17A and IL-17F, and in some cases are involved in recruiting neutrophils and macrophages, e.g., during an inflammatory reaction.

In particular embodiments, the recombinant receptor-dependent activity is the production and/or secretion of one or more of IL-1, IL-1β, IL-2, sIL-2Ra, IL-3, IL-5, IL-6, IL-7, IL-8, IL-10, IL-12, IL-13, IL 27, IL-33, IL-35, TNF, TNF alpha, CXCL2, CCL2, CCL3, CCL5, CCL17, CCL24, PGD2, LTB4, interferon gamma (IFN-γ), granulocyte macrophage colony stimulating factor (GM-CSF), macrophage inflammatory protein (MIP)-1a, MIP-1b, Flt-3L, fracktalkine, and/or IL-5. In certain embodiments, the recombinant receptor-dependent activity production or secretion of a Th17 cytokine. In some embodiments, the Th17 cytokine is GMCSF. In some embodiments, the recombinant receptor-dependent activity comprises production or secretion of a Th2 cytokine, wherein the Th2 cytokine is IL-4, IL-5, IL-10, or IL-13.

In certain embodiments, the recombinant receptor-dependent activity is the production or secretion of a proinflammatory cytokine. Proinflammatory cytokines play a role in initiating the inflammatory response and to regulate the host defense against pathogens mediating the innate immune response. Proinflammatory cytokines include, but are not limited to, interleukins (IL), interleukin-1-beta (IL-1), interleukin-3 (IL-3), interleukin-5 (IL-5), interleukin-6 (IL-6), interleukin-13 (IL-13), tumor necrosis factor (TNF), CXC-chemokine ligand 2 (CXCL2), CC-chemokine ligand 2 (CCL2), CC-chemokine ligand 3 (CCL3), CC-chemokine ligand 5 (CCL5), CC-chemokine ligand 17 (CCL17), CC-chemokine ligand 24 (CCL24), prostaglandin D2 (PGD2) and leukotriene B4 (LTB4) as well as IL-33.). In some embodiments, the CAR dependent activity is production and or secretion of an interleukin and/or a TNF family member. In particular embodiments, the CAR dependent activity is production and or secretion of IL-1, IL-6, IL-8, and IL-18, TNF-alpha or a combination thereof.

In particular embodiments the recombinant receptor-dependent activity is secretion of IL-2, IFN-gamma, TNF-alpha or a combination thereof.

In some embodiments, the phenotype (e.g., recombinant receptor-dependent activity) is or includes the production of a cytokine. In certain embodiments, the phenotype is or includes the production of more than one cytokine (e.g., polyfunctional). In certain embodiments, the recombinant receptor-dependent activity is or includes a lack of a production of one or more cytokines. In certain embodiments, the phenotype is or includes the production, or lack thereof, of one or more of IL-2, IL-5, IL-10, IL-13, IL-17, IFNG, or TNFA. In certain embodiments, the recombinant receptor-dependent activity is or includes the production, or lack thereof, of one or more of IL-2, IL-13, IFNG, or TNFA. In some embodiments, the recombinant receptor-dependent activity is the presence of a production, and/or the presence of a high level of production of the cytokine. In some embodiments, the phenotype is a low, reduced, or absent production of a cytokine.

In some embodiments, the phenotype is or includes the internal (intracellular) production of a cytokine, for example, as assessed in the presence of a stimulatory agent or under stimulatory conditions when secretion is prevented or inhibited. In some embodiments, the stimulatory agent is nonspecific stimulatory agent, e.g., a stimulatory agent that does not bind to an antigen binding domain, for example on a recombinant receptor (e.g., CAR). In some embodiments, the stimulatory agent is PMA/ionomycin, which can act as a nonspecific stimulatory agent. In some embodiments, the stimulatory agent is a specific stimulatory agent, e.g., is a stimulatory agent that is an antigen or an epitope thereof that is specific to the recombinant receptor (e.g., CAR), or is an antibody or fragment thereof that binds to and/or recognizes the recombinant receptor, or a combination thereof. In particular embodiments, the phenotype is or includes the lack or absence of an internal production of a cytokine. In certain embodiments, the phenotype is or includes the internal amount of one or more cytokines when the production of more than one cytokines as assessed with an ICS assay. In certain embodiments, the phenotype is or includes the internal amount of one or more of IL-2, IL-5, IL-13, IFNG, or TNFA as assessed with an ICS assay. In some embodiments, the phenotype is or includes a low internal amount or a lack of a detectable amount of one or more cytokines as assessed with an ICS assay. In certain embodiments, phenotype is or includes a low internal amount or a lack of a detectable amount of IL-2, IL-5, IL-13, IFNG, or TNFA as assessed with an ICS assay. In some embodiments, the phenotype includes assessment of multiple cytokines, e.g., by multiplexed assays or assays to assess polyfunctionality (see, e.g., Xue et al., (2017) Journal for ImmunoTherapy of Cancer 5:85). In some embodiments, the lack of cytokine expression is inversely correlated with or associated with activity and/or function of the cells and/or durability of response and progression free survival. In some embodiments, cells with reduced, minimal or no cytokine production, assessed according to any known method or method described herein, are reduced in the cell composition (e.g., output composition, therapeutic cell composition).

Particular embodiments contemplate that the phenotype may include the production of a cytokine or a lack of or a low amount of production for a cytokine. This may depend on several factors that include, but are not limited to, the identity of the cytokine, the assay performed to detect the cytokine, and the stimulatory agent or condition used with the assay. For example, in some embodiments it is contemplated that the phenotype is or includes a lack of, or a low level of IL-13 production as indicated by ICS while in some embodiments, the phenotype is or includes production of IFN-gamma as indicated by ICS.

In some embodiments, the phenotype is or includes production of one or more cytokines and either CD3⁺, CD4⁺, CD8⁺, CD3⁺/CAR⁺, CD4⁺/CAR⁺, CD8⁺/CAR⁺, annexin V⁻, annexin V⁻CD3⁺, annexin V⁻CD4⁺, annexin V⁻CD8⁺, annexin V⁻CD3⁺/CAR⁺, annexin V⁻CD4⁺/CAR⁺, annexin V⁻CD8⁺/CAR⁺, activated caspase 3⁻, activated caspase 3⁻/CD3⁺, activated caspase 3⁻/CD4⁺, activated caspase 3⁻/CD8⁺, activated caspase 3⁻/CD3⁺/CAR⁺, activated caspase 3⁻/CD4⁺/CAR⁺, or activated caspase 3⁻/CD8⁺/CAR⁺, or a combination thereof. In particular embodiments, the phenotype is or includes production of one or more cytokines in CD4⁺/CAR⁺ and/or CD8⁺/CAR⁺. In some embodiments, the one or more cytokines are IL-2, IFN-gamma, and/or TNF-alpha. In some embodiments, the phenotype is or includes production of IL-2 in CD4⁺/CAR⁺ cells. In some embodiments, the phenotype is or includes production of TNF-alpha in CD4⁺/CAR⁺ cells. In some embodiments, the phenotype is or includes production of IL-2 and TNF-alpha in CD4⁺/CAR⁺ cells. In some embodiments, the phenotype is or includes production of IL-2 and IFN-gamma in CD4⁺/CAR⁺ cells. In some embodiments, the phenotype is or includes production of TNF-alpha in CD8⁺/CAR⁺ cells. In some embodiments, the phenotype is or includes production of IFN-gamma and TNF-alpha in CD8⁺/CAR⁺ cells. In some embodiments, the phenotype is or includes production of IL-2 in activated caspase 3⁻/CD4⁺/CAR⁺ cells. In some embodiments, the phenotype is or includes production of TNF-alpha in activated caspase 3⁻/CD4⁺/CAR⁺ cells. In some embodiments, the phenotype is or includes production of IL-2 and TNF-alpha in activated caspase 3⁻/CD4⁺/CAR⁺ cells. In some embodiments, the phenotype is or includes production of IL-2 and IFN-gamma in activated caspase 3⁻/CD4⁺/CAR⁺ cells. In some embodiments, the phenotype is or includes production of TNF-alpha in activated caspase 3⁻/CD8⁺/CAR⁺ cells. In some embodiments, the phenotype is or includes production of IFN-gamma and TNF-alpha in activated caspase 3⁻/CD8⁺/CAR⁺ cells. In some embodiments, the phenotype is or includes production of TNF-alpha in annexin V⁻/CD4⁺/CAR⁺ cells. In some embodiments, the phenotype is or includes production of IL-2 and TNF-alpha in annexin V⁻/CD4⁺/CAR⁺ cells. In some embodiments, the phenotype is or includes production of IL-2 and IFN-gamma in annexin V⁻/CD4⁺/CAR⁺ cells. In some embodiments, the phenotype is or includes production of TNF-alpha in annexin V⁻/CD8⁺/CAR⁺ cells. In some embodiments, the phenotype is or includes production of IFN-gamma and TNF-alpha in annexin V⁻/CD8⁺/CAR⁺ cells. In some embodiments, the phenotypes described in this paragraph are positively correlated with durable response and progression free survival. Thus, in some embodiments, cells including these phenotypes are maximized or increased in the cell composition (e.g., output composition, therapeutic cell composition).

In some embodiments, the phenotype is or includes a lack of production of one or more cytokines. In certain embodiments, the phenotype is or includes a lack of a production of one or more cytokines and either CD3⁺, CD4⁺, CD8⁺, CD3⁺/CAR⁺, CD4⁺/CAR⁺, CD8⁺/CAR⁺, annexin V⁻, annexin V⁻CD3⁺, annexin V⁻CD4⁺, annexin V⁻CD8⁺, annexin V⁻CD3⁺/CAR⁺, annexin V⁻CD4⁺/CAR⁺, annexin V⁻CD8⁺/CAR⁺, activated caspase 3⁻, activated caspase 3⁻/CD3⁺, activated caspase 3⁻/CD4⁺, activated caspase 3⁻/CD8⁺, activated caspase 3⁻/CD3⁺/CAR⁺, activated caspase 3⁻/CD4⁺/CAR⁺, or activated caspase 3⁻/CD8⁺/CAR⁺, or a combination thereof. In some embodiments, the one or more cytokines are IL-2, IFN-gamma, and/or TNF-alpha. In some embodiments, the phenotype is or includes the lack of production of IL-2 in activated caspase 3⁻/CD4⁺/CAR⁺ cells. In some embodiments, the phenotype is or includes the lack of production of TNF-alpha in activated caspase 3⁻/CD4⁺/CAR⁺ cells. In some embodiments, the phenotype is or includes the lack of production of IL-2 and TNF-alpha in activated caspase 3⁻/CD4⁺/CAR⁺ cells. In some embodiments, the phenotype is or includes the lack of production of IL-2 and IFN-gamma in activated caspase 3⁻/CD4⁺/CAR⁺ cells. In some embodiments, the phenotype is or includes the lack of production of TNF-alpha in activated caspase 3⁻/CD8⁺/CAR⁺ cells. In some embodiments, the phenotype is or includes the lack of production of INF-gamma and TNF-alpha in activated caspase 3⁻/CD8⁺/CAR⁺ cells. In some embodiments, the phenotypes described in this paragraph are negatively correlated with durable response and progression free survival.

In particular embodiments, the phenotype is or includes the presence or absence of an internal amount of one or more of IL-2, IL-13, IFN-gamma, or TNF-alpha as assessed with an ICS assay and one or more specific markers for a subset of cells or cells of a particular cell type. In some embodiments, the phenotype is or includes production, or lack thereof, of one or more of IL-2, IL-13, IFN-gamma, or TNF-alpha and CD4⁺/CAR⁺ and/or CD8⁺/CAR⁺. In certain embodiments, the phenotype is or includes production of IL-2 and CD4⁺/CAR⁺ and/or CD8⁺/CAR⁺. In some embodiments, the phenotype is or includes a lack of or low production of IL-2 and CD4⁺/CAR⁺ and/or CD8⁺/CAR⁺. In some embodiments, the phenotype is or includes production of IL-13 and CD4⁺/CAR⁺ and/or CD8⁺/CAR⁺. In some embodiments, the phenotype is or includes production of IL-13 and CD4⁺/CAR⁺ and/or CD8⁺/CAR⁺. In certain embodiments, the phenotype is or includes the lack of or low production of IL⁻13 and CD4⁺/CAR⁺ and/or CD8⁺/CAR⁺. In some embodiments, the phenotype is or includes production of IFN-gamma and CD4⁺/CAR⁺ and/or CD8⁺/CAR⁺. In certain embodiments, the phenotype is or includes production of TNF-alpha and CD4⁺/CAR⁺ and/or CD8⁺/CAR⁺. In certain embodiments, the phenotype is or includes a lack of or low production of TNF-alpha and CD4⁺/CAR⁺ and/or CD8⁺/CAR⁺.

Any one or more of the phenotypes, alone or in combination, can be assessed or determined in accord with the provided methods. In some embodiments, the phenotype is CD3⁺, CD3⁺/CAR⁺, CD4⁺/CAR⁺, CD8⁺/CAR⁺, or a combination thereof.

In certain embodiments, the phenotype is or includes CD3⁺. In certain embodiments, the phenotype is or includes CD3⁺/CAR⁺. In some embodiments, the phenotype is or includes CD8⁺/CAR⁺. In certain embodiments, the phenotype is or includes CD4+/CAR+.

In particular embodiments, the phenotype is or includes Annexin⁻/CD3⁺/CAR⁺. In some embodiments, the phenotype is or includes Annexin⁻/CD4⁺/CAR⁺. In particular embodiments, the phenotype is Annexin⁻/CD8⁺/CAR.

In particular embodiments, the phenotype is or includes a lack of or a low amount of intracellular IL-2 and CD4⁺/CAR⁺. In particular embodiments, the phenotype is a lack of or a low amount of intracellular IL-13 and CD4⁺/CAR⁺. In some embodiments, the phenotype is a lack of or a low amount of intracellular expression of IL-13 and CD8⁺/CAR⁺ cells. In particular embodiments, the phenotype is a lack of or a low amount of intracellular TNF-alpha CD4⁺/CAR⁺.

In certain embodiments, the phenotype is or includes CD8⁺/CAR⁺. In certain embodiments, the phenotype is or includes annexin⁻/CD8⁺/CAR⁺.

In some embodiments, the phenotype comprises an indicator of production of one or a combination of cytokines, optionally non-specific to the antigen or the recombinant receptor and/or that is polyclonally produced, wherein the one or more cytokines is IL-2, IL-13, IL-17, IFN-gamma or TNF-alpha. In some embodiments, the indicator of production is measured in an assay, optionally an intracellular cytokine staining assay, comprising incubating a sample of the T cell composition with a polyclonal agent, an antigen-specific agent or an agent that binds the recombinant receptor, optionally CAR. In some embodiments, the agent is or comprises PMA and ionomycin or is or comprises a T cell receptor or T cell receptor complex agonist. In some embodiments, the phenotype comprises a naïve phenotype or a memory phenotype, optionally wherein the memory phenotype comprises a T effector memory phenotype, a T central memory phenotype, or a T effector memory phenotype expressing CD45RA (Temra).

In some embodiments, the recombinant receptor-dependent (e.g., CAR) activity is a measure of the production or accumulation of a proinflammatory cytokine, optionally, one of or a combination of TNF-alpha, IFN-gamma, and IL-2. In some embodiments, the recombinant receptor-dependent (e.g., CAR) activity is a measure of the production or accumulation of a combination of TNF-alpha, IFN-gamma, and IL-2, and IL-17. In some embodiments, the recombinant receptor-dependent (e.g., CAR) activity is a measure of the production or accumulation of IFN-gamma, and IL-2. In some embodiments, the recombinant receptor-dependent (e.g., CAR) activity is a measure of the production or accumulation of IFN-gamma, TNFA, and IL-2. In some embodiments, the recombinant receptor-dependent (e.g., CAR) activity is a measure of the production or accumulation of IFN-gamma and TNFA.

In some embodiments, the recombinant receptor activity is recombinant receptor-specific killing (e.g., cytolytic behavior). In some embodiments, the cytolytic activity of engineered CD8+ cells is assessed (e.g., quantified). In some embodiments, recombinant receptor-dependent cytolytic activity is assessed by exposing, incubating, and/or contacting cells expressing the recombinant receptor, or a cell composition containing cells that express the recombinant receptor, with a target cell that expresses the antigen and/or an epitope that is bound by and/or recognized by the recombinant receptor. The cytolytic activity can be measured by directly or indirectly measuring the target cell number over time. For example, the target cells may be incubated with a detectable marker prior to being incubated with recombinant receptor expressing cells, such a marker that is detectable then the target cell is lysed, or a detectable marker that is detectable in viable target cells. These readouts provide direct or indirect of target cell number and/or target cell death, and can be measured at different time points during the assay. A reduction of target cell number and/or an increase of target cell death indicate the cytolytic activity of the cells. Suitable methods for performing cytolytic assays are known in the art, and include, but are not limited to chromium-51 release assays, non-radioactive chromium assays, flow cytometric assays that use fluorescent dyes such as carboxyfluorescein succinimidyl ester (CFSE), PKH-2, and PKH-26. In some cases, cytolytic activity is also referred to herein as cell lysis.

In certain embodiments, the recombinant receptor, e.g., CAR, dependent cytolytic activity is measured by incubating the cell composition that contains cells expressing the recombinant receptor with target cells that express an antigen or an epitope thereof the is bound by or recognized by the recombinant receptor. In certain embodiments, the recombinant receptor is a CAR.

In some embodiments, the measurement of the activity is compared to a control. In certain embodiments, the control is a culture of target cells that are not incubated with the cell composition. In some embodiments, the control is a measurement from a control cell composition that does not contain CAR+ cells that are incubated with the target cells at the same ratio.

In certain embodiments, the measurement of the cytolytic activity assay is the number of target cells that are viable at a time point during or at the end of the incubation. In certain embodiments, the measurement is an amount of a marker of target cell death, e.g., chromium-51, that is released during the incubation. In some embodiments, the measurement is an amount of target cell death that is determined by subtracting the amount of target cells in the co-incubation at a given time point from the amount of target cells of the control that was incubated alone. In some embodiments, the measurement is the percentage of target cells that remain at a time point compared to the starting amount of target cells. In particular embodiments, the measurement is the amount of cells killed over an amount of time. In certain embodiments, the measurement is the amount of cells killed per each cell of the cell composition. In some embodiments, the measurement is the amount of cells killed per cell, or the amount of cells killed per a set number or reference of cells, for example but not limited to, the amount of target cells killed per 100 cells, per 10³ cells, per 10⁴ cells, per 10⁵ cells, per 10⁶ cells, per 10⁷ cells, per 10⁸ cells, per 10⁹ cells, or per 10¹⁰ cells of the composition. In particular embodiments, the measurement is the amount of cells killed per each CAR+ cell, CAR+/CD8+ cell, or Annexin−/CAR+/CD8+ cell, or a reference or set number thereof, of the cell composition. In certain embodiments, the measurement is the amount of cells killed over an amount of time per cell of the cell composition. In particular embodiments, the measurement is the amount of cells killed over an amount of time per CAR+ cells, CAR+/CD8+ cells, or Annexin−/CAR+/CD8+ cells of the cell composition.

In some embodiments, the cell phenotype includes assessing the genomic integration of transgene sequences, such as transgene sequences encoding a recombinant receptor, e.g., a CAR. In some embodiments, the cell phenotype is an integrated copy number, e.g., vector copy number, which is the copy number of the transgene sequence integrated into the chromosomal DNA or genomic DNA of cells. In some embodiments, the vector copy number can be expressed as an average or mean copy number. In some aspects, the vector copy number of a particular integrated transgene includes the number of integrants (containing transgene sequences) per cell. In some embodiments, the vector copy number of a particular integrated transgene includes the number of integrants (containing transgene sequences) per diploid genome. In some aspects, the vector copy number of transgene sequence is expressed as the number of integrated transgene sequences per cell. In some aspects, the vector copy number of transgene sequence is expressed as the number of integrated transgene sequences per diploid genome. In some embodiments, the copy number is an average or mean copy number per diploid genome or per cell among the population of cells.

In some embodiments, the therapeutic cell composition feature is the clonality of the cells of the therapeutic cell composition. In some embodiments, assessing the clonality of the population of T cells is an assessment of clonal diversity of the population of T cells. In some embodiments, the T cells are polyclonal or multiclonal. Clonality, such as polyclonality of said therapeutic cell composition of T cells is a measure of the breadth of the response of the population to a given antigen. In some aspects, the therapeutic cell composition can be assessed by measuring the number of different epitopes recognized by antigen-specific cells. This can be carried out using standard techniques for generating and cloning antigen-specific T cells in vitro. In some embodiments, the T cells are polyclonal (or multiclonal) with no single clonotypic population predominating in the population of naïve-like T cells.

In the context of a population of T cells, such as of the therapeutic cell composition, in some aspects, the signature of polyclonality refers to a population of T cells that has multiple and broad antigen specificity. In some embodiments, polyclonality relates to a population of T cells that exhibits high diversity in the TCR repertoire. In some cases, diversity of the TCR repertoire is due to V(D)J recombination events that, in some respects, are triggered by selection events to self and foreign antigens. In some embodiments, a population of T cells that is diverse or polyclonal is a population of T cells in which analysis indicates the presence of a plurality of varied or different TCR transcripts or products present in the population. In some embodiments, a population of T cells that exhibits high or relatively high clonality is a population of T cells in which the TCR repertoire is less diverse. In some embodiments, T cells are oligoclonal if analysis indicates the presence of several, such as two or three, TCR transcripts or products in a population of T cells. In some embodiments, monoclonality refers to a population of T cells that is of low diversity. In some embodiments, T cells are monoclonal if analysis indicates the presence of a single TCR transcript or product in a population of T cells.

The clonality of the cells in the therapeutic cell composition, such as T cells is, in some examples, determined by clonal sequencing, such as next-generation sequencing, or spectratype analysis. In some aspects, next-generation sequencing methods can be employed, using genomic DNA or cDNA from T cells, to assess the TCR repertoire, including sequences encoding the complementarity-determining region 3 (CDR3). In some embodiments, whole transcriptome sequencing by RNA-seq can be employed. In some embodiments, single-cell sequencing methods can be used.

In some embodiments, clonality, such as polyclonality, can be assessed or determined by spectratype analysis (a measure of the TCR Vβ, Vα, Vγ, or Vδ chain hypervariable region repertoire). Spectratype analysis distinguishes rearranged variable genes of a particular size, not sequence. Thus, it is understood that a single peak could represent a population of T cells expressing any one of a limited number of rearranged TCR variable genes (Vβ, Vα, Vγ, or Vδ) comprising any one of the 4 potential nucleotides (adenine (a), guanine (g), cytosine (c), or thymine (t)) or a combination of the 4 nucleotides at the junctional region. A population of T cells is considered polyclonal when the Vβ spectratype profile for a given TCR Vβ, Vα, Vγ, or Vδ family has multiple peaks, typically 5 or more predominant peaks and in most cases with Gaussian distribution. Polyclonality can also be defined by generation and characterization of antigen-specific clones to an antigen of interest. In the context of a population of T cells, such as of the therapeutic cell composition, monoclonality refers to a population of T cells that has a single specificity as defined by spectratype analysis (a measure of the TCR Vβ, Vα, Vγ, or Vδ chain hypervariable region repertoire). A population of T cells is considered monoclonal (or mono-specific) when the Vβ, Vα, Vγ, and/or Vδ spectratype profile for a given TCR Vβ, Vα, Vγ, and/or Vδ family has a single predominant peak.

In some embodiments, the methods for assessing clonality can include various features of the methods as described in International Publication Nos. WO2012/048341, WO2014/144495, WO2017/053902, WO2016044227, WO2016176322 and WO2012048340 each incorporated by reference in their entirety. In some embodiments, such methods can be used to obtain sequence information about a target polynucleotide of interest within a cell, such as a TCR. The target genes can be obtained from genomic DNA or mRNA of a cell from a sample or population of cells. The sample or population of cells can include immune cells. For example, for target TCR molecules, the genes encoding chains of a TCR can be obtained from genomic DNA or mRNA of immune cells or T cells. In some embodiments, the starting material is RNA from T cells composed of genes that encode for a chain of a TCR.

In some embodiments, the Shannon index is applied to the clonality as a threshold to filter clones (“Shannon-adjusted clonality”), see, Chaara et al. (2018) Front Immunol 9:1038). In some embodiments, the therapeutic cell composition feature is the clonality of the CD4+ cells of the therapeutic cell composition. In some embodiments, the therapeutic cell composition feature is the clonality of the CD8+ cells of the therapeutic cell composition.

In some embodiments, the therapeutic cell composition feature is a dose. In some embodiments, the dose is a single dose of CD4+ and CD8+ engineered cells. In some embodiments, the single dose comprises administering CD4+ engineered cells and CD8+ engineered cells to a subject separately. In some embodiments, the single dose includes administering 25×10⁶ CD8+CAR+ T cells and 25×10⁶ CD4+CAR+ T cells separately to the subject. In some embodiments, the single dose includes administering 50×10⁶ CD8+CAR+ T cells and 50×10⁶ CD4+CAR+ T cells separately to the subject. In some embodiments, the single dose includes administering 75×10⁶ CD8+CAR+ T cells and 75×10⁶ CD4+CAR+ T cells separately to the subject.

In some embodiments, the therapeutic cell composition features include any one or more or all therapeutic cell composition features, including phenotypes and recombinant-receptor dependent activity, described herein. In some embodiments, the therapeutic cell composition features include one or more of CAS3−/CCR7−/CD27−, CAS3−/CCR7−/CD27+, CAS3−/CCR7+, CAS3−/CCR7+/CD27−,CAS3−/CCR7+/CD27+, CAS3−/CD27+, CAS3−/CD28+, CAS3−/CD28+/CD27−, CAS3−/CD28+/CD27+, CAS3−/CCR7−/CD45RA−, CAS3−/CCR7−/CD45RA+, CAS3−/CCR7+/CD45RA−, CAS3−/CCR7+/CD45RA+, CAS+/CD3+/CAR+, CD3+/CAR+, CD3+, CAR+, Clonality, EGFRt+, cytokine−, IFNG+, IFNg+/IL2, IFNg+/IL17+/TNFa+, IFNg+/IL2+/IL17+/TNFa+, IFNg+/IL2+/TNFa+, CAR+/IFNg+, IFNg+/TNFa+, CAR+/IL2+, IL2+/TNFa+, Cell lysis, CAR+/TNFa+, viable cell concentration, vector copy number (VCN), EGFRt+ vector copy number, viability, GMCSF+/CD19+, IFNG+/CD19+, IL10+/CD19+, IL13+/CD19+, IL2+/CD19+, IL4+/CD19+, IL5+/CD19+, IL6+/CD19+, MIP1A+/CD19+, MIP1B+/CD19+, sCD137+/CD19+, TNFa+/CD19+, dose, dose level, percent viable cells dosed, total nonviable cells dosed, total viable cells dosed, total dose.

In some embodiments, the therapeutic cell composition features include one or more of CAS3−/CCR7−/CD27−/CD4+, CAS3−/CCR7−/CD27+/CD4+, CAS3−/CCR7+/CD4+, CAS3−/CCR7+/CD27−/CD4+,CAS3−/CCR7+/CD27+/CD4+, CAS3−/CD27+/CD4+, CAS3−/CD28+/CD4+, CAS3−/CD28+/CD27−/CD4+, CAS3−/CD28+/CD27+/CD4+, CAS3−/CCR7−/CD45RA−/CD4+, CAS3−/CCR7−/CD45RA+/CD4+, CAS3−/CCR7+/CD45RA−/CD4+, CAS3−/CCR7+/CD45RA+/CD4+, CAS+/CD3+/CAR+/CD4+, CD3+/CAR+/CD4+, CD3+/CD4+, CAR+/CD4+, clonality of CD4+ cells, EGFRt+/CD4+, cytokine−/CD4+, IFNG+/CD4+, IFNg+/IL2/CD4+, IFNg+/IL17+/TNFa+/CD4+, IFNg+/IL2+/IL17+/TNFa+/CD4+, IFNg+/IL2+/TNFa+/CD4+, CAR+/IFNg+/CD4+, IFNg+/TNFa+/CD4+, CAR+/IL2+/CD4+, IL2+/TNFa+/CD4+, cell lysis by CD4+, CAR+/TNFa+/CD4+, viable cell concentration of CD4+ cells, vector copy number of CD4+, EGFRt+ vector copy number of CD4+ cells, viability of CD4+ cells, GMCSF+/CD19+/CD4+, IFNG+/CD19+/CD4+, IL10+/CD19+/CD4+, IL13+/CD19+/CD4+, IL2+/CD19+/CD4+, IL4+/CD19+/CD4+, IL5+/CD19+/CD4+, IL6+/CD19+/CD4+, MIP1A+/CD19+/CD4+, MIP1B+/CD19+/CD4+, sCD137+/CD19+/CD4+, TNFa+/CD19+/CD4+, dose of CD4+ cells, dose level of CD4+ cells, percent viable cells dosed of CD4+ cells, total nonviable cells dosed of CD4+ cells, total viable cells dosed of CD4+ cells, total dose of CD4+ cells.

In some embodiments, the therapeutic cell composition features include one or more of CAS3−/CCR7−/CD27−/CD8+, CAS3−/CCR7−/CD27+/CD8+, CAS3−/CCR7+/CD8+, CAS3−/CCR7+/CD27−/CD8+,CAS3−/CCR7+/CD27+/CD8+, CAS3−/CD27+/CD8+, CAS3−/CD28+/CD8+, CAS3−/CD28+/CD27−/CD8+, CAS3−/CD28+/CD27+/CD8+, CAS3−/CCR7−/CD45RA−/CD8+, CAS3−/CCR7−/CD45RA+/CD8+, CAS3−/CCR7+/CD45RA−/CD8+, CAS3−/CCR7+/CD45RA+/CD8+, CAS+/CD3+/CAR+/CD8+, CD3+/CAR+/CD8+, CD3+/CD8+, CAR+/CD8+, Clonality of CD8+, EGFRt+/CD8+, cytokine−/CD8+, IFNG+/CD8+, IFNg+/IL2/CD8+, IFNg+/IL17+/TNFa+/CD8+, IFNg+/IL2+/IL17+/TNFa+/CD8+, IFNg+/IL2+/TNFa+/CD8+, CAR+/IFNg+/CD8+, IFNg+/TNFa+/CD8+, CAR+/IL2+/CD8+, IL2+/TNFa+/CD8+, cell lysis by CD8+ cells, CAR+/TNFa+/CD8+, viable cell concentration of CD8+ cells, vector copy number of CD8+ cells, EGFRt+ vector copy number of CD8+ cells, viability of CD8+ cells, GMCSF+/CD19+/CD8+, IFNG+/CD19+/CD8+, IL10+/CD19+/CD8+, IL13+/CD19+/CD8+, IL2+/CD19+/CD8+, IL4+/CD19+/CD8+, IL5+/CD19+/CD8+, IL6+/CD19+/CD8+, MIP1A+/CD19+/CD8+, MIP1B+/CD19+/CD8+, sCD137+/CD19+/CD8+, TNFa+/CD19+/CD8+, dose of CD8+ cells, dose level of CD8+, percent viable cells dosed of CD8+ cells, total nonviable cells dosed of CD8+ cells, total viable cells dosed of CD8+ cells, total dose of CD8+ cells.

In some embodiments, the therapeutic cell composition features include one or more of CAS3−/CCR7−/CD27−, CAS3−/CCR7−/CD27+, CAS3−/CCR7+, CAS3−/CCR7+/CD27−,CAS3−/CCR7+/CD27+, CAS3−/CD27+, CAS3−/CD28+, CAS3−/CD28+/CD27−, CAS3−/CD28+/CD27+, CAS3−/CCR7−/CD45RA−, CAS3−/CCR7−/CD45RA+, CAS3−/CCR7+/CD45RA−, CAS3−/CCR7+/CD45RA+, CAS+/CD3+/CAR+, CD3+/CAR+, CD3+, CAR+, clonality, EGFRt+, cytokine−, IFNG+, IFNg+/IL2, IFNg+/IL17+/TNFa+, IFNg+/IL2+/IL17+/TNFa+, IFNg+/IL2+/TNFa+, CAR+/IFNg+, IFNg+/TNFa+, CAR+/IL2+, IL2+/TNFa+, Cell lysis, CAR+/TNFa+, viable cell concentration, vector copy number, EGFRt+ Vector Copy Number, Viability, GMCSF+/, IFNG+/, IL10+/, IL13+/, IL2+/, IL4+/, IL5+/, IL6+/, MIP1A+/, MIP1B+/, sCD137+/, TNFa+/, dose, dose level, percent viable cells dosed, total nonviable cells dosed, total viable cells dosed, Total dose.

In some embodiments, the therapeutic cell composition features include one or more of CAS3−/CCR7−/CD27−/CD4+, CAS3−/CCR7−/CD27+/CD4+, CAS3−/CCR7+/CD4+, CAS3−/CCR7+/CD27−/CD4+,CAS3−/CCR7+/CD27+/CD4+, CAS3−/CD27+/CD4+, CAS3−/CD28+/CD4+, CAS3−/CD28+/CD27−/CD4+, CAS3−/CD28+/CD27+/CD4+, CAS3−/CCR7−/CD45RA−/CD4+, CAS3−/CCR7−/CD45RA+/CD4+, CAS3−/CCR7+/CD45RA−/CD4+, CAS3−/CCR7+/CD45RA+/CD4+, CAS+/CD3+/CAR+/CD4+, CD3+/CAR+/CD4+, CD3+/CD4+, CAR+/CD4+, clonality of CD4+ cells, EGFRt+/CD4+, cytokine−/CD4+, IFNG+/CD4+, IFNg+/IL2/CD4+, IFNg+/IL17+/TNFa+/CD4+, IFNg+/IL2+/IL17+/TNFa+/CD4+, IFNg+/IL2+/TNFa+/CD4+, CAR+/IFNg+/CD4+, IFNg+/TNFa+/CD4+, CAR+/IL2+/CD4+, IL2+/TNFa+/CD4+, cell lysis by CD4+, CAR+/TNFa+/CD4+, viable cell concentration of CD4+ cells, vector copy number of CD4+ cells, EGFRt+ vector copy number of CD4+, viability of CD4+, GMCSF+/CD4+, IFNG+/CD4+, IL10+/CD4+, IL13+/CD4+, IL2+/CD4+, IL4+/CD4+, IL5+/CD4+, IL6+/CD4+, MIP1A+/CD4+, MIP1B+/CD4+, sCD137+/CD4+, TNFa+/CD4+, dose of CD4+ cells, dose level of CD4+ cells, percent viable cells dosed of CD4+ cells, total nonviable cells dosed of CD4+ cells, total viable cells dosed of CD4+ cells, and total dose of CD4+ cells.

In some embodiments, the therapeutic cell composition features include one or more of CAS3−/CCR7−/CD27−/CD8+, CAS3−/CCR7−/CD27+/CD8+, CAS3−/CCR7+/CD8+, CAS3−/CCR7+/CD27−/CD8+,CAS3−/CCR7+/CD27+/CD8+, CAS3−/CD27+/CD8+, CAS3−/CD28+/CD8+, CAS3−/CD28+/CD27−/CD8+, CAS3−/CD28+/CD27+/CD8+, CAS3−/CCR7−/CD45RA−/CD8+, CAS3−/CCR7−/CD45RA+/CD8+, CAS3−/CCR7+/CD45RA−/CD8+, CAS3−/CCR7+/CD45RA+/CD8+, CAS+/CD3+/CAR+/CD8+, CD3+/CAR+/CD8+, CD3+/CD8+, CAR+/CD8+, clonality of CD8+ cells, EGFRt+/CD8+, cytokine−/CD8+, IFNG+/CD8+, IFNg+/IL2/CD8+, IFNg+/IL17+/TNFa+/CD8+, IFNg+/IL2+/IL17+/TNFa+/CD8+, IFNg+/IL2+/TNFa+/CD8+, CAR+/IFNg+/CD8+, IFNg+/TNFa+/CD8+, CAR+/IL2+/CD8+, IL2+/TNFa+/CD8+, cell lysis by CD8+, CAR+/TNFa+/CD8+, viable cell concentration of CD8+ cells, vector copy number of CD8+ cells, EGFRt+ vector copy number of CD8+, viability of CD8+, GMCSF+/CD8+, IFNG+/CD8+, IL10+/CD8+, IL13+/CD8+, IL2+/CD8+, IL4+/CD8+, IL5+/CD8+, IL6+/CD8+, MIP1A+/CD8+, MIP1B+/CD8+, sCD137+/CD8+, TNFa+/CD8+, dose of CD8+ cells, dose level of CD8+ cells, percent viable cells dosed of CD8+ cells, total nonviable cells dosed of CD8+ cells, total viable cells dosed of CD8+ cells, and total dose of CD8+ cells.

In some embodiments, the therapeutic cell composition features include one or more of CAS3−/CCR7−/CD27−/CD8+, CAS3−/CCR7−/CD27+/CD8+, CAS3−/CCR7+/CD8+, CAS3−/CCR7+/CD27−/CD8+,CAS3−/CCR7+/CD27+/CD8+, CAS3−/CD27+/CD8+, CAS3−/CD28+/CD8+, CAS3−/CD28+/CD27−/CD8+, CAS3−/CD28+/CD27+/CD8+, CAS3−/CCR7−/CD45RA−/CD8+, CAS3−/CCR7−/CD45RA+/CD8+, CAS3−/CCR7+/CD45RA−/CD8+, CAS3−/CCR7+/CD45RA+/CD8+, CAS+/CD3+/CAR+/CD8+, CD3+/CAR+/CD8+, CD3+/CD8+, CAR+/CD8+, clonality of CD8+ cells, EGFRt+/CD8+, cytokine−/CD8+, IFNG+/CD8+, IFNg+/IL2/CD8+, IFNg+/IL17+/TNFa+/CD8+, IFNg+/IL2+/IL17+/TNFa+/CD8+, IFNg+/IL2+/TNFa+/CD8+, CAR+/IFNg+/CD8+, IFNg+/TNFa+/CD8+, CAR+/IL2+/CD8+, IL2+/TNFa+/CD8+, cell lysis by CD8+, CAR+/TNFa+/CD8+, viable cell concentration of CD8+ cells, vector copy number of CD8+ cells, EGFRt+ vector copy number of CD8+, viability of CD8+, GMCSF+/CD8+, IFNG+/CD8+, IL10+/CD8+, IL13+/CD8+, IL2+/CD8+, IL4+/CD8+, IL5+/CD8+, IL6+/CD8+, MIP1A+/CD8+, MIP1B+/CD8+, sCD137+/CD8+, TNFa+/CD8+, dose of CD8+ cells, dose level of CD8+ cells, percent viable cells dosed of CD8+ cells, total nonviable cells dosed of CD8+ cells, total viable cells dosed of CD8+ cells, total dose of CD8+ cells, CAS3−/CCR7−/CD27−/CD4+, CAS3−/CCR7−/CD27+/CD4+, CAS3−/CCR7+/CD4+, CAS3−/CCR7+/CD27−/CD4+,CAS3−/CCR7+/CD27+/CD4+, CAS3−/CD27+/CD4+, CAS3−/CD28+/CD4+, CAS3−/CD28+/CD27−/CD4+, CAS3−/CD28+/CD27+/CD4+, CAS3−/CCR7−/CD45RA−/CD4+, CAS3−/CCR7−/CD45RA+/CD4+, CAS3−/CCR7+/CD45RA−/CD4+, CAS3−/CCR7+/CD45RA+/CD4+, CAS+/CD3+/CAR+/CD4+, CD3+/CAR+/CD4+, CD3+/CD4+, CAR+/CD4+, clonality of CD4+ cells, EGFRt+/CD4+, cytokine−/CD4+, IFNG+/CD4+, IFNg+/IL2/CD4+, IFNg+/IL17+/TNFa+/CD4+, IFNg+/IL2+/IL17+/TNFa+/CD4+, IFNg+/IL2+/TNFa+/CD4+, CAR+/IFNg+/CD4+, IFNg+/TNFa+/CD4+, CAR+/IL2+/CD4+, IL2+/TNFa+/CD4+, cell lysis by CD4+, CAR+/TNFa+/CD4+, viable cell concentration of CD4+ cells, vector copy number of CD4+ cells, EGFRt+ vector copy number of CD4+, viability of CD4+, GMCSF+/CD4+, IFNG+/CD4+, IL10+/CD4+, IL13+/CD4+, IL2+/CD4+, IL4+/CD4+, IL5+/CD4+, IL6+/CD4+, MIP1A+/CD4+, MIP1B+/CD4+, sCD137+/CD4+, TNFa+/CD4+, dose of CD4+ cells, dose level of CD4+ cells, percent viable cells dosed of CD4+ cells, total nonviable cells dosed of CD4+ cells, total viable cells dosed of CD4+ cells, and total dose of CD4+ cells.

In some embodiments, the therapeutic cell composition features include any one or more of the therapeutic cell composition features shown in Table E4 below. In some of any of the above embodiments, the percentage, number, and/or proportion of cells having a phenotype as described above, is determined, measured, obtained, detected, observed, and/or identified. In certain embodiments, the number of cells of the phenotype is the total amount of cells of the phenotype of the cell composition. In some embodiments, the number of the cells of the phenotype may be expressed as a frequency, ratio, and/or a percentage of cells of the phenotype present in the therapeutic cell composition. . In some embodiments, the therapeutic cell composition feature is a frequency, ratio, and/or a percentage of cells having the phenotype or recombinant receptor-dependent activity described herein.

4. Clinical Responses

The methods provided herein are useful for determining features, such as subject features, input composition features, and therapeutic cell composition features as described herein, associated with clinical responses in a subject following treatment with a therapeutic cell composition. Various types of clinical responses are contemplated herein, including, but not limited to complete response (CR), a partial response (PR), overall response rate (ORR), objective response (OR), progression free survival (PFS), a durable response (e.g., durability of response DOR), toxicity response, and/or a pharmacokinetic response.

In some embodiments, the clinical response is a complete response (CR). As used herein, CR refers to the disappearance of all signs of a disease or condition in a subject in response to a treatment for the disease or condition. Thus, in some embodiments, the provided methods can be used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a CR following treatment with the treatment regimen.

In some embodiments, the clinical response is a complete response (CR), such as described using the Lugano criteria involves a complete metabolic response and a complete radiologic response at various measurable sites. In some aspects, these sites include lymph nodes and extralymphatic sites, wherein a CR is described as a score of 1, 2, or 3 with or without a residual mass on the 5-point scale, when PET-CT is used.

In some embodiments, the clinical response is a partial response (PR). As used herein, PR refers to a decrease in the extent of a disease or condition in a subject in response to a treatment for the disease or condition. Thus, in some embodiments, the provided methods can be used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a PR following treatment with the treatment regimen.

In some aspects, the clinical response is a partial response (PR; also known in some cases as partial remission) as described using the Lugano criteria and involves a partial metabolic and/or radiological response at various measurable sites. In some aspects, these sites include lymph nodes and extralymphatic sites, wherein a PR is described as a score of 4 or 5 with reduced uptake compared with baseline and residual mass(es) of any size, when PET-CT is used.

In some embodiments, the clinical response is progression-free survival (PFS). As used herein, PFS refer to the length of time during and after the treatment of a disease or condition that a patient lives with the disease, but the disease or condition does not get worse. Thus, in some embodiments, the provided methods can be used to determine or predict, prior to treatment, the PFS of a subject to be treated with a treatment regimen, e.g., a predetermined treatment regimen that includes a therapeutic cell composition, including a therapeutic cell composition produced from an input composition comprising T cells selected from the subject, following treatment with the treatment regimen. In some embodiments, the provided methods can be used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a PFS of certain duration following treatment with the treatment regimen. In some embodiments, the certain duration is, is about, is greater than, or is greater than about 1, 2, 3, 4, 5, 6, 7, 8, 9, 10, 11, 12, 18, or 24 months. In some embodiments, the certain duration is greater than 3 months.

In some embodiments, the clinical response is an objective response (OR). In some embodiments, the OR is the best objective response binarized by partial and complete response rates. Thus, in some embodiments, the provided methods can be used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit an OR following treatment with the treatment regimen.

In some embodiments, the clinical response is progression-free survival (PFS) which is described as the length of time during and after the treatment of a disease, such as cancer, that a subject lives with the disease but it does not get worse. In some embodiments, the clinical response is an objective response (OR). In some embodiments, the OR is the best objective response binarized by partial and complete response rates. In some embodiments, the clinical response is an objective response rate (ORR; also known in some cases as overall response rate), which is described as the proportion of patients who achieved CR or PR. In some aspects, the clinical response is overall survival (OS), described as the length of time from either the date of diagnosis or the start of treatment for a disease, such as cancer, that subjects diagnosed with the disease are still alive. In some embodiments, the clinical response is an event-free survival (EFS), described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the clinical response is a measure of the duration of response (DOR). As used herein, DOR refers to the amount of time from documentation of response to treatment to disease progression. Thus, in some embodiments, the provided methods can be used to determine or predict, prior to treatment, the DOR of a subject to be treated with a treatment regimen, e.g., a predetermined treatment regimen that includes a therapeutic cell composition, including a therapeutic cell composition produced from an input composition comprising T cells selected from the subject, following treatment with the treatment regimen. In some embodiments, the provided methods can be used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a durable response following treatment with the treatment regimen. In some embodiments, a durable response is a DOR of, of about, of greater than, or of greater than about 1, 2, 3, 4, 5, 6, 7, 8, 9, 10, 11, 12, 18, or 24 months. In some embodiments, a durable response is a DOR of greater than 3 months.

In some embodiments, the provided methods can be used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a DOR of certain duration following treatment with the treatment regimen. In some embodiments, the certain duration is, is about, is greater than, or is greater than about 1, 2, 3, 4, 5, 6, 7, 8, 9, 10, 11, 12, 18, or 24 months. In some embodiments, the certain duration is greater than 3 months.

In some embodiments, the clinical response is the measure of duration of response (DOR), which includes the time from documentation of tumor response to disease progression. In some embodiments, the clinical response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the clinical response is based on the RECIST criteria used to determine objective tumor response; in some aspects, in solid tumors. (Eisenhauer et al., European Journal of Cancer 45 (2009) 228-247.) In some embodiments, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In some embodiments, the clinical response includes pharmacokinetics of administered cells. For example, the pharmacokinetics of adoptively transferred cells are determined to assess the availability, e.g., bioavailability of the administered cells. Methods for determining the pharmacokinetics of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentjens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated epidermal growth factor receptor (EGFRt) in some cases can be co-expressed with a transgene of interest (a CAR or TCR) in transduced cells (see, e.g., U.S. Pat. No. 8,802,374). EGFRt may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the clinical response is a pharmacokinetic response. In some embodiments, the pharmacokinetic response is the number of CAR+ T cells in a sample obtained from the subject, e.g., blood, determined at a period of time after administration of the cell therapy. In some embodiments, the clinical response is the maximum concentration of CAR+ T cells identified in a sample, e.g., blood, obtained from a subject previously treated with the therapeutic cell composition. In some embodiments, the pharmacokinetic response is exposure, determined by the area under the curve (AUC) of CAR+ T cells identified in samples, e.g., blood samples, obtained from a subject previously treated with the therapeutic cell composition. In some embodiments, the pharmacokinetic response is CAR+ T cell expansion. In some embodiments, the pharmacokinetic response is CAR+ T cell persistence. In some embodiments, the pharmacokinetic response is CAR+ T cell exhaustion. In some embodiments, a pharmacokinetic response is a target pharmacokinetic response. For example, the target pharmacokinetic response may be a target measure of maximum CAR+ T cells (Cₘₐₓ) in a blood sample obtained from the subject at a period of time after administration of the cell therapy, a target measure of exposure determined by AUC, and/or a target time to peak concentration of CAR+ T cells (Tₘₐₓ). In some embodiments, AUC is determined over a period of 0 to 28 days from the time of administration of the therapeutic cell composition, e.g., AUC₀₋₂₈. In some embodiments, the methods provided herein can determine whether a subject treated with a therapeutic cell composition provided herein will achieve a target pharmacokinetic response.

In some embodiments, the clinical response is a toxicity response. In some embodiments, the clinical response is cytokine release syndrome (CRS) or severe CRS (sCRS). CRS, e.g., sCRS. CRS or sCRS can occur in some cases following adoptive T cell therapy and administration to subjects of other biological products. See Davila et al., Sci Transl Med 6, 224ra25 (2014); Brentjens et al., Sci. Transl. Med. 5, 177ra38 (2013); Grupp et al., N. Engl. J. Med. 368, 1509-1518 (2013); and Kochenderfer et al., Blood 119, 2709-2720 (2012); Xu et al., Cancer Letters 343 (2014) 172-78.

Typically, CRS is caused by an exaggerated systemic immune response mediated by, for example, T cells, B cells, NK cells, monocytes, and/or macrophages. Such cells may release a large amount of inflammatory mediators such as cytokines and chemokines. Cytokines may trigger an acute inflammatory response and/or induce endothelial organ damage, which may result in microvascular leakage, heart failure, or death. Severe, life-threatening CRS can lead to pulmonary infiltration and lung injury, renal failure, or disseminated intravascular coagulation. Other severe, life-threatening toxicities can include cardiac toxicity, respiratory distress, neurologic toxicity and/or hepatic failure. In some aspects, fever, especially high fever (≥38.5° C. or ≥101.3° F.), is associated with CRS or risk thereof. In some cases, features or symptoms of CRS mimic infection. In some embodiments, infection is also considered in subjects presenting with CRS symptoms, and monitoring by cultures and empiric antibiotic therapy can be administered. Other symptoms associated with CRS can include cardiac dysfunction, adult respiratory distress syndrome, renal and/or hepatic failure, coagulopathies, disseminated intravascular coagulation, and capillary leak syndrome.

In some embodiments, the clinical response is the grade of CRS. In some embodiments, the clinical response is a severe CRS. In some embodiments, the clinical response is the absence of severe CRS (e.g., moderate or mild CRS). Tables 1 and 2 below shows criteria reflective of CRS grade.

TABLE 1
Exemplary Grading Criteria for CRS

| Grade | Description of Symptoms |
|---|---|
| 1 | Not life-threatening, require only symptomatic treatment |
| Mild | such as antipyretics and anti-emetics (e.g., fever, nausea, |
| | fatigue, headache, myalgias, malaise) |
| 2 | Require and respond to moderate intervention: |
| Moderate | Oxygen requirement < 40%, or |
| | Hypotension responsive to fluids or low dose of a single |
| | vasopressor, or |
| | Grade 2 organ toxicity (by CTCAE v4.0) |
| 3 | Require and respond to aggressive intervention: |
| Severe | Oxygen requirement ≥ 40%, or |
| | Hypotension requiring high dose of a single vasopressor |
| | (e.g., norepinephrine ≥ 20 μg/kg/min, dopamine > 10 |
| | μg/kg/min, phenylephrine ≥ 200 μg/kg/min, or |
| | epinephrine > 10 μg/kg/min), |
| | or |
| | Hypotension requiring multiple vasopressors (e.g., |
| | vasopressin + one of the above agents, or combination |
| | vasopressors equivalent to ≥20 μg/kg/min norepinephrine), or |
| | Grade 3 organ toxicity or Grade 4 transaminitis (by CTCAE |
| | v4.0) |
| 4 | Life-threatening: |
| Life- | Requirement for ventilator support, or |
| threatening | Grade 4 organ toxicity (excluding transaminitis) |
| 5 | Death |
| Fatal | |

TABLE 2
Exemplary Grading Criteria for CRS

| | Grade | | | Grade 4 (life- |
|---|---|---|---|---|
| Symptoms/Signs | 1 (mild) | Grade 2 (moderate) | Grade 3 (severe) | threatening) |
| | | CRS grade is defined by the most severe | | |
| | | symptom (excluding fever) | | |
| Temperature ≥ | Any | Any | Any | Any |
| 38.5° C./101.3° F. | | | | |
| Systolic blood | N/A | Responds to fluid or single | Needs high-dose | Life-threatening |
| pressure ≤ 90 mm | | low-dose vasopressor | or multiple | |
| Hg | | | vasopressors | |
| Need for oxygen | N/A | FiO2 < 40% | FiO₂ ≥ 40% | Needs ventilator |
| to reach SaO₂ > | | | | support |
| 90% | | | | |
| Organ toxicity | N/A | Grade 2 | Grade 3 or | Grade 4 |
| | | | transaminitis | (excluding |
| | | | | transaminitis) |

In some embodiments, the clinical response is or is associated with neurotoxicity. In some embodiments, symptoms associated with a clinical risk of neurotoxicity include confusion, delirium, aphasia, expressive aphasia, obtundation, myoclonus, lethargy, altered mental status, convulsions, seizure-like activity, seizures (optionally as confirmed by electroencephalogram (EEG)), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute-Common Toxicity Criteria version 4.03 (NCI-CTCAE v4.03).

In some embodiments, the clinical response is mild or moderate neurotoxicity, e.g., grade 1 or 2 as set forth in Table 3 below. In some embodiments, the clinical response is severe neurotoxicity, which includes neurotoxicity with a grade of 3 or greater, such as set forth in Table 3.

TABLE 3
Exemplary Grading Criteria for neurotoxicity

| Grade | Description of Symptoms |
|---|---|
| 1 | Mild or asymptomatic symptoms |
| Asymptomatic or Mild | |
| 2 | Presence of symptoms that limit instrumental activities of daily living |
| Moderate | (ADL), such as preparing meals, shopping for groceries or clothes, |
| | using the telephone, managing money |
| 3 | Presence of symptoms that limit self-care ADL, such as bathing, |
| Severe | dressing and undressing, feeding self, using the toilet, taking |
| | medications |
| 4 | Symptoms that are life-threatening, requiring urgent intervention |
| Life-threatening | |
| 5 | Death |
| Fatal | |

Thus, in some embodiments, the provided methods can be used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a toxicity response following treatment with the treatment regimen. In some embodiments, the toxicity response is CRS (e.g., grade 1 or higher CRS). In some embodiments, the toxicity response is mild CRS (e.g., grade 2 or less CRS). In some embodiments, the toxicity response is severe CRS (e.g., grade 3 or higher CRS).

In some embodiments, the toxicity response is a neurotoxicity (e.g., grade 1 or higher neurotoxicity). In some embodiments, the toxicity response is a mild neurotoxicity (e.g., grade 2 or less neurotoxicity). In some embodiments, the toxicity response is a severe neurotoxicity (e.g., grade 3 or higher neurotoxicity).

In some embodiments, the clinical response is one or more or all of log₁₀AUC for CD3+ cells, log₁₀ maximum concentration for CD3+ cells (Cmax), time of peak concentration of CD3+ cells (Tmax), log₁₀AUC for CD4+ cells, log₁₀ maximum concentration for CD4+ cells (Cmax), time of peak concentration of CD4+ cells (Tmax), log₁₀AUC for CD8+ cells, log₁₀ maximum concentration for CD8+ cells (Cmax), time of peak concentration of CD8+ cells (Tmax), overall log₁₀AUC, overall log₁₀ maximum concentration (Cmax), time of peak concentration of cells overall (Tmax), objective response, complete response, progression free survival, durability of response, grade 3 or greater neurotoxicity, any neurotoxic response, grade 3 or greater cytokine release syndrome, and any cytokine release syndrome. In some embodiments, Cmax, Tmax, and AUC are determine by flow cytometry and/or polymerase chain reaction. In some embodiments, AUC is determine from 0-28 days from administering the therapeutic cell composition. In some cases, for example when determined according to a random survival forests model, the progress free survival and durability of response include a time measure, e.g., time to event (PFS or DOR).

B. Supervised Machine Learning Methods

It is contemplated that the clinical responses of a subject to treatment with a therapeutic cell composition (e.g., engineered T cell composition) can, in some cases, depend upon many factors, including, but not limited to, the features of the subject to be treated, the features of the therapeutic cell composition administered to the subject, and the features of the input composition, from which the therapeutic cell composition is produced (see, e.g., Section I.A). Thus, in some embodiments, to identify the features associated with subject clinical response, the features may be used as input to machine learning models capable of identifying features of importance for determining clinical response.

Further to identifying features associated with clinical responses, in some embodiments, the machine learning models may be used to determine clinical responses of a subject following treatment with a therapeutic cell composition prior to treating the subject, where the determination of the clinical outcome is based on the subject features, therapeutic cell features, and the input composition features. As described in Section I.B.4 below, determining the clinical response of a subject to treatment with a therapeutic cell composition prior to administering the cell composition can inform the strategy for treating the subject. For example, if a subject is determined (e.g., predicted) to have a negative clinical response (e.g., toxicity response), the treatment regimen (e.g., a predetermined treatment regimen) may be altered so as to result in a positive clinical response (e.g., complete response, durable response, absence of toxicity response).

Machine learning models contemplated for use according to the methods described herein include transparent models. For example, the machine learning models can be interrogated or queried to determine how the model made a particular determination (e.g., prediction). In some embodiments, the machine learning model is a random forests model. In some embodiments, the machine learning model is a random survival forests model. Advantages of using random forests and random survival forests models include the ability to interrogate or query the models to identify how, e.g., which features, the model used to determine a subject's clinical response to a cell therapy, e.g., therapeutic cell composition. In some embodiments, the features used to determine the clinical response are considered features associated with the clinical response. In some embodiments, identifying features used to determine a subject's clinical response comprises assessing feature importance, for example as described herein.

1. Random Forests

In some embodiments, the machine learning model used to identify features associated with clinical responses is a random forests model. In some embodiments, the random forests model is also used to determine clinical responses of a subject treated with a therapeutic cell composition. A random forests is an example of an ensemble learning method. A random forests can comprise a plurality of decision trees, to which are applied one or more inputs, e.g., features, such as subject features, therapeutic cell composition features, input composition features, to generate one or more corresponding outputs, e.g., classifications, for example, clinical responses. In some embodiments, the random forests are classifiers. In some embodiments, the random forests perform regression.

In some embodiments, decision trees in the random forests model accept as input, subject features, therapeutic cell composition features, and input composition features, and as output, generate a classification of a clinical response. (Preferably, the individual decision trees are sufficiently uncorrelated such that common inputs applied to the plurality of decision trees will result in a diversity of outputs.) In some embodiments, the subject features, therapeutic cell composition features, and input composition features are applied to the plurality of individual decision trees, and the corresponding outputs are reconciled to generate an output of the random forests model. For example, in some embodiments, the output of a random forest may correspond to a classification output by a majority of the individual decision trees.

Various methods of training a random forests are familiar to those skilled in the art and are within the scope of the disclosure. For example, in some embodiments, a random forests model can be trained on a dataset including subject features, therapeutic cell composition features, and input composition features by applying randomly sampled input data (e.g., subject features, therapeutic cell composition features, and input composition features) to individual decision trees of the random forests. This training method promotes diversity among the individual decision trees, which can improve the accuracy of the random forest. It will be appreciated by those skilled in the art that many suitable configurations of random forests can be utilized as appropriate; the disclosure is not limited to any type or configuration of random forests, its constituent decision trees, any method of training decision trees, or any method of training any of the above.

In some embodiments, the random forests model is trained using supervised learning to determine (e.g., classify or predict) clinical responses based on subject features, therapeutic cell composition features, and input composition features. In some cases, the random forests model can be trained on a set of data including subject features, therapeutic cell composition features, input composition features, and the corresponding clinical responses, and the accuracy of the model tested on a different set of data not used for training the model, which includes subject features, therapeutic cell composition features, and input composition features, where the clinical responses are known.

In some embodiments, for example when datasets for training and testing a model are limited in size, for example as described below, the model can be trained using bootstrap aggregation. In some embodiments, for example when datasets for training and testing a model are limited in size, for example as described below, the model can be evaluated using cross validation. In some embodiments, the random forests model is evaluated using cross validation. In some embodiments, the random forests model is evaluated using k-fold cross validation. In some embodiments, the random forests model is evaluated using 10-fold cross validation. In some embodiments, the random forests model is evaluated using nested cross validation.

In some embodiments, the dataset used to train the random forests model includes features (subject features, therapeutic cell composition features, input composition features) obtained from or from about 500, 400, 300, 200, 150, 100, 50, 25, 15, or 10 subjects. In some embodiments, the dataset used to train the random forests model includes features (subject features, therapeutic cell composition features, input composition features) obtained from or from about 100 to 500, 100 to 400, 100 to 300, 100 to 200, or 100 to 150 subjects. In some embodiments, the dataset used to train the random forests model includes features (subject features, therapeutic cell composition features, input composition features) obtained from, from about, or from less than 500, 400, 300, 200, 150, 100 subjects. In some embodiments, the dataset used to train the random forests model includes features (subject features, therapeutic cell composition features, input composition features) obtained from, from about, or from less than 300, 200, 150, 100 subjects. In some embodiments, the dataset used to train the random forests model includes features (subject features, therapeutic cell composition features, input composition features) obtained from, from about, or from less than 200 subjects. In some embodiments, the dataset used to train the random forests model includes features (subject features, therapeutic cell composition features, input composition features) obtained from, from about, or from less than 150 subjects. In some embodiments, the dataset used to train the random forests model includes features (subject features, therapeutic cell composition features, input composition features) obtained from, from about, or from less than 100 subjects. In some embodiments, the dataset used evaluate the model is obtained from, for about, or from less than any number of subjects described in this paragraph. In some embodiments, the subject are subject participating in a clinical trial.

a. Feature Importance

In some embodiments, the random forests models described herein (e.g., trained, tested, evaluated random forests model) can be queried to identify features associated with clinical responses. In some embodiments, identifying features associated with clinical responses includes determining an importance measure for each of the features used to in the model. Importance measures can be assessed using a variety of techniques, including, but not limited to, a permutation importance measure where values of individual features (e.g., one feature at a time) are permuted and a decrease in prediction accuracy is calculated, determining a mean decrease in the Gini index of node impurity by splits on an individual feature, determining a mean minimal depth (e.g., the mean depth where a feature is used for splitting), determining a total number of trees in which a split on the feature occurs, determining a total number of nodes that use the feature for splitting, determining a total number of trees in which the feature is used for splitting the root node, and determining a p-value for a one-sided binomial test. In some embodiments, the importance measure is or includes any of the aforementioned importance measures. In some embodiments, the importance measure is a permutation importance measure. In some embodiments, the importance measure is a mean minimal depth. In some embodiments, the importance measure is the total number of trees wherein the feature splits a root node.

In some embodiments, the features associated with a clinical response are the features identified by a magnitude of an importance measure. In some embodiments, the features can be rank ordered by importance measure value (e.g., magnitude). For example, in some cases, features can be rank ordered from largest to smallest importance measure values, where the importance measure assessed is the same for each feature (e.g., permutation importance measure, mean minimal depth, number of trees where the features splits the root node). In some embodiments, the features associated with the clinical response are identified by rank ordering the features by importance measure values, where the importance measure assessed is the same for each feature (e.g., permutation importance measure, mean minimal depth, number of trees where the features splits the root node). In some embodiments, the features associated with a clinical response are the first 10, 9, 8, 7, 6, 5, 4, 3, 2, or 1 features identified by rank ordering. In some embodiments, the first 10, 9, 8, 7, 6, 5, 4, 3, 2, or 1 features identified by rank ordering include the features with largest importance measures. In some embodiments, the features associated with a clinical response are the first 10 features identified by rank ordering of importance measures. In some embodiments, the features associated with a clinical response are the first 5 features identified by rank ordering importance measure values. In some embodiments, the features associated with a clinical response are the first 3 features identified by rank ordering importance measures values. In some embodiments, the features associated with a clinical response are the first 2 features identified by rank ordering importance measures values. In some embodiments, the features associated with a clinical response is the first feature identified by rank ordering importance measures values.

In some embodiments, more than one or a combination of importance measures can be used to identify features associated with clinical outcome. In some embodiments, the combination of importance measure is or comprises a permutation importance measure, the mean minimal depth, the number of trees where the features splits the root node, and a p-value, or any combination thereof. In some embodiments, the combination of importance measure is or comprises a permutation importance measure, the mean minimal depth, and the number of trees where the features splits the root node.

b. Predicting Clinical Outcome

The random forests models described herein may also be used to determine clinical responses of a subject to treatment with a therapeutic cell composition, prior to treating the subject with the therapeutic cell composition. In some embodiments, assessing the determined clinical responses of the subject can be used to inform treatment of the subject. For example, if a subject is determined (e.g., predicted) to have negative clinical response, e.g., toxicity, poor or reduced pharmacokinetics compared to a target response, lack of CR, PR, or DOR, an alteration to a predetermined treatment regimen, for example as described in Section I.B.4 below, can be made. On the other hand, if a subject is determined (e.g., predicted) to have positive clinical responses, e.g., CR, PR, DOR, a pharmacokinetic response that reflects or is greater than a target pharmacokinetic response, no or mild toxicity, a predetermined treatment regimen may be administered.

In some embodiments, the trained random forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a CR following treatment with the treatment regimen.

In some embodiments, the trained random forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a PR following treatment with the treatment regimen.

In some embodiments, the trained random forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit an OR following treatment with the treatment regimen.

In some embodiments, the trained random forests model is used to determine or predict, prior to treatment, the DOR of a subject to be treated with a treatment regimen, e.g., a predetermined treatment regimen that includes a therapeutic cell composition, including a therapeutic cell composition produced from an input composition comprising T cells selected from the subject, following treatment with the treatment regimen. In some embodiments, the trained random forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a durable response, e.g., a DOR of greater than three months, following treatment with the treatment regimen.

In some embodiments, the trained random forests model is used to determine or predict, prior to treatment, the PFS of a subject to be treated with a treatment regimen, e.g., a predetermined treatment regimen that includes a therapeutic cell composition, including a therapeutic cell composition produced from an input composition comprising T cells selected from the subject, following treatment with the treatment regimen. In some embodiments, the trained random forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a PFS of certain duration, e.g., a PFS of greater than three months, following treatment with the treatment regimen.

In some embodiments, the trained random forests model is used to determine or predict, prior to treatment, the pharmacokinetic response of a subject to be treated with a treatment regimen, e.g., a predetermined treatment regimen that includes a therapeutic cell composition, including a therapeutic cell composition produced from an input composition comprising T cells selected from the subject, following treatment with the treatment regimen. In some embodiments, the trained random forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a pharmacokinetic response greater than a target pharmacokinetic response, following treatment with the treatment regimen. In some embodiments, the pharmacokinetic response is a measure of maximum CAR+ T cell concentration (Cₘₐₓ) in a blood sample obtained from the subject at a period of time after administration of the treatment regimen. In some embodiments, the pharmacokinetic response is a measure of exposure to CAR+ T cells, for instance exposure over or over about 28 days following administration of the treatment regimen and/or as determined by AUC of the CAR+ T cell concentration-time curve following administration of the treatment regimen. In some embodiments, the pharmacokinetic response is the time to peak concentration of CAR+ T cells (Tₘₐₓ).

In some embodiments, the trained random forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a toxicity response following treatment with the treatment regimen. In some embodiments, the toxicity response is CRS. In some embodiments, the toxicity response is severe CRS, e.g., grade 3 or higher CRS. In some embodiments is a neurotoxicity. In some embodiments, the toxicity response is severe neurotoxicity, e.g., grade 3 or higher neurotoxicity.

2 Random Survival Forests

In some embodiments, the machine learning model used to identify features associated with clinical responses and to determine clinical responses of a subject treated with a therapeutic cell composition is a random survival forests model. A random survival forest is an example of an ensemble learning method useful for analyzing right-censored survival data. As with the random forests described above, random survival forests can comprise a plurality of decision trees, each of which is applied to one or more inputs, e.g., features, to generate one or more corresponding outputs, e.g., classifications. Generation of a random survival forests model generally follows that of random forests, however, the random survival forests model includes a time and a censor indicator, wherein the censor indicator denotes the presence (e.g., 1) or absence (e.g., 0) of an event at a given time point, t. Thus, the splitting rule for growing the trees of the random survival forests accounts for the censoring feature. The random survival forests models provided herein are generated based on the occurrence of clinical responses over time.

In some embodiments, the random survival forests model is trained using supervised learning to estimate a clinical response function and cumulative hazard function, e.g., for a given clinical response, based on subject features, therapeutic cell composition features, and input composition features. In some cases, the random survival forests model can be trained on a set of data including subject features, therapeutic cell composition features, input composition features, and the corresponding clinical responses, and the accuracy of the model tested on a different set of data not used for training the model, which includes subject features, therapeutic cell composition features, and input composition features, where the clinical responses are known.

In some embodiments, for example when datasets for training and testing a model are limited in size, for example as described below, the model can be evaluated using cross validation. In some embodiments, the random survival forests model is evaluated using cross validation. In some embodiments, the random survival forests model is evaluated using k-fold cross validation. In some embodiments, the random survival forests model is evaluated using 10-fold cross validation. In some embodiments, the random survival forests model is evaluated using nested cross validation.

In some embodiments, the dataset used to train the random survival forests model includes features (subject features, therapeutic cell composition features, input composition features) obtained from or from about 500, 400, 300, 200, 150, 100, 50, 25, 15, or 10 subjects. In some embodiments, the dataset used to train the random survival forests model includes features (subject features, therapeutic cell composition features, input composition features) obtained from or from about 100 to 500, 100 to 400, 100 to 300, 100 to 200, or 100 to 150 subjects. In some embodiments, the dataset used to train the random survival forests model includes features (subject features, therapeutic cell composition features, input composition features) obtained from, from about, or from less than 500, 400, 300, 200, 150, 100 subjects. In some embodiments, the dataset used to train the random survival forests model includes features (subject features, therapeutic cell composition features, input composition features) obtained from, from about, or from less than 300, 200, 150, 100 subjects. In some embodiments, the data set used to train the random survival forests model includes features (subject features, therapeutic cell composition features, input composition features) obtained from, from about, or from less than 200 subjects. In some embodiments, the data set used to train the random survival forests model includes features (subject features, therapeutic cell composition features, input composition features) obtained from, from about, or from less than 150 subjects. In some embodiments, the data set used to train the random survival forests model includes features (subject features, therapeutic cell composition features, input composition features) obtained from, from about, or from less than 100 subjects. In some embodiments, the dataset used evaluate the model is obtained from, for about, or from less than any number of subjects described in this paragraph. In some embodiments, the subject are subject participating in a clinical trial.

a. Feature Importance

In some embodiments, the random survival forests models described herein (e.g., trained, tested, evaluated random survival forests model) can be queried to identify features associated with clinical responses. In some embodiments, identifying features associated with clinical responses (e.g., clinical response and cumulative hazard functions) includes determining an importance measure for each of the features used to in the model. Similar to random forests, importance measures can be assessed using a variety of techniques, including, but not limited to, a permutation importance measure where values of individual features (e.g., one feature at a time) are permuted and a decrease in prediction accuracy is calculated, determining a mean decrease in the Gini index of node impurity by splits on an individual feature, determining a mean minimal depth (e.g., the mean depth where a feature is used for splitting), determining a total number of trees in which a split on the feature occurs, determining a total number of nodes that use the feature for splitting, determining a total number of trees in which the feature is used for splitting the root node, and determining a p-value for a one-sided binomial test. In some embodiments, the decrease in prediction accuracy, for example when using permutation, is with respect to a concordance index. In some embodiments, the importance measure is or includes any of the aforementioned importance measures. In some embodiments, the importance measure is a permutation importance measure. In some embodiments, the importance measure is a mean minimal depth. In some embodiments, the importance measure is the total number of trees wherein the feature splits a root node.

In some embodiments, the features associated with a clinical response are the features identified by a magnitude of an importance measure. In some embodiments, the features can be rank ordered by importance measure value (e.g., magnitude). For example, in some cases, features can be rank ordered from largest to smallest importance measure values, where the importance measure assessed is the same for each feature (e.g., permutation importance measure, mean minimal depth, number of trees where the features splits the root node). In some embodiments, the features associated with the clinical response are identified by rank ordering the features by importance measure values, where the importance measure assessed is the same for each feature (e.g., permutation importance measure, mean minimal depth, number of trees where the features splits the root node). In some embodiments, the features associated with a clinical response are the first 10, 9, 8, 7, 6, 5, 4, 3, 2, or 1 features identified by rank ordering. In some embodiments, the first 10, 9, 8, 7, 6, 5, 4, 3, 2, or 1 features identified by rank ordering include the features with largest importance measures. In some embodiments, the features associated with a clinical response are the first 10 features identified by rank ordering of importance measures. In some embodiments, the features associated with a clinical response are the first 5 features identified by rank ordering importance measure values. In some embodiments, the features associated with a clinical response are the first 3 features identified by rank ordering importance measures values. In some embodiments, the features associated with a clinical response are the first 2 features identified by rank ordering importance measures values. In some embodiments, the features associated with a clinical response is the first feature identified by rank ordering importance measures values.

In some embodiments, more than one or a combination of importance measures can be used to identify features associated with clinical outcome. In some embodiments, the combination of importance measure is or comprises a permutation importance measure, the mean minimal depth, the number of trees where the features splits the root node, and a p-value, or any combination thereof. In some embodiments, the combination of importance measure is or comprises a permutation importance measure, the mean minimal depth, and the number of trees where the features splits the root node.

b. Predicting Clinical Outcome

The random survival forests models described herein may also be used to determine, e.g., estimate, clinical responses of a subject to treatment with a therapeutic cell composition, prior to treating the subject with the therapeutic cell composition. In some embodiments, assessing the determined clinical responses of the subject can be used to inform treatment of the subject. For example, if a subject is determined (e.g., predicted) to have negative clinical response, e.g., toxicity, poor or reduced pharmacokinetics compared to a target response, lack of CR, PR, or DOR, an alteration to a predetermined treatment regimen, for example as described in Section I.B.4 below, can be made. On the other hand, if a subject is determined (e.g., predicted) to have positive clinical responses, e.g., CR, PR, DOR, a pharmacokinetic response that reflects or is greater than a target pharmacokinetic response, no or mild toxicity, a predetermined treatment regimen may be administered.

In some embodiments, the trained random survival forests model is used to determine or predict, prior to treatment, the DOR of a subject to be treated with a treatment regimen, e.g., a predetermined treatment regimen that includes a therapeutic cell composition, including a therapeutic cell composition produced from an input composition comprising T cells selected from the subject, following treatment with the treatment regimen. In some embodiments, the trained random survival forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a durable response, e.g., a DOR of greater than three months, following treatment with the treatment regimen.

In some embodiments, the trained random survival forests model is used to determine or predict, prior to treatment, the PFS of a subject to be treated with a treatment regimen, e.g., a predetermined treatment regimen that includes a therapeutic cell composition, including a therapeutic cell composition produced from an input composition comprising T cells selected from the subject, following treatment with the treatment regimen. In some embodiments, the trained random survival forests model is used to determine or predict, prior to treatment, if a subject to be treated with a treatment regimen, e.g., a predetermined treatment regimen that includes a therapeutic cell composition, including a therapeutic cell composition produced from an input composition comprising T cells selected from the subject, will exhibit a PFS of certain duration, e.g., a PFS of greater than three months, following treatment with the treatment regimen.

I. Preprocessing

In some embodiments, the subject features, therapeutic cell composition features, and input composition features are preprocessed. Data preprocessing is an important step in model creation to avoid creating models that produce misleading or inaccurate results. In some embodiments, preprocessing prevents out-of-range values, missing values, impossible data combinations, highly correlated variables and the like from being incorporated into the model. In some cases, preprocessing leads to the identification of informative features. For example, preprocessing may be used to remove features with little or no variance, features with low dynamic range (e.g., <5% over distribution), features that are highly correlated, or features with missing values, or replace missing values, such that the remaining features are informative, discriminating, and independent (e.g., informative features). In some embodiments, features with little or no scientific relevance are removed. For example, certain features associated with manufacturing the therapeutic cell composition but with low scientific relevance may be removed. In some embodiments, the features identified by preprocessing are informative features.

In some embodiments, the machine learning models, e.g., random forests, random survival forests, are trained on the informative features identified by preprocessing. In some embodiments, the machine learning models, e.g., random forests, random survival forests, are trained using supervised learning on the informative features identified by preprocessing. The generation, training, testing, and validation of the random forests models and the random survival forests models described in Sections I.B.1a-b and I.B.2a-b, respectively, may occur using informative features identified by preprocessing. Further, when the random forests or random survival forests used to determine (e.g., predict, estimate) clinical responses of a subject to be treated with a therapeutic cell composition, prior to treating the subject, the features used as input to the models are informative features identified preprocessing. In some embodiments, the informative features used for training the models and the informative features for determining clinical responses in subjects prior to treatment with a therapeutic cell composition are the same informative features.

In some embodiments, preprocessing to identify informative features includes one or more or all of removing features, e.g., subject features, input composition features, and therapeutic cell composition features, having zero or near zero variance, removing features with greater than 70% of values missing, removing highly correlated features (e.g., |ρ|>0.7) from the dataset, and imputing values for features with less than 70% of values missing, where the missing values are replaced with either the mean or mode. In some embodiments, preprocessing to identify informative features is or includes removing features having zero or near zero variance. In some embodiments, preprocessing to identify informative features is or includes removing features with greater than 70% of values missing. In some embodiments, preprocessing to identify informative features is or includes removing highly correlated features (|ρ|>0.7). In some embodiments, preprocessing to identify informative features is or includes imputing values for features with less than 70% of values missing, where the missing values are replaced with either the mean or mode. In some embodiments, the one or more preprocessing steps described herein result in a dataset comprising informative features.

In some embodiments, preprocessing to identify informative features includes one or more or all of removing features, e.g., subject features, input composition features, and therapeutic cell composition features, having greater than, than about, or 50% of the data missing; removing subject features, input composition features, and therapeutic cell composition features having zero variance or greater than, than about or 95% of data values equal to a single value and fewer than 0.1n unique values, where n=number of samples; imputing missing data by multivariate imputation by chained equations for subject features, input composition features, and therapeutic cell composition features; identifying covariate clusters, the covariate clusters comprising sets of subject features, input composition features, and therapeutic cell composition features and combinations thereof with correlation coefficients of greater than, about, or equal to 0.5 (e.g., |ρ|≥0.5), and iteratively selecting subject features, input composition features, and therapeutic cell composition features from the covariate cluster, where the selected subject features, input composition features, and therapeutic cell composition features have the lowest mean absolute correlation with all remaining subject features, input composition features, and therapeutic cell composition features.

In some embodiments, preprocessing to identify informative features includes one or more or all of removing features, e.g., subject features, input composition features, and therapeutic cell composition features, having greater than, than about, or 60% of the data missing; removing subject features, input composition features, and therapeutic cell composition features having zero variance or greater than, than about or 95% of data values equal to a single value and fewer than 0.1n unique values, where n=number of samples; imputing missing data by multivariate imputation by chained equations for subject features, input composition features, and therapeutic cell composition features; identifying covariate clusters, the covariate clusters comprising sets of subject features, input composition features, and therapeutic cell composition features and combinations thereof with correlation coefficients of greater than, about, or equal to 0.5 (e.g., |ρ|≥0.5), and iteratively selecting subject features, input composition features, and therapeutic cell composition features from the covariate cluster, where the selected subject features, input composition features, and therapeutic cell composition features have the lowest mean absolute correlation with all remaining subject features, input composition features, and therapeutic cell composition features.

In some embodiments, identifying covariate clusters includes computing a heterogeneous correlation matrix, comprising Pearson product-moment correlations between numeric features, polyserial correlations between numeric and ordinal features, and polychoric correlations between ordinal features. Correlations are computed between each pair of features using all complete pairs of observations on those features. Covariate clusters are defined as sets of features with correlation coefficients >0.5, and representative features are iteratively selected as those in each cluster that exhibit the lowest mean absolute correlation with all other remaining features in the dataset. In some of any embodiments, the correlation coefficient is the absolute value of rho (e.g., |ρ|).

In some embodiments, preprocessing to identify informative features includes removing features, e.g., subject features, input composition features, and therapeutic cell composition features, having greater than, than about, or 50% of the data missing. In some embodiments, preprocessing to identify informative features includes removing features, e.g., subject features, input composition features, and therapeutic cell composition features, having greater than, than about, or 60% of the data missing. In some embodiments, preprocessing to identify informative features includes removing features, e.g., subject features, input composition features, and therapeutic cell composition features having zero variance or greater than, than about or 95% of data values equal to a single value and fewer than 0.1n unique values, where n=number of samples. In some embodiments, preprocessing to identify informative features includes imputing missing data by multivariate imputation by chained equations for subject features, input composition features, and therapeutic cell composition features. In some embodiments, preprocessing to identify informative features includes identifying covariate clusters, the covariate clusters comprising sets of subject features, input composition features, and therapeutic cell composition features and combinations thereof with correlation coefficients of greater than, about, or equal to 0.5 (e.g., |ρ|≥0.5), and iteratively selecting subject features, input composition features, and therapeutic cell composition features from the covariate cluster, where the selected subject features, input composition features, and therapeutic cell composition features have the lowest mean absolute correlation with all remaining subject features, input composition features, and therapeutic cell composition features.

In some embodiments, preprocessing to identify informative features is or includes removing features with greater than 40, 50, 60, 70, or 80% of values missing. In some embodiments, preprocessing to identify informative features is or includes removing features with or with about 40 to 80% of values missing. In some embodiments, preprocessing to identify informative features is or includes removing features with or with about 40 to 70% of values missing. In some embodiments, preprocessing to identify informative features is or includes removing features with or with about 40 to 60% of values missing. In some embodiments, preprocessing to identify informative features is or includes removing features with or with about 40 to 50% of values missing. In some embodiments, preprocessing to identify informative features is or includes removing features with greater than 40% of values missing. In some embodiments, preprocessing to identify informative features is or includes removing features with greater than 50% of values missing. In some embodiments, preprocessing to identify informative features is or includes removing features with greater than 60% of values missing. In some embodiments, preprocessing to identify informative features is or includes removing features with greater than 70% of values missing. In some embodiments, preprocessing to identify informative features is or includes removing features with greater than 80% of values missing. In some embodiments, preprocessing to identify informative features is or includes removing highly correlated features. In some embodiments, highly correlated features are features having an absolute value of rho greater than or equal to or about 0.4, 0.5, 0.6, 0.7, 0.8, or 0.9. In some embodiments, highly correlated features are features having an absolute value of rho of greater than or equal to or about 0.4. In some embodiments, highly correlated features are features having an absolute value of rho of greater than or equal to or about 0.5. In some embodiments, highly correlated features are features having an absolute value of rho of greater than or equal to or about 0.6. In some embodiments, highly correlated features are features having an absolute value of rho of greater than or equal to or about 0.7. In some embodiments, highly correlated features are features having an absolute value of rho of greater than or equal to or about 0.8. In some embodiments, highly correlated features are features having an absolute value of rho of greater than or equal to or about 0.9.

In some embodiments, one or more (e.g., any combination) of preprocessing steps described herein (e.g., Section I.B.3) is used to identify informative features. In some embodiments, the one or more preprocessing steps described herein result in a dataset comprising informative features. In some embodiments, the informative features comprise one or more subject features, one or more therapeutic cell composition features, and one or more input composition features. In some embodiments, the informative features comprise one or more subject features, one or more therapeutic cell composition features, or one or more input composition features.

4. Methods of Treatment

In some embodiments, understanding the relationship (e.g., association) between features, e.g., subject features, therapeutic cell composition features, and input composition features, with clinical responses in a subject, as well as an ability to determine or predict clinical responses in a subject to treatment with a therapeutic cell composition prior to treatment can inform treatment strategy. For example, treatment regimens, e.g., predetermined treatment regimens, may be altered or maintained depending on the anticipated clinical response. In some embodiments, maintaining the predetermined treatment regimen or altering the treatment regimen may be useful generating positive clinical responses, e.g., CR, PR, DOR, no toxicity.

a. Combination Treatment

In some embodiments, if the subject to be treated is determined (e.g., predicted) to have clinical responses not including CR, PR, DOR of a certain length, progression free survival of a certain length, or target pharmacokinetics, a treatment strategy that includes an additional treatment may be considered. In some embodiments, the therapeutic cell compositions (e.g., CD4+, CD8+ therapeutic T cell compositions) are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the therapeutic cell compositions (e.g., CD4+, CD8+ therapeutic T cell compositions) are co-administered with another therapy sufficiently close in time such that the therapeutic cell composition populations enhance the effect of one or more additional therapeutic agents, or vice versa. In some embodiments, the therapeutic cell compositions (e.g., CD4+, CD8+ therapeutic T cell compositions) are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration.

In some embodiments, the combination therapy includes administration of a kinase inhibitor, such as a BTK inhibitor (e.g., ibrutinib or acalibrutinib); an inhibitor or a tryptophan metabolism and/or kynurenine pathway, such as an inhibitor of indoleamine 2,3-dioxygenase-1 (IDO1) (e.g., epacadostat); an immunomodulatory agent, such as an immunomodulatory imide drug (IMiD), including a thalidomide or thalidomide derivative (e.g., lenalidomide, pomalidomide, or pomnalidomide); or a check point inhibitor, such as an anti-PD-L1 antibody (e.g., durvalumumab).

Exemplary combination therapies and methods are described in published international applications WO 2018/085731, WO 2018/102785, WO 2019/213184, WO 2018/071873, WO 2018/102786, WO 2018/204427, WO 2019/152743, which are incorporated by reference in their entirety.

b. Determining Dosing and Administration

In some embodiments, if the subject to be treated is determined (e.g., predicted) to have clinical responses not including CR, PR, DOR of a certain length, progression free survival of a certain length, or target pharmacokinetics, a treatment strategy that optimizes dose may be considered. The therapeutic composition or a dose thereof, in some embodiments contains the cells in amounts effective to treat or prevent the disease or condition, such as a therapeutically effective or prophylactically effective amount. In some embodiments, the composition includes the cells in an amount effective to reduce burden of the disease or condition. In some embodiments, the composition includes cells in an amount that provides more consistent outcome, e.g., response and/or safety outcomes, among a group of subjects administered the composition, and/or more consistent pharmacokinetic parameters. In some embodiments, the composition includes the cells in an amount effective to promote durable response and/or progression free survival. In some aspects, the provided methods involve assessing a therapeutic composition containing T cells for cell phenotypes, and determining doses based on such outcomes.

In some embodiments, the dose is determined to encompass a relatively consistent number, proportion, ratio and/or percentage of engineered cells having a particular phenotype in one or more particular compositions. In some aspects, the consistency is associated with or related to a relatively consistent activity, function, pharmacokinetic parameters, toxicity outcome and/or response outcome. In some aspects, in a plurality of subjects, compositions and/or doses the numbers, proportion, ratio and/or percentage, are relatively consistent, e.g., the number or ratio of cells that have a particular phenotype, e.g., express CCR7 (CCR7⁺) or, that produce a cytokine, for example, produce IL-2, TNF-alpha, or IFN-gamma, in the composition or unit dose, varies by no more than 40%, by no more than 30%, by no more than 20%, by no more than 10% or by no more than 5%. In some aspects the number or ratio of cells that have a particular phenotype, e.g., express CCR7 (CCR7⁺), in the composition or unit dose, varies by no more than 20% or no more than 10% or no more than 5% from an average of said number or ratio in a plurality of T cell compositions produced by the process and/or varies from such average by no more than one standard deviation or varies by no more than 20% or no more than 10% or no more than 5% among a plurality of T cell compositions or doses determined. In some embodiments, the plurality of subjects includes at least 10 subjects, such as at least 15, at least 20, at least 25, at least 30, at least 40, at least 50, at least 60, at least 70, at least 80, at least 90, at least 100 or more subjects.

In some aspects, the dose, e.g., one or more unit dose(s) is determined based on the number, percentage, ratio, frequency and/or proportion of a particular subset of engineered T cells, e.g., cells having a particular phenotype, such as particular surface marker phenotype. In some aspects, the cell phenotype is determined based on expression and/or absence of expression of particular cell markers, e.g., surface markers. In some aspects, the cell marker includes markers indicative of viability and/or apoptotic state of the cells. In some aspects, exemplary markers include CD3, CD4, CD8, CCR7, CD27, CD45RA, annexin V, or activated caspase 3. In some aspects, an exemplary marker is CCR7. In some aspects, an exemplary marker is CD27. In some aspects, exemplary markers include CCR7 and/or CD27. In some aspects, exemplary markers include CCR7, CD27 and/or CD45RA.

In some embodiments, provided are methods involving administering to a subject one or more unit doses of a therapeutic T cell composition, such as any described herein and/or any unit dose determined by the methods provided herein.

In some embodiments, provided are methods involving administering to a subject having a disease or condition a unit dose of a T cell composition comprising cells comprising a recombinant receptor, such as a chimeric antigen receptor (CAR), that specifically binds to an antigen associated with the disease or condition, wherein either a defined number of total recombinant receptor-expressing cells (receptor+) of the therapeutic composition, total CD8⁺ recombinant receptor-expressing cells (receptor⁺/CD8⁺) are administered and/or a unit dose of such cells is administered in which the unit dose contains a defined number, percentage, ratio, frequency and/or proportion of cells with a certain phenotype, e.g., CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8+, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, CCR7⁻/CD27⁻/CD8⁺.

In some embodiments, the unit dose of cells comprises a defined number of recombinant receptor-expressing CD8⁺ T cells that express C-C chemokine receptor type 7 (CCR7) (receptor⁺/CD8⁺/CCR7⁺ cells) and/or a defined number of recombinant receptor-expressing CD4⁺ T cells that express CCR7 (receptor⁺/CD4⁺/CCR7⁺ cells) and/or a defined ratio of receptor⁺/CD8⁺/CCR7⁺ cells to receptor⁺/CD4⁺/CCR7⁺ cells and/or a defined ratio of receptor⁺/CD8⁺/CCR7⁺ cells and/or receptor⁺/CD4⁺/CCR7⁺ cells to another subset of cells in the composition. In some embodiments, the unit dose of cells comprises a defined number of CD8⁺/CCR7⁺ cells. In some embodiments, the unit dose of cells comprises a defined number of CD4⁺/CCR7⁺ cells. In some embodiments, the defined number or ratio is further based on expression or absence of expression of CD27 and/or CD45RA on the cells.

In some embodiments, the unit dose of cells comprises a defined number of recombinant receptor-expressing CD8⁺ T cells that express cluster of differentiation 27 (CD27) (receptor⁺/CD8⁺/CD27⁺ cells) and/or a defined number of recombinant receptor-expressing CD4⁺ T cells that express CD27 (receptor⁺/CD4⁺/CD27⁺ cells) and/or a defined ratio of receptor⁺/CD8⁺/CD27⁺ cells to receptor⁺/CD4⁺/CD27⁺ cells and/or a defined ratio of receptor⁺/CD8⁺/CD27⁺ cells and/or receptor⁺/CD4⁺/CD27⁺ cells to another subset of cells in the composition. In some embodiments, the unit dose of cells comprises a defined number of CD8⁺/CD27⁺ cells. In some embodiments, the unit dose of cells comprises a defined number of CD4⁺/CD27⁺ cells. In some embodiments, the defined number or ratio is further based on expression or absence of expression of CCR7 and/or CD45RA on the cells.

In some embodiments, the unit dose of cells comprises a defined number of recombinant receptor-expressing CD8⁺ T cells that express CCR7 and CD27 (receptor⁺/CD8⁺/CCR7⁺/CD27⁺ cells) and/or a defined number of recombinant receptor-expressing CD4⁺ T cells that express CCR7 and CD27 (receptor⁺/CD4⁺/CCR7⁺/CD27⁺ cells) and/or a defined ratio of receptor⁺/CD8⁺/CCR7⁺/CD27⁺ cells to receptor⁺/CD4⁺/CCR7⁺/CD27⁺ cells and/or a defined ratio of receptor⁺/CD8⁺/CCR7⁺/CD27⁺ cells and/or receptor⁺/CD4⁺/CCR7⁺/CD27⁺ cells to another subset of cells in the composition. In some embodiments, the unit dose of cells comprises a defined number of CD8⁺/CCR7⁺/CD27⁺ cells. In some embodiments, the unit dose of cells comprises a defined number of CD4⁺/CCR7⁺/CD27⁺ cells. In some embodiments, the defined number or ratio is further based on expression or absence of expression of CD45RA on the cells.

In some embodiments, the number of cells in the unit dose is the number of cells or number of recombinant receptor-expressing or CAR-expressing cells, or number, percentage, ratio, frequency and/or proportion of such cells of a certain phenotype, e.g., cells that express or do not express one or more markers selected from CD3 CD4, CD8, CCR7, CD27, CD45RA, annexin V, or activated caspase 3, that it is desired to administer to a particular subject in a dose, such as a subject from which the cells have been derived. In some embodiments, the number of cells in the unit dose is the number of cells or number of recombinant receptor-expressing or CAR-expressing cells, or number, percentage, ratio, frequency and/or proportion of such cells of a certain phenotype, e.g., CCR7⁺, CD27⁺, CD45RA⁺, CD45RA⁻, CD4⁺, CD8⁺, CD3⁺, apoptosis marker negative (e.g., Annexin V⁻ or Caspase 3⁻) cells, or cells that are positive or negative for one or more of any of the foregoing.

In some embodiments, the number of cells in the unit dose is the number of cells or number of recombinant receptor-expressing or CAR-expressing cells, or number, percentage, ratio and/or proportion of such cells of a certain phenotype, e.g., CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8+, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4+, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, CCR7⁻/CD27⁻/CD8⁺; and apoptosis marker negative (e.g., Annexin V⁻ or Caspase 3⁻) cells, that it is desired to administer to a particular subject in a dose, such as a subject from which the cells have been derived. In some embodiments, the unit dose contains a defined number of cells or number of recombinant receptor-expressing or CAR-expressing cells, or number, percentage, ratio and/or proportion of such cells of a certain phenotype e.g., CCR7⁺/CD4⁺, CCR7⁺/CD8⁺, CD27⁺/CD4⁺, CD27⁻/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4+, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, CCR7⁻/CD27⁻/CD8⁺; and apoptosis marker negative (e.g., Annexin V⁻ or Caspase 3⁻) cells, and/or any subset thereof.

In some embodiments, the unit dose is determined based on the number of cells or cell type(s) and/or a frequency, ratio, and/or percentage of cells or cell types, e.g., individual populations, phenotypes, or subtypes, in the cell composition, such as those with the phenotypes of annexin V⁻/CCR7⁺/CAR⁺; annexin V⁻/CCR7⁺/CAR⁺/CD4⁺; annexin V⁻/CCR7⁺/CAR⁺/CD8⁺; annexin V⁻/CD27⁺/CAR⁺; annexin V⁻/CD27⁺/CAR⁺/CD4⁺; annexin V⁻/CD27⁺/CAR⁺/CD8⁺; annexin V⁻/CCR7⁺/CD27⁺/CAR⁺; annexin V⁻/CCR7⁺/CD27⁺/CAR⁺/CD4⁺; annexin V⁻/CCR7⁺/CD27⁺/CAR⁺/CD8⁺; annexin V⁻/CCR7⁺/CD45RA⁻/CAR⁺; annexin V⁻/CCR7⁺/CD45RA⁻/CAR⁺/CD4⁺; annexin V⁻/CCR7⁺/CD45RA⁻/CAR⁺/CD8⁺; annexin V⁻/CCR7⁻/CD45RA⁻/CAR⁺; annexin V⁻/CCR7⁻/CD45RA⁻/CAR⁺/CD4⁺; annexin V⁻/CCR7⁻/CD45RA⁻/CAR⁺/CD8⁺; annexin V⁻/CCR7⁻/CD27⁻/CAR⁺, annexin V⁻/CCR7⁻/CD27⁻/CAR⁺/CD4⁺; annexin V⁻/CCR7⁻/CD27⁻/CAR⁺/CD8⁺; activated caspase 3⁻/CCR7⁺/CAR⁺; activated caspase 3⁻/CCR7⁺/CAR⁺/CD4⁺; activated caspase 3⁻/CCR7⁺/CAR⁺/CD8⁺; activated caspase 3⁻/CD27⁺/CAR⁺; activated caspase 3⁻/CD27⁺/CAR⁺/CD4⁺; activated caspase 3⁻/CD27⁺/CAR⁺/CD8⁺; activated caspase 3⁻/CCR7⁺/CD27⁺/CAR⁺; activated caspase 3⁻/CCR7⁺/CD27⁺/CAR⁺/CD4⁺; activated caspase 3⁻/CCR7⁺/CD27⁺/CAR⁺/CD8⁺; activated caspase 3⁻/CCR7⁺/CD45RA⁻/CAR⁺; activated caspase 3⁻/CCR7⁺/CD45RA⁻/CAR⁺/CD4⁺; activated caspase 3⁻/CCR7⁺/CD45RA⁻/CAR⁺/CD8⁺; activated caspase 3⁻/CCR7⁻/CD45RA⁻/CAR⁺; activated caspase 3⁻/CCR7⁻/CD45RA⁻/CAR⁺/CD4⁺; activated caspase 3⁻/CCR7⁻/CD45RA⁻/CAR⁺/CD8⁺; activated caspase 3⁻/CCR7⁻/CD27⁻/CAR⁺; activated caspase 3⁻/CCR7⁻/CD27⁻/CAR⁺/CD4⁺; and/or activated caspase 3⁻/CCR7⁻/CD27⁻/CAR⁺/CD8⁺; or a combination thereof.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells, each inclusive. In some embodiments, the unit dose comprises no more than about 1×10⁸, no more than about 5×10⁷, no more than about 1×10⁷, no more than about 5×10⁶, no more than about 1×10⁶, or no more than about 5×10⁵ total receptor⁺/CD8⁺ cells or total receptor⁺/CD4⁺ cells, total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells.

In some embodiments, the unit dose comprises between at or about 5×10⁵ and at or about 5×10⁷, between at or about 1×10⁶ and at or about 1×10⁷, or between at or about 5×10⁶ and at or about 1×10⁷ total receptor⁺/CD8⁺/CCR7⁺ cells or receptor⁺/CD4⁺/CCR7⁺ cells, each inclusive. In some embodiments, the unit dose comprises at least or at least about 5×10⁷, 1×10⁷, 5×10⁶, 1×10⁶, or at least about 5×10⁵ total receptor⁺/CD8⁺/CCR7⁺ cells or receptor⁺/CD4⁺/CCR7⁺ cells.

In some embodiments, the unit dose comprises between at or about 5×10⁵ and at or about 5×10⁷, between at or about 1×10⁶ and at or about 1×10⁷, or between at or about 5×10⁶ and at or about 1×10⁷ total receptor⁺/CD8⁺/CD27⁺ cells or receptor⁺/CD4⁺/CD27⁺ cells, each inclusive. In some embodiments, the unit dose comprises at least or at least about 5×10⁷, 1×10⁷, 5×10⁶, 1×10⁶, or at least about 5×10⁵ total receptor⁺/CD8⁺/CD27⁺ cells or receptor⁺/CD4⁺/CD27⁺ cells.

In some embodiments, the unit dose comprises at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD8⁺/CCR7⁺ cells and/or at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD4⁺/CCR7⁺ cells, each inclusive. In some embodiments, the unit dose comprises between at or about 3×10⁶ and at or about 2.5×10⁷, between at or about 4×10⁶ and at or about 2×10⁷, or between at or about 5×10⁶ and at or about 1×10⁷ total receptor⁺/CD8⁺/CCR7⁺ cells and/or between at or about 3×10⁶ and at or about 2.5×10⁷, between at or about 4×10⁶ and at or about 2×10⁷, or between at or about 5×10⁶ and at or about 1×10⁷ total receptor⁺/CD4⁺/CCR7 + cells, each inclusive.

In some embodiments, the unit dose comprises at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD8⁺/CD27⁺ cells and/or at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD4⁺/CD27⁺ cells, each inclusive. In some embodiments, the unit dose comprises unit dose comprises between at or about 3×10⁶ and at or about 2.5×10⁷, between at or about 4×10⁶ and at or about 2×10⁷, or between at or about 5×10⁶ and at or about 1×10⁷ total receptor⁺/CD8⁺/CD27⁺ cells and/or between at or about 3×10⁶ and at or about 2.5×10⁷, between at or about 4×10⁶ and at or about 2×10⁷, or between at or about 5×10⁶ and at or about 1×10⁷ total receptor⁺/CD4⁺/CD27⁺ cells, each inclusive.

In some embodiments, the unit dose comprises between at or about 5×10⁵ and at or about 5×10⁷, between at or about 1×10⁶ and at or about 1×10⁷, or between at or about 5×10⁶ and at or about 1×10⁷ total receptor⁺/CD8+/CCR7⁺/CD27⁺ cells or receptor⁺/CD4⁺/CCR7⁺/CD27⁺ cells, each inclusive. In some embodiments, the unit dose comprises at least or at least at or about 5×10⁷, 1×10⁷, 5×10⁶, 1×10⁶, or at least at or about 5×10⁵ total receptor⁺/CD8⁺/CCR7⁺/CD27⁺ cells or receptor⁺/CD4⁺/CCR7⁺/CD27⁺ cells.

In some embodiments, the unit dose comprises at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD8⁺/CCR7⁺/CD27⁺ cells and/or at least at or about 1×10⁶, 2×10⁶, 3×10⁶, 4×10⁶, 5×10⁶, 6×10⁶, 7×10⁶, 8×10⁶, 9×10⁶, or 1×10⁷ total receptor⁺/CD4⁺/CCR7⁺/CD27⁺ cells, each inclusive. In some embodiments, the unit dose comprises between at or about 3×10⁶ and at or about 2.5×10⁷, between at or about 4×10⁶ and at or about 2×10⁷, or between at or about 5×10⁶ and at or about 1×10⁷ total receptor⁺/CD8⁺/CCR7⁺/CD27⁺ cells and/or between at or about 3×10⁶ and at or about 2.5×10⁷, between at or about 4×10⁶ and at or about 2×10⁷, or between at or about 5×10⁶ and at or about 1×10⁷ total receptor⁺/CD4⁺/CCR7⁺/CD27⁺ cells, each inclusive.

In some embodiments, the unit dose of cells comprises a defined ratio of receptor⁺/CD8⁺/CCR7⁺ cells to receptor⁺/CD4⁺/CCR7⁺ cells, which ratio optionally is or is approximately 1:1 or is between approximately 1:3 and approximately 3:1.

In some embodiments, the unit dose of cells comprises a defined ratio of receptor⁺/CD8⁺/CD27⁺ cells to receptor⁺/CD4⁺/CD27⁺ cells, which ratio optionally is or is approximately 1:1 or is between approximately 1:3 and approximately 3:1.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺/CD27⁺ cells, or total receptor⁺/CD4⁺/CCR7⁺/CD27⁺ cells, each inclusive. In some embodiments, the unit dose comprises no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD8⁺ cells or total receptor⁺/CD4⁺ cells, total receptor⁺/CD8⁺/CCR7⁺/CD27⁺ cells, or total receptor⁺/CD4⁺/CCR7⁺/CD27⁺ cells.

In some embodiments, the unit dose of cells comprises a defined ratio of receptor⁺/CD8⁺/CCR7⁺/CD27⁺ cells to receptor⁺/CD4⁺/CCR7⁺/CD27⁺ cells, which ratio optionally is or is approximately 1:1 or is between approximately 1:3 and approximately 3:1.

In some embodiments, the unit dose comprises between at or about 1×10⁵ and at or about 5×10⁸, between at or about 1×10⁵ and at or about 1×10⁸, between at or about 5×10⁵ and at or about 1×10⁷, or between at or about 1×10⁶ and at or about 1×10⁷ total CD3⁺ cells that express the recombinant receptor (receptor⁺/CD3⁺ cells) or total CD3⁺ cells, each inclusive. In some embodiments, the unit dose comprises no more than at or about 5×10⁸, no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD3⁺ cells or total CD3⁺ cells.

In some embodiments, the total number of CD3⁺ cells, total number of receptor⁺/CD3⁺ cells, total number of receptor⁺/CD8⁺ cells, total number of receptor⁺/CD4⁺ cells, total number of receptor⁺/CD8⁺/CCR7⁺ cells, total number of receptor⁺/CD4⁺/CCR7⁺ cells, total number of receptor⁺/CD8⁺/CD27⁺ cells, total number of receptor⁺/CD4⁺/CD27⁺ cells, total number of receptor⁺/CD8⁺/CCR7⁺/CD27⁺ cells, total number of receptor⁺/CD4⁺/CCR7⁺/CD27⁺ cells, total number of receptor⁺/CD8⁺/CCR7⁺/CD45RA⁻ cells and/or receptor⁺/CD4⁺/CCR7⁺/CD45RA⁻ cells is the total number of such cells that are live or viable. In some embodiments, the total number of CD3⁺ cells, total number of receptor⁺/CD3⁺ cells, total number of receptor⁺/CD8⁺ cells, total number of receptor⁺/CD4⁺ cells, total number of receptor⁺/CD8⁺/CCR7⁺ cells, total number of receptor⁺/CD4⁺/CCR7⁺ cells, total number of receptor⁺/CD8⁺/CD27⁺ cells, total number of receptor⁺/CD4⁺/CD27⁺ cells, total number of receptor⁺/CD8⁺/CCR7⁺/CD27⁺ cells, total number of receptor⁺/CD4⁺/CCR7⁺/CD27⁺ cells, total number of receptor⁺/CD8⁺/CCR7⁺/CD45RA⁻ cells and/or receptor⁺/CD4⁺/CCR7⁺/CD45RA⁻ cells is the total number of such cells that do not express an apoptotic marker and/or is the total number of such cells that are apoptotic marker negative (−), wherein the apoptotic marker is Annexin V or activated Caspase 3.

In some embodiments, in any of the composition comprising T cells expressing a recombinant receptor provided herein, at least at or about, or at or about, 15%, 20%, 30%, 40%, 50%, 60%, 70%, 80%, 85%, 90%, 91%, 92%, 93%, 94%, 95%, 96%, 97%, 98%, 99% or 100% of the total number of T cells in the composition (or of the total number of T cells in the composition expressing the recombinant receptor), are surface positive for CCR7 and/or CD27.

In some embodiments, in any of the composition comprising T cells expressing a recombinant receptor provided herein, at least at or about, or at or about, 70%, 80%, 85%, 90%, 91%, 92%, 93%, 94%, 95%, 96%, 97%, 98%, 99% or 100% of the total number of T cells in the composition (or of the total number of T cells in the composition expressing the recombinant receptor), are able to produce a cytokine selected from interleukin 2 (IL-2) and/or TNF-alpha. In some embodiments, the T cell able to produce IL-2 and/or TNF-alpha is a CD4+ T cell.

In some embodiments, in any of the composition comprising T cells expressing a recombinant receptor provided herein, at least at or about, or at or about, 15%, 20%, 30%, 40%, 50%, 60%, 70%, 80% or 90% of the total receptor⁺ cells in the unit dose, or between at or about 15% and at or about 90%, between at or about 20% and at or about 80%, between at or about 30% and at or about 70%, or between at or about 40% and at or about 60%, each inclusive, of the total receptor⁺ cells in the unit dose are receptor⁺/CD8⁺/CCR7⁺ or receptor⁺/CD8⁺/CD27⁺. In some embodiments, in any of the composition comprising T cells expressing a recombinant receptor provided herein, at least at or about 15%, 20%, 30%, 40%, 50%, 60%, 70%, 80% or 90% of the total receptor⁺ cells in the unit dose, or between at or about 15% and at or about 90%, between at or about 20% and at or about 80%, between at or about 30% and at or about 70%, or between at or about 40% and at or about 60%, each inclusive, of the total receptor⁺ cells in the unit dose are receptor⁺/CD4⁺/CCR7⁺ or receptor⁺/CD4⁺/CD27⁺. In some embodiments, at least at or about 15%, 20%, 30%, 40%, 50%, 60%, 70%, 80% or 90% of the total receptor⁺ cells in the unit dose, or between at or about 15% and at or about 90%, between at or about 20% and at or about 80%, between at or about 30% and at or about 70%, or between at or about 40% and at or about 60%, each inclusive, of the total receptor⁺ cells in the unit dose are receptor⁺/CD8⁺/CCR7⁺/CD27⁺, receptor⁺/CD8⁺/CCR7⁺/CD45RA⁻, receptor⁺/CD4⁺/CCR7⁺/CD27⁺ or receptor⁺/CD4⁺/CCR7⁺/CD45RA⁻.

In some embodiments, in any of the composition comprising T cells expressing a recombinant receptor provided herein, at least at or about 50%, 60%, 70%, 80% or 90% of the total receptor⁺/CD8⁺ cells in the composition or unit dose are or the unit dose, or between at or about 50% and at or about 90%, between at or about 60% and at or about 90%, between at or about 70% and at or about 80%, each inclusive, of the total receptor⁺/CD8⁺ cells in the composition or the unit dose are receptor⁺/CD8⁺/CCR7⁺ or receptor⁺/CD8⁺/CD27^{+.} or receptor⁺/CD8⁺/CCR7⁺/CD27⁺. In some embodiments, in any of the composition comprising T cells expressing a recombinant receptor provided herein, at least at or about 50%, 60%, 70%, 80% or 90% of the total receptor⁺/CD4⁺ cells in the composition or unit dose are or the unit dose, or between at or about 50% and at or about 90%, between at or about 60% and at or about 90%, between at or about 70% and at or about 80%, each inclusive, of the total receptor⁺/CD4⁺ cells in the composition or the unit dose are receptor⁺/CD4⁺/CCR7⁺ or receptor⁺/CD4⁺/CD27^{+.} or receptor⁺/CD4⁺/CCR7⁺/CD27⁺. receptor^{+/CD}8⁺/CCR7⁺/CD27⁺, receptor⁺/CD8⁺/CCR7⁺/CD45RA⁻, receptor⁺/CD4⁺/CCR7⁺/CD27⁺ or receptor⁺/CD4⁺/CCR7⁺/CD45RA⁻. In some embodiments, at least at or about 50%, 60%, 70%, 80% or 90% of the total receptor⁺/CD8⁺ cells in the composition are receptor⁺/CD8⁺/CCR7⁺/CD27⁺; or at least at or about 15%, 20%, 30%, 40%, 50%, 60%, 70%, 80% or 90% of the total receptor⁺/CD4⁺ cells in the composition are receptor⁺/CD4⁺/CCR7⁺/CD27⁺.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells, each inclusive. In some embodiments, the unit dose comprises no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD8⁺ cells or total receptor⁺/CD4⁺ cells, total receptor⁺/CD8⁺/CCR7⁺ cells, total receptor⁺/CD4⁺/CCR7⁺ cells, total receptor⁺/CD8⁺/CD27⁺ cells, or total receptor⁺/CD4⁺/CD27⁺ cells.

In some embodiments, the unit dose of cells comprises a defined ratio of receptor⁺/CD8⁺/CCR7⁺ cells to receptor⁺/CD4⁺/CCR7⁺ cells, which ratio optionally is or is approximately 1:1 or is between approximately 1:3 and approximately 3:1.

In some embodiments, the unit dose comprises between at or about 1×10⁵ and at or about 1×10⁸, between at or about 5×10⁵ and at or about 1×10⁷, or between at or about 1×10⁶ and at or about 1×10⁷ total CD8⁺ cells that express the recombinant receptor (receptor⁺/CD8⁺ cells) or total CD4⁺ cell that express the recombinant receptor (receptor⁺/CD4⁺ cells), total receptor⁺/CD8⁺/CCR7⁺/CD27⁺ cells, or total receptor⁺/CD4⁺/CCR7⁺/CD27⁺ cells, each inclusive. In some embodiments, the unit dose comprises no more than at or about 1×10⁸, no more than at or about 5×10⁷, no more than at or about 1×10⁷, no more than at or about 5×10⁶, no more than at or about 1×10⁶, or no more than at or about 5×10⁵ total receptor⁺/CD8⁺ cells or total receptor⁺/CD4⁺ cells, total receptor⁺/CD8⁺/CCR7⁺/CD27⁺ cells, or total receptor⁺/CD4⁺/CCR7⁺/CD27⁺ cells.

In some embodiments, the unit dose of cells comprises a defined ratio of receptor⁺/CD8⁺/CCR7⁺/CD27⁺ cells to receptor⁺/CD4⁺/CCR7⁺/CD27⁺ cells, which ratio optionally is or is approximately 1:1 or is between approximately 1:3 and approximately 3:1.

In some embodiments, the provided methods involve administering a dose containing a defined number of cells. In some embodiments, the dose, such as the defined number of cells, such as a defined number of CAR⁺ cells that are CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, or CCR7⁻/CD27⁻/CD8⁺, is between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷. In some embodiments, such dose, such as such defined number of cells refers to the total recombinant-receptor expressing cells in the administered composition. In some aspects, the defined number of recombinant receptor-expressing cells that are administered are cells that are apoptotic marker negative (−) and optionally wherein the apoptotic marker is Annexin V or activated Caspase 3.

In some embodiments, the dose of cells of the unit dose contains a number of cells, such as a defined number of cells, between at least or at least about 5×10⁶, 6×10⁶, 7×10⁶, 8×10⁶, 9×10⁶, 10×10⁶ and about 15×10⁶ recombinant-receptor expressing cells, such as recombinant-receptor expressing cells that are CCR7⁺/CD4⁺, CCR7⁺/CD8⁺, CD27⁺/CD4⁺, CD27⁺/CD8⁺, CD45RA⁺/CD4⁺, CD45RA⁺/CD8⁺, CCR7⁻/CD4⁺, CCR7⁻/CD8⁺, CD27⁻/CD4⁺, CD27⁻/CD8⁺, CD45RA⁻/CD4⁺, CD45RA⁻/CD8⁺, CCR7⁺/CD27⁺/CD4⁺, CCR7⁺/CD27⁺/CD8⁺, CCR7⁺/CD45RA⁻/CD4⁺, CCR7⁺/CD45RA⁻/CD8⁺, CCR7⁻/CD45RA⁻/CD4⁺, CCR7⁻/CD45RA⁻/CD8⁺, CCR7⁻/CD27⁻/CD4⁺, or CCR7⁻/CD27⁻/CD8⁺, and/or that are apoptotic marker negative (−) and CD8⁺, optionally wherein the apoptotic marker is Annexin V or activated Caspase 3.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g., antigen-expressing, such as CAR-expressing cells) per kilogram body weight of the subject (cells/kg), such as no more than at or about 3×10⁵cells/kg, no more than at or about 4×10⁵ cells/kg, no more than at or about 5×10⁵ cells/kg, no more than at or about 6×10⁵cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g., antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, at or about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments. In some embodiments, the dose of cells is a flat dose of cells or fixed dose of cells such that the dose of cells is not tied to or based on the body surface area or weight of a subject.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total recombinant receptor (e.g., CAR)-expressing cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁸ total such cells, or the range between any two of the foregoing values.

In some embodiments, the dose of genetically engineered cells comprises from at or about 1×10⁵ to at or about 5×10⁸ total CAR-expressing (CAR-expressing) T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells. In some embodiments, the dose of genetically engineered cells comprises from or from about 2.5×10⁷ to at or about 1.5×10⁸ total CAR-expressing T cells, such as from or from about 5×10⁷ to or to about 1×10⁸ total CAR-expressing T cells.

In some embodiments, the dose of genetically engineered cells comprises at least at or about 1×10⁵ CAR-expressing cells, at least at or about 2.5×10⁵ CAR-expressing cells, at least at or about 5×10⁵ CAR-expressing cells, at least at or about 1×10⁶ CAR-expressing cells, at least at or about 2.5×10⁶ CAR-expressing cells, at least at or about 5×10⁶ CAR-expressing cells, at least at or about 1×10⁷ CAR-expressing cells, at least at or about 2.5×10⁷ CAR-expressing cells, at least at or about 5×10⁷ CAR-expressing cells, at least at or about 1×10⁸ CAR-expressing cells, at least at or about 1.5×10⁸ CAR-expressing cells, at least at or about 2.5×10⁸ CAR-expressing cells, or at least at or about 5×10⁸ CAR-expressing cells.

In some embodiments, the cell therapy comprises administration of a dose comprising a number of cell from or from about 1×10⁵ to or to about 5×10⁸ total recombinant receptor-expressing cells, total T cells, or total peripheral blood mononuclear cells (PBMCs), from or from about 5×10⁵ to or to about 1×10⁷ total recombinant receptor-expressing cells, total T cells, or total peripheral blood mononuclear cells (PBMCs) or from or from about 1×10⁶ to or to about 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3⁺ or CD8⁺, in some cases also recombinant receptor-expressing (e.g., CAR⁺) CD3+ or CD8+ cells. In some embodiments, the number is with reference to the total number of CD4⁺ and CD8⁺, in some cases recombinant receptor-expressing (e.g., CAR⁺) CD4+ and CD8+ cells.

In some embodiments, the cell therapy comprises administration of a dose comprising a number of cell from or from about 1×10⁵ to or to about 5×10⁸ CD3⁺ or CD8⁺ total T cells or CD3⁺ or CD8⁺ recombinant receptor-expressing cells, from or from about 5×10⁵ to or to about 1×10⁷ CD3⁺ or CD8⁺ total T cells or CD3⁺ or CD8⁺ recombinant receptor-expressing cells, or from or from about 1×10⁶ to or to about 1×10⁷ CD3⁺ or CD8⁺ total T cells or CD3⁺ or CD8⁺ recombinant receptor-expressing cells, each inclusive. In some embodiments, the cell therapy comprises administration of a dose comprising a number of cell from or from about 1×10⁵ to or to about 5×10⁸ CD4⁺ and CD8⁺ total T cells or CD4⁺ and CD8⁺ recombinant receptor-expressing cells, from or from about 5×10⁵ to or to about 1×10⁷ CD4⁺ and CD8⁺ total T cells or CD4⁺ and CD8⁺ recombinant receptor-expressing cells, or from or from about 1×10⁶ to or to about 1×10⁷ CD4⁺ and CD8⁺ total T cells or CD4⁺ and CD8⁺ recombinant receptor-expressing cells, each inclusive.

In some embodiments, the cell therapy comprises administration of a dose comprising a number of cell from or from about 1×10⁵ to or to about 5×10⁸ total CD3⁺/CAR⁺ or CD8⁺/CAR⁺ cells, from or from about 5×10⁵ to or to about 1×10⁷ total CD3⁺/CAR⁺ or CD8⁺/CAR⁺ cells, or from or from about 1×10⁶ to or to about 1×10⁷ total CD3⁺/CAR⁺ or CD8⁺/CAR⁺ cells, each inclusive. In some embodiments, the cell therapy comprises administration of a dose comprising a number of cell from or from about 1×10⁵ to or to about 5×10⁸ total CD4⁺/CAR⁺ and CD8⁺/CAR⁺ cells, from or from about 5×10⁵ to or to about 1×10⁷ total CD4⁺/CAR⁺ and CD8⁺/CAR⁺ cells, or from or from about 1×10⁶ to or to about 1×10⁷ total CD4⁺/CAR⁺ and CD8⁺/CAR⁺ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 5×10⁸ total recombinant receptor (e.g., CAR)-expressing CD8+ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8+ T cells, from or from about 1×10⁷ to or to about 5×10⁷ total recombinant receptor-expressing CD8+ T cells, from or from about 1×10⁷ to or to about 0.25×10⁸ total recombinant receptor-expressing CD8+ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD8+ T cells.

In some embodiments, for example, where the subject is human, the CD4+ T cells of the dose, including in a dose including CD4+ and CD8+ T cells, includes between at or about 1×10⁶ and at or about 5×10⁸ total recombinant receptor (e.g., CAR)-expressing CD4+ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD4+ T cells, from or from about 1×10⁷ to or to about 5×10⁷ total recombinant receptor-expressing CD4+ T cells, from or from about 1×10⁷ to or to about 0.25×10⁸ total recombinant receptor-expressing CD4+ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD4+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8⁺- and CD4⁺-enriched populations, respectively, e.g., CD4⁺ and/or CD8⁺ T cells each individually including cells genetically engineered to express the recombinant receptor. In some embodiments, the administration of the dose comprises administration of a first composition comprising a dose of CD8⁺ T cells or a dose of CD4⁺ T cells and administration of a second composition comprising the other of the dose of CD4⁺ T cells and the CD8⁺ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some embodiments, the first composition, e.g., first composition of the dose, comprises CD4⁺ T cells. In some embodiments, the first composition, e.g., first composition of the dose, comprises CD8⁺ T cells. In some embodiments, the first composition is administered prior to the second composition. In some embodiments, the second composition, e.g., second composition of the dose, comprises CD4+ T cells. In some embodiments, the second composition, e.g., second composition of the dose, comprises CD8+ T cells.

In some embodiments, the dose or composition of cells includes a defined or target ratio of CD4⁺ cells expressing a recombinant receptor to CD8⁺ cells expressing a recombinant receptor and/or of CD4⁺ cells to CD8⁺ cells, which ratio optionally is approximately 1:1 or is between approximately 1:3 and approximately 3:1, such as approximately 1:1. In some aspects, the administration of a composition or dose with the target or desired ratio of different cell populations (such as CD4⁺:CD8⁺ ratio or CAR⁺CD4⁺:CAR⁺CD8⁺ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g., consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios. For example, in some embodiments, the desired ratio (e.g., ratio of CD4⁺ to CD8⁺ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1), such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered. In some embodiments, the size of the dose is determined based upon predicted output cell composition attributes. In some of any of the above embodiments, the dose may be a predetermined dose and/or a predetermined regimen. In some embodiments, the size of the dose, concentration of the dose, and/or frequency of administering the dose may be modified to achieve positive clinical outcome (e.g., response). In some embodiments, altering the dose size, concentration, and/or frequency of administration results in altering a predetermined dose and/or treatment regime.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

### II. METHODS FOR GENERATING ENGINEERED T CELLS

In some embodiments, the methods of identifying features associated with clinical response to a therapeutic cell composition or methods for determining a response to treatment with a therapeutic cell composition prior to treatment are used in connection with generating a therapeutic composition of engineered cells (e.g., output composition), such as engineered CD4+ T cells and/or engineered CD8+ T cells, that express a recombinant protein, e.g., a recombinant receptor such as a T cell receptor (TCR) or a chimeric antigen receptor (CAR). In some embodiments, the methods provided herein are used in connection with manufacturing, generating, or producing a cell therapy, and may be used in connection with additional processing steps, such as steps for the isolation, separation, selection, activation or stimulation, transduction, washing, suspension, dilution, concentration, and/or formulation of the cells. In some embodiments, the methods of generating or producing engineered cells, e.g., engineered CD4+ T cells and/or engineered CD8+ T cells, include one or more of isolating cells from a subject, preparing, processing, incubating under stimulating conditions, and/or engineering (e.g., transducing) the cells. In some embodiments, the method includes processing steps carried out in an order in which: input cells, e.g., primary cells, are first isolated, such as selected or separated, from a biological sample; input cells are incubated under stimulating conditions, engineered with vector particles, e.g., viral vector particles, to introduce a recombinant polynucleotide into the cells, e.g., by transduction or transfection; cultivating the engineered cells, e.g., transduced cells, such as to expand the cells; and collecting, harvesting, and/or filling a container with all or a portion of the cells for formulating the cells in an output composition. In some embodiments, CD4+ and CD8+ T cells are manufactured independently from one another, e.g., in separate input compositions, but the process for manufacturing includes the same processing steps. In some embodiments, CD4+ and CD8+ T cells are manufactured together, e.g., in the same input composition. In some embodiments, the features of the selected cells (e.g., input composition) are determined and used as input to machine learning model, e.g., random forest model, random survival forests model provided herein.

In some embodiments, the cells of the generated output composition (e.g., therapeutic cell composition) are re-introduced into the same subject, before or after cryopreservation. In some embodiments, the features of the engineered cells of the therapeutic cell composition are determined and used as input to machine learning model, e.g., random forest model, random survival forests model provided herein. In some embodiments, the output compositions of engineered cells (e.g., therapeutic cell composition) are suitable for use in a therapy, e.g., an autologous cell therapy.). Exemplary manufacturing methods are described in published international patent application, publication no. WO 2019/089855, the contents of which are incorporated herein by reference in their entirety.

A. Samples and Cell preparations

In particular embodiments, the provided methods are used in connection with isolating, selecting, and/or enriching cells from a biological sample to generate one or more input compositions of enriched cells, e.g., T cells. In some embodiments, the provided methods include isolation of cells or compositions thereof from biological samples, such as those obtained from or derived from a subject, such as one having a particular disease or condition or in need of a cell therapy or to which cell therapy will be administered. In some embodiments, features of the subject, for example as described in Section I-A and I-A.1 above, to be treated are determined or obtained and used a input to machine learning models provided herein. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the preparation methods include steps for freezing, e.g., cryopreserving, the cells, either before or after isolation, selection and/or enrichment and/or incubation for transduction and engineering, and/or after cultivation and/or harvesting of the engineered cells. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9. 0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to or to about −80° C. at a rate of or of about 1° per minute and stored in the vapor phase of a liquid nitrogen storage tank.

In some embodiments, isolation of the cells or populations includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g., magnetic beads, that are coated with a selection agent (e.g., antibody) specific to the marker of the cells. The particles (e.g., beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g., bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g., is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from 5 minutes to 6 hours or from about 5 minutes to about 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm), such as at an RCF at the sample or wall of the chamber or other container of from 80 g to 100 g or from about 80 g to about 100 g (e.g., at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

Such separation steps can be based on positive selection, in which the cells having bound the reagents, e.g., antibody or binding partner, are retained for further use, and/or negative selection, in which the cells having not bound to the reagent, e.g., antibody or binding partner, are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the process steps further include negative and/or positive selection of the incubated and cells, such as using a system or apparatus that can perform an affinity-based selection. In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively. Multiple rounds of the same selection step, e.g., positive or negative selection step, can be performed. In certain embodiments, the positively or negatively selected fraction subjected to the process for selection, such as by repeating a positive or negative selection step. In some embodiments, selection is repeated twice, three times, four times, five times, six times, seven times, eight times, nine times or more than nine times. In certain embodiments, the same selection is performed up to five times. In certain embodiments, the same selection step is performed three times.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. In certain embodiments, one or more separation steps are repeated and/or performed more than once. In some embodiments, the positively or negatively selected fraction resulting from a separation step is subjected to the same separation step, such as by repeating the positive or negative selection step. In some embodiments, a single separation step is repeated and/or performed more than once, for example, to increase the yield of positively selected cells, to increase the purity of negatively selected cells, and/or to further remove the positively selected cells from the negatively selected fraction. In certain embodiments, one or more separation steps are performed and/or repeated two times, three times, four times, five times, six times, seven times, eight times, nine times, ten times, or more than ten times. In certain embodiments, the one or more selection steps are performed and/or repeated between one and ten times, between one and five times, or between three and five times. In certain embodiments, one or more selection steps are repeated three times.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some embodiments, such cells are selected by incubation with one or more antibody or binding partner that specifically binds to such markers. In some embodiments, the antibody or binding partner can be conjugated, such as directly or indirectly, to a solid support or matrix to effect selection, such as a magnetic bead or paramagnetic bead. For example, CD3+, CD28+ T cells can be positively selected using CD3/CD28 conjugated magnetic beads (e.g., DYNABEADS® M-450 CD3/CD28 T Cell Expander, and/or ExpACT® beads).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood.1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+and CD62L− subsets of CD8+ peripheral blood lymphocytes. PBMC can be enriched for or depleted of CD62L-−CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L.

Such selections in some aspects are carried out simultaneously and in other aspects are carried out sequentially, in either order. In some aspects, the same CD4 expression-based selection step used in preparing the CD8+ T cell population or subpopulation, also is used to generate the CD4+ T cell population or sub-population, such that both the positive and negative fractions from the CD4-based separation are retained and used in subsequent steps of the methods, optionally following one or more further positive or negative selection steps. In some embodiments, the selection for the CD4+ T cell population and the selection for the CD8+ T cell population are carried out simultaneously. In some embodiments, the CD4+ T cell population and the selection for the CD8+ T cell population are carried out sequentially, in either order. In some embodiments, methods for selecting cells can include those as described in published U.S. App. No. US20170037369. In some embodiments, the selected CD4+ T cell population and the selected CD8+ T cell population may be combined subsequent to the selecting. In some aspects, the selected CD4+ T cell population and the selected CD8+ T cell population may be combined in a bioreactor bag as described herein. In some embodiments, the selected CD4+ T cell population and the selected CD8+ T cell population are separately processed, whereby the selected CD4+ T cell population is enriched in CD4+ T cells and incubated with a stimulatory reagent (e.g., anti-CD3/anti-CD28 magnetic beads), transduced with a viral vector encoding a recombinant protein (e.g., CAR) and cultivated under conditions to expand T cells and the selected CD8+ T cell population is enriched in CD8+ T cell and incubated with a stimulatory reagent (e.g., anti-CD3/anti-CD28 magnetic beads), transduced with a viral vector encoding a recombinant protein (e.g., CAR), such as the same recombinant protein as for engineering of the CD4+ T cells from the same donor, and cultivated under conditions to expand T cells, such as in accord with the provided methods.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In a particular example, a sample of PBMCs or other white blood cell sample is subjected to selection of CD4+ T cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells may be sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, or CD4+ T cells. In some embodiments, central memory CD4+ T cells are CD62L+and CD45RO+. In some embodiments, effector CD4+ T cells are CD62L− and CD45RO−.

In one example, to enrich for CD4+ T cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the incubated sample or composition of cells to be separated is incubated with a selection reagent containing small, magnetizable or magnetically responsive material, such as magnetically responsive particles or microparticles, such as paramagnetic beads (e.g., such as Dynalbeads or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. Many well-known magnetically responsive materials for use in magnetic separation methods are known, e.g., those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 also may be used.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some aspects, separation is achieved in a procedure in which the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotech, Auburn, Calif.). Magnetic Activated Cell Sorting (MACS), e.g., CliniMACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labeled and depleted from the heterogeneous population of cells.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In some embodiments, features of the one or more input compositions are assessed, for example as described in Sections I-A and I-A-2. In some embodiments, the features are cell phenotypes. In some embodiments, the cell phenotypes, are quantified to provide a number, percentage, proportion, and/or ratio of cells having an attribute in the input composition. In some embodiments, the features are used as input to machine learning models provided herein.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In particular embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

In some embodiments, the one or more input compositions of enriched T cells are frozen, e.g., cryopreserved and/or cryofrozen, after isolation, selection and/or enrichment. In some embodiments, the one or more input compositions of frozen e.g., cryopreserved and/or cryofrozen, prior to any steps of incubating, activating, stimulating, engineering, transducing, transfecting, cultivating, expanding, harvesting, and/or formulating the composition of cells. In particular embodiments, the one or more cryofrozen input compositions are stored, e.g., at or at about −80° C., for between 12 hours and 7 days, between 24 hours and 120 hours, or between 2 days and 5 days. In particular embodiments, the one or more cryofrozen input compositions are stored at or at about −80° C., for an amount of time of less than 10 days, 9 days, 8 days, 7 days, 6 days, or 5 days, 4 days, 3 days, 2 days, or 1 day. In some embodiments, the one or more cryofrozen input compositions are stored at or at about −80° C., for or for about 1 day, 2 days, 3 days, 4 days, 5 days, or 6 days.

B. Activation and Stimulation of Cells

In some embodiments, the provided methods are used in connection with incubating cells under stimulating conditions. In some embodiments, the stimulating conditions include conditions that activate or stimulate, and/or are capable of activating or stimulating a signal in the cell, e.g., a CD4+ T cell or CD8+ T cell, such as a signal generated from a TCR and/or a coreceptor. In some embodiments, the stimulating conditions include one or more steps of culturing, cultivating, incubating, activating, propagating the cells with and/or in the presence of a stimulatory reagent, e.g., a reagent that activates or stimulates, and/or is capable of activating or stimulating a signal in the cell. In some embodiments, the stimulatory reagent stimulates and/or activates a TCR and/or a coreceptor. In particular embodiments, the stimulatory reagent is a reagent described in Section II-B-1.

In certain embodiments, one or more compositions of enriched T cells are incubated under stimulating conditions prior to genetically engineering the cells, e.g., transfecting and/or transducing the cell such as by a technique provided in Section II-C. In particular embodiments, one or more compositions of enriched T cells are incubated under stimulating conditions after the one or more compositions have been isolated, selected, enriched, or obtained from a biological sample. In particular embodiments, the one or more compositions are input compositions. In particular embodiments, the one or more input compositions have been previously cryofrozen and stored, and are thawed prior to the incubation.

In certain embodiments, the one or more compositions of enriched T cells are or include two separate compositions, e.g., separate input compositions, of enriched T cells. In particular embodiments, two separate compositions of enriched T cells, e.g., two separate compositions of enriched T cells selected, isolated, and/or enriched from the same biological sample, are separately incubated under stimulating conditions. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells are separately incubated under stimulating conditions.

In some embodiments, a single composition of enriched T cells is incubated under stimulating conditions. In certain embodiments, the single composition is a composition of enriched CD4+ T cells. In some embodiments, the single composition is a composition of enriched CD4+ and CD8+ T cells that have been combined from separate compositions prior to the incubation.

In some embodiments, the composition of enriched CD4+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are incubated under stimulating conditions. In certain embodiments, separate stimulated compositions of enriched CD4+ and enriched CD8+ T cells are combined into a single composition after the incubation has been performed and/or completed. In some embodiments, separate stimulated compositions of stimulated CD4+ and stimulated CD8+ T cells are separately processed after the incubation has been performed and/or completed, whereby the stimulated CD4+ T cell population (e.g., incubated with stimulatory an anti-CD3/anti-CD28 magnetic bead stimulatory reagent) is transduced with a viral vector encoding a recombinant protein (e.g., CAR) and cultivated under conditions to expand T cells and the stimulated CD8+ T cell population (e.g., incubated with stimulatory an anti-CD3/anti-CD28 magnetic bead stimulatory reagent) is transduced with a viral vector encoding a recombinant protein (e.g., CAR), such as the same recombinant protein as for engineering of the CD4+ T cells from the same donor, and cultivated under conditions to expand T cells, such as in accord with the provided methods.

In some embodiments, the incubation under stimulating conditions can include culture, cultivation, stimulation, activation, propagation, including by incubation in the presence of stimulating conditions, for example, conditions designed to induce proliferation, expansion, activation, and/or survival of cells in the population, to mimic antigen exposure, and/or to prime the cells for genetic engineering, such as for the introduction of a recombinant antigen receptor. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, the stimulation and/or incubation under stimulating conditions is carried out in accordance with techniques such as those described in U.S. Pat. No. 6,040,1 77 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, the cells, e.g., T cells, compositions of cells, and/or cell populations, such as CD4⁺ and CD8⁺ T cells or compositions, populations, or subpopulations thereof, are expanded by adding to the culture-initiating composition feeder cells, such as non-dividing peripheral blood mononuclear cells (PBMCs) (e.g., such that the resulting population of cells contains at least about 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. In some embodiments, a temperature shift is effected during culture, such as from 37 degrees Celsius to 35 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, populations of CD4⁺ and CD8⁺ that are antigen specific can be obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen. Naive T cells may also be used.

In particular embodiments, the stimulating conditions include incubating, culturing, and/or cultivating the cells with a stimulatory reagent. In particular embodiments, the stimulatory reagent is a reagent described in Section I-B-1. In certain embodiments, the stimulatory reagent contains or includes a bead. An exemplary stimulatory reagent is or includes anti-CD3/anti-CD28 magnetic beads. In certain embodiments, the start and/or initiation of the incubation, culturing, and/or cultivating cells under stimulating conditions occurs when the cells come into contact with and/or are incubated with the stimulatory reagent. In particular embodiments, the cells are incubated prior to, during, and/or subsequent to genetically engineering the cells, e.g., introducing a recombinant polynucleotide into the cell such as by transduction or transfection.

In some embodiments, the composition of enriched T cells are incubated at a ratio of stimulatory reagent and/or beads, e.g., anti-CD3/anti-CD28 magnetic beads, to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of stimulatory reagent and/or beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

In particular embodiments, incubating the cells at a ratio of less than 3:1 or less than 3 stimulatory reagents, e.g., anti-CD3/anti-CD28 magnetic beads. per cell, such as a ratio of 1:1, reduces the amount of cell death that occurs during the incubation, e.g., such as by activation-induced cell death. In some embodiments, the cells are incubated with the stimulatory reagent, e.g., anti-CD3/anti-CD28 magnetic beads, at a ratio of beads to cells of less than 3 (or 3:1 or less than 3 beads per cell). In particular embodiments, incubating the cells at a ratio of less than 3:1 or less than 3 beads per cell, such as a ratio of 1:1, reduces the amount of cell death that occurs during the incubation, e.g., such as by activation-induced cell death.

In particular embodiments, the composition of enriched T cells is incubated with the stimulatory reagent, e.g., anti-CD3/anti-CD28 magnetic beads, at a ratio of less than 3:1 stimulatory reagents and/or beads per cell, such as a ratio of 1:1, and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programmed cell death, or apoptosis, during or at least 1 day, 2 days, 3 days, 4 days, 5 days, 6 days, 7 days, or more than 7 days after the incubation is complete. In particular embodiments, the composition of enriched T cells is incubated with the stimulatory reagent at a ratio of less than 3:1 stimulatory reagents and/or beads per cell, e.g., a ratio of 1:1, and less than 50%, less than 40%, less than 30%, less than 25%, less than 20%, less than 15%, less than 10%, less than 5%, less than 1%, less than 0.1% or less than 0.01% of the cells undergo activation induced cell death during the incubation.

In certain embodiments, the composition of enriched T cells is incubated with the stimulatory reagent, e.g., anti-CD3/anti-CD28 magnetic beads, at a ratio of less than 3:1 beads per cell, e.g., a ratio of 1:1, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing an exemplary and/or alternative process where the composition of enriched T cells in incubated with the stimulatory reagent at a ratio of 3:1 or greater.

In some embodiments, the composition of enriched T cells incubated with the stimulatory reagent comprises from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the composition of enriched T cells incubated with the stimulatory reagent comprises about 0.5×10⁶ cells/mL, 1×10⁶ cells/mL, 1.5×10⁶ cells/mL, 2×10⁶ cells/mL, 2.5×10⁶ cells/mL, 3×10⁶ cells/mL, 3.5×10⁶ cells/mL, 4×10⁶ cells/mL, 4.5×10⁶ cells/mL, 5×10⁶ cells/mL, 5.5×10⁶ cells/mL, 6×10⁶ cells/mL, 6.5×10⁶ cells/mL, 7×10⁶ cells/mL, 7.5×10⁶ cells/mL, 8×10⁶ cells/mL, 8.5×10⁶ cells/mL, 9×10⁶ cells/mL, 9.5×10⁶ cells/mL, or 10×10⁶ cells/mL, such as about 2.4×10⁶ cells/mL.

In some embodiments, the composition of enriched T cells is incubated with the stimulatory reagent at a temperature from about 25 to about 38° C., such as from about 30 to about 37° C., for example at or about 37° C.±2° C. In some embodiments, the composition of enriched T cells is incubated with the stimulatory reagent at a CO₂ level from about 2.5% to about 7.5%, such as from about 4% to about 6%, for example at or about 5%±0.5%. In some embodiments, the composition of enriched T cells is incubated with the stimulatory reagent at a temperature of or about 37° C. and/or at a CO₂ level of or about 5%.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes IL-2. In some embodiments, the stimulating conditions include incubating composition of enriched T cells, such as enriched CD4+ T cells or enriched CD8+ T cells, in the presence of a stimulatory reagent, e.g., anti-CD3/anti-CD28 magnetic beads, as described and in the presence or one or more recombinant cytokines.

In particular embodiments, the composition of enriched CD4+ T cells are incubated with IL-2, e.g., recombinant IL-2. Without wishing to be bound by theory, particular embodiments contemplate that CD4+ T cells that are obtained from some subjects do not produce, or do not sufficiently produce, IL-2 in amounts that allow for growth, division, and expansion throughout the process for generating a composition of output cells, e.g., engineered cells suitable for use in cell therapy. In some embodiments, incubating a composition of enriched CD4+ T cells under stimulating conditions in the presence of recombinant IL-2 increases the probability or likelihood that the CD4+ T cells of the composition will continue to survive, grow, expand, and/or activate during the incubation step and throughout the process. In some embodiments, incubating the composition of enriched CD4+ T cells in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold as compared to an alternative and/or exemplary method that does not incubate the composition of enriched CD4+ T cells in the presence of recombinant IL-2.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength e.g., WHO 1st International Standard for Human IL-2, 86/504. International Units are the only recognized and standardized method to report biological activity units that are published and are derived from an international collaborative research effort. In particular embodiments, the IU for composition, sample, or source of a cytokine may be obtained through product comparison testing with an analogous WHO standard product. For example, in some embodiments, the IU/mg of a composition, sample, or source of human recombinant IL-2, IL-7, or IL-15 is compared to the WHO standard IL-2 product (NIBSC code: 86/500), the WHO standard IL-17 product (NIBSC code: 90/530) and the WHO standard IL-15 product (NIBSC code: 95/554), respectively.

In some embodiments, the biological activity in IU/mg is equivalent to (ED₅₀ in ng/ml)⁻¹×10⁶. In particular embodiments, the ED₅₀ of recombinant human IL-2 or IL-15 is equivalent to the concentration required for the half-maximal stimulation of cell proliferation (XTT cleavage) with CTLL-2 cells. In certain embodiments, the ED₅₀ of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94; hereby incorporated by reference in their entirety.

In particular embodiments, a composition of enriched CD8+ T cells is incubated under stimulating conditions in the presence of IL-2 and/or IL-15. In certain embodiments, a composition of enriched CD4+ T cells is incubated under stimulating conditions in the presence of IL-2, IL-7, and/or IL-15. In some embodiments, the IL-2, IL-7, and/or IL-15 are recombinant. In certain embodiments, the IL-2, IL-7, and/or IL-15 are human In particular embodiments, the one or more cytokines are or include human recombinant IL-2, IL-7, and/or IL-15. In some aspects, the incubation of the enriched T cell composition also includes the presence of a stimulatory reagent, e.g., anti-CD3/anti-CD28 magnetic beads.

In some embodiments, the cells are incubated with a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is incubated with IL-2, e.g., human recombinant IL-2, at a concentration between 1 IU/ml and 200 IU/ml, between 10 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is incubated with recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml. In some embodiments, the composition of enriched T cells is incubated in the presence of or of about 85 IU/ml recombinant IL-2. In some embodiments, the composition incubated with recombinant IL-2 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-2 may also be incubated with recombinant IL-7 and/or recombinant IL-15, such as in amounts described. In some embodiments, an enriched CD8+ T cell composition incubated with recombinant IL-2 may also be incubated with recombinant IL-15, such as in amounts described.

In some embodiments, a composition of enriched T cells is incubated with recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 IU/ml, or between 550 IU/ml and 650 IU/ml. In particular embodiments, the composition of enriched T cells is incubated with recombinant IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is incubated in the presence of or of about 600 IU/ml of recombinant IL-7. In some embodiments, the composition incubated with recombinant IL-7 is enriched for a population of T cells, e.g., CD4+ T cells. In some embodiments, an enriched CD4+ T cell composition incubated with recombinant IL-7 may also be incubated with recombinant IL-2 and/or recombinant IL-15, such as in amounts described. In particular embodiments, the composition of enriched T cells is enriched for CD4+ T cells, where CD8+ T cells are not enriched for and/or where CD8+ T cells are negatively selected for or depleted from the composition. In some embodiments, an enriched CD8+ T cell composition is not incubated with recombinant IL-7.

In some embodiments, a composition of enriched T cells is incubated with recombinant IL-15, e.g., human recombinant IL-15, at a concentration between 0.1 IU/ml and 100 IU/ml, between 1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is incubated with recombinant IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is incubated in or in about 10 IU/ml of recombinant IL-15. In some embodiments, the composition incubated with recombinant IL-15 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-15 may also be incubated with recombinant IL-7 and/or recombinant IL-2, such as in amounts described. In some embodiments, an enriched CD8+ T cell composition incubated with recombinant IL-15 may also be incubated with recombinant IL-2, such as in amounts described.

In particular embodiments, the cells, such as enriched CD4+ T cells and/or enriched CD8+ T cells, are incubated with the stimulatory reagent in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthatione, cystamine and cysstathionine, and/or glycine-glycine-histidine. In some aspects, the incubation of the enriched T cell composition, such as enriched CD4+ T cells and/or enriched CD8+ T cells, with an antioxidant also includes the presence of a stimulatory reagent, e.g., anti-CD3/anti-CD28 magnetic beads, and one or more recombinant cytokines, such as described.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetylcysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, incubating the cells, such as enriched CD4+ T cells and/or enriched CD8+ T cells, under stimulating conditions includes incubating the cells in the presence of one or more antioxidants. In particular embodiments, the cells are stimulated with the stimulatory reagent in the presence of one or more antioxidants. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the one or more antioxidants is or includes N-acetyl cysteine (NAC). In some embodiments, incubating the cells, such as enriched CD4+ T cells and/or enriched CD8+ T cells, under stimulating conditions includes incubating the cells in the presence of NAC. In particular embodiments, the cells are stimulated with the stimulatory reagent in the presence of NAC. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1-500 μg/ml and 2 mg/ml, 500 μg/ml and 5 mg/ml, between 1 mg/ml and 10 mg/ml, or between 1 mg/ml and 100 mg/ml of NAC. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are incubated with or with about 0.8 mg/ml.

In particular embodiments, incubating the composition of enriched T cells, such as enriched CD4+ T cells and/or enriched CD8+ T cells, in the presence of one or more antioxidants, e.g., NAC, reduces the activation in the cells as compared to cells that are incubated in alternative and/or exemplary processes without the presence of antioxidants. In certain embodiments, the reduced activation is measured by the expression of one or more activation markers in the cell. In certain embodiments, markers of activation include, but are not limited to, increased intracellular complexity (e.g., as determined by measuring side scatter (SSC), increased cell size (e.g., as determined by measuring cell diameter and/or forward scatter (FSC), increased expression of CD27, and/or decreased expression of CD25. In some embodiments, the cells of the composition have negative, reduced, or low expression and/or degree of markers of activation when examined during or after the incubation, engineering, transduction, transfection, expansion, or formulation, or during or after any stage of the process occurring after the incubation. In some embodiments the cells of the composition have negative, reduced, or low expression and/or degree of markers of activation after the process is completed. In particular embodiments, the cells of the output composition have negative, reduced, or low expression and/or degree of markers of activation.

In some embodiments, flow cytometry is used to determine relative size of cells. In particular embodiments, the FSC and SSC parameters are used to analyze cells and distinguish the cells from one another based off of size and internal complexity. In particular embodiments, a particle or bead of a known size can be measured as a standard to determine the actual size of cells. In some embodiments, flow cytometry is used in combination with a stain, e.g., a labeled antibody, to measure or quantify the expression of a surface protein, such as a marker of activation, e.g., CD25 or CD27.

In some embodiments, the composition of enriched T cells, such as enriched CD4+ T cells and/or enriched CD8+ T cells, is incubated in the presence of one or more antioxidants e.g., NAC, and the cell diameter reduced by at least 0.25 μm, 0.5 μm, 0.75 μm, 1.0 μm, 1.5 μm, 2 μm, 2.5 μm, 3 μm, 3.5 μm, 4 μm, 4.5 μm, 5 μm, or more than 5 μm as compared to cells incubated in an alternative and/or exemplary process where the incubation is not performed in the presence of an antioxidant. In particular embodiments, the composition of enriched T cells is incubated in the presence of one or more antioxidants e.g., NAC, and the cell size, as measured by the FSC is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In some embodiments, the composition of enriched T cells, such as enriched CD4+ T cells and/or enriched CD8+ T cells, is incubated in the presence of one or more antioxidants e.g., NAC, and the intracellular complexity, as measured by the SSC, is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In particular embodiments, the composition of enriched T cells, such as enriched CD4+ T cells and/or enriched CD8+ T cells, is incubated in the presence of one or more antioxidants e.g., NAC, and the expression of CD27, e.g., as measured by the flow cytometry, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In certain embodiments, the composition of enriched T cells, such as enriched CD4+ T cells and/or enriched CD8+ T cells, is incubated in the presence of one or more antioxidants, e.g., NAC, and the expression of CD25, e.g., as measured by the flow cytometry, is increased by at least 1%, at least 5%, at least 10%, at least 20%, at least 30%, at least 40%, at least 50%, at least 60%, at least 70%, at least 75%, at least 80%, at least 85%, at least 90%,at least 100%, at least 150%, at least 1-fold, at least 2-fold, at least 3-fold, at least 4-fold, at least 5-fold, at least 10-fold, at least 25-Fold, at least 50-fold, or at least 100-fold as compared to cells incubated in an alternative and/or exemplary process where the incubation is not performed in the presence of an antioxidant.

In particular embodiments, incubating the composition of enriched T cells, such as enriched CD4+ T cells and/or enriched CD8+ T cells, in the presence of one or more antioxidants, e.g., NAC, increases the expansion, e.g., during the incubation or cultivation step or stage as described in Section I-D. In some embodiments, a composition of enriched cells achieves a 2-fold, a 2.5 fold, a 3 fold, a 3.5 fold, a 4 fold, a 4.5 fold a 5 fold, a 6 fold, a 7 fold, an 8 fold, a nine fold, a 10-fold, or greater than a 10 fold expansion within 14 days, 12 days, 10 days, 9 days, 8 days, 7 days, 6 days, 5 days, 4 days, or within 3 days of the start of the cultivation. In some embodiments, the composition of enriched T cells is incubated in the presence of one or more antioxidants and the cells of the compositions undergo at least 10%, at least a 20%, at least a 30%, at least a 40%, at least a 50%, at least a 60%, at least a 70%, at least a 75%, at least an 80%, at least an 85%, at least a 90%, at least a 100%, at least a 150%, at least a 1-fold, at least a 2-fold, at least a 3-fold, at least a 4-fold, at least a 5-fold, at least a 10-fold faster rate of expansion during the cultivation than cultivated cells that were incubated in an alternative and/or exemplary process where the incubation is not performed in the presence of an antioxidant.

In particular embodiments, incubating the composition of enriched cells, such as enriched CD4+ T cells and/or enriched CD8+ T cells, in the presence of one or more antioxidants, e.g., NAC, reduces the amount of cell death, e.g., by apoptosis. In some embodiments, the composition of enriched T cells is incubated in the presence of a one or more antioxidants, e.g., NAC, and at least 50%, at least 60%, at least 70%, at least 75%, at least 80%, at least 85%, at least 90%, at least 95%, at least 99%, or at least 99.9% of the cells survive, e.g., do not undergo apoptosis, during or at least 1 day, 2 days, 3 days, 4 days, 5 days, 6 days, 7 days, or more than 7 days after the incubation is complete. In some embodiments, the composition is incubated in the presence of one or more antioxidants, e.g., NAC, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing an exemplary and/or alternative process where cells are not incubated in the presence or one or more antioxidants.

In particular embodiments, the composition of enriched T cells, such as enriched CD4+ T cells and/or enriched CD8+ T cells, is incubated in the presence of one or more antioxidants e.g., NAC, and caspase expression, e.g., caspase 3 expression, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In some embodiments, the compositions or cells, such as enriched CD4+ T cells and/or enriched CD8+ T cells, are incubated in the presence of stimulating conditions or a stimulatory agent, such as described. Such conditions include those designed to induce proliferation, expansion, activation, and/or survival of cells in the population, to mimic antigen exposure, and/or to prime the cells for genetic engineering, such as for the introduction of a recombinant antigen receptor. Exemplary stimulatory reagents, such as anti-CD3/anti-CD28 magnetic beads, are described below. The incubation with the stimulatory reagent may also be carried out in the presence of one or more stimulatory cytokine, such as in the presence of one or more of recombinant IL-2, recombinant IL-7 and/or recombinant IL-15 and/or in the presence of at least one antioxidant such as NAC, such as described above. In some embodiments, a composition of enriched CD4+ T cells are incubated under stimulatory conditions with a stimulatory agent, recombinant IL-2, recombinant IL-7, recombinant IL-15 and NAC, such as in amounts as described. In some embodiments, a composition of enriched CD8+ T cells are incubated under stimulatory conditions with a stimulatory agent, recombinant IL-2, recombinant IL-15 and NAC, such as in amounts as described.

In some embodiments, the conditions for stimulation and/or activation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g., is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g., in a tube or bag with periodic shaking or rotation. In some embodiments, the incubation is performed with the addition of an incubation buffer to the cells and stimulating agent to achieve a target volume with incubation of the reagent of, for example, about 10 mL to about 200 mL, or about 20 mL to about 125 mL, such as at least or about at least or about 10 mL, 20 mL, 30 mL, 40 mL, 50 mL, 60 mL, 70 mL, 80 mL, 90 mL, 100 mL, 105 mL, 110 mL, 115 mL, 120 mL, 125 mL, 130 mL, 135 mL, 140 mL, 145 mL, 150 mL, 160 mL, 170 mL, 180 mL, 190 mL, or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm), such as at an RCF at the sample or wall of the chamber or other container of from 80 g to 100 g or from about 80 g to about 100 g (e.g., at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours, 18 hours and 30 hours, or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the cells are cultured, cultivated, and/or incubated under stimulating conditions prior to and/or during a step for introducing a polynucleotide, e.g., a polynucleotide encoding a recombinant receptor, to the cells, e.g., by transduction and/or transfection, such as described by Section I-C. In certain embodiments the cells are cultured, cultivated, and/or incubated under stimulating conditions for an amount of time between 30 minutes and 2 hours, between 1 hour and 8 hours, between 1 hour and 6 hours, between 6 hours and 12 hours, between 12 hours and 18 hours, between 16 hours and 24 hours, between 12 hours and 36 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days prior to the genetic engineering. In some embodiments, the cells are incubated for or for about 2 days prior to the engineering.

In certain embodiments, the cells are incubated with and/or in the presence of the stimulatory reagent prior to and/or during genetically engineering the cells. In certain embodiments the cells are incubated with and/or in the presence of the stimulatory reagent for an amount of time between 12 hours and 36 hours, between 24 hours and 48 hours, between 24 hours and 72 hours, between 42 hours and 54 hours, between 60 hours and 120 hours between 96 hours and 120 hours, between 90 hours and between 2 days and 7 days, between 3 days and 8 days, between 1 day and 8 days, between 4 days and 6 days, or between 4 days and 5 days. In particular embodiments, the cells are cultured, cultivated, and/or incubated under stimulating conditions prior to and/or during genetically engineering the cells for an amount of time of less than 10 days, 9 days, 8 days, 7 days, 6 days, or 5 days, 4 days, or for an amount of time less than 168 hours, 162 hours, 156 hours, 144 hours, 138 hours, 132 hours, 120 hours, 114 hours, 108 hours, 102 hours, or 96 hours. In particular embodiments, the cells are incubated with and/or in the presence of the stimulatory reagent for or for about 4 days, 5 days, 6 days, or 7 days. In some embodiments, the cells are incubated with and/or in the presence of the stimulatory reagent for or for about 4 days. In particular embodiments, the cells are incubated with and/or in the presence of the stimulatory reagent for or for about 5 days. In certain embodiments, the cells are incubated with and/or in the presence of the stimulatory reagent for less than 7 days.

In some embodiments, incubating the cells under stimulating conditions includes incubating the cells with a stimulatory reagent that is described in Section II-B-1. In some embodiments, the stimulatory reagent contains or includes a bead, such as a paramagnetic bead, and the cells are incubated with the stimulatory reagent at a ratio of less than 3:1 (beads:cells), such as a ratio of 1:1. In particular embodiments, the cells are incubated with the stimulatory reagent in the presence of one or more cytokines and/or one or more antioxidants. In some embodiments, a composition of enriched CD4+ T cells is incubated with the stimulatory reagent at a ratio of 1:1 (beads:cells) in the presence of recombinant IL-2, IL-7, IL-15, and NAC. In certain embodiments, a composition of enriched CD8+ T cells is incubated with the stimulatory reagent at a ratio of 1:1 (beads:cells) in the presence of recombinant IL-2, IL-15, and NAC. In some embodiments, the stimulatory reagent is removed and/or separated from the cells at, within, or within about 6 days, 5 days, or 4 days from the start or initiation of the incubation, e.g., from the time the stimulatory reagent is added to or contacted with the cells.

1. Stimulatory Reagents

In some embodiments, incubating a composition of enriched cells under stimulating conditions is or includes incubating and/or contacting the composition of enriched cells with a stimulatory reagent that is capable of activating and/or expanding T cells. In some embodiments, the stimulatory reagent is capable of stimulating and/or activating one or more signals in the cells. In some embodiments, the one or more signals are mediated by a receptor. In particular embodiments, the one or more signals are or are associated with a change in signal transduction and/or a level or amount of secondary messengers, e.g., cAMP and/or intracellular calcium, a change in the amount, cellular localization, confirmation, phosphorylation, ubiquitination, and/or truncation of one or more cellular proteins, and/or a change in a cellular activity, e.g., transcription, translation, protein degradation, cellular morphology, activation state, and/or cell division. In particular embodiments, the stimulatory reagent activates and/or is capable of activating one or more intracellular signaling domains of one or more components of a TCR complex and/or one or more intracellular signaling domains of one or more costimulatory molecules.

In certain embodiments, the stimulatory reagent contains a particle, e.g., a bead, that is conjugated or linked to one or more agents, e.g., biomolecules, that are capable of activating and/or expanding cells, e.g., T cells. In some embodiments, the one or more agents are bound to a bead. In some embodiments, the bead is biocompatible, i.e., composed of a material that is suitable for biological use. In some embodiments, the beads are non-toxic to cultured cells, e.g., cultured T cells. In some embodiments, the beads may be any particles which are capable of attaching agents in a manner that permits an interaction between the agent and a cell.

In some embodiments, a stimulatory reagent contains one or more agents that are capable of activating and/or expanding cells, e.g., T cells, that are bound to or otherwise attached to a bead, for example to the surface of the bead. In certain embodiments, the bead is a non-cell particle. In particular embodiments, the bead may include a colloidal particle, a microsphere, nanoparticle, a magnetic bead, or the like. In some embodiments the beads are agarose beads. In certain embodiments, the beads are sepharose beads.

In particular embodiments, the stimulatory reagent contains beads that are monodisperse. In certain embodiments, beads that are monodisperse comprise size dispersions having a diameter standard deviation of less than 5% from each other.

In some embodiments, the bead contains one or more agents, such as an agent that is coupled, conjugated, or linked (directly or indirectly) to the surface of the bead. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of methods known and available in the art. The attachment may be covalent, noncovalent, electrostatic, or hydrophobic and may be accomplished by a variety of attachment means, including for example, a chemical means, a mechanical means, or an enzymatic means. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the stimulatory reagent contains a bead and one or more agents that directly interact with a macromolecule on the surface of a cell. In certain embodiments, the bead (e.g., a paramagnetic bead) interacts with a cell via one or more agents (e.g., an antibody) specific for one or more macromolecules on the cell (e.g., one or more cell surface proteins). In certain embodiments, the bead (e.g., a paramagnetic bead) is labeled with a first agent described herein, such as a primary antibody (e.g., an anti-biotin antibody) or other biomolecule, and then a second agent, such as a secondary antibody (e.g., a biotinylated anti-CD3 antibody) or other second biomolecule (e.g., streptavidin), is added, whereby the secondary antibody or other second biomolecule specifically binds to such primary antibodies or other biomolecule on the particle.

In some embodiments, the stimulatory reagent contains one or more agents (e.g., antibody) that is attached to a bead (e.g., a paramagnetic bead) and specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD3OL, LIGHT, IL-2R, IL-12R, IL-1R, IL-15R; IFN-gammaR, TNF-alphaR, IL-4R, IL-10R, CD18/CDl la (LFA-1), CD62L (L-selectin), CD29/CD49d (VLA-4), Notch ligand (e.g., Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, an agent (e.g., antibody) attached to the bead specifically binds to one or more of the following macromolecules on a cell (e.g., a T cell): CD28, CD62L, CCR7, CD27, CD127, CD3, CD4, CD8, CD45RA, and/or CD45RO.

In some embodiments, one or more of the agents attached to the bead is an antibody. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the stimulatory reagent is an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species). In some embodiments, the agent is an antibody that binds to and/or recognizes one or more components of a T cell receptor. In particular embodiments, the agent is an anti-CD3 antibody. In certain embodiments, the agent is an antibody that binds to and/or recognizes a co-receptor. In some embodiments, the stimulatory reagent comprises an anti-CD28 antibody. In some embodiments, the bead has a diameter of greater than about 0.001 μm, greater than about 0.01 μm, greater than about 0.1 μm, greater than about 1.0 μm, greater than about 10 μm, greater than about 50 μm, greater than about 100 μm or greater than about 1000 μm and no more than about 1500 μm. In some embodiments, the bead has a diameter of about 1.0 μm to about 500 μm, about 1.0 μm to about 150 μm, about 1.0 μm to about 30 μm, about 1.0 μm to about 10 μm, about 1.0 μm to about 5.0 μm, about 2.0 μm to about 5.0 μm, or about 3.0 μm to about 5.0 μm. In some embodiments, the bead has a diameter of about 3 μm to about 5 μm. In some embodiments, the bead has a diameter of at least or at least about or about 0.001 μm, 0.01 μm, 0.1 μm, 0.5 μm, 1.0 μm, 1.5 μm, 2.0 μm,2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 12 μm, 14 μm, 16 μm, 18 μm or 20 μm. In certain embodiments, the bead has a diameter of or about 4.5 μm. In certain embodiments, the bead has a diameter of or about 2.8 μm.

In some embodiments, the beads have a density of greater than 0.001 g/cm³, greater than 0.01 g/cm³, greater than 0.05 g/cm³, greater than 0.1 g/cm³, greater than 0.5 g/cm³, greater than 0.6 g/cm³, greater than 0.7 g/cm³, greater than 0.8 g/cm³, greater than 0.9 g/cm³, greater than 1 g/cm³, greater than 1.1 g/cm³, greater than 1.2 g/cm³, greater than 1.3 g/cm³, greater than 1.4 g/cm³, greater than 1.5 g/cm³, greater than 2 g/cm³, greater than 3 g/cm³, greater than 4 g/cm³, or greater than 5 g/cm³. In some embodiments, the beads have a density of between about 0.001 g/cm³ and about 100 g/cm³, about 0.01 g/cm³ and about 50 g/cm³, about 0.1 g/cm³ and about 10 g/cm³, about 0.1 g/cm³ and about 0.5 g/cm³, about 0.5 g/cm³ and about 1 g/cm³, about 0.5 g/cm³ and about 1.5 g/cm³, about 1 g/cm³ and about 1.5 g/cm³, about 1 g/cm³ and about 2 g/cm³, or about 1 g/cm³ and about 5 g/cm³. In some embodiments, the beads have a density of about 0.5 g/cm³, about 0.5 g/cm³, about 0.6 g/cm³, about 0.7 g/cm³, about 0.8 g/cm³, about 0.9 g/cm³, about 1.0 g/cm³, about 1.1 g/cm³, about 1.2 g/cm³, about 1.3 g/cm³, about 1.4 g/cm³, about 1.5 g/cm³, about 1.6 g/cm³, about 1.7 g/cm³, about 1.8 g/cm³, about 1.9 g/cm³, or about 2.0 g/cm³. In certain embodiments, the beads have a density of about 1.6 g/cm³. In particular embodiments, the beads or particles have a density of about 1.5 g/cm³. In certain embodiments, the particles have a density of about 1.3 g/cm³.

In certain embodiments, a plurality of the beads has a uniform density. In certain embodiments, a uniform density comprises a density standard deviation of less than 10%, less than 5%, or less than 1% of the mean bead density.

In some embodiments, the beads have a surface area of between about 0.001 m² per each gram of particles (m²/g) to about 1,000 m²/g, about 0.010 m²/g to about 100 m²/g, about 0.1 m²/g to about 10 m²/g, about 0.1 m²/g to about 1 m²/g, about 1 m²/g to about 10 m²/g, about 10 m²/g to about 100 m²/g, about 0.5 m²/g to about 20 m²/g, about 0.5 m²/g to about 5 m²/g, or about 1 m²/g to about 4 m²/g. In some embodiments, the particles or beads have a surface area of about 1 m²/g to about 4 m²/g.

In some embodiments, the bead contains at least one material at or near the bead surface that can be coupled, linked, or conjugated to an agent. In some embodiments, the bead is surface functionalized, i.e. comprises functional groups that are capable of forming a covalent bond with a binding molecule, e.g., a polynucleotide or a polypeptide. In particular embodiments, the bead comprises surface-exposed carboxyl, amino, hydroxyl, tosyl, epoxy, and/or chloromethyl groups. In particular embodiments, the beads comprise surface exposed agarose and/or sepharose. In certain embodiments, the bead surface comprises attached stimulatory reagents that can bind or attach binding molecules. In particular embodiments, the biomolecules are polypeptides. In some embodiments, the beads comprise surface exposed protein A, protein G, or biotin.

In some embodiments, the bead reacts in a magnetic field. In some embodiments, the bead is a magnetic bead. In some embodiments, the magnetic bead is paramagnetic. In particular embodiments, the magnetic bead is superparamagnetic. In certain embodiments, the beads do not display any magnetic properties unless they are exposed to a magnetic field.

In particular embodiments, the bead comprises a magnetic core, a paramagnetic core, or a superparamagnetic core. In some embodiments, the magnetic core contains a metal. In some embodiments, the metal can be, but is not limited to, iron, nickel, copper, cobalt, gadolinium, manganese, tantalum, zinc, zirconium or any combinations thereof. In certain embodiments, the magnetic core comprises metal oxides (e.g., iron oxides), ferrites (e.g., manganese ferrites, cobalt ferrites, nickel ferrites, etc.), hematite and metal alloys (e.g., CoTaZn). In some embodiments, the magnetic core comprises one or more of a ferrite, a metal, a metal alloy, an iron oxide, or chromium dioxide. In some embodiments, the magnetic core comprises elemental iron or a compound thereof. In some embodiments, the magnetic core comprises one or more of magnetite (Fe3O4), maghemite (γFe2O3), or greigite (Fe3S4). In some embodiments, the inner core comprises an iron oxide (e.g., Fe₃O₄).

In certain embodiments, the bead contains a magnetic, paramagnetic, and/or superparamagnetic core that is covered by a surface functionalized coat or coating. In some embodiments, the coat can contain a material that can include, but is not limited to, a polymer, a polysaccharide, a silica, a fatty acid, a protein, a carbon, agarose, sepharose, or a combination thereof. In some embodiments, the polymer can be a polyethylene glycol, poly (lactic-co-glycolic acid), polyglutaraldehyde, polyurethane, polystyrene, or a polyvinyl alcohol. In certain embodiments, the outer coat or coating comprises polystyrene. In particular embodiments, the outer coating is surface functionalized.

In some embodiments, the stimulatory reagent comprises a bead that contains a metal oxide core (e.g., an iron oxide core) and a coat, wherein the metal oxide core comprises at least one polysaccharide (e.g., dextran), and wherein the coat comprises at least one polysaccharide (e.g., amino dextran), at least one polymer (e.g., polyurethane) and silica. In some embodiments the metal oxide core is a colloidal iron oxide core. In certain embodiments, the one or more agents include an antibody or antigen-binding fragment thereof. In particular embodiments, the one or more agents include an anti-CD3 antibody and an anti-CD28 antibody or antigen-binding fragments thereof. In some embodiments, the stimulatory reagent comprises an anti-CD3 antibody, anti-CD28 antibody, and an anti-biotin antibody. In some embodiments, the stimulatory reagent comprises an anti-biotin antibody. In some embodiments, the bead has a diameter of about 3 μm to about 10 μm. In some embodiments, the bead has a diameter of about 3 μm to about 5 μm. In certain embodiments, the bead has a diameter of about 3.5 μm.

In some embodiments, the stimulatory reagent comprises one or more agents that are attached to a bead comprising a metal oxide core (e.g., an iron oxide inner core) and a coat (e.g., a protective coat), wherein the coat comprises polystyrene. In certain embodiments, the beads are monodisperse, paramagnetic (e.g., superparamagnetic) beads comprising a paramagnetic (e.g., superparamagnetic) iron core, e.g., a core comprising magnetite (Fe3O4) and/or maghemite (₇Fe2O3) c and a polystyrene coat or coating. In some embodiments, the bead is non-porous. In some embodiments, the beads contain a functionalized surface to which the one or more agents are attached. In certain embodiments, the one or more agents are covalently bound to the beads at the surface. In some embodiments, the one or more agents include an antibody or antigen-binding fragment thereof. In some embodiments, the one or more agents include an anti-CD3 antibody and an anti-CD28 antibody. In some embodiments, the stimulatory reagent is or comprises anti-CD3/anti-CD28 magnetic beads, In some embodiments, the one or more agents include an anti-CD3 antibody and/or an anti-CD28 antibody, and an antibody or antigen fragment thereof capable of binding to a labeled antibody (e.g., biotinylated antibody), such as a labeled anti-CD3 or anti-CD28 antibody. In certain embodiments, the beads have a density of about 1.5 g/cm³ and a surface area of about 1 m²/g to about 4 m²/g. In particular embodiments; the beads are monodisperse superparamagnetic beads that have a diameter of about 4.5 μm and a density of about 1.5 g/cm³. In some embodiments, the beads the beads are monodisperse superparamagnetic beads that have a mean diameter of about 2.8 μm and a density of about 1.3 g/cm³.

In some embodiments, the composition of enriched T cells is incubated with stimulatory reagent a ratio of beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

2 Removal of the Stimulatory Reagent from Cells

In certain embodiments, the stimulatory reagent, e.g., anti-CD3/anti-CD28 magnetic beads, is removed and/or separated from the cells. Without wishing to be bound by theory, particular embodiments contemplate that the binding and/or association between a stimulatory reagent and cells may, in some circumstances, be reduced over time during the incubation. In certain embodiments, one or more agents may be added to reduce the binding and/or association between the stimulatory reagent and the cells. In particular embodiments, a change in cell culture conditions, e.g., media temperature of pH, may reduce the binding and/or association between the stimulatory reagent and the cells. Thus, in some embodiments, the stimulatory reagent may be removed from an incubation, cell culture system, and/or a solution separately from the cells, e.g., without removing the cells from the incubation, cell culture system, and/or a solution as well.

Methods for removing stimulatory reagents (e.g., stimulatory reagents that are or contain particles such as bead particles or magnetizable particles) from cells are known. In some embodiments, the use of competing antibodies, such as non-labeled antibodies, can be used, which, for example, bind to a primary antibody of the stimulatory reagent and alter its affinity for its antigen on the cell, thereby permitting for gentle detachment. In some cases, after detachment, the competing antibodies may remain associated with the particle (e.g., bead particle) while the unreacted antibody is or may be washed away and the cell is free of isolating, selecting, enriching and/or activating antibody. Exemplary of such a reagent is DETACaBEAD (Friedl et al. 1995; Entschladen et al. 1997). In some embodiments, particles (e.g., bead particles) can be removed in the presence of a cleavable linker (e.g., DNA linker), whereby the particle-bound antibodies are conjugated to the linker (e.g., CELLection, Dynal). In some cases, the linker region provides a cleavable site to remove the particles (e.g., bead particles) from the cells after isolation, for example, by the addition of DNase or other releasing buffer. In some embodiments, other enzymatic methods can also be employed for release of a particle (e.g., bead particle) from cells. In some embodiments, the particles (e.g., bead particles or magnetizable particles) are biodegradable.

In some embodiments, the stimulatory reagent is magnetic, paramagnetic, and/or superparamagnetic, and/or contains a bead that is magnetic, paramagnetic, and/or superparamagnetic, and the stimulatory reagent may be removed from the cells by exposing the cells to a magnetic field. Examples of suitable equipment containing magnets for generating the magnetic field include DynaMag CTS (Thermo Fisher), Magnetic Separator (Takara) and EasySep Magnet (Stem Cell Technologies).

In particular embodiments, the stimulatory reagent is removed or separated from the cells prior to the completion of the provided methods, e.g., prior to harvesting, collecting, and/or formulating engineered cells produced by the methods provided herein. In some embodiments, the stimulatory reagent is removed and/or separated from the cells prior to engineering, e.g., transducing or transfecting, the cells. In particular embodiments, the stimulatory reagent is removed and/or separated from the cells after the step of engineering the cells. In certain embodiments, the stimulatory reagent is removed prior to the cultivation of the cells, e.g., prior to the cultivation of the engineered, e.g., transfected or transduced, cells under conditions to promote proliferation and/or expansion.

In certain embodiments, the stimulatory reagent is separated and/or removed from the cells after an amount of time. In particular embodiments, the amount of time is an amount of time from the start and/or initiation of the incubation under stimulating conditions. In particular embodiments the start of the incubation is considered at or at about the time the cells are contacted with the stimulatory reagent and/or a media or solution containing the stimulatory reagent. In particular embodiments, the stimulatory reagent is removed or separated from the cells within or within about 10 days, 9 days, 8 days, 7 days, 6 days, 5 days, 4 days, 3 days, or 2 days after the start or initiation of the incubation. In particular embodiments, the stimulatory reagent is removed and/or separated from the cells at or at about 9 days, 8 days, 7 days, 6 days, 5 days, 4 days, 3 days, or 2 days after the start or initiation of the incubation. In certain embodiments, the stimulatory reagent is removed and/or separated from the cells at or at about 168 hours, 162 hours, 156 hours, 144 hours, 138 hours, 132 hours, 120 hours, 114 hours, 108 hours, 102 hours, or 96 hours after the start or initiation of the incubation. In particular embodiments, the stimulatory reagent is removed and/or separated from the cells at or at about 5 days after the start and/or initiation of the incubation. In some embodiments, the stimulatory reagent is removed and/or separated from the cells at or at about 4 days after the start and/or initiation of the incubation.

C. Engineering Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the methods provided herein are used in association with engineering one or more compositions of enriched T cells. In certain embodiments, the engineering is or includes the introduction of a polynucleotide, e.g., a recombinant polynucleotide encoding a recombinant protein. In particular embodiments, the recombinant proteins are recombinant receptors, such as any described in Section III. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In certain embodiments, one or more compositions of enriched T cells are engineered, e.g., transduced or transfected, prior to cultivating the cells, e.g., under conditions that promote proliferation and/or expansion, such as by a method provided in Section II-D. In particular embodiments, one or more compositions of enriched T cells are engineered after the one or more compositions have been stimulated, activated, and/or incubated under stimulating conditions, such as described in methods provided in Section II-B. In particular embodiments, the one or more compositions are stimulated compositions. In particular embodiments, the one or more stimulated compositions have been previously cryofrozen and stored, and are thawed prior to engineering.

In certain embodiments, the one or more compositions of stimulated T cells are or include two separate stimulated compositions of enriched T cells. In particular embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells, such as following incubation under stimulating conditions as described above, are genetically engineered separately. In some embodiments, a single composition of enriched T cells is genetically engineered. In certain embodiments, the single composition is a composition of enriched CD4+ T cells. In some embodiments, the single composition is a composition of enriched CD4+ and CD8+ T cells that have been combined from separate compositions prior to the engineering.

In some embodiments, the composition of enriched CD4+ T cells, such as stimulated CD4+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated CD8+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that, such as stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are genetically engineered, e.g., transduced or transfected. In certain embodiments, separate engineered compositions of enriched CD4+ and enriched CD8+ T cells are combined into a single composition after the genetic engineering has been performed and/or completed. In particular embodiments, separate compositions of enriched CD4+ and CD8+ T cells, such as separate compositions of stimulated CD4+ and CD8+ T cells are separately engineered and are separately processed for cultivation and/or expansion of T cells after the genetic engineering and been performed and/or completed.

In some embodiments, the introduction of a polynucleotide, e.g., a recombinant polynucleotide encoding a recombinant protein, is carried out by contacting enriched CD4+ or CD8+ T cells, such as stimulated CD4+ or CD8+ T cells, with a viral particles containing the polynucleotide. In some embodiments, contacting can be effected with centrifugation, such as spinoculation (e.g., centrifugal inoculation). In some embodiments, the composition containing cells, viral particles and reagent can be rotated, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g., at or about or at least at or about 100 g, 200 g, 300 g, 400 g, 500 g, 1000 g, 1500 g, 2000 g, 2500 g, 3000 g or 3200 g), such as at or about 693 g, as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured). In some embodiments, at least a portion of the contacting, incubating, and/or engineering of the cells, e.g., cells from an stimulated composition of enriched CD4+ T cell or enriched CD8+ T cells, with the virus is performed with a rotation of between about 100 g and 3200 g, 1000 g and 2000 g, 1000 g and 3200 g, 500 g and 1000 g, 400 g and 1200 g, 600 g and 800 g, 600 and 700 g, or 500 g and 700 g. In some embodiments, the rotation is between 600 g and 700 g, e.g., at or about 693 g.

In certain embodiments, at least a portion of the engineering, transduction, and/or transfection is performed with rotation, e.g., spinoculation and/or centrifugation. In some embodiments, the rotation is performed for, for about, or for at least or about 5 minutes, 10 minutes, 15 minutes, 30 minutes, 60 minutes, 90 minutes, 1 hour, 2 hours, 3 hours, 4 hours, 6 hours, 8 hours, 12 hours, 24 hours, 48 hours, 72 hours, 2 days, 3 days, 4 days, 5 days, 6 days, or for at least 7 days. In some embodiments, the rotation is performed for or for about 60 minutes. In certain embodiments, the rotation is performed for about 30 minutes. In some embodiments, the rotation performed for about 30 minutes at between 600 g and 700 g, e.g., at or about 693 g.

In certain embodiments, the number of viable cells to be engineered, transduced, and/or transfected ranges from about 5×10⁶ cells to about 100×10⁷ cells, such as from about 10×10⁶ cells to about 100×10⁶ cells, from about 100×10⁶ cells to about 200×10⁶ cells, from about 200×10⁶ cells to about 300×10⁶ cells, from about 300×10⁶ cells to about 400×10⁶ cells, from about 400×10⁶ cells to about 500×10⁶ cells, or from about 500×10⁶ cells to about 100×10⁷ cells. In particular examples, the number of viable cells to be engineered, transduced, and/or transfected is about or less than about 300×10⁶ cells.

In certain embodiments, at least a portion of the engineering, transduction, and/or transfection is conducted at a volume (e.g., the spinoculation volume) from about 5 mL to about 100 mL, such as from about 10 mL to about 50 mL, from about 15 mL to about 45 mL, from about 20 mL to about 40 mL, from about 25 mL to about 35 mL, or at or at about 30 mL. In certain embodiments, the cell pellet volume after spinoculation ranges from about 1 mL to about 25 mL, such as from about 5 mL to about 20 mL, from about 5 mL to about 15 mL, from about 5 mL to about 10 mL, or at or at about 10 mL.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under stimulating conditions, such as by any of the methods described in Section II-B.

In some embodiments, methods for genetic engineering are carried out by contacting one or more cells of a composition with a nucleic acid molecule encoding the recombinant protein, e.g., recombinant receptor. In some embodiments, the contacting can be effected with centrifugation, such as spinoculation (e.g., centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the system is included with and/or placed into association with other instrumentation, including instrumentation to operate, automate, control and/or monitor aspects of the transduction step and one or more various other processing steps performed in the system, e.g., one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, the composition containing cells and composition containing viral vector particles, and optionally air, can be combined or mixed prior to providing the compositions to the cavity. In some embodiments, the composition containing cells and composition containing viral vector particles, and optionally air, are provided separately and combined and mixed in the cavity. In some embodiments, a composition containing cells, a composition containing viral vector particles, and optionally air, can be provided to the internal cavity in any order. In any of such some embodiments, a composition containing cells and viral vector particles is the input composition once combined or mixed together, whether such is combined or mixed inside or outside the centrifugal chamber and/or whether cells and viral vector particles are provided to the centrifugal chamber together or separately, such as simultaneously or sequentially.

In some embodiments, intake of a volume of gas, such as air, occurs prior to the incubating the cells and viral vector particles, such as rotation, in the transduction method. In some embodiments, intake of the volume of gas, such as air, occurs during the incubation of the cells and viral vector particles, such as rotation, in the transduction method.

In some embodiments, the liquid volume of the cells or viral vector particles that make up the transduction composition, and optionally the volume of air, can be a predetermined volume. The volume can be a volume that is programmed into and/or controlled by circuitry associated with the system.

In some embodiments, intake of the transduction composition, and optionally gas, such as air, is controlled manually, semi-automatically and/or automatically until a desired or predetermined volume has been taken into the internal cavity of the chamber. In some embodiments, a sensor associated with the system can detect liquid and/or gas flowing to and from the centrifuge chamber, such as via its color, flow rate and/or density, and can communicate with associated circuitry to stop or continue the intake as necessary until intake of such desired or predetermined volume has been achieved. In some aspects, a sensor that is programmed or able only to detect liquid in the system, but not gas (e.g., air), can be made able to permit passage of gas, such as air, into the system without stopping intake. In some such embodiments, a non-clear piece of tubing can be placed in the line near the sensor while intake of gas, such as air, is desired. In some embodiments, intake of gas, such as air, can be controlled manually.

In aspects of the provided methods, the internal cavity of the centrifuge chamber is subjected to high speed rotation. In some embodiments, rotation is effected prior to, simultaneously, subsequently or intermittently with intake of the liquid input composition, and optionally air. In some embodiments, rotation is effected subsequent to intake of the liquid input composition, and optionally air. In some embodiments, rotation is by centrifugation of the centrifugal chamber at a relative centrifugal force at the inner surface of side wall of the internal cavity and/or at a surface layer of the cells of at or about or at least at or about 800 g, 1000 g, 1100 g, 1500, 1600 g, 1800 g, 2000 g, 2200 g, 2500 g, 3000 g, 3500 g or 4000 g. In some embodiments, rotation is by centrifugation at a force that is greater than or about 1100 g, such as by greater than or about 1200 g, greater than or about 1400 g, greater than or about 1600 g, greater than or about 1800 g, greater than or about 2000 g, greater than or about 2400 g, greater than or about 2800 g, greater than or about 3000 g or greater than or about 3200 g. In some embodiments, rotation is by centrifugation at a force that is or is about 1600 g.

In some embodiments, the method of transduction includes rotation or centrifugation of the transduction composition, and optionally air, in the centrifugal chamber for greater than or about 5 minutes, such as greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes. In some embodiments, the transduction composition, and optionally air, is rotated or centrifuged in the centrifugal chamber for greater than 5 minutes, but for no more than 60 minutes, no more than 45 minutes, no more than 30 minutes or no more than 15 minutes. In particular embodiments, the transduction includes rotation or centrifugation for or for about 60 minutes.

In some embodiments, the method of transduction includes rotation or centrifugation of the transduction composition, and optionally air, in the centrifugal chamber for between or between about 10 minutes and 60 minutes, 15 minutes and 60 minutes, 15 minutes and 45 minutes, 30 minutes and 60 minutes or 45 minutes and 60 minutes, each inclusive, and at a force at the internal surface of the side wall of the internal cavity and/or at a surface layer of the cells of at least or greater than or about 1000 g, 1100 g, 1200 g, 1400 g, 1500 g, 1600 g, 1800 g, 2000 g, 2200 g, 2400 g, 2800 g, 3200 g or 3600 g. In particular embodiments, the method of transduction includes rotation or centrifugation of the transduction composition, e.g., the cells and the viral vector particles, at or at about 1600 g for or for about 60 minutes.

In some embodiments, the gas, such as air, in the cavity of the chamber is expelled from the chamber. In some embodiments, the gas, such as air, is expelled to a container that is operably linked as part of the closed system with the centrifugal chamber. In some embodiments, the container is a free or empty container. In some embodiments, the air, such as gas, in the cavity of the chamber is expelled through a filter that is operably connected to the internal cavity of the chamber via a sterile tubing line. In some embodiments, the air is expelled using manual, semi-automatic or automatic processes. In some embodiments, air is expelled from the chamber prior to, simultaneously, intermittently or subsequently with expressing the output composition containing incubated cells and viral vector particles, such as cells in which transduction has been initiated or cells have been transduced with a viral vector, from the cavity of the chamber.

In some embodiments, the transduction and/or other incubation is performed as or as part of a continuous or semi-continuous process. In some embodiments, a continuous process involves the continuous intake of the cells and viral vector particles, e.g., the transduction composition (either as a single pre-existing composition or by continuously pulling into the same vessel, e.g., cavity, and thereby mixing, its parts), and/or the continuous expression or expulsion of liquid, and optionally expelling of gas (e.g., air), from the vessel, during at least a portion of the incubation, e.g., while centrifuging. In some embodiments, the continuous intake and continuous expression are carried out at least in part simultaneously. In some embodiments, the continuous intake occurs during part of the incubation, e.g., during part of the centrifugation, and the continuous expression occurs during a separate part of the incubation. The two may alternate. Thus, the continuous intake and expression, while carrying out the incubation, can allow for a greater overall volume of sample to be processed, e.g., transduced.

In some embodiments, the incubation is part of a continuous process, the method including, during at least a portion of the incubation, effecting continuous intake of said transduction composition into the cavity during rotation of the chamber and during a portion of the incubation, effecting continuous expression of liquid and, optionally expelling of gas (e.g., air), from the cavity through the at least one opening during rotation of the chamber.

In some embodiments, the semi-continuous incubation is carried out by alternating between effecting intake of the composition into the cavity, incubation, expression of liquid from the cavity and, optionally expelling of gas (e.g., air) from the cavity, such as to an output container, and then intake of a subsequent (e.g., second, third, etc.) composition containing more cells and other reagents for processing, e.g., viral vector particles, and repeating the process. For example, in some embodiments, the incubation is part of a semi-continuous process, the method including, prior to the incubation, effecting intake of the transduction composition into the cavity through said at least one opening, and subsequent to the incubation, effecting expression of fluid from the cavity; effecting intake of another transduction composition comprising cells and the viral vector particles into said internal cavity; and incubating the another transduction composition in said internal cavity under conditions whereby said cells in said another transduction composition are transduced with said vector. The process may be continued in an iterative fashion for a number of additional rounds. In this respect, the semi-continuous or continuous methods may permit production of even greater volume and/or number of cells.

In some embodiments, a portion of the transduction incubation is performed in the centrifugal chamber, which is performed under conditions that include rotation or centrifugation.

In some embodiments, the method includes an incubation in which a further portion of the incubation of the cells and viral vector particles is carried out without rotation or centrifugation, which generally is carried out subsequent to the at least portion of the incubation that includes rotation or centrifugation of the chamber. In certain embodiments, the incubation of the cells and viral vector particles is carried out without rotation or centrifugation for at least 1 hour, 6 hours, 12 hours, 24 hours, 32 hours, 48 hours, 60 hours, 72 hours, 90 hours, 96 hours, 3 days, 4 days, 5 days, or greater than 5 days. In certain embodiments, the incubation is carried out for or for about 72 hours.

In some such embodiments, the further incubation is effected under conditions to result in integration of the viral vector into a host genome of one or more of the cells. It is within the level of a skilled artisan to assess or determine if the incubation has resulted in integration of viral vector particles into a host genome, and hence to empirically determine the conditions for a further incubation. In some embodiments, integration of a viral vector into a host genome can be assessed by measuring the level of expression of a recombinant protein, such as a heterologous protein, encoded by a nucleic acid contained in the genome of the viral vector particle following incubation. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

In some embodiments, the composition containing cells, the vector, e.g., viral particles, and reagent can be rotated, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g., at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, during at least a part of the genetic engineering, e.g., transduction, and/or subsequent to the genetic engineering the cells are transferred to the bioreactor bag assembly for culture of the genetically engineered cells, such as for cultivation or expansion of the cells, as described above.

In certain embodiments, a composition of enriched T cells in engineered, e.g., transduced or transfected, in the presence of a transduction adjuvant. In some embodiments, a composition of enriched T cells is engineered in the presence of one or more polycations. In some embodiments, a composition of enriched T cells is transduced, e.g., incubated with a viral vector particle, in the presence of one or more transduction adjuvants. In particular embodiments, a composition of enriched T cells is transfected, e.g., incubated with a non-viral vector, in the presence of one or more transduction adjuvants. In certain embodiments, the presence of one or more transduction adjuvants increases the efficiency of gene delivery, such as by increasing the amount, portion, and/or percentage of cells of the composition that are engineered (e.g., transduced or transfected). In certain embodiments, the presence of one or more transduction adjuvants increases the efficiency of transfection. In certain embodiments, the presence of one or more transduction adjuvants increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold more cells of a composition are engineered to contain or express the recombinant transduction adjuvants in the presence of a polycation as compared to an alternative and/or exemplary method of engineering cells without the presence of a transduction adjuvant.

In some embodiments, the composition of enriched cells are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of a transduction adjuvant. In certain embodiments, transduction adjuvants suitable for use with the provided methods include, but are not limited to polycations, fibronectin or fibronectin-derived fragments or variants, RetroNectin, and combinations thereof.

In some embodiments, the cells are engineered in the presence of a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is engineered in the presence of IL-2, e.g., human recombinant IL-2, at a concentration between 1 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is engineered in the presence of recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml. In some embodiments, the composition of enriched T cells is engineered in the presence of or of about 85 IU/ml. In some embodiments, the population of T cells is a population of CD4+ T cells. In particular embodiments, the composition of enriched T cells is enriched for CD4+ T cells, where CD8+ T cells are not enriched for and/or where CD8+ T cells are negatively selected for or depleted from the composition. In particular embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition.

In some embodiments, a composition of enriched T cells is engineered in the presence of recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 IU/ml, or between 550 IU/ml and 650 IU/ml. In particular embodiments, the composition of enriched T cells is engineered in the presence of IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is engineered in the presence of or of about 600 IU/ml of IL-7. In some embodiments, the composition engineered in the presence of recombinant IL-7 is enriched for a population of T cells, e.g., CD4+ T cells. In particular embodiments, the composition of enriched T cells is enriched for CD4+ T cells, where CD8+ T cells are not enriched for and/or where CD8+ T cells are negatively selected for or depleted from the composition.

In some embodiments, a composition of enriched T cells is engineered in the presence of recombinant IL-15, e.g., human recombinant IL-15, at a concentration between 0.1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is engineered in the presence of IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is engineered in or in about 10 IU/ml of IL-15. In some embodiments, the composition of enriched T cells is incubated in or in about 10 IU/ml of recombinant IL-15. In some embodiments, the composition engineered in the presence of recombinant IL-15 is enriched for a population of T cells, e.g., CD4+ T cells and/or CD8+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition.

In particular embodiments, a composition of enriched CD8+ T cells is engineered in the presence of IL-2 and/or IL-15. In certain embodiments, a composition of enriched CD4+ T cells is engineered in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cells are engineered in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthatione, cystamine and cystathionine, and/or glycine-glycine-histidine.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetylcysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, the cells are engineered in the presence of one or more antioxidants. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the cells are engineered in the presence of NAC. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1,500 μg/ml and 2 mg/ml, 500 μg/ml and 5 mg/ml, between 1 mg/ml and 10 mg/ml, or between 1 mg/ml and 100 mg/ml of NAC. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are engineered with or with about 0.8 mg/ml.

In some embodiments, a composition of enriched T cells, such as stimulated T cells, e.g., stimulated CD4+ T cells or stimulated CD8+ T cells, is engineered in the presence of one or more polycations. In some embodiments, a composition of enriched T cells, such as stimulated T cells, e.g., stimulated CD4+ T cells or stimulated CD8+ T cells, is transduced, e.g., incubated with a viral vector particle, in the presence of one or more polycations. In particular embodiments, a composition of enriched T cells, such as stimulated T cells, e.g., stimulated CD4+ T cells or stimulated CD8+ T cells, is transfected, e.g., incubated with a non-viral vector, in the presence of one or more polycations. In certain embodiments, the presence of one or more polycations increases the efficiency of gene delivery, such as by increasing the amount, portion, and/or percentage of cells of the composition that are engineered (e.g., transduced or transfected). In certain embodiments, the presence of one or more polycations increases the efficiency of transfection. In certain embodiments, the presence of one or more polycations increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold more cells of a composition are engineered to contain or express the recombinant polynucleotide in the presence of a polycation as compared to an alternative and/or exemplary method of engineering cells without the presence of a polycation.

In certain embodiments, the composition of enriched cells, e.g., the composition of enriched CD4+ T cells or enriched CD8+ T cells, such as stimulated T cells thereof, is engineered in the presence of a low concentration or amount of a polycation, e.g., relative to an exemplary and/or alternative method of engineering cells in the presence of a polycation. In certain embodiments, the composition of enriched cells, such as stimulated T cells, e.g., stimulated CD4+ T cells or stimulated CD8+ T cells, is engineered in the presence of less than 90%, less than 80%, less than 75%, less than 70%, less than 60%, less than 50%, less than 40%, less than 30%, less than 25%, less than 20%, less than 10%, less than 5%, less than 1%, less than 0.1%, of less than 0.01% of the amount and/or concentration of the polycation of an exemplary and/or alternative process for engineering cells. In some embodiments, the composition of enriched cells, such as stimulated T cells, e.g., stimulated CD4+ T cells or stimulated CD8+ T cells, are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of the polycation. In particular embodiments, the composition of enriched cells, such as stimulated T cells, e.g., stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, or 50 μg/ml, of the polycation.

In particular embodiments, engineering the composition of enriched cells, such as stimulated T cells, e.g., stimulated CD4+ T cells or stimulated CD8+ T cells, in the presence of a polycation reduces the amount of cell death, e.g., by necrosis, programmed cell death, or apoptosis. In some embodiments, the composition of enriched T cells, such as stimulated T cells, e.g., stimulated CD4+ T cells or stimulated CD8+ T cells, is engineered in the presence of a low amount of a polycation, e.g., less than 100 μg/ml, 50 μg/ml, or 10 μg/ml, and at least 50%, at least 60%, at least 70%, at least 75%, at least 80%, at least 85%, at least 90%, at least 95%, at least 99%, or at least 99.9% of the cells survive, e.g., do not undergo necrosis, programmed cell death, or apoptosis, during or at least 1 day, 2 days, 3 days, 4 days, 5 days, 6 days, 7 days, or more than 7 days after the engineering step is complete. In some embodiments, the composition is engineered in the presence of a low concentration or amount of polycation as compared to the alternative and/or exemplary method of engineering cells in the presence of higher amount or concentration of polycation, e.g., more than 50 μg/ml, 100 μg/ml, 500 μg/ml, or 1,000 μg/ml, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing the exemplary and/or alternative process.

In some embodiments, the polycation is positively-charged. In certain embodiments, the polycation reduces repulsion forces between cells and vectors, e.g., viral or non-viral vectors, and mediates contact and/or binding of the vector to the cell surface. In some embodiments, the polycation is polybrene, DEAE-dextran, protamine sulfate, poly-L-lysine, or cationic liposomes.

In particular embodiments, the polycation is protamine sulfate. In some embodiments, the composition of enriched T cells, such as stimulated T cells, e.g., stimulated CD4+ T cells or stimulated CD8+ T cells, are engineered in the presence of less than or about 500 μg/ml, less than or about 400 μg/ml, less than or about 300 μg/ml, less than or about 200 μg/ml, less than or about 150 μg/ml, less than or about 100 μg/ml, less than or about 90 μg/ml, less than or about 80 μg/ml, less than or about 75 μg/ml, less than or about 70 μg/ml, less than or about 60 μg/ml, less than or about 50 μg/ml, less than or about 40 μg/ml, less than or about 30 μg/ml, less than or about 25 μg/ml, less than or about 20 μg/ml, or less than or about 15 μg/ml, or less than or about 10 μg/ml of protamine sulfate. In particular embodiments, the composition of enriched cells, such as stimulated T cells, e.g., stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 75 μg/ml, 80 μg/ml, 85 μg/ml, 90 μg/ml, 95 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, or 150 μg/ml of protamine sulfate.

In some embodiments, the engineered composition of enriched CD4+ T cells, such as stimulated T cells, e.g., stimulated CD4+ T cells, includes at least 40, at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated T cells, e.g., stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated T cells, e.g., stimulated CD8+ T cells, that is engineered includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells, such as stimulated T cells, e.g., stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, engineering the cells includes a culturing, contacting, or incubation with the vector, e.g., the viral vector of the non-viral vector. In certain embodiments, the engineering includes culturing, contacting, and/or incubating the cells with the vector is performed for, for about, or for at least 4 hours, 6 hours, 8 hours, 12 hours, 16 hours, 18 hours, 24 hours, 30 hours, 36 hours, 40 hours, 48 hours, 54 hours, 60 hours, 72 hours, 84 hours, 1 day, 2 days, 3 days, 4 days, 5 days, 6 days, or 7 days, or more than 7 days. In particular embodiments, the engineering includes culturing, contacting, and/or incubating the cells with the vector for or for about 24 hours, 36 hours, 48 hours, 60 hours, 72 hours, or 84 hours, or for or for about 2 days, 3 days, 4 days, or 5 days. In some embodiments, the engineering step is performed for or for about 24 hours, 36 hours, 48 hours, 60 hours, 72 hours, or 84 hours. In certain embodiments, the engineering is performed for about 60 hours or about 84 hours, for or for about 72 hours, or for or for about 2 days.

In some embodiments, the engineering is performed at a temperature from about 25 to about 38° C., such as from about 30 to about 37° C., from about 36 to about 38° C., or at or about 37° C.±2° C. In some embodiments, the composition of enriched T cells is engineered at a CO₂ level from about 2.5% to about 7.5%, such as from about 4% to about 6%, for example at or about 5%±0.5%. In some embodiments, the composition of enriched T cells is engineered at a temperature of or about 37° C. and/or at a CO₂ level of or about 5%.

In some embodiments, the cells, e.g., the CD4+ and/or the CD8+ T cells, are cultivated, after one or more steps are performed for genetic engineering, e.g., transducing or transfection the cells to contain a polynucleotide encoding a recombinant receptor. In some embodiments, the cultivation may include culture, incubation, stimulation, activation, expansion, and/or propagation. In some such embodiments, the further cultivation is effected under conditions to result in integration of the viral vector into a host genome of one or more of the cells. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In some embodiments, the further incubation is carried out at temperatures greater than room temperature, such as greater than or greater than about 25° C., such as generally greater than or greater than about 32° C., 35° C. or 37° C. In some embodiments, the further incubation is effected at a temperature of at or about 37° C.±2° C., such as at a temperature of at or about 37° C.

In some embodiments, the further incubation is performed under conditions for stimulation and/or activation of cells, which conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent (e.g., stimulatory and/or accessory agents), e.g., ligand, which is capable of activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, and/or an agent that promotes a costimulatory signal, such as one specific for a T cell costimulatory receptor, e.g., anti-CD3, anti-CD28, or anti-41-BB, for example, optionally bound to solid support such as a bead, and/or one or more cytokines. Among the stimulating agents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti-CD28 antibody to the culture medium. In some embodiments, the stimulating agents include IL-2 and/or IL-15, for example, an IL-2 concentration of at least about 10 units/mL.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL, at least about 50 units/mL, at least about 100 units/mL or at least about 200 units/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, the further incubation is carried out in the same container or apparatus in which the contacting occurred. In some embodiments, the further incubation is carried out without rotation or centrifugation, which generally is carried out subsequent to the at least portion of the incubation done under rotation, e.g., in connection with centrifugation or spinoculation. In some embodiments, the further incubation is carried out outside of a stationary phase, such as outside of a chromatography matrix, for example, in solution.

In some embodiments, the further incubation is carried out in a different container or apparatus from that in which the contacting occurred, such as by transfer, e.g., automatic transfer, of the cell composition into a different container or apparatus subsequent to contacting with the viral particles and reagent.

In some embodiments, the further culturing or incubation, e.g., to facilitate ex vivo expansion, is carried out of for greater than or greater than about 24 hours, 2 days, 3 days, 4 days, 5 days, 6 days, 7 days, 8 days, 9 days, 10 days, 11 days, 12 days, 13 days or 14 days. In some embodiments, the further culturing or incubation is carried out for no more than 6 days, no more than 5 days, no more than 4 days, no more than 3 days, no more than 2 days or no more than 24 hours.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the methods provided herein do not include further culturing or incubation, e.g., do not include ex vivo expansion step, or include a substantially shorter ex vivo expansion step.

In some embodiments, the stimulatory reagent is removed and/or separated from the cells prior to the engineering. In particular embodiments, the stimulatory reagent is removed and/or separated from the cells after the engineering. In certain embodiments, the stimulatory agent is removed and/or separated from the cells subsequent to the engineering and prior to cultivating the engineered cells, .e.g., under conditions that promote proliferation and/or expansion. In certain embodiments, the stimulatory reagent is a stimulatory reagent that is described in Section II-B-1. In particular embodiments, the stimulatory reagent is removed and/or separated from the cells as described in Section II-B-2.

1. Vectors and Methods

Also provided are one or more polynucleotides (e.g., nucleic acid molecules) encoding recombinant receptors, vectors for genetically engineering cells to express such receptors in accord with provided methods for producing the engineered cells. In some embodiments, the vector contains the nucleic acid encoding the recombinant receptor. In particular embodiments, the vector is a viral vector a non-viral vector. In some cases, the vector is a viral vector, such as a retroviral vector, e.g., a lentiviral vector or a gammaretroviral vector.

In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 10 and encoded by the nucleotide sequence set forth in SEQ ID NO: 9, the CD8 alpha signal peptide set forth in SEQ ID NO: 11, or the CD33 signal peptide set forth in SEQ ID NO: 12.

In some embodiments, the vectors include viral vectors, e.g., retroviral or lentiviral, non-viral vectors or transposons, e.g., Sleeping Beauty transposon system, vectors derived from simian virus 40 (SV40), adenoviruses, adeno-associated virus (AAV), lentiviral vectors or retroviral vectors, such as gamma-retroviral vectors, retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV) or adeno-associated virus (AAV).

In some embodiments, the viral vector or the non-viral DNA contains a nucleic acid that encodes a heterologous recombinant protein. In some embodiments, the heterologous recombinant molecule is or includes a recombinant receptor, e.g., an antigen receptor, SB-transposons, e.g., for gene silencing, capsid-enclosed transposons, homologous double stranded nucleic acid, e.g., for genomic recombination or reporter genes (e.g., fluorescent proteins, such as GFP) or luciferase).

a. Viral Vector Particles

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

In some embodiments, the provided methods involve methods of transducing cells by contacting, e.g., incubating, a cell composition comprising a plurality of cells with a viral particle. In some embodiments, the cells to be transfected or transduced are or comprise primary cells obtained from a subject, such as cells enriched and/or selected from a subject.

In some embodiments, the concentration of cells to be transduced of the composition is from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10 ⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or about at least or about 30 minutes, 1 hour, 2 hours, 6 hours, 12 hours, 24 hours, 36 hours or more.

In some embodiments, contacting is performed in solution. In some embodiments, the cells and viral particles are contacted in a volume of from 0.5 mL to 500 mL or from about 0.5 mL to about 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

b. Non-Viral Vectors

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, recombinant nucleic acids are transferred into T cells via transposons. Transposons (transposable elements), are mobile segments of DNA that can move from one locus to another within genomes. These elements move via a conservative, “cut-and-paste” mechanism: the transposase catalyzes the excision of the transposon from its original location and promotes its reintegration elsewhere in the genome. Transposase-deficient elements can be mobilized if the transposase is provided by another transposase gene. Thus, transposons can be utilized to incorporate a foreign DNA into a host genome without the use of a viral transduction system. Examples of transposons suitable for use with mammalian cells, e.g., human primary leukocytes, include but are not limited to Sleeping Beauty and PiggyBacs.

Transposon-based transfection is a two-component system consisting of a transposase and a transposon. In some embodiments, the system comprises a transposon is engineered to comprise a foreign DNA (also referred herein as cargo DNA), e.g., a gene encoding a recombinant receptor, that is flanked by inverted repeat/direct repeat (IR/DR) sequences that are recognized by an accompanying transposase. In some embodiments, a non-viral plasmid encodes a transposase under the control of a promoter. Transfection of the plasmid into a host cell results in a transitory expression of the transposase, thus for an initial period following transfection, the transposase is expressed at sufficiently levels to integrate the transposon into the genomic DNA. In some embodiments, the transposase itself is not integrated into the genomic DNA, and therefor expression of the transposase decreases over time. In some embodiments, the transposase expression is expressed by the host cell at levels sufficient to integrate a corresponding transposon for less than about 4 hours, less than about 8 hours, less than about 12 hours, less than about 24 hours, less than about 2 days, less than about 3 days, less than about 4 days, less than about 5 days, less than about 6 days, less than about 7 days, less than about 2 weeks, less than about 3 weeks, less than about 4 weeks, less than about weeks, or less than about 8 weeks. In some embodiments, the cargo DNA that is introduced into the host's genome is not subsequently removed from the host's genome, at least because the host dose not express an endogenous transposase capable of excising the cargo DNA.

Sleeping Beauty (SB) is a synthetic member of the Tc/1-mariner superfamily of transposons, reconstructed from dormant elements harbored in the salmonid fish genome. SB transposon-based transfection is a two-component system consisting of a transposase and a transposon containing inverted repeat/direct repeat (IR/DR) sequences that result in precise integration into a TA dinucleotide. The transposon is designed with an expression cassette of interest flanked by IR/DRs. The SB transposase binds specific binding sites that are located on the IR of the Sleeping beauty transposon. The SB transposase mediates integration of the transposon, a mobile element encoding a cargo sequence flanked on both sides by inverted terminal repeats that harbor binding sites for the catalytic enzyme (SB). Stable expression results when SB inserts gene sequences into vertebrate chromosomes at a TA target dinucleotide through a cut-and-paste mechanism. This system has been used to engineer a variety of vertebrate cell types, including primary human peripheral blood leukocytes. In some embodiments, the cells are contacted, incubated, and/or treated with an SB transposon comprising a cargo gene, e.g., a gene encoding a recombinant receptor or a CAR, flanked by SB IR sequences. In particular embodiments, the cells to be transfected are contacted, incubated, and/or treated with a plasmid comprising an SB transposon comprising a cargo gene, e.g., a gene encoding a CAR, flanked by SB IR sequences. In certain embodiments, the plasmid further comprises a gene encoding an SB transposase that is not flanked by SB IR sequences.

PiggyBac (PB) is another transposon system that can be used to integrate cargo DNA into a host's, e.g., a human's, genomic DNA. The PB transposase recognizes PB transposon-specific inverted terminal repeat sequences (ITRs) located on both ends of the transposon and efficiently moves the contents from the original sites and efficiently integrates them into TTAA chromosomal sites. The PB transposon system enables genes of interest between the two ITRs in the PB vector to be mobilized into target genomes. The PB system has been used to engineer a variety of vertebrate cell types, including primary human cells. In some embodiments, the cells to be transfected are contacted, incubated, and/or treated with an PB transposon comprising a cargo gene, e.g., a gene encoding a CAR, flanked by PB IR sequences. In particular embodiments, the cells to be transfected are contacted, incubated, and/or treated with a plasmid comprising a PB transposon comprising a cargo gene, e.g., a gene encoding a CAR, flanked by PB IR sequences. In certain embodiments, the plasmid further comprises a gene encoding an SB transposase that is not flanked by PB IR sequences.

In some embodiments, the various elements of the transposon/transposase the employed in the subject methods, e.g., SB or PB vector(s), may be produced by standard methods of restriction enzyme cleavage, ligation and molecular cloning. One protocol for constructing the subject vectors includes the following steps. First, purified nucleic acid fragments containing desired component nucleotide sequences as well as extraneous sequences are cleaved with restriction endonucleases from initial sources, e.g., a vector comprising the transposase gene. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g., sequences corresponding to the various elements of the subject vectors, as described above is produced. Where desired, the circular molecules so constructed are then amplified in a prokaryotic host, e.g., E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc. An example of how to construct the vectors employed in the subject methods is provided in the Experimental section, infra. The preparation of a representative Sleeping Beauty transposon system is also disclosed in WO 98/40510 and WO 99/25817).

In some embodiments, transduction with transposons is performed with a plasmid that comprises a transposase gene and a plasmid that comprises a transposon that contains a cargo DNA sequence that is flanked by inverted repeat/direct repeat (IR/DR) sequences that are recognized by the transposase. In certain embodiments, the cargo DNA sequence encodes a heterologous protein, e.g., a recombinant T cell receptor or a CAR. In some embodiments, the plasmid comprises transposase and the transposon. In some embodiments, the transposase is under control of a ubiquitous promoter, or any promoter suitable to drive expression of the transposase in the target cell. Ubiquitous promoters include, but are not limited to, EF1a, CMB, SV40, PGK1, Ubc, human β-actin, CAG, TRE, UAS, Ac5, CaMKIIa, and U6. In some embodiments, the cargo DNA comprises a selection cassette allowing for the selection of cells with stable integration of the cargo DNA into the genomic DNA. Suitable selection cassettes include, but are not limited to, selection cassettes encoding a kanamycin resistance gene, spectinomycin resistance gene, streptomycin resistance gene, ampicillin resistance gene, carbenicillin resistance gene, hygromycin resistance gene, bleomycin resistance gene, erythromycin resistance gene, and polymyxin B resistance gene.

In some embodiments, the components for transduction with a transposon, e.g., plasmids comprising an SB transposase and SB transposon, are introduced into the target cell. Any convenient protocol may be employed, where the protocol may provide for in vitro or in vivo introduction of the system components into the target cell, depending on the location of the target cell. For example, where the target cell is an isolated cell, the system may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

In some embodiments, the SB transposon and the SB transposase source are introduced into a target cell of a multicellular organism, e.g., a mammal or a human, under conditions sufficient for excision of the inverted repeat flanked nucleic acid from the vector carrying the transposon and subsequent integration of the excised nucleic acid into the genome of the target cell. Some embodiments further comprise a step of ensuring that the requisite transposase activity is present in the target cell along with the introduced transposon. Depending on the structure of the transposon vector itself, i.e. whether or not the vector includes a region encoding a product having transposase activity, the method may further include introducing a second vector into the target cell which encodes the requisite transposase activity.

In some embodiments, the amount of vector nucleic acid comprising the transposon and the amount of vector nucleic acid encoding the transposase that is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed, e.g., the particular ex vivo administration protocol that is employed.

Once the vector DNA has entered the target cell in combination with the requisite transposase, the nucleic acid region of the vector that is flanked by inverted repeats, i.e. the vector nucleic acid positioned between the Sleeping Beauty transposase recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell. In particular embodiments, the vector is integrated into the genomes of at least 1%, at least 2%, at least 3%, at least 4%, at least 5%, at least 6% at least 7% at least 8%, at least 9%, at least 10%, at least 15%, or at least 20% of the cells that are transfected with the SB transposon and/or SB transposase. In some embodiments, integration of the nucleic acid into the target cell genome is stable, i.e., the vector nucleic acid remains present in the target cell genome for more than a transient period of time and is passed on a part of the chromosomal genetic material to the progeny of the target cell.

In certain embodiments, the transposons are used to integrate nucleic acids, i.e. polynucleotides, of various sizes into the target cell genome. In some embodiments, the size of DNA that is inserted into a target cell genome using the subject methods ranges from about 0.1 kb to 200 kb, from about 0.5 kb to 100 kb, from about 1.0 kb to about 8.0 kb, from about 1.0 to about 200 kb, from about 1.0 to about 10 kb, from about 10 kb to about 50 kb, from about 50 kb to about 100 kb, or from about 100 kb to about 200 kb. In some embodiments, the size of DNA that is inserted into a target cell genome using the subject methods ranges from about from about 1.0 kb to about 8.0 kb. In some embodiments, the size of DNA that is inserted into a target cell genome using the subject methods ranges from about 1.0 to about 200 kb. In particular embodiments, the size of DNA that is inserted into a target cell genome using the subject methods ranges from about 1.0 kb to about 8.0 kb.

D. Cultivation and/or Expansion of Cells

In some embodiments, the provided methods include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. In some embodiments, the cultivation produces one or more cultivated compositions of enriched T cells.

In certain embodiments, one or more compositions of enriched T cells, including stimulated and transduced T cells, such as separate compositions of such CD4+ and CD8+ T cells, are cultivated, e.g., under conditions that promote proliferation and/or expansion, prior to formulating the cells. In some aspects, the methods of cultivation, such as for promoting proliferation and/or expansion include methods provided herein, such as in Section II-D. In particular embodiments, one or more compositions of enriched T cells are cultivated after the one or more compositions have been engineered, e.g., transduced or transfected. In particular embodiments, the one or more compositions are engineered compositions. In particular embodiments, the one or more engineered compositions have been previously cryofrozen and stored, and are thawed prior to cultivating.

In certain embodiments, the one or more compositions of engineered T cells are or include two separate compositions of enriched T cells. In particular embodiments, two separate compositions of enriched T cells, e.g., two separate compositions of enriched T cells selected, isolated, and/or enriched from the same biological sample, that are introduced with a recombinant receptor (e.g., CAR), are separately cultivated under conditions that promote proliferation and/or expansion of the cells. In some embodiments, the conditions are stimulating conditions. In certain embodiments, the two separate compositions include a composition of enriched CD4+ T cells, such as engineered CD4+ T cells that were introduced with the nucleic acid encoding the recombinant receptor and/or that express the recombinant receptor. In particular embodiments, the two separate compositions include a composition of enriched CD8+ T cells, such as engineered CD8+ T cells that were introduced with the nucleic acid encoding the recombinant receptor and/or that express the recombinant receptor. In some embodiments, two separate compositions of enriched CD4+ T cells and enriched CD8+ T cells, such as engineered CD4+ T cells and engineered CD8+ T cells, are separately cultivated, e.g., under conditions that promote proliferation and/or expansion. In some embodiments, a single composition of enriched T cells is cultivated. In certain embodiments, the single composition is a composition of enriched CD4+ T cells. In some embodiments, the single composition is a composition of enriched CD4+ and CD8+ T cells that have been combined from separate compositions prior to the cultivation.

In some embodiments, the composition of enriched CD4+ T cells, such as engineered CD4+ T cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express the recombinant receptor and/or have been transduced or transfected with the recombinant polynucleotide encoding the recombinant receptor. In certain embodiments, the composition of enriched CD4+ T cells that is cultivated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as engineered CD8+ t cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In particular embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide encoding the recombinant receptor. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells, such as separate compositions of engineered CD4+ and engineered CD8+ T cells, are combined into a single composition and are cultivated, e.g., under conditions that promote proliferation and/or expansion. In certain embodiments, separate cultivated compositions of enriched CD4+ and enriched CD8+ T cells are combined into a single composition after the cultivation has been performed and/or completed. In particular embodiments, separate compositions of enriched CD4+ and CD8+ T cells, such as separate compositions of engineered CD4+ and engineered CD8+ T cells, are separately cultivated, e.g., under conditions that promote proliferation and/or expansion.

In some embodiments, the cells, e.g., the engineered cells are cultivated in a volume of media that is, is about, or is at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL, or 2,400 mL. In some embodiments, the cells are cultivated at an initial volume that is later adjusted to a different volume. In particular embodiments, the volume is later adjusted during the cultivation. In particular embodiments, the volume is increased from the initial volume during the cultivation. In certain embodiments, the volume is increased when the cells achieve a density during the cultivation. In certain embodiment, the initial volume is or is about 500 mL.

In particular embodiments, the volume is increased from the initial volume when the cells achieve a density or concentration during the cultivation. In particular embodiments, the volume is increased when the cells achieve a density and/or concentration of, of about, or of at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the volume is increased from the initial volume when the cells achieve a density and/or concentration of, of at least, or of about 0.6×10⁶ cells/ml. In some embodiments, the density and/or concentration is of viable cells in the culture. In particular embodiments, the volume is increased when the cells achieve a density and/or concentration of, of about, or of at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, the volume is increased from the initial volume when the viable cells achieve a density and/or concentration of, of at least, or of about 0.6×10⁶ viable cells/ml. In some embodiments, density and/or concentration of the cells or viable cells can be determined or monitored during the cultivation, such as by using methods as described, including optical methods, including digital holography microscopy (DHM) or differential digital holography microscopy (DDHM).

In some embodiments, the cells achieve a density and/or concentration, and the volume is increased by, by about, or by at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL In some embodiments, the volume is increased by 500 mL In particular embodiments, the volume is increased to a volume of, of about, or of at least 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL. In certain embodiments, the volume is increased to a volume of 1,000 mL. In certain embodiments, the volume is increase at a rate of, of at least, or of about 5 mL, 10 mL, 20 mL, 25 mL, 30 mL, 40 mL, 50 mL, 60 mL, 70 mL, 75 mL, 80 mL, 90 mL, or 100 mL, every 1, 2, 3, 4, 5, 6, 7, 8, 9, or 10 minutes. In certain embodiments, the rate is or is about 50 mL every 8 minutes.

In some embodiments, a composition of enriched T cells, such as engineered T cells, is cultivated under conditions that promote proliferation and/or expansion. In some embodiments, such conditions may be designed to induce proliferation, expansion, activation, and/or survival of cells in the population. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In some embodiments, the cultivation is performed under conditions that generally include a temperature suitable for the growth of primary immune cells, such as human T lymphocytes, for example, at least about 25 degrees Celsius, generally at least about 30 degrees, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g., cultivation or expansion, results in a desired or threshold density, concentration, number or dose of cells. In some embodiments, the incubation is carried out for a time period until the culture, e.g., cultivation or expansion, results in a desired or threshold density, concentration, number or dose of viable cells. In some embodiments, the incubation is greater than or greater than about or is for about or 24 hours, 48 hours, 72 hours, 96 hours, 5 days, 6 days, 7 days, 8 days, 9 days or more. In some embodiments, density, concentration and/or number or dose of the cells can be determined or monitored during the cultivation, such as by using methods as described, including optical methods, including digital holography microscopy (DHM) or differential digital holography microscopy (DDHM).

In some embodiments, the stimulatory reagent is removed and/or separated from the cells prior to the cultivation. In certain embodiments, the stimulatory agent is removed and/or separated from the cells subsequent to the engineering and prior to cultivating the engineered cells, e.g., under conditions that promote proliferation and/or expansion. In some embodiments, the stimulatory reagent is a stimulatory reagent that is described herein, e.g., in Section II-B-1. In particular embodiments, the stimulatory reagent is removed and/or separated from the cells as described herein, e.g., in Section II-B-2.

In particular embodiments, a composition of enriched T cells, such as engineered T cells, for example separate compositions of engineered CD4+ T cells and engineered CD8+ T cells, is cultivated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In particular embodiments, the composition of enriched CD4+ T cells, such as engineered CD4+ T cells, is cultivated with recombinant IL-2. In some embodiments, cultivating a composition of enriched CD4+ T cells, such as engineered CD4+ T cells, in the presence of recombinant IL-2 increases the probability or likelihood that the CD4+ T cells of the composition will continue to survive, grow, expand, and/or activate during the cultivation step and throughout the process. In some embodiments, cultivating the composition of enriched CD4+ T cells, such as engineered CD4+ T cells, in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, or at least 200% CD4+ as compared to an alternative and/or exemplary method that does not cultivate the composition of enriched CD4+ T cells in the presence of recombinant IL-2.

In some embodiments, the cells, such as separate compositions of engineered CD4+ T cells and engineered CD8+ T cells, are cultivated with a cytokine, e.g., a recombinant human cytokine, at a concentration of between 1 IU/ml and 2,000 IU/ml, between 10 IU/ml and 100 IU/ml, between 50 IU/ml and 500 IU/ml, between 100 IU/ml and 200 IU/ml, between 500 IU/ml and 1400 IU/ml, between 250 IU/ml and 500 IU/ml, or between 500 IU/ml and 2,500 IU/ml.

In some embodiments, a composition of enriched of T cells, such as separate compositions of engineered CD4+ T cells and CD8+ T cells, is cultivated with recombinant IL-2, e.g., human recombinant IL-2, at a concentration between 2 IU/ml and 500 IU/ml, between 10 IU/ml and 250 IU/ml, between 100 IU/ml and 500 IU/ml, or between 100 IU/ml and 400 IU/ml. In particular embodiments, the composition of enriched T cells is cultivated with IL-2 at a concentration at or at about 50 IU/ml, 75 IU/ml, 100 IU/ml, 125 IU/ml, 150 IU/ml, 175 IU/ml, 200 IU/ml, 225 IU/ml, 250 IU/ml, 300 IU/ml, or 400 IU/ml. In some embodiments, the composition of enriched T cells is cultivated with recombinant IL-2 at a concentration of 200 IU/ml. In some embodiments, the composition of enriched T cells is a composition of enriched CD4+ T cells, such as a composition of engineered CD4+ T cells. In particular embodiments, the composition of enriched T cells is a composition of enriched CD8+ T cells, such as a composition of engineered CD8+ T cells.

In some embodiments, a composition of enriched T cells, such as separate compositions of engineered CD4+ T cells and CD8+ T cells, is cultivated with IL-7, e.g., human recombinant IL-7, at a concentration between 10 IU/ml and 5,000 IU/ml, between 500 IU/ml and 2,000 IU/ml, between 600 IU/ml and 1,500 IU/ml, between 500 IU/ml and 2,500 IU/ml, between 750 IU/ml and 1,500 IU/ml, or between 1,000 IU/ml and 2,000 IU/ml. In particular embodiments, the composition of enriched T cells is cultivated with IL-7 at a concentration at or at about 100 IU/ml, 200 IU/ml, 300 IU/ml, 400 IU/ml, 500 IU/ml, 600 IU/ml, 700 IU/ml, 800 IU/ml, 900 IU/ml, 1,000 IU/ml, 1,200 IU/ml, 1,400 IU/ml, or 1,600 IU/ml. In some embodiments, the cells are cultivated in the presence of recombinant IL-7 at a concertation of or of about 1,200 IU/ml. In some embodiments, the composition of enriched T cells is a composition of enriched CD4+ T cells, such as engineered CD4+ T cells.

In some embodiments, a composition of enriched T cells, such as separate compositions of engineered CD4+ T cells and CD8+ T cells, is cultivated with IL-15, e.g., human recombinant IL-15, at a concentration between 0.1 IU/ml and 200 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 00 IU/ml. In particular embodiments, the composition of enriched T cells is cultivated with IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, 50 IU/ml, 100 IU/ml, or 200 IU/ml. In particular embodiments, a composition of enriched T cells is cultivated with recombinant IL-15 at a concentration of 20 IU/ml. In some embodiments, the composition of enriched T cells is a composition of enriched CD4+ T cells, such as engineered CD4+ T cells. In particular embodiments, the composition of enriched T cells is a composition of enriched CD8+ T cells, such as engineered CD8+ T cells.

In particular embodiments, a composition of enriched CD8+ T cells, such as engineered CD8+ T cells, is cultivated in the presence of IL-2 and/or IL-15, such as in amounts as described. In certain embodiments, a composition of enriched CD4+ T cells, such as engineered CD4+ T cells, is cultivated in the presence of IL-2, IL-7, and/or IL-15, such as in amounts as described. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cultivation is performed in a closed system. In certain embodiments, the cultivation is performed in a closed system under sterile conditions. In particular embodiments, the cultivation is performed in the same closed system as one or more steps of the provided systems. In some embodiments the composition of enriched T cells is removed from a closed system and placed in and/or connected to a bioreactor for the cultivation. Examples of suitable bioreactors for the cultivation include, but are not limited to, GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the cells during at least a portion of the cultivation step.

In some embodiments, cells cultivated while enclosed, connected, and/or under control of a bioreactor undergo expansion during the cultivation more rapidly than cells that are cultivated without a bioreactor, e.g., cells that are cultivated under static conditions such as without mixing, rocking, motion, and/or perfusion. In some embodiments, cells cultivated while enclosed, connected, and/or under control of a bioreactor reach or achieve a threshold expansion, cell count, and/or density within 14 days, 10 days, 9 days, 8 days, 7 days, 6 days, 5 days, 4 days, 3 days, 2 days, 60 hours, 48 hours, 36 hours, 24 hours, or 12 hours. In some embodiments, cells cultivated while enclosed, connected, and/or under control of a bioreactor reach or achieve a threshold expansion, cell count, and/or density at least 50%, at least 60%, at least 70%, at least 80%, at least 90%, at least 95%, at least 100%, at least 150%, at least 1-fold, at least 2-fold, at least 3-fold, at least 4-fold, at least 5-fold than cells cultivated in an exemplary and/or alternative process where cells are not cultivated while enclosed, connected, and/or under control of a bioreactor.

In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 17°, 16°, 15°, 14°, 13°, 12°, 11°, 10°, 9°, 8°, 7°, 6°, 5°, 4°, 3°, 2° or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 1 12, 13, 14 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, the bioreactor maintains the temperature at or near 37° C. and CO₂ levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the cultivation is performed with perfusion, such as with a rate of 290 ml/day, 580 ml/day, and/or 1160 ml/day, e.g., depending on the timing in relation to the start of the cultivation and/or density of the cultivated cells. In some embodiments, at least a portion of the cell culture expansion is performed with a rocking motion, such as at an angle of between 5° and 10°, such as 6°, at a constant rocking speed, such as a speed of between 5 and 15 RPM, such as 6 RPM or 10 RPM.

In some embodiments, the at least a portion of the cultivation step is performed under constant perfusion, e.g., a perfusion at a slow steady rate. In some embodiments, the perfusion is or include an outflow of liquid e.g., used media, and an inflow of fresh media. In certain embodiments, the perfusion replaces used media with fresh media. In some embodiments, at least a portion of the cultivation is performed under perfusion at a steady rate of or of about or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day.

In particular embodiments, cultivation is started under conditions with no perfusion, and perfusion started after a set and/or predetermined amount of time, such as or as about or at least 12 hours, 24 hours, 36 hours, 48 hours, 60 hours, 72 hours, or more than 72 hours after the start or initiation of the cultivation. In particular embodiments, perfusion is started when the density or concentration of the cells reaches a set or predetermined density or concentration. In some embodiments, the perfusion is started when the cultivated cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In particular embodiments, perfusion is started when the density or concentration of viable cells reaches a set or predetermined density or concentration. In some embodiments, the perfusion is started when the cultivated viable cells reach a density or concentration of, of about, or at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml.

In particular embodiments, the perfusion is performed at different speeds during the cultivation. For example, in some embodiments, the rate of the perfusion depends on the density and/or concentration of the cultivated cells. In certain embodiments, the rate of perfusion is increased when the cells reach a set or predetermined density or concentration. The perfusion rate may change, e.g., change from one steady perfusion rate to an increased steady perfusion rate, once, twice, three times, four times, five times, more than five times, more than ten times, more than 15 times, more than 20 times, more than 25 times, more than 50 times, or more than 100 times during the cultivation. In some embodiments, the steady perfusion rate increases when the cells reach a set or predetermined cell density or concentration of, of about, or at least 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the steady perfusion rate increases when the cells reach a set or predetermined viable cell density or concentration of, of about, or at least 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, density and/or concentration of the cells or of the viable cells during the cultivation, such as under perfusion, can be determined or monitored, such as by using methods as described, including optical methods, including digital holography microscopy (DHM) or differential digital holography microscopy (DDHM).

In some embodiments, cultivation is started under conditions with no perfusion, and, perfusion is started when the density or concentration of the cells reaches a set or predetermined density or concentration. In some embodiments, the perfusion is started at a rate of, of about, or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml.

In certain embodiments, at least part of the cultivation is performed with perfusion at a certain rate, and the perfusion rate is increased to, to about, or to at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the perfusion is performed when the cells are cultivated in a volume of, of about, or at least 300 mL, 400 mL, 500 mL, 600 mL, 700 mL, 800 mL, 900 mL, or 1000 mL. In some embodiments, the volume is 1000 mL.

In certain embodiments, cultivation is started under conditions with either no perfusion or perfusion at a certain rate, and the perfusion rate is increased to, to about, or to at 290 ml/day when the density or concentration of the cells reaches a concentration of, of about, or of at least 0.61×10⁶ cells/ml. In certain embodiments, the cells are perfused at a rate of, of about, or at least 290 ml/day when the density or concentration of the cells reaches a concentration of, of about, or of at least 0.61×10⁶ cells/ml when the cells are cultivated at a volume of, of about, or at least 1000 mL. In some embodiments, the perfusion rate is increased to, to about, or to at 580 ml/day when the density or concentration of the cells reaches a concentration of, of about, or of at least 0.81×10⁶ cells/ml. In certain embodiments, the perfusion rate is increased to, to about, or to at 1160 ml/day when the density or concentration of the cells reaches a concentration of, of about, or of at least 1.01×10⁶ cells/ml. In some embodiments, the perfusion rate is increased to, to about, or to at 1160 ml/day when the density or concentration of the cells reaches a concentration of, of about, or of at least 1.2×10⁶ cells/ml.

In aspects of the provided embodiments, the rate of perfusion, including the timing of when it is started or increased as described herein and above, is determined from assessing density and/or concentration of the cells or assessing the density and/or concentration of viable cells during the cultivation. In some embodiments, density and/or concentration of the cells can be determined using methods as described, including optical methods, including digital holography microscopy (DHM) or differential digital holography microscopy (DDHM).

In some embodiments, a composition of enriched cells, such as engineered T cells, e.g., engineered CD4+ T cells or engineered CD8+ T cells, is cultivated in the presence of a surfactant. In particular embodiments, cultivating the cells of the composition reduces the amount of shear stress that may occur during the cultivation, e.g., due to mixing, rocking, motion, and/or perfusion. In particular embodiments, the composition of enriched T cells, such as engineered T cells, e.g., engineered CD4+ T cells or engineered CD8+ T cells, is cultivated with the surfactant and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programmed cell death, or apoptosis, during or at least 1 day, 2 days, 3 days, 4 days, 5 days, 6 days, 7 days, or more than 7 days after the cultivation is complete. In particular embodiments, the composition of enriched T cells, such as engineered T cells, e.g., engineered CD4+ T cells or engineered CD8+ T cells, is cultivated in the presence of a surfactant and less than 50%, less than 40%, less than 30%, less than 25%, less than 20%, less than 15%, less than 10%, less than 5%, less than 1%, less than 0.1% or less than 0.01% of the cells undergo cell death, e.g., programmed cell death, apoptosis, and/or necrosis, such as due to shearing or shearing-induced stress.

In particular embodiments, a composition of enriched T cells, such as engineered T cells, e.g., engineered CD4+ T cells or engineered CD8+ T cells, is cultivated in the presence of between 0.1 μl/ml and 10.0 μl/ml, between 0.2 μl/ml and 2.5 μl/ml, between 0.5 μl/ml and 5 μl/ml, between 1 μl/ml and 3 μl/ml, or between 2 μl/ml and 4 μl/ml of the surfactant. In some embodiments, the composition of enriched T cells, such as engineered T cells, e.g., engineered CD4+ T cells or engineered CD8+ T cells, is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2.0 μl/ml, 2.5 μl/ml, 5.0 μl/ml, 10 μl/ml, 25 μl/ml, or 50 μl/ml of the surfactant. In certain embodiments, the composition of enriched T cells is cultivated in the presence of or of about 2 μl/ml of the surfactant.

In some embodiments, a surfactant is or includes an agent that reduces the surface tension of liquids and/or solids. For example, a surfactant includes a fatty alcohol (e.g., steryl alcohol), a polyoxyethylene glycol octylphenol ether (e.g., Triton X-100), or a polyoxyethylene glycol sorbitan alkyl ester (e.g., polysorbate 20, 40, 60). In certain embodiments the surfactant is selected from the group consisting of Polysorbate 80 (PS80), polysorbate 20 (PS20), poloxamer 188 (P188). In an exemplary embodiment, the concentration of the surfactant in chemically defined feed media is about 0.0025% to about 0.25% (v/v) of PS80; about 0.0025% to about 0.25% (v/v) of PS20; or about 0.1% to about 5.0% (w/v) of P188.

In some embodiments, the surfactant is or includes an anionic surfactant, a cationic surfactant, a zwitterionic surfactant, or a nonionic surfactant added thereto. Suitable anionic surfactants include but are not limited to alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl glycerol, phosphatidyl inosine, phosphatidylinositol, diphosphatidylglycerol, phosphatidylserine, phosphatidic acid and their salts, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate).

In some embodiments, suitable nonionic surfactants include: glyceryl esters, polyoxyethylene fatty alcohol ethers, polyoxyethylene sorbitan fatty acid esters (polysorbates), polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethyl cellulose, hydroxypropyl cellulose, hydroxypropylmethyl cellulose, noncrystalline cellulose, polysaccharides including starch and starch derivatives such as hydroxyethylstarch (HES), polyvinyl alcohol, and polyvinylpyrrolidone. In certain embodiments, the nonionic surfactant is a polyoxyethylene and polyoxypropylene copolymer and preferably a block copolymer of propylene glycol and ethylene glycol. Such polymers are sold under the tradename POLOXAMER, also sometimes referred to as PLURONIC® F68 or Kolliphor® P188. Among polyoxyethylene fatty acid esters is included those having short alkyl chains. One example of such a surfactant is SOLUTOL® HS 15, polyethylene-660-hydroxystearate.

In some embodiments, suitable cationic surfactants may include, but are not limited to, natural phospholipids, synthetic phospholipids, quaternary ammonium compounds, benzalkonium chloride, cetyltrimethyl ammonium bromide, chitosans, lauryl dimethyl benzyl ammonium chloride, acyl carnitine hydrochlorides, dimethyl dioctadecyl ammomium bromide (DDAB), dioleyoltrimethyl ammonium propane (DOTAP), dimyristoyl trimethyl ammonium propane (DMTAP), dimethyl amino ethane carbamoyl cholesterol (DC-Chol), 1,2-diacylglycero-3-(O-alkyl) phosphocholine, O-alkylphosphatidylcholine, alkyl pyridinium halides, or long-chain alkyl amines such as, for example, n-octylamine and oleylamine

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof. The phospholipid, whether anionic, zwitterionic or a mixture of phospholipids, may be salted or desalted, hydrogenated or partially hydrogenated or natural semi-synthetic or synthetic.

In certain embodiments, the surfactant is poloxamer, e.g., poloxamer 188. In some embodiments, a composition of enriched T cells is cultivated in the presence of between 0.1 μl/ml and 10.0 μl/ml, between 0.2 μl/ml and 2.5 μl/ml, between 0.5 μl/ml and 5 μl/ml, between 1 μl/ml and 3 μl/ml, or between 2 μl/ml and 4 μl/ml of poloxamer. In some embodiments, the composition of enriched T cells is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2.0 μl/ml, 2.5 μl/ml, 5.0 μl/ml, 10 μl/ml, 25 μl/ml, or 50 μl/ml of the surfactant. In certain embodiments, the composition of enriched T cells is cultivated in the presence of or of about 2 μl/ml of poloxamer.

In particular embodiments, the cultivation ends, such as by harvesting cells, when cells achieve a threshold amount, concentration, and/or expansion. In particular embodiments, the cultivation ends when the cell achieve or achieve about or at least a 1.5-fold expansion, a 2-fold expansion, a 2.5-fold expansion, a 3-fold expansion, a 3.5-fold expansion, a 4-fold expansion, a 4.5-fold expansion, a 5-fold expansion, a 6-fold expansion, a 7-fold expansion, a 8-fold expansion, a 9-fold expansion, a 10-fold expansion, or greater than a 10-fold expansion, e.g., with respect and/or in relation to the amount of density of the cells at the start or initiation of the cultivation. In some embodiments, the threshold expansion is a 4-fold expansion, e.g., with respect and/or in relation to the amount of density of the cells at the start or initiation of the cultivation.

In some embodiments, the cultivation ends, such as by harvesting cells, when the cells achieve a threshold total amount of cells, e.g., threshold cell count. In some embodiments, the cultivation ends when the cells achieve a threshold total nucleated cell (TNC) count. In some embodiments, the cultivation ends when the cells achieve a threshold viable amount of cells, e.g., threshold viable cell count. In some embodiments, the threshold cell count is or is about or is at least of 50×10⁶ cells, 100×10⁶ cells, 200×10⁶ cells, 300×10⁶ cells, 400×10⁶ cells, 600×10⁶ cells, 800×10⁶ cells, 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, 5000×10⁶ cells, 10,000×10⁶ cells, 12,000×10⁶ cells, 15,000×10⁶ cells or 20,000×10⁶ cells, or any of the foregoing threshold of viable cells. In particular embodiments, the cultivation ends when the cells achieve a threshold cell count. In some embodiments, the cultivation ends at, at about, or within 6 hours, 12 hours, 24 hours, 36 hours, 1 day, 2 days, 3 days, 4 days, 5 days, 6 days, or 7 or more days, after the threshold cell count is achieved. In particular embodiments, the cultivation is ended at or about 1 day after the threshold cell count is achieved. In certain embodiments, the threshold density is, is about, or is at least 0.1×10⁶ cells/ml, 0.5×10⁶ cells/ml, 1×10⁶ cells/ml, 1.2×10⁶ cells/ml, 1.5×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml, or any of the foregoing threshold of viable cells. In particular embodiments, the cultivation ends when the cells achieve a threshold density. In some embodiments, the cultivation ends at, at about, or within 6 hours, 12 hours, 24 hours, 36 hours, 1 day, 2 days, 3 days, 4 days, 5 days, 6 days, or 7 or more days, after the threshold density is achieved. In particular embodiments, the cultivation is ended at or about 1 day after the threshold density is achieved.

In some embodiments, the cultivation step is performed for the amount of time required for the cells to achieve a threshold amount, density, and/or expansion. In some embodiments, the cultivation is performed for or for about, or for less than, 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In particular embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In certain embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks.

In certain embodiments, the cultivation step is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the cultivation step is performed until 1 day after the cells achieve a threshold cell count of or of about 1200×10⁶ cells and are cultured for a minimum of 10 days, and/or until 1 day after the cells achieve a threshold cell count of or of about 5000×10⁶ cells. In some embodiments, the cultivation step is performed until 1 day after the cells achieve a threshold cell count of or of about 1200×10⁶ cells and are cultured for a minimum of 9 days, and/or until 1 day after the cells achieve a threshold cell count of or of about 5000×10⁶ cells. In some embodiments, the cultivation step is performed until 1 day after the cells achieve a threshold cell count of or of about 1000×10⁶ cells and are cultured for a minimum of 8 days, and/or until 1 day after the cells achieve a threshold cell count of or of about 4000×10⁶ cells. In certain embodiments, the cultivation is an expansion step and is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the expansion step is performed until 1 day after the cells achieve a threshold cell count of or of about 1200×10⁶ cells and are expanded for a minimum of 10 days, and/or until 1 day after the cells achieve a threshold cell count of or of about 5000×10⁶ cells. In some embodiments, the expansion step is performed until 1 day after the cells achieve a threshold cell count of or of about 1200×10⁶ cells and are expanded for a minimum of 9 days, and/or until 1 day after the cells achieve a threshold cell count of or of about 5000×10⁶ cells. In some embodiments, the expansion step is performed until 1 day after the cells achieve a threshold cell count of or of about 1000×10⁶ cells and are expanded for a minimum of 8 days, and/or until 1 day after the cells achieve a threshold cell count of or of about 4000×10⁶ cells. In some embodiments, the expansion step is performed until 1 day after the cells achieve a threshold cell count of or of about 1400×10⁶ cells and are expanded for a minimum of 5 days, and/or until 1 day after the cells achieve a threshold cell count of or of about 4000×10⁶ cells.

In some embodiments, the cultivation is performed for at least a minimum amount of time. In some embodiments, the cultivation is performed for at least 14 days, at least 12 days, at least 10 days, at least 7 days, at least 6 days, at least 5 days, at least 4 days, at least 3 days, at least 2 days, at least 36 hours, at least 24 hours, at least 12 hours, or at least 6 hours, even if the threshold is achieved prior to the minimum amount of time. In some embodiments, increasing the minimum amount of time that the cultivation is performed, may, in some cases, reduce the activation and/or reduce the level or one or more activation markers, in the cultivated cells, formulated cells, and/or cells of the output composition. In some embodiments, the minimum cultivation time counts from a determined point an exemplary process (e.g., a selection step; a thaw step; and/or an activation step) to the day the cells are harvested.

In aspects of the provided embodiments, the density and/or concentration of the cells or of the viable cells during the cultivation is monitored or carried out during the cultivation, such as until a threshold amount, density, and/or expansion is achieved as described. In some embodiments such methods include those as described, including optical methods, including digital holography microscopy (DHM) or differential digital holography microscopy (DDHM).

In certain embodiments, the cultivated cells are output cells. In some embodiments, a composition of enriched T cells, such as engineered T cells, that has been cultivated is an output composition of enriched T cells. In particular embodiments, CD4+ T cells and/or CD8+ T cells that have been cultivated are output CD4+ and/or CD8+ T cells. In particular embodiments, a composition of enriched CD4+ T cells, such as engineered CD4+ T cells, that has been cultivated is an output composition of enriched CD4+ T cells. In some embodiments, a composition of enriched CD8+ T cells, such as engineered CD8+ T cells, that has been cultivated is an output composition of enriched CD8+ T cells.

In some embodiments, the cells are cultivated under conditions that promote proliferation and/or expansion in presence of one or more cytokines. In particular embodiments, at least a portion of the cultivation is performed with constant mixing and/or perfusion, such as mixing or perfusion controlled by a bioreactor. In some embodiments, the cells are cultivated in the presence or one or more cytokines and with a surfactant, e.g., poloxamer, such as poloxamer 188, to reduce shearing and/or shear stress from constant mixing and/or perfusion. In some embodiments, a composition of enriched CD4+ T cells, such as engineered CD4+ T cells, is cultivated in the presence of recombinant IL-2, IL-7, IL-15, and poloxamer, wherein at least a portion of the cultivating is performed with constant mixing and/or perfusion. In certain embodiments, a composition of enriched CD8+ T cells, such as engineered CD8+ T cells, is cultivated in the presence of recombinant IL-2, IL-15, and poloxamer, wherein at least a portion of the cultivating is performed with constant mixing and/or perfusion. In some embodiments, the cultivation is performed until the cells reach as threshold expansion of at least 4-fold e.g., as compared to the start of the cultivation.

1. Monitoring Cells During Cultivation

In some embodiments, the cells are monitored during the cultivation step. Monitoring may be performed, for example, to ascertain (e.g., measure, quantify) cell morphology, cell viability, cell death, and/or cell concentration (e.g., viable cell concentration). In some embodiments, the monitoring is performed manually, such as by a human operator. In some embodiments, the monitoring is performed by an automated system. The automated system may require minimal or no manual input to monitor the cultivated cells. In some embodiments, the monitoring is performed both manually and by an automated system.

In certain embodiments, the cells are monitored by an automated system requiring no manual input. In some embodiments, the automated system is compatible with a bioreactor, for example a bioreactor as described herein, such that cells undergoing cultivation can be removed from the bioreactor, monitored, and subsequently returned to the bioreactor. In some embodiments, the monitoring and cultivation occur in a closed loop configuration. In some aspects, in a closed loop configuration, the automated system and bioreactor remain sterile. In embodiments, the automated system is sterile. In some embodiments, the automated system is an in-line system.

In some embodiments, the automated system includes the use of optical techniques (e.g., microscopy) for detecting cell morphology, cell viability, cell death, and/or cell concentration (e.g., viable cell concentration). Any optical technique suitable for determining, for example, cell features, viability, and concentration are contemplated herein. Non-limiting examples of useful optical techniques include bright field microscopy, fluorescence microscopy, differential interference contrast (DIC) microscopy, phase contrast microscopy, digital holography microscopy (DHM), differential digital holography microscopy (DDHM), or a combination thereof. Differential digital holography microscopy, DDHM, and differential DHM may be used herein interchangeably. In certain embodiments, the automated system includes a differential digital holography microscope. In certain embodiments, the automated system includes a differential digital holography microscope including illumination means (e.g., laser, led). Descriptions of DDHM methodology and use may be found, for example, in U.S. Pat. No. 7,362,449; EP 1,631,788; U.S. Pat. Nos. 9,904,248; and 9,684,281, which are incorporated herein by reference in their entirety.

DDHM permits label-free, non-destructive imaging of cells, resulting in high-contrast holographic images. The images may undergo object segmentation and further analysis to obtain a plurality of morphological features that quantitatively describe the imaged objects (e.g., cultivated cells, cellular debris). As such, various features (e.g., cell morphology, cell viability, cell concentration) may be directly assessed or calculated from DDHM using, for example, the steps of image acquisition, image processing, image segmentation, and feature extraction. In some embodiments, the automated system includes a digital recording device to record holographic images. In some embodiments, the automated system includes a computer including algorithms for analyzing holographic images. In some embodiments, the automated system includes a monitor and/or computer for displaying the results of the holographic image analysis. In some embodiments, the analysis is automated (i.e., capable of being performed in the absence of user input). An example of a suitable automated system for monitoring cells during the cultivating step includes, but is not limited to, Ovizio iLine F (Ovizio Imaging Systems NV/SA, Brussels, Belgium).

In certain embodiments, the monitoring is performed continuously during the cultivation step. In some embodiments, the monitoring is performed in real-time during the cultivation step. In some embodiments, the monitoring is performed at discrete time points during the cultivation step. In some embodiments, the monitoring is performed at least every 15 minutes for the duration of the cultivating step. In some embodiments, the monitoring is performed at least every 30 minutes for the duration of the cultivating step. In some embodiments, the monitoring is performed at least every 45 minutes for the duration of the cultivating step. In some embodiments, the monitoring is performed at least every hour for the duration of the cultivating step. In some embodiments, the monitoring is performed at least every 2 hours for the duration of the cultivating step. In some embodiments, the monitoring is performed at least every 4 hours for the duration of the cultivating step. In some embodiments, the monitoring is performed at least every 6 hours for the duration of the cultivating step. In some embodiments, the monitoring is performed at least every 8 hours for the duration of the cultivating step. In some embodiments, the monitoring is performed at least every 10 hours for the duration of the cultivating step. In some embodiments, the monitoring is performed at least every 12 hours for the duration of the cultivating step. In some embodiments, the monitoring is performed at least every 14 hours for the duration of the cultivating step. In some embodiments, the monitoring is performed at least every 16 hours for the duration of the cultivating step. In some embodiments, the monitoring is performed at least every 18 hours for the duration of the cultivating step. In some embodiments, the monitoring is performed at least every 20 hours for the duration of the cultivating step. In some embodiments, the monitoring is performed at least every 22 hours for the duration of the cultivating step. In some embodiments, the monitoring is performed at least once a day for the duration of the cultivating step. In some embodiments, the monitoring is performed at least once every second day for the duration of the cultivating step. In some embodiments, the monitoring is performed at least once every third day for the duration of the cultivating step. In some embodiments, the monitoring is performed at least once every fourth day for the duration of the cultivating step. In some embodiments, the monitoring is performed at least once every fifth day for the duration of the cultivating step. In some embodiments, the monitoring is performed at least once every sixth day for the duration of the cultivating step. In some embodiments, the monitoring is performed at least once every seventh day for the duration of the cultivating step. In some embodiments, the monitoring is performed at least once every eighth day for the duration of the cultivating step. In some embodiments, the monitoring is performed at least once every ninth day for the duration of the cultivating step. In some embodiments, the monitoring is performed at least once every tenth day for the duration of the cultivating step. In some embodiments, the monitoring is performed at least once during the cultivating step.

In some embodiments, features of the cells that can be determined by the monitoring, including using optical techniques such as DHM or DDHM, include cell viability, cell concentration, cell number and/or cell density. In some embodiments, cell viability is characterized or determined. In some embodiments, cell concentration, density and/or number is characterized or determined. In some embodiments, viable cell concentration, viable cell number and/or viable cell density is characterized or determined. In some embodiments, the cultivated cells are monitored by the automated system until a threshold of expansion is reached, such as described above. In some embodiments, once a threshold of expansion is reached, the cultivated cells are harvested, such as by automatic or manual methods, for example, by a human operator. The threshold of expansion may depend on the total concentration, density and/or number of cultured cells determined by the automated system. Alternatively, the threshold of expansion may depend on the viable cell concentration, density and/or number.

In some embodiments, the harvested cells are formulated as described, such as in the presence of a pharmaceutically acceptable carrier. In some embodiments, the harvested cells are formulated in the presence of a cryoprotectant.

E. Formulating the Cells

In some embodiments, the provided methods for manufacturing, generating or producing a cell therapy and/or engineered cells may include formulation of cells, such as formulation of genetically engineered cells resulting from the provided processing steps prior to or after the incubating, engineering, and cultivating, and/or one or more other processing steps as described. In some embodiments, the provided methods associated with formulation of cells include processing transduced cells, such as cells transduced and/or expanded using the processing steps described above, in a closed system. In some embodiments, the dose of cells comprising cells engineered with a recombinant antigen receptor, e.g., CAR or TCR, is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods.

In some cases, the cells are processed in one or more steps (e.g., carried out in the centrifugal chamber and/or closed system) for manufacturing, generating or producing a cell therapy and/or engineered cells may include formulation of cells, such as formulation of genetically engineered cells resulting from the provided transduction processing steps prior to or after the culturing, e.g., cultivation and expansion, and/or one or more other processing steps as described. In some cases, the cells can be formulated in an amount for dosage administration, such as for a single unit dosage administration or multiple dosage administration. In some embodiments, the provided methods associated with formulation of cells include processing transduced cells, such as cells transduced and/or expanded using the processing steps described above, in a closed system.

In certain embodiments, one or more compositions of enriched T cells, such as engineered and cultivated T cells, e.g., output T cells, therapeutic cell composition, are formulated. In particular embodiments, one or more compositions of enriched T cells, such as engineered and cultivated T cells, e.g., output T cells, therapeutic cell composition, are formulated after the one or more compositions have been engineered and/or cultivated. In particular embodiments, the one or more compositions are input compositions. In some embodiments, the one or more input compositions have been previously cryofrozen and stored, and are thawed prior to the incubation.

In certain embodiments, the one or more therapeutic compositions of enriched T cells, such as engineered and cultivated T cells, e.g., output T cells, are or include two separate compositions, e.g., separate engineered and/or cultivated compositions, of enriched T cells. In particular embodiments, two separate therapeutic compositions of enriched T cells, e.g., two separate compositions of enriched CD4+ T cells and CD8+ T cells selected, isolated, and/or enriched from the same biological sample, separately engineered and separately cultivated, are separately formulated. In certain embodiments, the two separate therapeutic cell compositions include a composition of enriched CD4+ T cells, such as a composition of engineered and/or cultivated CD4+ T cells. In particular embodiments, the two separate therapeutic cell compositions include a composition of enriched CD8+ T cells, such as a composition of engineered and/or cultivated CD8+ T cells. In some embodiments, two separate therapeutic compositions of enriched CD4+ T cells and enriched CD8+ T cells, such as separate compositions of engineered and cultivated CD4+ T cells and engineered and cultivated CD8+ T cells, are separately formulated. In some embodiments, a single therapeutic composition of enriched T cells is formulated. In certain embodiments, the single therapeutic composition is a composition of enriched CD4+ T cells, such as a composition of engineered and/or cultivated CD4+ T cells. In some embodiments, the single therapeutic composition is a composition of enriched CD4+ and CD8+ T cells that have been combined from separate compositions prior to the formulation.

In some embodiments, separate therapeutic compositions of enriched CD4+ and CD8+ T cells, such as separate compositions of engineered and cultivated CD4+ and CD8+ T cells, are combined into a single therapeutic composition and are formulated. In certain embodiments, separate formulated therapeutic compositions of enriched CD4+ and enriched CD8+ T cells are combined into a single therapeutic composition after the formulation has been performed and/or completed. In particular embodiments, separate therapeutic compositions of enriched CD4+ and CD8+ T cells, such as separate compositions of engineered and cultivated CD4+ and CD8+ T cells, are separately formulated as separate compositions.

In some embodiments, the therapeutic composition of enriched CD4+ T cells, such as an engineered and cultivated CD4+ T cells, e.g., output CD4+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express a recombinant receptor and/or have been transduced or transfected with the recombinant polynucleotide. In certain embodiments, the therapeutic composition of enriched CD4+ T cells, such as an engineered and cultivated CD4+ T cells, e.g., output CD4+ T cells, that is formulated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the therapeutic composition of enriched CD8+ T cells, such as an engineered and cultivated CD8+ T cells, e.g., output CD8+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the therapeutic composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide. In certain embodiments, the therapeutic composition of enriched CD8+ T cells, such as an engineered and cultivated CD8+ T cells, e.g., output CD8+ T cells, that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, features of the one or more therapeutic compositions are assessed, for example as described in Sections I-A and I-A-3, prior to formulation. In some embodiments, the features are cell phenotypes and recombinant receptor-dependent activity. In some embodiments, the cell phenotypes and recombinant receptor-dependent activity, are quantified to provide a number, percentage, proportion, and/or ratio of cells having an attribute in the therapeutic cell composition. In some embodiments, the features are used as input to a machine learning process provided herein.

In certain embodiments, the formulated cells are output cells. In some embodiments, a formulated therapeutic composition of enriched T cells, such as a formulated composition of engineered and cultivated T cells, is an output composition of enriched T cells. In particular embodiments, the formulated CD4+ T cells and/or formulated CD8+ T cells are the output CD4+ and/or CD8+ T cells. In particular embodiments, a formulated composition of enriched CD4+ T cells is an output composition of enriched CD4+ T cells. In some embodiments, a formulated composition of enriched CD8+ T cells is an output composition of enriched CD8+ T cells.

In some embodiments, cells can be formulated into a container, such as a bag or vial. In some embodiments, the cells are formulated between 0 days and 10 days, between 0 and 5 days, between 2 days and 7 days, between 0.5 days, and 4 days, or between 1 day and 3 days after the cells after the threshold cell count, density, and/or expansion has been achieved during the cultivation. In certain embodiments, the cells are formulated at or at or about or within 12 hours, 18 hours, 24 hours, 1 day, 2 days, or 3 days after the threshold cell count, density, and/or expansion has been achieved during the cultivation. In some embodiments, the cells are formulated within or within about 1 day after the threshold cell count, density, and/or expansion has been achieved during the cultivation.

Particular embodiments contemplate that cells are in a more activated state at early stages during the cultivation than at later stages during the cultivation. Further, in some embodiments, it may be desirable to formulate cells that are in a less activated state than the peak activation that occurs or may occur during the cultivation. In certain embodiments, the cells are cultivated for a minimum duration or amount of time, for example, so that cells are harvested in a less activated state than if they were formulated at an earlier time point during the cultivation, regardless of when the threshold is achieved. In some embodiments, the cells are cultivated between 1 day and 3 days after the threshold cell count, density, and/or expansion has been achieved during the cultivation. In certain embodiments, the cells achieve the threshold cell count, density, and/or expansion and remain cultivated for a minimum time or duration prior to the formulation. In some embodiments, cells that have achieved the threshold are not formulated until they have been cultivated for a minimum duration and/or amount of time, such as a minimum time or duration of between 1 day and 14 days, 2 days and 7 days, or 3 days and 6 days, or a minimum time or duration of the cultivation of or of about 2 days, 3 days, 4 days, 5 days, 6 days, 7 days, or more than 7 days. In some embodiments, the minimum time or duration of the cultivation is between 3 days and 6 days.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cell are formulated with a cryopreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9. 0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the therapeutic composition of enriched T cells, e.g., T cells that have been stimulated, engineered, and/or cultivated, are formulated, cryofrozen, and then stored for an amount of time. In certain embodiments, the formulated, cryofrozen cells are stored until the cells are released for infusion. In particular embodiments, the formulated cryofrozen cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryofrozen and stored for, for about, or for less than 1 days, 2 days, 3 days, 4 days, 5 days, 6 days, or 7 days. In certain embodiments, the cells are thawed and administered to a subject after the storage. In certain embodiments, the cells are stored for or for about 5 days.

In some embodiments, the formulation is carried out using one or more processing step including washing, diluting or concentrating the cells, such as the cultured or expanded cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or expanded cells. In some embodiments, the volume of formulation buffer is from 10 mL to 1000 mL or from about 10 mL to about 1000 mL, such as at least or about at least or about 50 mL, 100 mL, 200 mL, 300 mL, 400 mL, 500 mL, 600 mL, 700 mL, 800 mL, 900 mL or 1000 mL

In some embodiments, such processing steps for formulating a cell composition is carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as the vials of the biomedical material vessels described herein, that is operably linked as part of a closed system with the centrifugal chamber. In some embodiments, the biomedical material vessels are configured for integration and/or operable connection and/or is integrated or operably connected, to a closed system or device that carries out one or more processing steps. In some embodiments, the biomedical material vessel is connected to a system at an output line or output position. In some cases, the closed system is connected to the vial of the biomedical material vessel at the inlet tube. Exemplary close systems for use with the biomedical material vessels described herein include the Sepax® and Sepax® 2 system.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of vials, can be sterilely connected to one or more, generally two or more, such as at least 3, 4, 5, 6, 7, 8 or more of the ports of the multi-port output. For example, in some embodiments, one or more containers, e.g., biomedical material vessels, can be attached to the ports, or to fewer than all of the ports. Thus, in some embodiments, the system can effect expression of the output composition into a plurality of vials of the biomedical material vessels.

In some aspects, cells can be expressed to the one or more of the plurality of output containers, e.g., vials of the biomedical material vessels, in an amount for dosage administration, such as for a single unit dosage administration or multiple dosage administration. For example, in some embodiments, the vials of the biomedical material vessels, may each contain the number of cells for administration in a given dose or fraction thereof. Thus, each vial, in some aspects, may contain a single unit dose for administration or may contain a fraction of a desired dose such that more than one of the plurality of vials, such as two of the vials, or 3 of the vials, together constitutes a dose for administration.

Thus, the vials described herein, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, each of the containers, e.g., bags of vials individually comprises a unit dose of the cells. Thus in some embodiments, each of the containers comprises the same or approximately or substantially the same number of cells. In some embodiments, each unit dose contains at least or about at least 1×10⁶, 2×10⁶, 5×10⁶, 1×10⁷, 5×10⁷, or 1×10⁸ engineered cells, total cells, T cells, or PBMCs. In some embodiments, the volume of the formulated cell composition in each container, e.g., bag or vial, is 10 mL to 100 mL, such as at least or about at least or about 20 mL, 30 mL, 40 mL, 50 mL, 60 mL, 70 mL, 80 mL, 90 mL or 100 mL. In some embodiments, the cells in the container, e.g., bag or vials, can be cryopreserved. In some embodiments, the container, e.g., vials, can be stored in liquid nitrogen until further use.

In some embodiments, such cells produced by the method, or a composition comprising such cells, are administered to a subject for treating a disease or condition.

### III. RECOMBINANT RECEPTORS FOR GENETIC ENGINEERING

In some embodiments, the cells, e.g., CD4+ cells, CD8+ cells, of the therapeutic cell composition are engineered cells encoding a recombinant protein. In some embodiments, the engineered cells of the therapeutic cell composition contain or express, a recombinant protein, such as a recombinant receptor, e.g., a chimeric antigen receptor (CAR), or a T cell receptor (TCR). In certain embodiments, the methods for manufacturing or engineering described produce and/or are capable of producing cells, or populations or compositions containing and/or enriched for cells, that are engineered to express or contain a recombinant protein such as a recombinant receptor.

In some aspects, the encoded recombinant receptor is a chimeric antigen receptor (CAR) or a recombinant T cell receptor (TCR). Among the recombinant receptors are chimeric receptors, antigen receptors and receptors containing one or more component of chimeric receptors or antigen receptors. The recombinant receptors may include those containing ligand-binding domains or binding fragments thereof and intracellular signaling domains or regions. In some embodiments, the recombinant receptors encoded by the engineered cells include functional non-TCR antigen receptors, chimeric antigen receptors (CARs), chimeric autoantibody receptor (CAAR), recombinant T cell receptors (TCRs) and regions, domains or components of any of the foregoing, including one or more polypeptide chains of a multi-chain recombinant receptor. The recombinant receptor, such as a CAR, generally includes the extracellular antigen (or ligand) binding domain linked to one or more intracellular signaling components, in some aspects via linkers and/or transmembrane domain(s). In some embodiments, exemplary recombinant receptors expressed from the engineered cell include multi-chain receptors that contain two or more receptor polypeptides, which, in some cases, contain different components, domains or regions. In some aspects, the recombinant receptor contains two or more polypeptides that together comprise a functional recombinant receptor. In some aspects, the multi-chain receptor is a dual-chain receptor, comprising two polypeptides that together comprise a functional recombinant receptor. In some embodiments, the recombinant receptor is a TCR comprising two different receptor polypeptides, for example, a TCR alpha (TCRα) and a TCR beta (TCRβ) chain; or a TCR gamma (TCRγ) and a TCR delta (TCRδ) chain. In some embodiments, the recombinant receptor is a multi-chain receptor in which one or more of the polypeptides regulates, modifies or controls the expression, activity or function of another receptor polypeptide. In some aspects, multi-chain receptors allows spatial or temporal regulation or control of specificity, activity, antigen (or ligand) binding, function and/or expression of the receptor.

A. Chimeric Antigen Receptors (CARs)

In some embodiments, the encoded recombinant receptor is a chimeric antigen receptor (CAR) with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain or region (also interchangeably called an intracellular signaling domain or region), such as a cytoplasmic (intracellular) region capable of inducing a primary activation signal in a T cell, for example, a cytoplasmic signaling domain or region of a T cell receptor (TCR) component (e.g., a cytoplasmic signaling domain or region of a zeta chain of a CD3-zeta (CD3) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g., antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g., CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the CAR contains an antibody or an antigen-binding fragment (e.g., scFv) that specifically recognizes an antigen, such as an intact antigen, expressed on the surface of a cell.

In some embodiments, the antigen (or a ligand) is a tumor antigen or cancer marker. In some embodiments, the antigen (or a ligand) the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen Al (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the CAR is an anti-BCMA CAR that is specific for BCMA, e.g., human BCMA. Chimeric antigen receptors containing anti-BCMA antibodies, including mouse anti-human BCMA antibodies and human anti-human antibodies, and cells expressing such chimeric receptors have been previously described. See Carpenter et al., Clin Cancer Res., 2013, 19(8):2048-2060, WO 2016/090320, WO2016090327, WO2010104949A2 and WO2017173256. In some embodiments, the anti-BCMA CAR contains an antigen-binding domain, such as an scFv, containing a variable heavy (V_{H}) and/or a variable light (V_{L}) region derived from an antibody described in WO 2016/090320 or WO2016090327. In some embodiments, the antigen-binding domain, such as an scFv, contains a V_{H} set forth in SEQ ID NO: 13 and a V_{L} set forth in SEQ ID NO:14. In some embodiments, the antigen-binding domain, such as an scFv, contains a V_{H} set forth in SEQ ID NO: 15 and a V_{L} set forth in SEQ ID NO:16. In some embodiments, the antigen-binding domain, such as an scFv, contains a VH set forth in SEQ ID NO: 17 and a V_{L} set forth in SEQ ID NO: 18. In some embodiments, the antigen-binding domain, such as an scFv, contains a V_{H} set forth in SEQ ID NO: 19 and a V_{L} set forth in SEQ ID NO:20. In some embodiment the antigen-binding domain, such as an scFv, contains a V_{H} set forth in SEQ ID NO: 21 and a V_{L} set forth in SEQ ID NO: 22. In some embodiments, the antigen-binding domain, such as an scFv, contains a V_{H} set forth in SEQ ID NO: 23 and a V_{L} set forth in SEQ ID NO: 24. In some embodiments, the antigen-binding domain, such as an scFv, contains a V_{H} set forth in SEQ ID NO: 25 and a V_{L} set forth in SEQ ID NO: 26. In some embodiments, the V_{H} or V_{L} has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing V_{H} or V_{L} sequences, and retains binding to BCMA. In some embodiments, the V_{H} region is amino-terminal to the V_{L} region. In some embodiments, the V_{H} region is carboxy-terminal to the V_{L} region.

In some embodiments, the antibody or an antigen-binding fragment (e.g., scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a V_{H} and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the CAR is an anti-CD19 CAR that is specific for CD19, e.g., human CD19. In some embodiments the scFv and/or V_{H} domains is derived from FMC63. FMC63 generally refers to a mouse monoclonal IgG1 antibody raised against Nalm-1 and −16 cells expressing CD19 of human origin (Ling, N. R., et al. (1987). Leucocyte typing III. 302). In some embodiments, the FMC63 antibody comprises a CDR-H1 and a CDR-H2 set forth in SEQ ID NOS: 30 and 31 respectively, and a CDR-H3 set forth in SEQ ID NO: 32 or 33 and a CDR-L1 set forth in SEQ ID NO: 27 and a CDR-L2 set forth in SEQ ID NO: 28 or 34 and a CDR-L3 sequences set forth in SEQ ID NO: 29 or 35. In some embodiments, the FMC63 antibody comprises a heavy chain variable region (V_{H}) comprising the amino acid sequence of SEQ ID NO: 36 and a light chain variable region (V_{L}) comprising the amino acid sequence of SEQ ID NO: 37.

In some embodiments, the scFv comprises a variable light chain containing a CDR-L1 sequence of SEQ ID NO:27, a CDR-L2 sequence of SEQ ID NO:28, and a CDR-L3 sequence of SEQ ID NO:29 and/or a variable heavy chain containing a CDR-H1 sequence of SEQ ID NO:30, a CDR-H2 sequence of SEQ ID NO:31, and a CDR-H3 sequence of SEQ ID NO:32. In some embodiments, the scFv comprises a variable heavy chain region set forth in SEQ ID NO:36 and a variable light chain region set forth in SEQ ID NO:37. In some embodiments, the variable heavy and variable light chains are connected by a linker. In some embodiments, the linker is set forth in SEQ ID NO:38. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:39 or a sequence that exhibits at least 85%, 86%, 87%, 88%, 89%, 90%, 91%, 92%, 93%, 94%, 95%, 96%, 97%, 98%, or 99% sequence identity to SEQ ID NO:39. In some embodiments, the scFv comprises the sequence of amino acids set forth in SEQ ID NO:40 or a sequence that exhibits at least 85%, 86%, 87%, 88%, 89%, 90%, 91%, 92%, 93%, 94%, 95%, 96%, 97%, 98%, or 99% sequence identity to SEQ ID NO:40.

In some embodiments the scFv is derived from SJ25C1. SJ25C1 is a mouse monoclonal IgG1 antibody raised against Nalm-1 and −16 cells expressing CD19 of human origin (Ling, N. R., et al. (1987). Leucocyte typing III. 302). In some embodiments, the SJ25C1 antibody comprises a CDR-H1, a CDR-H2 and a CDR-H3 sequence set forth in SEQ ID NOS: 41-43, respectively, and a CDR-L1, a CDR-L2 and a CDR-L3 sequence set forth in SEQ ID NOS: 44-46, respectively. In some embodiments, the SJ25C1 antibody comprises a heavy chain variable region (V_{H}) comprising the amino acid sequence of SEQ ID NO: 47 and a light chain variable region (V_{L}) comprising the amino acid sequence of SEQ ID NO: 48. In some embodiments, the scFv comprises a variable light chain containing a CDR-L1 sequence of SEQ ID NO:44, a CDR-L2 sequence of SEQ ID NO: 45, and a CDR-L3 sequence of SEQ ID NO:46 and/or a variable heavy chain containing a CDR-H1 sequence of SEQ ID NO:41, a CDR-H2 sequence of SEQ ID NO:42, and a CDR-H3 sequence of SEQ ID NO:43. In some embodiments, the scFv comprises a variable heavy chain region set forth in SEQ ID NO:47 and a variable light chain region set forth in SEQ ID NO:48. In some embodiments, the variable heavy and variable light chain are connected by a linker. In some embodiments, the linker is set forth in SEQ ID NO:49. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:50 or a sequence that exhibits at least 85%, 86%, 87%, 88%, 89%, 90%, 91%, 92%, 93%, 94%, 95%, 96%, 97%, 98%, or 99% sequence identity to SEQ ID NO:50.

In some embodiments, the CAR is an anti-CD20 CAR that is specific for CD20. In some embodiments, the scFv contains a V_{H} and a V_{L} derived from an antibody or an antibody fragment specific to CD20. In some embodiments, the antibody or antibody fragment that binds CD20 is an antibody that is or is derived from Rituximab, such as is Rituximab scFv.

In some embodiments, the CAR is an anti-CD22 CAR that is specific for CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as is m971 scFv.

In some embodiments, the CAR is an anti-GPRCSD CAR that is specific for GPRCSD. In some embodiments, the scFv contains a V_{H} and a V_{L} derived from an antibody or an antibody fragment specific to GPRCSD. In some embodiments, the antibody or antibody fragment that binds GPRCSD is or contains a V_{H} and a V_{L} from an antibody or antibody fragment set forth in International Patent Applications, Publication Number WO 2016/090329 and WO 2016/090312.

In some embodiments, the antibody is an antigen-binding fragment, such as a scFv, that includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. Exemplary linkers include linkers having various numbers of repeats of the sequence GGGGS (4GS; SEQ ID NO:51) or GGGS (3GS; SEQ ID NO:52), such as between 2, 3, 4, and 5 repeats of such a sequence. Exemplary linkers include those having or consisting of an sequence set forth in SEQ ID NO:53 (GGGGSGGGGSGGGGS), SEQ ID NO:54 (GSTSGSGKPGSGEGSTKG) or SEQ ID NO: 55 (SRGGGGSGGGGSGGGGSLEMA).

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In some embodiments, the CAR contains a TCR-like antibody, such as an antibody or an antigen-binding fragment (e.g., scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning α chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see, e.g., US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See, e.g., US published application No. US20020150914, US2014/0294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known, in some cases, to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known, in some cases, to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 4, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 4
Boundaries of CDRs according to various numbering schemes.

| CDR | Kabat | Chothia | AbM | Contact |
|---|---|---|---|---|
| CDR-L1 | L24-L34 | L24-L34 | L24-L34 | L30-L36 |
| CDR-L2 | L50-L56 | L50-L56 | L50-L56 | L46-L55 |
| CDR-L3 | L89-L97 | L89-L97 | L89-L97 | L89-L96 |
| CDR-H1 | H31-H35B | H26- | H26-H35B | H30-H35B |
| (Kabat Numbering¹) | | H32 . . . 34 | | |
| CDR-H1 | H31-H35 | H26-H32 | H26-H35 | H30-H35 |
| (Chothia | | | | |
| Numbering²) | | | | |
| CDR-H2 | H50-H65 | H52-H56 | H50-H58 | H47-H58 |
| CDR-H3 | H95-H102 | H95-H102 | H95-H102 | H93-H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method, or other known schemes. In other cases, the particular amino acid sequence of a CDR or FR is given.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Among the antibodies included in the provided CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antigen-binding domain in the provided CARs is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the recombinant receptor such as the CAR, such as the antibody portion thereof, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain The spacer can be of a length that provides for increased responsiveness of the cell following antigen binding, as compared to in the absence of the spacer.

In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al. (2013) Clin. Cancer Res., 19:3153, Hudecek et al. (2015) Cancer Immunol Res. 3(2): 125-135 or International Pat. App. Pub. No. WO2014031687, U.S. Pat. No. 8,822,647 or US2014/0271635. In some embodiments, the spacer includes a sequence of an immunoglobulin hinge region, a C_{H}2 and C_{H}3 region. In some embodiments, one of more of the hinge, C_{H}2 and C_{H}3 is derived all or in part from IgG4 or IgG2. In some cases, the hinge, C_{H}2 and C_{H}3 is derived from IgG4. In some aspects, one or more of the hinge, C_{H}2 and C_{H}3 is chimeric and contains sequence derived from IgG4 and IgG2. In some examples, the spacer contains an IgG4/2 chimeric hinge, an IgG2/4 C_{H}2, and an IgG4 CH3 region.

In some embodiments, the spacer can be derived all or in part from IgG4 and/or IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the spacer can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N to Q substitution at a position corresponding to position 177 in the C_{H}2 region of the IgG4 heavy chain constant region sequence set forth in SEQ ID NO: 70 (Uniprot Accession No. P01861; position corresponding to position 297 by EU numbering and position 79 of the hinge-C_{H}2-C_{H}3 spacer sequence set forth in SEQ ID NO:56 or an N to Q substitution at a position corresponding to position 176 in the C_{H}2 region of the IgG2 heavy chain constant region sequence set forth in SEQ ID NO: 57 (Uniprot Accession No. P01859; position corresponding to position 297 by EU numbering).

In some aspects, the spacer contains only a hinge region of an IgG, such as only a hinge of IgG4 or IgG1, such as the hinge only spacer set forth in SEQ ID NO:58, and is encoded by the sequence set forth in SEQ ID NO: 60. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:59. In some embodiments, the spacer is an Ig hinge, e.g., an IgG4 hinge, linked to a C_{H}3 domain only, such as set forth in SEQ ID NO:56. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 61. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 56-61.

The antigen recognition domain generally is linked to one or more intracellular signaling components, such as signaling components that mimic stimulation or activation through an antigen receptor complex, such as a TCR complex, in the case of a CAR, and/or signal via another cell surface receptor. Thus, in some embodiments, the antigen binding component (e.g., antibody) is linked to one or more transmembrane and intracellular signaling regions. In some embodiments, the transmembrane domain is fused to the extracellular domain In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the ROR1-binding antibody is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling region and/or transmembrane portion of a costimulatory receptor, such as CD28, 4-1BB, OX40, DAP10, and ICOS. In some aspects, the same CAR includes both the signaling region and costimulatory components.

In some embodiments, the signaling region is included within one CAR, whereas the costimulatory component is provided by another CAR recognizing another antigen. In some embodiments, the CARs include activating or stimulatory CARs, and costimulatory CARs, both expressed on the same cell (see WO2014/055668).

In certain embodiments, the intracellular signaling region comprises a CD28 transmembrane and signaling domain linked to a CD3 (e.g., CD3-zeta) intracellular domain In some embodiments, the intracellular signaling region comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain disposed between the extracellular domain and the intracellular signaling region.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 62 or a sequence of amino acids that exhibits at least or at least about85%, 86%, 87%, 88%, 89%, 90%, 91%, 92%, 93%, 94%, 95%, 96%, 97%, 98%, 99% or more sequence identity to SEQ ID NO:62; in some embodiments, the transmembrane-domain containing portion of the recombinant receptor comprises the sequence of amino acids set forth in SEQ ID NO: 63 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular signaling region comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 64 or 65 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 64 or 65. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 66 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 66.

In some embodiments, the intracellular signaling region comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 67, 68 or 69 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 67, 68 or 69.

In some aspects, the spacer contains only a hinge region of an IgG, such as only a hinge of IgG4 or IgG1, such as the hinge only spacer set forth in SEQ ID NO:58. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:59. In some embodiments, the spacer is an Ig hinge, e.g., an IgG4 hinge, linked to a C_{H}3 domain only, such as set forth in SEQ ID NO:56. In some embodiments, the spacer is or comprises a glycine-serine rich sequence or other flexible linker such as known flexible linkers.

B. T Cell Receptors (TCRs)

In some embodiments, the encoded recombinant receptor is a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable a and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the a and chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g., CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g., cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable a domain, a constant a domain and a first dimerization motif attached to the C-terminus of the constant a domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See, e.g., Soo Hoo, W. F. et al. PNAS (USA) 89, 4759 (1992); Wülfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see, e.g., International published PCT No. WO 03/020763). In some embodiments, a scTCR is a non-disulfide linked truncated TCR in which heterologous leucine zippers fused to the C-termini thereof facilitate chain association (see, e.g., International published PCT No. WO99/60120). In some embodiments, a scTCR contain a TCRα variable domain covalently linked to a TCRβ variable domain via a peptide linker (see, e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)s-P- wherein P is proline, G is glycine and S is serine (SEQ ID NO:22). In some embodiments, the linker has the sequence

(SEQ ID NO: 23)
GSADDAKKDAAKKDGKS

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, nucleic acid or nucleic acids encoding a TCR, such as a and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are co-expressed. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g., lentiviral, vector.

### IV. METHODS OF ADMINISTRATION

In some aspects, the therapeutic cell composition comprising engineered cells, e.g., engineered CD4+ and CD8+ cells, can be used in connection with a method of treatment, e.g., including administering any of the engineered cells or compositions containing engineered cells that have been assessed using the methods provided herein.

In some embodiments, the engineered cells expressing a recombinant receptor or compositions comprising the same, are useful in a variety of therapeutic, diagnostic and prophylactic indications. For example, the engineered cells or compositions comprising the engineered cells, e.g., therapeutic cell compositions, are useful in treating a variety of diseases and disorders in a subject. Methods and uses include therapeutic methods and uses, for example, involving administration of the engineered cells, or compositions containing the same, to a subject having a disease, condition, or disorder, such as a tumor or cancer. In some embodiments, the engineered cells or compositions assessed or evaluated using the embodiments provided herein are administered in an effective amount to effect treatment of the disease or disorder. Uses include uses of the engineered cells or compositions in such methods and treatments, and in the preparation of a medicament in order to carry out such therapeutic methods. In some embodiments, the methods, e.g., therapeutic methods, are carried out by administering the assessed or evaluated engineered cells, or compositions comprising the same, to the subject having or suspected of having the disease or condition. In some embodiments, these methods thereby treat the disease or condition or disorder in the subject.

In some aspects, the engineered cells or engineered cell composition can be administered to a subject, such as a subject that has a disease or disorder. Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g., causes, exacerbates or otherwise is involved in such disease, condition, or disorder. Exemplary diseases and conditions can include diseases or conditions associated with malignancy or transformation of cells (e.g., cancer), autoimmune or inflammatory disease, or an infectious disease, e.g., caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen _{1B} (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, 0-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen Al (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT) vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen, such as a viral antigen (e.g., a viral antigen from HIV, HCV, HBV), bacterial antigens, and/or parasitic antigens.

In some embodiments, the antibody or an antigen-binding fragment (e.g., scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the disease or condition is a B cell malignancy. In some embodiments, the B cell malignancy is a leukemia or a lymphoma. In some aspects, the disease or condition is acute lymphoblastic leukemia (ALL), adult ALL, chronic lymphoblastic leukemia (CLL), non-Hodgkin lymphoma (NHL), or Diffuse Large B-Cell Lymphoma (DLBCL). In some cases, the disease or condition is an NHL, such as or including an NHL that is an aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B). In some aspects, the recombinant receptor, such as a CAR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30, or combinations thereof.

In some embodiments, the disease or condition is a myeloma, such as a multiple myeloma. In some aspects, the recombinant receptor, such as a CAR, specifically binds to an antigen associated with the disease or condition or expressed in cells of the environment of a lesion associated with the multiple myeloma. Antigens targeted by the receptors in some embodiments include antigens associated with multiple myeloma. In some aspects, the antigen, e.g., the second or additional antigen, such as the disease-specific antigen and/or related antigen, is expressed on multiple myeloma, such as B cell maturation antigen (BCMA), G protein-coupled receptor class C group 5 member D (GPRC5D), CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, β2-Microglobulin, HM1.24, IGF-1R, IL-6R, TRAIL-R1, and the activin receptor type IIA (ActRIIA). See Benson and Byrd, J. Clin. Oncol. (2012) 30(16): 2013-15; Tao and Anderson, Bone Marrow Research (2011):924058; Chu et al., Leukemia (2013) 28(4):917-27; Garfall et al., Discov Med. (2014) 17(91):37-46. In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603477, 8,008,450; U.S. Pub. No. US20120189622 or US20100260748; and/or International PCT Publication Nos. WO2006099875, WO2009080829 or WO2012092612 or WO2014210064. In some embodiments, such antibodies or antigen-binding fragments thereof (e.g., scFv) are contained in multispecific antibodies, multispecific chimeric receptors, such as multispecific CARs, and/or multispecific cells.

In some embodiments, the disease or disorder is associated with expression of G protein-coupled receptor class C group 5 member D (GPRCSD) and/or expression of B cell maturation antigen (BCMA).

In some embodiments, the disease or disorder is a B cell-related disorder. In some of any of the provided embodiments of the provided methods, the disease or disorder associated with BCMA is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

In some embodiments, the disease or disorder is a cancer. In some embodiments, the cancer is a GPRC5D-expressing cancer. In some embodiments, the cancer is a plasma cell malignancy and the plasma cell malignancy is multiple myeloma (MM) or plasmacytoma. In some embodiments, the cancer is multiple myeloma (MM). In some embodiments, the cancer is a relapsed/refractory multiple myeloma.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the subject is administered a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the subject is administered a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known. See, for instance, Wadwa et al., J. Drug Targeting 3: 1 1 1 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

A. Dosing

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g., antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g., antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments.

In some embodiments, the dose of cells is a flat dose of cells or fixed dose of cells such that the dose of cells is not tied to or based on the body surface area or weight of a subject. In some embodiments, such values refer to numbers of recombinant receptor-expressing cells; in other embodiments, they refer to number of T cells or PBMCs or total cells administered.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸, at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total recombinant receptor (e.g., CAR)-expressing cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁸, 8×10⁸, or 1.2×10⁹ total such cells, or the range between any two of the foregoing values.

In some embodiments, the dose of genetically engineered cells comprises from at or about 1×10⁵ to at or about 5×10⁸ total CAR-expressing (CAR+) T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells. In some embodiments, the dose of genetically engineered cells comprises from or from about 2.5×10⁷ to at or about 1.5×10⁸ total CAR-expressing T cells, such as from or from about 5×10⁷ to or to about 1×10⁸ total CAR-expressing T cells.

In some embodiments, the dose of genetically engineered cells comprises at least at or about 1×10⁵ CAR-expressing cells, at least at or about 2.5×10⁵ CAR-expressing cells, at least at or about 5×10⁵ CAR-expressing cells, at least at or about 1×10⁶ CAR-expressing cells, at least at or about 2.5×10⁶ CAR-expressing cells, at least at or about 5×10⁶ CAR-expressing cells, at least at or about 1×10⁷ CAR-expressing cells, at least at or about 2.5×10⁷ CAR-expressing cells, at least at or about 5×10⁷ CAR-expressing cells, at least at or about 1×10⁸ CAR-expressing cells, at least at or about 1.5×10⁸ CAR-expressing cells, at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to or to about 5×10⁸ total recombinant receptor-expressing cells, total T cells, or total peripheral blood mononuclear cells (PBMCs), from or from about 5×10⁵ to or to about 1×10⁷ total recombinant receptor-expressing cells, total T cells, or total peripheral blood mononuclear cells (PBMCs) or from or from about 1×10⁶ to or to about 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3-expressing or CD8-expressing, in some cases also recombinant receptor-expressing (e.g., CAR-expressing) cells. In some embodiments, the cell therapy comprises administration of a dose comprising a number of cell from or from about 1×10⁵ to or to about 5×10⁸ CD3-expressing or CD8-expressing total T cells or CD3-expressing or CD8-expressing recombinant receptor-expressing cells, from or from about 5×10⁵ to or to about 1×10⁷ CD3-expressing or CD8-expressing total T cells or CD3-expressing or CD8-expressing recombinant receptor-expressing cells, or from or from about 1×10⁶ to or to about 1×10⁷ CD3-expressing or CD8-expressing total T cells or CD3-expressing or CD8-expressing recombinant receptor-expressing cells, each inclusive. In some embodiments, the cell therapy comprises administration of a dose comprising a number of cell from or from about 1×10⁵ to or to about 5×10⁸ total CD3-expressing/CAR-expressing or CD8-expressing/CAR-expressing cells, from or from about 5×10⁵ to or to about 1×10⁷ total CD3-expressing/CAR-expressing or CD8-expressing/CAR-expressing cells, or from or from about 1×10⁶ to or to about 1×10⁷ total CD3-expressing/CAR-expressing or CD8-expressing/CAR-expressing cells, each inclusive.

In some embodiments, the T cells of the dose include CD4+ T cells, CD8+ T cells or CD4+ and CD8+ T cells.

In some embodiments, for example, where the subject is human, the CD8⁺ T cells of the dose, including in a dose including CD4⁺ and CD8⁺ T cells, includes between at or about 1×10⁶ and at or about 5×10⁸ total recombinant receptor (e.g., CAR)-expressing CD8⁺ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8⁺ T cells, from or from about 1×10⁷ to or to about 5×10⁷ total recombinant receptor-expressing CD8⁺ T cells, from or from about 1×10⁷ to or to about 0.25×10⁸ total recombinant receptor-expressing CD8⁺ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD8⁺ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8⁺- and CD4⁺-enriched populations, respectively, e.g., CD4⁺ and/or CD8⁺ T cells each individually including cells genetically engineered to express the recombinant receptor. In some embodiments, the administration of the dose comprises administration of a first composition comprising a dose of CD8⁺ T cells or a dose of CD4⁺ T cells and administration of a second composition comprising the other of the dose of CD4⁺ T cells and the CD8⁺ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some composition, the first composition, e.g., first composition of the dose, comprises CD4⁺ T cells. In some composition, the first composition, e.g., first composition of the dose, comprises CD8⁺ T cells. In some embodiments, the first composition is administered prior to the second composition.

In some embodiments, the dose or composition of cells includes a defined or target ratio of CD4⁺ cells expressing a recombinant receptor to CD8⁺ cells expressing a recombinant receptor and/or of CD4⁺ cells to CD8⁺ cells, which ratio optionally is approximately 1:1 or is between approximately 1:3 and approximately 3:1, such as approximately 1:1. In some aspects, the administration of a composition or dose with the target or desired ratio of different cell populations (such as CD4⁺:CD8⁺ ratio or CAR⁺CD4⁺:CAR⁺CD8⁺ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g., consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered. In some embodiments, administration of one or more additional doses is determined based on the clinical responses determined according the methods provided herein.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### VI. EXEMPLARY EMBODIMENTS

Among the provided embodiments are:

1. A method of identifying features associated with a clinical response, the method comprising:

(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a CAR; and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;

(b) preprocessing the features to identify informative features, the informative features comprising a subset of the features comprising one or more of the subject features, one or more the input composition features, and one or more of the therapeutic cell composition features;

(c) obtaining clinical responses from each of the plurality of subjects following treatment with one of the plurality of therapeutic compositions;

(d) applying the informative features and the obtained clinical responses from the plurality of subjects as input to train a random forests model using supervised learning; and

(e) identifying from the trained random forests model the informative features associated with the clinical responses.

2. A method of identifying features associated with a clinical response, the method comprising:

(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;

(b) preprocessing the features to identify informative features, the informative features comprising a subset of the features comprising one or more of the subject features, one or more of the input composition features, and one or more of the therapeutic cell composition features;

(c) obtaining clinical responses over time from each of the plurality of subjects following treatment with one of the plurality of therapeutic compositions;

(d) applying the informative features and clinical responses from the plurality of subjects as input to train a random survival forests model using supervised learning; and

(e) identifying from the trained random survival forests model the informative features associated with the clinical responses.

3. The method of embodiment 1 or embodiment 2, wherein identifying the informative features associated with the clinical responses comprises determining an importance measure for each of the informative features.

4. The method of embodiment 3, wherein the importance measure comprises a permutation importance measure, a mean minimal depth, and/or a total number of trees from the random forests wherein the informative feature splits a root node.

5. The method of embodiment 3, wherein the importance measure comprises a permutation importance measure, a mean minimal depth, and/or a total number of trees from the random survival forests wherein the informative feature splits a root node.

6. The method of any of embodiments 3-5, wherein the importance measure is the permutation importance measure.

7. The method of any of embodiments 3-5, wherein the importance measure is the mean minimal depth.

8. The method of embodiment 3 or embodiment 4, wherein the importance measure is the total number of trees from the random forests wherein the informative feature splits a root node.

9. The method of embodiment 3 or embodiment 5, wherein the importance measure is the total number of trees from the random survival forests wherein the informative feature splits a root node.

10. The method of any of embodiments 3-10, wherein the informative features associated with the clinical responses are the first 10, 9, 8, 7, 6, 5, 4, 3, 2, or 1 informative features identified by rank ordering of the importance measure for each of the informative features, wherein the importance measure is the same for each informative feature.

11. The method of any of embodiments 3-10, wherein the informative features associated with the clinical responses are the first 5 informative features identified by rank ordering values of the importance measure for each of the informative features, wherein the importance measure is the same for each informative feature.

12. The method of any of embodiments 3-11, wherein the informative features associated with the clinical responses is the first informative feature identified by rank ordering values of the importance measure for each of the informative features, wherein the importance measure is the same for each informative feature.

13. A method of determining a clinical response, the method comprising:

(a) receiving features comprising:
(i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from an input composition, wherein the input composition comprises T cells selected from a sample from the subject, wherein the T cells are used for producing the therapeutic cell composition comprising T cells comprising a chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject; and

(b) applying the features as input to a random forests model trained to determine, based on informative features identified by preprocessing, clinical responses of the subject to treatment with the therapeutic cell composition prior to treating the subject with the therapeutic cell composition, wherein the features applied as input are the same informative features as those used to train the random forests model.

14. A method of determining a clinical response, the method comprising:

(a) receiving features comprising:
(i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from an input composition, wherein the input composition comprises T cells selected from a sample from the subject, wherein the T cells are used for producing the therapeutic cell composition comprising T cells comprising a chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject; and

(b) applying the features as input to a random survival forests model trained to determine, based on informative features identified by preprocessing, clinical responses of the subject to treatment with the therapeutic cell composition prior to treating the subject with the therapeutic cell composition, wherein the features applied as input are the same informative features as those used to train the random survival forests model.

15. A method of treating a subject, the method comprising:

(a) selecting T cells from a sample from a subject to produce an input composition comprising T cells;

(b) determining features comprising:
(i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from an input composition, wherein the input composition comprises T cells selected from a sample from the subject, wherein the T cells are used for producing the therapeutic cell composition comprising T cells comprising a chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject; and

(c) applying the features as input to a random forests model trained to determine, based on informative features identified by preprocessing, clinical responses of the subject to treatment with the therapeutic cell composition prior to treating the subject with the therapeutic cell composition, wherein the features applied as input are the same informative features as those used to train the random forests model; and

administering a treatment to the subject wherein:
(1) if the subject is determined to have a clinical response selected from the group consisting of a complete response (CR), a partial response (PR), a durable response of greater than 3 months, progression free survival (PFS) for more than 3 months, objective response (OR), a desired pharmacokinetic response that is or is greater than a target pharmacokinetic response, and no or a mild toxicity response (optionally wherein the toxicity is grade 2 or less CRS or grade 2 or less neurotoxicity), a predetermined treatment regimen comprising the therapeutic cell composition is administered; or
(2) if the subject is determined to have a clinical response selected from the group consisting of a toxicity response (optionally wherein the toxicity response is a severe cytokine release syndrome or severe neurotoxicity), a reduced pharmacokinetics response compared to a target pharmacokinetic response, progressive disease (PD), a durable response of less than 3 months, and PFS of less than 3 months a, administering to the subject a treatment regimen comprising the therapeutic cell composition that is altered compared to the predetermined treatment regimen comprising the therapeutic cell composition.

16. A method of treating a subject, the method comprising:

(a) selecting T cells from a sample from a subject to produce an input composition comprising T cells;

(b) determining features comprising:
(i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from an input composition, wherein the input composition comprises T cells selected from a sample from the subject, wherein the T cells are used for producing the therapeutic cell composition comprising T cells comprising a chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject; and

(c) applying the features as input to a random survival forests model trained to determine, based on informative features identified by preprocessing, clinical responses in the subject to be treated with the therapeutic cell composition prior to treating the subject with the therapeutic cell composition, wherein the features applied as input are the same informative features as those used to train the random survival forests model; and

administering a treatment to the subject wherein:
(1) if the subject is determined to have a clinical response selected from the group consisting of a complete response (CR), a partial response (PR), a durable response of greater than 3 months, progression free survival (PFS) for more than 3 months, objective response (OR), a desired pharmacokinetic response that is or is greater than a target pharmacokinetic response, and no or a mild toxicity response (optionally wherein the toxicity is grade 2 or less CRS or grade 2 or less neurotoxicity), a predetermined treatment regimen comprising the therapeutic cell composition is administered; or
(2) if the subject is determined to have a clinical response selected from the group consisting of a toxicity response (optionally wherein the toxicity response is a severe cytokine release syndrome or severe neurotoxicity), a reduced pharmacokinetics response compared to a target pharmacokinetic response, progressive disease (PD), a durable response of less than 3 months, and PFS of less than 3 months a, administering to the subject a treatment regimen comprising the therapeutic cell composition that is altered compared to the predetermined treatment regimen comprising the therapeutic cell composition.

17. The method of embodiment 13 or embodiment 15, wherein the random forests model is trained using supervised training, the supervised training comprising:

(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;

(b) preprocessing the features to identify informative features, the informative features comprising a subset of the features comprising one or more subject features, one or more input composition features, and one or more therapeutic cell composition features;

(c) obtaining clinical responses from each of the plurality of subjects following treatment with one of the plurality of therapeutic compositions;

(d) applying the informative features from a plurality of subjects and the obtained clinical responses as input to train a random forests model.

18. The method of embodiment 14 or embodiment 16, wherein the random survival forests model is trained using supervised training, the supervised training comprising:

(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;

(b) preprocessing the features to identify informative features, the informative features comprising a subset of the features comprising one or more subject features, one or more input composition features, and one or more therapeutic cell composition features;

(c) obtaining clinical responses over time from each of the plurality of subjects following treatment with one of the plurality of therapeutic compositions;

(d) applying the informative features and clinical responses from the plurality of subjects as input to train a random survival forests model using supervised learning.

19. A method of developing a random forests model comprising:

(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;

(b) preprocessing the features to identify informative features, the informative features comprising a subset of the features comprising one or more subject features, one or more input composition features, and one or more therapeutic cell composition features;

(c) obtaining clinical responses from each of the plurality of subjects following treatment with one of the plurality of therapeutic compositions;

(d) applying the informative features from a plurality of subjects and the obtained clinical responses as input to train a random forests model.

20. A method of developing a random survival forests model comprising:

(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;

(b) preprocessing the features to identify informative features, the informative features comprising a subset of the features comprising one or more subject features, one or more input composition features, and one or more therapeutic cell composition features;

(c) obtaining clinical responses over time from each of the plurality of subjects following treatment with one of the plurality of therapeutic compositions;

(d) applying the informative features and clinical responses from the plurality of subjects as input to train a random survival forests model using supervised learning.

21. The method of any of embodiments 1-12 and 17-20, wherein each of the plurality of subjects is administered one of the plurality of therapeutic cell compositions, wherein the one therapeutic cell composition administered to the subject is the therapeutic cell composition produced from the input composition of the sample from the subject.

22. The method of any of embodiments 1-21, wherein the preprocessing to identify informative features comprises one or more of:

a) removing subject features, input composition features, and therapeutic cell composition features having greater than, than about, or 50% of the data missing;

b) removing subject features, input composition features, and therapeutic cell composition features having zero variance or greater than, than about or 95% of data values equal to a single value and/or fewer than 0.1n unique values, wherein n=number of samples;

c) imputing missing data for subject features, input composition features, and therapeutic cell composition features by multivariate imputation by chained equations;

d) identifying covariate clusters, the covariate clusters comprising sets of subject features, input composition features, and therapeutic cell composition features and combinations thereof, with correlation coefficients of greater than, about, or equal to 0.5, and iteratively selecting subject features, input composition features, and therapeutic cell composition features from the covariate cluster, wherein the selected subject features, input composition features, and therapeutic cell composition features have the lowest mean absolute correlation with all remaining subject features, input composition features, and therapeutic cell composition features.

23. The method of any of embodiments 1-22, wherein the preprocessing to identify informative features comprises or is removing subject features, input composition features, and therapeutic cell composition features having greater than, about, or 50% of the data is missing.

24. The method of any of embodiments 1-23, wherein the preprocessing to identify informative features comprises or is removing subject features, input composition features, and therapeutic cell composition features having zero variance or greater than, about or 95% of data values equal to a single value and fewer than 0.1n unique values, wherein n=number of samples.

25. The method of any of embodiments 1-24, wherein the preprocessing to identify informative features comprises or is imputing missing data for subject features, input composition features, and therapeutic cell composition features by multivariate imputation by chained equations.

26. The method of any of embodiments 1-25, wherein the preprocessing to identify informative features comprises or is identifying covariate clusters, the covariate clusters comprising sets of subject features, input composition features, and therapeutic cell composition features and combinations thereof with correlation coefficients of greater than, about, or equal to 0.5, and iteratively selecting subject features, input composition features, and therapeutic cell composition features from the covariate cluster, wherein the selected subject features, input composition features, and therapeutic cell composition features have the lowest mean absolute correlation with all remaining subject features, input composition features, and therapeutic cell composition features.

27. The method of embodiment any of embodiments 1, 3, 4, 6-8, 10-13, 15, 17, 19 and 21-26, wherein the random forests model is evaluated using cross validation.

28. The method of embodiment any of embodiments 2, 3, 5-7, 9-12, 14, 16, 18, and 20-26, wherein the random survival forests model is evaluated using cross validation.

29. The method of embodiment 27 or embodiment 28, wherein the cross validation is or is at least 10-fold cross validation.

30. The method of any of embodiments 27 or 28, wherein the cross validation is nested cross validation.

31. The method of any of embodiments 1-12 and 17-30, wherein the plurality of subjects is or is about 500, 400, 300, 200, 150, 100, 50, 25, 15, or 10 subjects, or is any number between any of the foregoing.

32. The method of any of embodiments 1-12 and 17-31, wherein the plurality of subjects is, is about, or is greater than 10 subjects and less than 250 subjects.

33. The method of any of embodiments 1-12 and 17-33, wherein the plurality of subjects is, is about, or is greater than 20 subject and less than 200 subjects.

34. The method of any of embodiments 1-12 and 17-33, wherein the plurality of subjects is, is about, or is greater than 20 and less than 150 subjects.

35. The method of any of embodiments 1-12 and 17-34, wherein the plurality of subjects is, is about, or is greater than 20 subjects and less than 150 subjects.

36. The method of any of embodiments 1-12 and 17-35, wherein the plurality of subjects is, is about, or is greater than 20 subjects less than 100 subjects.

37. The method of any of embodiments 1-12 and 17-36, wherein the plurality of subjects are participating in a clinical trial.

38. The method of any of embodiments 1-37, wherein the subject features comprise one or more of subject attributes and clinical attributes.

39. The method of embodiment 38, wherein the subject attributes comprise one or more of age, weight, height, ethnicity, race, sex, and body mass index.

40. The method of embodiment 38 or embodiment 39, wherein the clinical attributes comprise one or more of biomarkers, disease diagnosis, disease burden, disease duration, disease grade, and treatment history.

41. The method of any of embodiments 1-40, wherein the input composition features comprise cell phenotypes.

42. The method of any of embodiments 1-41, wherein the therapeutic cell composition features comprise one or more of a cell phenotype, a recombinant receptor-dependent activity, and a dose.

43. The method of any of embodiments 1-42, wherein the clinical responses comprise one or more of a complete response (CR), a partial response (PR), a durable response, progression free survival (PFS), objective response (OR)a pharmacokinetic response that is or is greater than a target pharmacokinetic response, no or a mild toxicity response, a toxicity response, a reduced pharmacokinetics response compared to a target response, or a lack of CR, PR, durable response, OR, or PFS.

44. The method of any of embodiments 1-43, wherein the sample comprises a whole blood sample, a buffy coat sample, a peripheral blood mononuclear cell (PBMC) sample, an unfractionated T cell sample, a lymphocyte sample, a white blood cell sample, an apheresis product, or a leukapheresis product.

45. The method of any of embodiments 1-44, wherein the sample is an apheresis product or leukapheresis product.

46. The method of embodiment 45, wherein the apheresis product or leukapheresis product has been previously cryopreserved.

47. The method of any of embodiments 1-46, wherein the T cells comprise primary cells obtained from the subject.

48. The method of any of embodiments 1-47, wherein the T cells comprise CD3+, CD4+, and/or CD8+.

49. The method of any of embodiments 1-48, wherein the input composition comprises CD4+, CD8+, or CD4+ and CD8+ T cells and the therapeutic cell composition comprises CD4+, CD8+, or CD4+ and CD8+ T cells expressing a recombinant receptor and is produced from the input composition, wherein the input composition features comprise input composition features from the CD4+, CD8+, or CD4+ and CD8+ T cell compositions of the input composition, and the therapeutic cell composition features comprise therapeutic cell composition features from the CD4+, CD8+, or CD4+ and CD8+ T cells of the therapeutic composition.

50. The method of any of embodiments 1-48, wherein the input composition comprises separate compositions of CD4+ and CD8+ T cells and the therapeutic cell composition comprises separate compositions of CD4+ and CD8+ T cells expressing a recombinant receptor, and is produced from the respective CD4+ or CD8+ T cell composition of the input composition, wherein the input composition features comprise input composition features from the CD4+ and CD8+ T cell compositions of the input composition, and the therapeutic cell composition features comprise therapeutic cell composition features from the CD4+ and CD8+ T cells of each of the separate compositions of the therapeutic composition.

51. The method of any of embodiments 1-48, wherein the input composition comprises separate compositions of CD4+ and CD8+ T cells and the therapeutic cell composition comprises a mixed composition of CD4+ and CD8+ T cells expressing a recombinant receptor, and is produced from the separate CD4+ and CD8+ T cell compositions of the input composition, wherein the input composition features comprise input composition features from the separate CD4+ and CD8+ T cell compositions of the input composition, and the therapeutic cell composition features comprise therapeutic cell composition features from the mixed composition of CD4+ and CD8+ cells of the therapeutic composition.

52. The method of any of embodiments 1-51, wherein the recombinant receptor is a chimeric antigen receptor (CAR).

53. The method of any of embodiments 15-18 and 21-52, wherein the predetermined treatment regimen comprises or is a single treatment comprising administering:

a) 25×10⁶ CD8+CAR+ T cells and 25×10⁶ CD4+CAR+ T cells separately to the subject;

b) 50×10⁶ CD8+CAR+ T cells and 50×10⁶ CD4+CAR+ T cells separately to the subject; or

c) 75×10⁶ CD8+CAR+ T cells and 75×10⁶ CD4+CAR+ T cells separately to the subject.

54. The method of any of embodiments 15-18 and 21-52, wherein altering the predetermined treatment regimen comprises or is a single treatment comprising administering:

50×10⁶ CD8+CAR+ T cells and 50×10⁶ CD4+CAR+ T cells separately to the subject when the predetermined treatment regimen comprises or is a single treatment comprising administering 25×10⁶ CD8+CAR+ T cells and 25×10⁶ CD4+CAR+ T cells separately to the subject;

75×10⁶ CD8+CAR+ T cells and 75×10⁶ CD4+CAR+ T cells separately to the subject when the predetermined treatment regimen comprises or is a single treatment comprising administering 50×10⁶ CD8+CAR+ T cells and 50×10⁶ CD4+CAR+ T cells separately to the subject; or

75×10⁶ CD8+CAR+ T cells and 75×10⁶ CD4+CAR+ T cells separately to the subject when the predetermined treatment regimen comprises or is a single treatment comprising administering 25×10⁶ CD8+CAR+ T cells and 25×10⁶ CD4+CAR+ T cells separately to the subject.

55. The method of any of embodiments 15-18 and 21-52, wherein altering the predetermined treatment regimen comprises or is a single treatment comprising administering:

25×10⁶ CD8+CAR+ T cells and 25×10⁶ CD4+CAR+ T cells separately to the subject when the predetermined treatment regimen comprises or is a single treatment comprising administering 50×10⁶ CD8+CAR+ T cells and 50×10⁶ CD4+CAR+ T cells separately to the subject;

50×10⁶ CD8+CAR+ T cells and 50×10⁶ CD4+CAR+ T cells separately to the subject when the predetermined treatment regimen comprises or is a single treatment comprising administering 75×10⁶ CD8+CAR+ T cells and 75×10⁶ CD4+CAR+ T cells separately to the subject; or

25×10⁶ CD8+CAR+ T cells and 25×10⁶ CD4+CAR+ T cells separately to the subject when the predetermined treatment regimen comprises or is a single treatment comprising administering 75×10⁶ CD8+CAR+ T cells and 75×10⁶ CD4+CAR+ T cells separately to the subject.

56. The method of any of embodiments 15-18 and 21-52, wherein altering the predetermined treatment regimen comprises administering the therapeutic cell composition in combination with a second therapeutic agent.

### VII. EXAMPLES

The following examples are included for illustrative purposes only and are not intended to limit the scope of the invention.

### Example 1: Random Forest and Random Survival Forests for Assessing Clinical Response to Treatment with Therapeutic Cell Compositions

Random forests and random survival forests were trained to predict the clinical response of subjects with relapsed or refractory Large B-Cell Lymphoma to treatment with therapeutic cell compositions containing T cells engineered with a chimeric antigen receptor (CAR). The models were used to identify features, including subject attributes, therapeutic cell composition attributes, and attributes of starting material used to produce the therapeutic cell compositions, associated with clinical responses.

A. Subjects and Treatment

Subjects (n=172) with relapsed or refractory Large B-Cell Lymphoma (LBCL) were administered therapeutic cell compositions of engineered CD4+ T cells and engineered CD8+ T cells, each expressing the same anti-CD19 chimeric antigen receptor (CAR).

To produce the therapeutic cell compositions, CD4+ and CD8+ cells were separately selected by immunoaffinity-based selection from human peripheral blood mononuclear cells (PBMCs) that had been obtained by leukapheresis, generating separate enriched CD4+ and enriched CD8+ cell compositions (e.g., input compositions), which then were cryopreserved. The CD4+ and CD8+ compositions were subsequently thawed and separately underwent steps for stimulation, transduction, and expansion.

The thawed CD4+ and CD8+ cells were separately stimulated in the presence of paramagnetic polystyrene-coated beads coupled to anti-CD3 and anti-CD28 antibodies at a 1:1 bead to cell ratio. The stimulation was carried out in media containing human recombinant IL-2, human recombinant IL-15, and N-Acetyl Cysteine (NAC). The CD4+ cell media also included human recombinant IL-7.

Following the introduction of the beads, CD4+ and CD8+ cells were separately transduced with a lentiviral vector encoding the same anti-CD19 CAR. The CAR contained an anti-CD19 scFv derived from a murine antibody, an immunoglobulin spacer, a transmembrane domain derived from CD28, a costimulatory region derived from 4-1BB, and a CD3-zeta intracellular signaling domain The vector also encoded a truncated EGFR (EGFRt) that served as a surrogate marker for CAR expression that was connected to the CAR construct by a T2A sequence. The cells were transduced in the presence of 10 μg/ml protamine sulfate.

After transduction, the beads were removed from the cell compositions by exposure to a magnetic field. The CD4+ and CD8+ cell compositions were then separately cultivated for expansion with continual mixing and oxygen transfer by a bioreactor (Xuri W25 Bioreactor). Poloxamer was added to the media. Both cell compositions were cultivated in the presence of IL-2 and IL-15. The CD4+ cell media also included IL-7. The CD4+ and CD8+ cells were each cultivated, prior to harvest, to 4-fold expansion. One day after reaching the threshold, cells from each composition were separately harvested, formulated, and cryopreserved. The exemplary process is summarized in Table E1.

TABLE E1
Summary of the exemplary process for generating
CD4+ and CD8+ CAR-T cells

| Stage | CD4+ cells | CD8+ cells |
|---|---|---|
| Stimulation | anti-CD3/CD28 antibody | anti-CD3/CD28 antibody |
| (day 1-2) | conjugated beads | conjugated beads |
| | 1:1 bead to cell ratio | 1:1 bead to cell ratio |
| | media: IL-2, IL-7, IL-15, | media: IL-2, IL-15, and |
| | and NAC | NAC |
| Transduction | transduction adjuvant (e.g., | transduction adjuvant (e.g., |
| (day 2-5) | 10 μg/ml protamine sulfate) | 10 μg/ml protamine sulfate) |
| Bead removal | magnetic bead removal | magnetic bead removal |
| (day 5*) | | |
| Expansion | rocking motion bioreactor | rocking motion bioreactor |
| (day 5*- | and/or continuous mixing | and/or continuous mixing |
| Harvest) | media: IL-2, IL-7, IL-15, | media: IL-2, IL-15, and |
| | and poloxamer | poloxamer |

*Approximate

Subjects received lymphodepleting chemotherapy prior to treatment with the therapeutic cell compositions. In some cases, subjects were administered a bridging therapy after leukapheresis and before lymphodepleting chemotherapy to control disease.

The cryopreserved therapeutic cell compositions were thawed prior to intravenous administration. The therapeutic cell compositions were administered as a defined amount of formulated CD4⁺ CAR+ cells and formulated CD8⁺ CAR+ cells at a target ratio of approximately 1:1. Subjects were administered a single dose of CAR-expressing T cells (each single dose via separate infusions of CD4⁺ CAR-expressing T cells and CD8⁺ CAR-expressing T cells, respectively) as follows: a single dose of dose level 1 (DL1) containing 50×10⁶ total CAR-expressing T cells (n=5 subjects), a single dose of dose level 2 (DL2) containing 100×10⁶ total CAR-expressing T cells (n=126 subjects), or a single dose of dose level 3 (DL3) containing 150×10⁶ total CAR-expressing T cells (n=41 subjects). The target dose level and the numbers of T cell subsets administered are set forth in Table E2.

TABLE E2
Dose Schedules

| | | Target Dose of CAR+ T cells | | |
|---|---|---|---|---|
| Dose Level | Subjects Treated (n) | Total | CD8+ | CD4+ |
| DL1 | 5 | 50 × 10⁶ | 25 × 10⁶ | 25 × 10⁶ |
| DL2 | 126 | 100 × 10⁶ | 50 × 10⁶ | 50 × 10⁶ |
| DL3 | 41 | 150 × 10⁶ | 75 × 10⁶ | 75 × 10⁶ |

DL1, dose level 1 (single dose); DL2, dose level 2 (single dose); DL3, dose level 3 (single dose).

B. Feature Assessment and Preprocessing

Multivariate analyses of 321 features, including patient attributes, input composition attributes, and therapeutic cell composition attributes, were assessed in the 172 individuals treated with the therapeutic cell compositions as described above.

The 321 features underwent preprocessing to identify features for supervised training of the random forests and random survival forests. Features with near zero variance, features with greater than 70% of values missing, and highly correlated features (|ρ|≥0.7) were removed from the data set. For features with less than 70% of values missing, missing values were replaced with either the mean or mode.

The training data included the preprocessed features (e.g., predictors) and 20 responses (e.g., dependent variables). The responses included progression-free survival (PFS); overall response rate (ORR); objective response (OR); complete response (CR); safety: neurological events (NEs) grade ≥1 and grade ≥3 and cytokine release syndrome (CRS) grade ≥1 and grade ≥3; and pharmacokinetics endpoints: log₁₀ area under the curve (AUC), log₁₀ maximum concentration (Cₘₐₓ), and time to peak concentration (Tₘₐₓ).

C. Multivariate Supervised Learning

Random forests and random survival forests were trained and tested using ten-fold cross-validation. Feature importance and associated permutation-based p-values were used to assess the significance of the effects of features. Wilcoxon rank-sum test, Spearman correlation, and Cox proportional hazards model were used to support univariate analyses.

Multiple features including subject attributes and therapeutic cell composition attributes were found to be correlated with the clinical responses examined (e.g., dependent variables). Patient tumor burden and systemic inflammation were associated with CAR T cell expansion, efficacy, and safety. In general, higher tumor burden was associated with higher in vivo CAR T cell expansion but lower efficacy and higher risk for adverse events. Higher systemic inflammation at baseline was associated with higher risk for adverse events. In the drug product, increased antigen-specific cytokine levels were positively associated with expansion and efficacy, and higher frequencies of less differentiated central memory CAR T cells were associated with higher expansion and efficacy. Table E3 summarizes a subset of feature associations.

TABLE E3
Exemplary features associated with exemplary clinical response

| | Association Based on Random | P |
|---|---|---|
| Feature | (Survival) Forests | Value¹ |
| Subject features | | |
| Higher tumor burden (as | Lower likelihood of | 0.001 |
| measured by SPD pre- | achieving CR | |
| lymphodepletion) | Higher risk for CRS grade ≥ 1 | 0.012 |
| | Higher risk for NE grade ≥ 1 | 0.001 |
| | Higher CAR T cell expansion | 0.019 |
| | (log₁₀AUC of CD4+) | |
| High-grade lymphoma | Reduced PFS | 0.048 |
| and DLBCL, NOS as | | |
| compared to primary | | |
| mediastinal B-cell | | |
| lymphoma and | | |
| DLBCL transformed | | |
| from follicular | | |
| lymphoma | | |
| Increased cell turnover | Increased likelihood of CRS | 0.001 |
| (as indicated by | grade ≥ 1 | |
| elevated LDH levels) | Increased likelihood of NE | 0.001 |
| | grade ≥ 1 | |
| | Reduced efficacy (CR) | 0.001 |
| | Reduced efficacy (PFS) | 0.007 |
| Higher inflammation at | Increased risk of NE grade ≥ 3 | 0.001 |
| baseline (as indicated by | Increased risk of NE grade ≥ 1 | 0.001 |
| increased C-reactive | Increased risk of CRS | 0.001 |
| protein levels) | grade ≥ 1 | |
| Subject features | | |
| Older age | Reduced CAR T cell expansion | 0.021 |
| | (log₁₀Cₘₐₓ of CD8+) | |
| Patients with higher total | Elevated CAR T cell expansion | 0.029 |
| protein levels at screening | (log₁₀AUC) | |
| Patients requiring | Higher risk of CRS grade ≥ 1 | 0.004 |
| bridging tx before CAR | Higher risk of NE grade ≥ 3 | 0.001 |
| T cell infusion | Lower CR | 0.001 |
| Input composition | | |
| and therapeutic cell | | |
| composition features | | |
| More differentiated T cell | Reduced CAR T cell expansion | 0.035 |
| starting material | (log₁₀Cₘₐₓ of CD8+) | |
| Increased antigen-specific | Increased efficacy (OR) | 0.001 |
| cytokine production by | Increased efficacy (CR) | 0.006 |
| CD8+ CAR+ T cells | | |
| | Increased efficacy (OR, PFS) | <0.011 |
| Improved cell health in | Increased expansion | <0.023 |
| CAR T cell product | | |
| Increased antigen-specific | Higher CAR T expansion | <0.034 |
| cytokine secretion and | | |
| greater dose of viable | | |
| CAR T cells infused | | |
| Higher frequencies of | Higher CAR T cell expansion | 0.048 |
| central memory | (log₁₀Cₘₐₓ of CD4+) | |
| CAR T cells | Higher CAR T cell expansion | 0.031 |
| | (log₁₀AUC of CD8+) | |

¹Permutation-based p-values for variable importance in random (survival) forests (Altmann et al. 2010).
DLBCL, diffuse large B cell lymphoma; LDH, lactate dehydrogenase; NOS, not otherwise specified.

These data are supportive of the use of random forests and random survival forests to identify features associated with patient clinical outcome in response to treatment with therapeutic cell compositions.

### Example 2: Preprocessing Methods

The 321 features, including patient attributes, input composition attributes, and therapeutic cell composition attributes, assessed in 172 individuals treated with the therapeutic cell compositions as described in Example 1, were preprocessed to identify features for supervised training of random forests and random survival forests. The preprocessing consisted of four (4) sequential steps:
1. Removing features with >50% missing data;
2. Removing features with zero or near-zero variance, where near zero variance is defined by a feature having >95% of the data dominated by a single value and with fewer than 0.1n unique values (n=number of samples);
3. Imputing missing data using multivariate imputation by chained equations (mice); and
4. Identifying covariate clusters and selecting representative features.

Step 4 of preprocessing was performed by first computing a heterogeneous correlation matrix, consisting of Pearson product-moment correlations between numeric features, polyserial correlations between numeric and ordinal features, and polychoric correlations between ordinal features. Correlations were computed between each pair of variables using all complete pairs of observations on those variables. Covariate clusters were defined as sets of variables with correlation coefficients >0.5, e.g., |ρ|>0.5, and representative features were iteratively selected as those in each cluster that exhibited the lowest mean absolute correlation with all other remaining features in the dataset.

Preprocessing identified informative features on which the random forests and random survival forests were trained.

The training data included the identified informative features (e.g., predictors) and 20 clinical responses (e.g., dependent variables). The responses included progression-free survival (PFS); objective response (OR); complete response (CR); safety: neurological events (NEs) grade ≥1 and grade ≥3 and cytokine release syndrome (CRS) grade ≥1 and grade ≥3; and pharmacokinetics endpoints: log₁₀ area under the curve (AUC), log₁₀ maximum concentration (Cₘₐₓ), and time to peak concentration (Tₘₐₓ).

Feature importance and associated permutation-based p-values were used to assess the association of features with clinical responses.

### Example 3: Multivariate Supervised Learning to Identify Features Associated with Clinical Responses in Large B-Cell Lymphoma Therapeutic Cell Compositions

Multivariate supervised learning was performed to identify features that collectively affect clinical outcomes in subjects with relapsed or refractory Large B-Cell Lymphoma treated with therapeutic cell compositions containing T cells engineered with a chimeric antigen receptor (CAR), as described in Example 1 above. Random forests classification, regression, and random survival forests were used to identify features, including subject attributes, therapeutic cell composition attributes, and attributes of starting material (e.g., input composition) used to produce the therapeutic cell compositions, associated with clinical responses.

A. Feature Assessment and Preprocessing

A total of 321 features, including subject attributes, therapeutic cell composition attributes, and attributes of starting material (e.g., input compositions) used to produce the therapeutic cell compositions were assessed in the 172 individuals treated with the therapeutic cell compositions as described in Example 1.

An initial screen of the data removed features with low scientific relevance, for example features specific to manufacturing, and features with low dynamic range (e.g., where the entire distribution was below 5%). The initial screen identified 238 features, including 136 features related to therapeutic cell composition attributes and attributes of starting material used to produce the therapeutic cell compositions and 102 features related to subject attributes. Table E4 shows exemplary features.

TABLE E4
Exemplary features

| | | Additional feature |
|---|---|---|
| Feature Category | Feature | description |
| Input composition | CAS3−/CCR7−/CD27− | CD4 Input Composition |
| Input composition | CAS3−/CCR7−/CD27− | CD8 Input Composition |
| Input composition | CAS3−/CCR7−/CD27+ | CD4 Input Composition |
| Input composition | CAS3−/CCR7−/CD27+ | CD8 Input Composition |
| Input composition | CAS3−/CCR7+ | CD4 Input Composition |
| Input composition | CAS3−/CCR7+ | CD8 Input Composition |
| Input composition | CAS3−/CCR7+/CD27− | CD4 Input Composition |
| Input composition | CAS3−/CCR7+/CD27− | CD8 Input Composition |
| Input composition | CAS3−/CCR7+/CD27+ | CD4 Input Composition |
| Input composition | CAS3−/CCR7+/CD27+ | CD8 Input Composition |
| Input composition | CAS3−/CD27+ | CD4 Input Composition |
| Input composition | CAS3−/CD27+ | CD8 Input Composition |
| Input composition | CAS3−/CD28−/CD27− | CD4 Input Composition |
| Input composition | CAS3−/CD28−/CD27− | CD8 Input Composition |
| Input composition | CAS3−/CD28−/CD27+ | CD4 Input Composition |
| Input composition | CAS3−/CD28−/CD27+ | CD8 Input Composition |
| Input composition | CAS3−/CD28+ | CD4 Input Composition |
| Input composition | CAS3−/CD28+ | CD8 Input Composition |
| Input composition | CAS3−/CD28+/CD27− | CD4 Input Composition |
| Input composition | CAS3−/CD28+/CD27− | CD8 Input Composition |
| Input composition | CAS3−/CD28+/CD27+ | CD4 Input Composition |
| Input composition | CAS3−/CD28+/CD27+ | CD8 Input Composition |
| Input composition | CAS3−/CCR7−/CD45RA− | CD4 Input Composition |
| Input composition | CAS3−/CCR7−/CD45RA− | CD8 Input Composition |
| Input composition | CAS3−/CCR7−/CD45RA+ | CD4 Input Composition |
| Input composition | CAS3−/CCR7−/CD45RA+ | CD8 Input Composition |
| Input composition | CAS3−/CCR7+/CD45RA− | CD4 Input Composition |
| Input composition | CAS3−/CCR7+/CD45RA− | CD8 Input Composition |
| Input composition | CAS3−/CCR7+/CD45RA+ | CD4 Input Composition |
| Input composition | CAS3−/CCR7+/CD45RA+ | CD8 Input Composition |
| Input composition | CAS+ | CD4 Input Composition |
| Input composition | CAS+ | CD8 Input Composition |
| Input composition | CAS+/CD3+ | CD4 Input Composition |
| Input composition | CAS+/CD3+ | CD8 Input Composition |
| Input composition | CAS+/CD4+ | CD4 Input Composition |
| Input composition | CAS+/CD8+ | CD8 Input Composition |
| Input composition | CD4 clonality | CD4 Input Composition |
| Input composition | CD8 clonality | CD8 Input Composition |
| Therapeutic cell | CAS3−/CCR7−/CD27− | CD4 Therapeutic Cell |
| composition | | Composition |
| Therapeutic cell | CAS3−/CCR7−/CD27− | CD8 Therapeutic Cell |
| composition | | Composition |
| Therapeutic cell | CAS3−/CCR7−/CD27+ | CD4 Therapeutic Cell |
| composition | | Composition |
| Therapeutic cell | CAS3−/CCR7−/CD27+ | CD8 Therapeutic Cell |
| composition | | Composition |
| Therapeutic cell | CAS3−/CCR7+ | CD4 Therapeutic Cell |
| composition | | Composition |
| Therapeutic cell | CAS3−/CCR7+ | CD8 Therapeutic Cell |
| composition | | Composition |
| Therapeutic cell | CAS3−/CCR7+/CD27− | CD4 Therapeutic Cell |
| composition | | Composition |
| Therapeutic cell | CAS3−/CCR7+/CD27− | CD8 Therapeutic Cell |
| composition | | Composition |
| Therapeutic cell | CAS3−/CCR7+/CD27+ | CD4 Therapeutic Cell |
| composition | | Composition |
| Therapeutic cell | CAS3−/CCR7+/CD27+ | CD8 Therapeutic Cell |
| composition | | Composition |
| Therapeutic cell | CAS3−/CD27+ | CD4 Therapeutic Cell |
| composition | | Composition |
| Therapeutic cell | CAS3−/CD27+ | CD8 Therapeutic Cell |
| composition | | Composition |
| Therapeutic cell | CAS3−/CD28+ | CD8 Therapeutic Cell |
| composition | | Composition |
| Therapeutic cell | CAS3−/CD28+/CD27− | CD8 Therapeutic Cell |
| composition | | Composition |
| Therapeutic cell | CAS3−/CD28+/CD27+ | CD4 Therapeutic Cell |
| composition | | Composition |
| Therapeutic cell | CAS3−/CD28+/CD27+ | CD8 Therapeutic Cell |
| composition | | Composition |
| Therapeutic cell | CAS3−/CCR7−/CD45RA− | CD4 Therapeutic Cell |
| composition | | Composition |
| Therapeutic cell | CAS3−/CCR7−/CD45RA− | CD8 Therapeutic Cell |
| composition | | Composition |
| Therapeutic cell | CAS3−/CCR7−/CD45RA+ | CD4 Therapeutic Cell |
| composition | | Composition |
| Therapeutic cell | CAS3−/CCR7−/CD45RA+ | CD8 Therapeutic Cell |
| composition | | Composition |
| Therapeutic cell | CAS3−/CCR7+/CD45RA− | CD4 Therapeutic Cell |
| composition | | Composition |
| Therapeutic cell | CAS3−/CCR7+/CD45RA− | CD8 Therapeutic Cell |
| composition | | Composition |
| Therapeutic cell | CAS3−/CCR7+/CD45RA+ | CD4 Therapeutic Cell |
| composition | | Composition |
| Therapeutic cell | CAS3−/CCR7+/CD45RA+ | CD8 Therapeutic Cell |
| composition | | Composition |
| Therapeutic cell | CAS+/CD3+/CAR+ | CD4 Therapeutic Cell |
| composition | | Composition |
| Therapeutic cell | CAS+/CD3+/CAR+ | CD8 Therapeutic Cell |
| composition | | Composition |
| Therapeutic cell | CD3+/CAR+ | CD4 Therapeutic Cell |
| composition | | Composition |
| Therapeutic cell | CD3+/CAR+ | CD8 Therapeutic Cell |
| composition | | Composition |
| Therapeutic cell | CD3+/CD4+ | CD4 Therapeutic Cell |
| composition | | Composition |
| Therapeutic cell | CD3+/CD4+/CAR+ | CD4 Therapeutic Cell |
| composition | | Composition |
| Therapeutic cell | CD3+/CD8+ | CD8 Therapeutic Cell |
| composition | | Composition |
| Therapeutic cell | CD3+/CD8+/CAR+ | CD8 Therapeutic Cell |
| composition | | Composition |
| Therapeutic cell | CD4+/CAR+ | CD4 Therapeutic Cell |
| composition | | Composition |
| Therapeutic cell | CD4 clonality | CD4 Therapeutic Cell |
| composition | | Composition |
| Therapeutic cell | CD4+/EGFRt+ | CD4 Therapeutic Cell |
| composition | | Composition |
| Therapeutic cell | CD8+/CAR+ | CD8 Therapeutic Cell |
| composition | | Composition |
| Therapeutic cell | CD8 clonality | CD8 Therapeutic Cell |
| composition | | Composition |
| Therapeutic cell | CD4+/cytokine− | CD4 Therapeutic Cell |
| composition | | Composition |
| Therapeutic cell | CD8+/cytokine− | CD8 Therapeutic Cell |
| composition | | Composition |
| Therapeutic cell | IFNG+ (alphalisa assay) | CD4 Therapeutic Cell |
| composition | | Composition |
| Therapeutic cell | IFNG+ (alphalisa assay) | CD8 Therapeutic Cell |
| composition | | Composition |
| Therapeutic cell | IFNG+ (elisa) | CD4 Therapeutic Cell |
| composition | | Composition |
| Therapeutic cell | IFNG+ (elisa) | CD8 Therapeutic Cell |
| composition | | Composition |
| Therapeutic cell | IFNg+/IL2 | CD4 Therapeutic Cell |
| composition | | Composition |
| Therapeutic cell | IFNg+/IL2 | CD8 Therapeutic Cell |
| composition | | Composition |
| Therapeutic cell | IFNg+/IL17+/TNFa+ | CD4 Therapeutic Cell |
| composition | | Composition |
| Therapeutic cell | IFNg+/IL2+/IL17+/TNFa+ | CD8 Therapeutic Cell |
| composition | | Composition |
| Therapeutic cell | IFNg+/IL2+/TNFa+ | CD4 Therapeutic Cell |
| composition | | Composition |
| Therapeutic cell | IFNg+/IL2+/TNFa+ | CD8 Therapeutic Cell |
| composition | | Composition |
| Therapeutic cell | CD4+/CAR+/IFNg+ | CD4 Therapeutic Cell |
| composition | | Composition |
| Therapeutic cell | CD8+/CAR+/IFNg+ | CD8 Therapeutic Cell |
| composition | | Composition |
| Therapeutic cell | IFNg+/TNFa+ | CD4 Therapeutic Cell |
| composition | | Composition |
| Therapeutic cell | IFNg+/TNFa+ | CD8 Therapeutic Cell |
| composition | | Composition |
| Therapeutic cell | CD4+/CAR+/IL2+ | CD4 Therapeutic Cell |
| composition | | Composition |
| Therapeutic cell | CD8+/CAR+/IL2+ | CD8 Therapeutic Cell |
| composition | | Composition |
| Therapeutic cell | IL2+/TNFa+ | CD4 Therapeutic Cell |
| composition | | Composition |
| Therapeutic cell | IL2+/TNFa+ | CD8 Therapeutic Cell |
| composition | | Composition |
| Therapeutic cell | Cell lysis | Therapeutic Cell |
| composition | | Composition |
| Therapeutic cell | CD4+/CAR+/TNFa+ | CD4 Therapeutic Cell |
| composition | | Composition |
| Therapeutic cell | CD8+/CAR+/TNFa+ | CD8 Therapeutic Cell |
| composition | | Composition |
| Therapeutic cell | Viable Cell Concentration | CD4 Therapeutic Cell |
| composition | | Composition |
| Therapeutic cell | Viable Cell Concentration | CD8 Therapeutic Cell |
| composition | | Composition |
| Therapeutic cell | Vector Copy Number | CD4 Therapeutic Cell |
| composition | | Composition |
| Therapeutic cell | Vector Copy number | CD8 Therapeutic Cell |
| composition | | Composition |
| Therapeutic cell | EGFRt+ Vector Copy Number | CD4 Therapeutic Cell |
| composition | | Composition |
| Therapeutic cell | EGFRt+ Vector Copy Number | CD8 Therapeutic Cell |
| composition | | Composition |
| Therapeutic cell | Viability | CD4 Therapeutic Cell |
| composition | | Composition |
| Therapeutic cell | Viability | CD8 Therapeutic Cell |
| composition | | Composition |
| Therapeutic cell | GMCSF+/CD19+ | CD4 Therapeutic Cell |
| composition | | Composition |
| Therapeutic cell | GMCSF+/CD19+ | CD8 Therapeutic Cell |
| composition | | Composition |
| Therapeutic cell | IFNG+/CD19+ | CD4 Therapeutic Cell |
| composition | | Composition |
| Therapeutic cell | IFNG+/CD19+ | CD8 Therapeutic Cell |
| composition | | Composition |
| Therapeutic cell | IL10+/CD19+ | CD4 Therapeutic Cell |
| composition | | Composition |
| Therapeutic cell | IL13+/CD19+ | CD4 Therapeutic Cell |
| composition | | Composition |
| Therapeutic cell | IL13+/CD19+ | CD8 Therapeutic Cell |
| composition | | Composition |
| Therapeutic cell | IL2+/CD19+ | CD4 Therapeutic Cell |
| composition | | Composition |
| Therapeutic cell | IL2+/CD19+ | CD8 Therapeutic Cell |
| composition | | Composition |
| Therapeutic cell | IL4+/CD19+ | CD4 Therapeutic Cell |
| composition | | Composition |
| Therapeutic cell | IL4+/CD19+ | CD8 Therapeutic Cell |
| composition | | Composition |
| Therapeutic cell | IL5+/CD19+ | CD4 Therapeutic Cell |
| composition | | Composition |
| Therapeutic cell | IL5+/CD19+ | CD8 Therapeutic Cell |
| composition | | Composition |
| Therapeutic cell | IL6+/CD19+ | CD4 Therapeutic Cell |
| composition | | Composition |
| Therapeutic cell | IL6+/CD19+ | CD8 Therapeutic Cell |
| composition | | Composition |
| Therapeutic cell | MIP1A+/CD19+ | CD4 Therapeutic Cell |
| composition | | Composition |
| Therapeutic cell | MIP1A+/CD19+ | CD8 Therapeutic Cell |
| composition | | Composition |
| Therapeutic cell | MIP1B+/CD19+ | CD4 Therapeutic Cell |
| composition | | Composition |
| Therapeutic cell | MIP1B+/CD19+ | CD8 Therapeutic Cell |
| composition | | Composition |
| Therapeutic cell | SCD137+/CD19+ | CD4 Therapeutic Cell |
| composition | | Composition |
| Therapeutic cell | SCD137+/CD19+ | CD8 Therapeutic Cell |
| composition | | Composition |
| Therapeutic cell | TNFa+/CD19+ | CD4 Therapeutic Cell |
| composition | | Composition |
| Therapeutic cell | TNFa+/CD19+ | CD8 Therapeutic Cell |
| composition | | Composition |
| Therapeutic cell | CD4+ dose | CD4 Therapeutic Cell |
| composition | | Composition |
| Therapeutic cell | CD8+ dose | CD8 Therapeutic Cell |
| composition | | Composition |
| Therapeutic cell | Dose level | |
| composition | | |
| Therapeutic cell | percent viable cells dosed | |
| composition | | |
| Therapeutic cell | total nonviable cells dosed | |
| composition | | |
| Therapeutic cell | total viable cells dosed | |
| composition | | |
| Therapeutic cell | Total dose | |
| composition | | |
| Subject | Dosing arm | |
| Subject | Bridging Chemotherapy | |
| Subject | Bridging Chemotherapy and Radiotherapy | |
| Subject | Bridging Chemotherapy Systemic Treatment | |
| Subject | Cell Origin: | |
| | ABC (activated B-cell-like, or non-GCB) or | |
| | GCB (germinal center B-cell-like) | |
| Subject | relapsed or refractory following chemotherapy | |
| Subject | Type of Diagnosis | |
| Subject | Disease cohort | |
| Subject | Disease burden | |
| Subject | Relapsed or refractory disease | |
| Subject | Disease origin: | |
| | De novo DLBCL or other DLBCL | |
| Subject | Gender | |
| Subject | Therapeutic cell composition administration | |
| | route: infused | |
| Subject | Fold change in LDH | |
| Subject | Height | |
| Subject | Lesion Count | |
| Subject | Oxygen Saturation | |
| Subject | Temperature (° C.) | |
| Subject | Longest tumor diameter pre-treatment with | |
| | therapeutic cell composition | |
| Subject | Fold change in SPD | |
| Subject | SPD value pre-Lymphodepleting chemotherapy, | |
| | groups are <= 50 cm\hat{ }2 or > 50 cm\hat{ }2 | |
| Subject | BMI | BMI (kg/m\hat{ }2) |
| Subject | Weight | Weight (kg) |
| Subject | Sex | |
| Subject | Ethnicity | |
| Subject | Race | |
| Subject | Age | |
| Subject | IPI Score | |
| Subject | ECOG Score | Taken at screen |
| Subject | Disease Stage | |
| Subject | Disease burden based on pre-lymphodepleting | |
| | chemotherapy LDH | |
| Subject | Disease burden based on pre- lymphodepleting | |
| | chemotherapy SPD | |
| Subject | Subject had active CNS disease at time of | |
| | treatment | |
| Subject | Disease burden based on extranodal disease | |
| | classification | |
| Subject | Number of extranodal sites | |
| Subject | Disease burden based on bulky disease | |
| | classification | |
| Subject | Disease Histology | |
| Subject | Number of prior lines of therapy | |
| Subject | Number of prior lines of systemic therapy | |
| Subject | Prior allogenic hematopoietic stem cell | |
| | transplantation (allo-HSCT) | |
| Subject | Prior autologous hematopoietic stem cell | |
| | transplantation (auto-HSCT) | |
| Subject | Chemorefractory or chemosensitive disease type | |
| Subject | Bridging anticancer therapy for disease control | |
| Subject | Days from date of leukapheresis to first infusion | |
| Subject | Months from Diagnosis to treatment with | |
| | therapeutic cell composition | |
| Subject | Comorbidity: creatinine clearance (CrCl) prior | |
| | to lymphodepleting chemotherapy | |
| Subject | Comorbidity: left ventrical ejection fraction | |
| | (LVEF) at screening | |
| Subject | Baseline C Reactive Protein (CRP) | |
| Subject | Pre-leukapheresis Lymphocyte count (10\hat{ }9/L) | |
| Subject | Gene Double Expressor | |
| Subject | Gene Double Hit | |
| Subject | Gene Triple Hit | |
| Subject | Gene Double or Triple Hit | |
| Subject | Gene Double or Triple Hit or Double Expressor | |
| Subject | Albumin level | Taken at screen |
| Subject | Alkaline phosphatase level | Taken at screen |
| Subject | Basophils count | Taken at screen |
| Subject | Absolute Basophil count | Taken at screen |
| Subject | Direct Bilirubin | Taken at screen |
| Subject | Total Bilirubin | Taken at screen |
| Subject | Blood urea nitrogen level | Taken at screen |
| Subject | Calcium level | Taken at screen |
| Subject | Carbon dioxide level | Taken at screen |
| Subject | Chloride level | Taken at screen |
| Subject | Creatinine level | Taken at screen |
| Subject | Eosinophils count | Taken at screen |
| Subject | Eosinophils absolute count | Taken at screen |
| Subject | Glucose level | Taken at screen |
| Subject | Hematocrit level | Taken at screen |
| Subject | Hemoglobin level | Taken at screen |
| Subject | LDH level | Taken at screen |
| Subject | Lesion count | Taken at screen |
| Subject | Lymphocyte count | Taken at screen |
| Subject | Lymphocyte absolute count | Taken at screen |
| Subject | Magnesium level | Taken at screen |
| Subject | Monocyte absolute count | Taken at screen |
| Subject | Monocyte count | Taken at screen |
| Subject | Neutrophil absolute count | Taken at screen |
| Subject | Neutrophil count | Taken at screen |
| Subject | Phosphate level | Taken at screen |
| Subject | Platelet count | Taken at screen |
| Subject | Potassium level | Taken at screen |
| Subject | Total protein | Taken at screen |
| Subject | Red Blood Cell count | Taken at screen |
| Subject | Aspartate Aminotransferase (SGOT) | Taken at screen |
| Subject | Alanine Aminotransferase (SGPT) | Taken at screen |
| Subject | Sodium level | Taken at screen |
| Subject | Sum of products of diameters | Taken at screen |
| Subject | Triglycerides | Taken at screen |
| Subject | Longest tumor diameter | Taken at screen |
| Subject | Perpendicular tumor diameter | Taken at screen |
| Subject | Uric acid level | Taken at screen |
| Subject | White blood cell count | Taken at screen |

The 238 features were preprocessed to identify features for supervised training of the random forests and random survival forests models. Preprocessing consisted of four (4) steps:
1. Removing features with zero or near-zero variance, where near zero variance was defined by a feature having >95% of the data dominated by a single value and with fewer than 0.1n unique values (n=number of samples);
2. Removing features with >60% of values missing (e.g., not available);
3. Identifying highly correlated features (|ρ|>0.5) and selecting one representative feature; and
4. Imputing missing values using multivariate imputation by chained equations (mice; vanBuuren et al., Journal of Statistical Software. 2011; 45(3)).

Step 3 of preprocessing was performed by first computing a heterogeneous correlation matrix, consisting of Pearson product-moment correlations between numeric features, polyserial correlations between numeric and ordinal features, and polychoric correlations between ordinal features. Correlations were computed between each pair of variables using all complete pairs of observations on those variables. Covariate clusters were defined as sets of variables with correlation coefficients >0.5, and representative features were iteratively selected as those in each cluster that exhibit the lowest mean absolute correlation with all other remaining features in the dataset.

Preprocessing identified 76 representative independent features to be jointly considered. Of the 76 features, 33 of the features related to therapeutic cell composition attributes and attributes of starting material used to produce the therapeutic cell compositions and 47 features related to subject attributes.

The training data for the models included the preprocessed features (e.g., predictors) and 20 responses (e.g., dependent variables). The responses included: pharmacokinetic outcomes, including AUC and Cₘₐₓ; efficacy endpoints, such as progression-free survival (PFS), duration of response (DOR), complete response (CR), and objective response (OR); and safety endpoints, including any grade and severe cytokine release syndrome (CRS) and neurological events (NE). Table ES provides a list of exemplary outcomes considered.

TABLE E5
Clinical Outcomes as response variable.

| | |
|---|---|
| Pharmacokinetic Endpoints | AUC₀₋₂₈ (qPCR and flow cytometry) |
| | Cₘₐₓ (qPCR and flow cytometry) |
| Efficacy Endpoints | Progression-free survival (PFS) |
| | Duration of response (DOR) |
| | Complete response (CR) |
| | Objective response (OR)^{a} |
| Safety Endpoints | Any CRS (grade 0 vs ≥1) |
| | Any NE (grade 0 vs ≥1) |
| | Severe CRS (grades 0, 1, or 2 vs ≥3)^{b} |
| | Severe NE (grades 0, 1, or 2 vs ≥3)^{c} |

^{a}OR was the best objective response binarized by partial and complete response rates. PFS, CR, and OR were assessed by an Independent Review Committee.
^{b}CRS was graded according to the Lee criteria (Lee D W, et al. Blood. 2014; 124: 188-195).
^{c}NEs were based on investigator assessment and graded per National Cancer Institute Common Terminology Criteria for Adverse Eventsv4.03.
AUC₀₋₂₈, area under the concentration-time curve through 28 days after infusion;
Cₘₐₓ, maximum serum concentration;
CRS, cytokine release syndrome;
NE, neurological event;
qPCR, quantitative polymerase chain reaction.

B. Multivariate Supervised Learning

Random forests and random survival forests were trained using 10-fold cross validation to assess the importance of each feature in its contribution to clinical response.

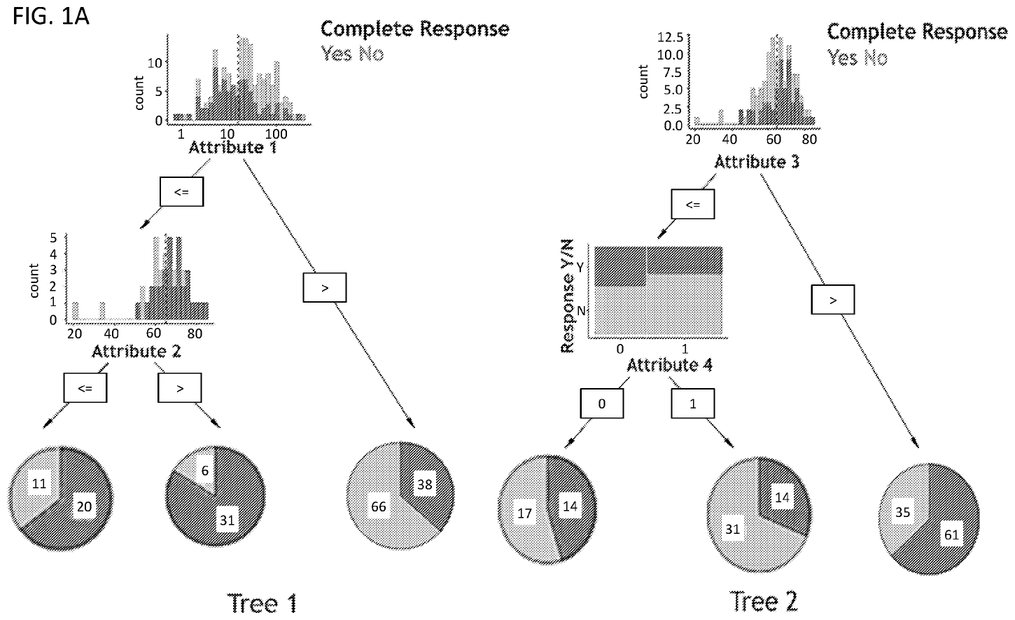

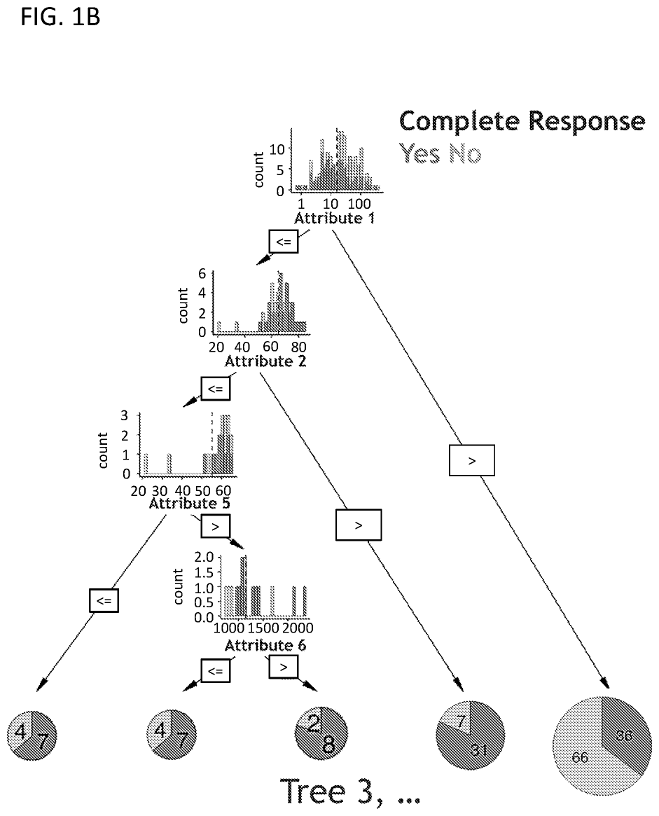

Random forests are composed of decision trees. Each tree was trained using a subset of patients and a subset of features to prevent overfitting (FIGS. 1A-1B). Hundreds of decision trees were constructed selecting subsets of the feature space to best separate the responses of interest, minimizing Gini impurity for classification, root mean-squared error for regression, or concordance index for survival.

The models were assessed to identify features important for determining clinical response. P values for significance of feature importance were assessed by permutation testing (Altmann A, et al. Bioinformatics. 2010; 26(10):1340-1347).

1. Pharmacokinetic Response

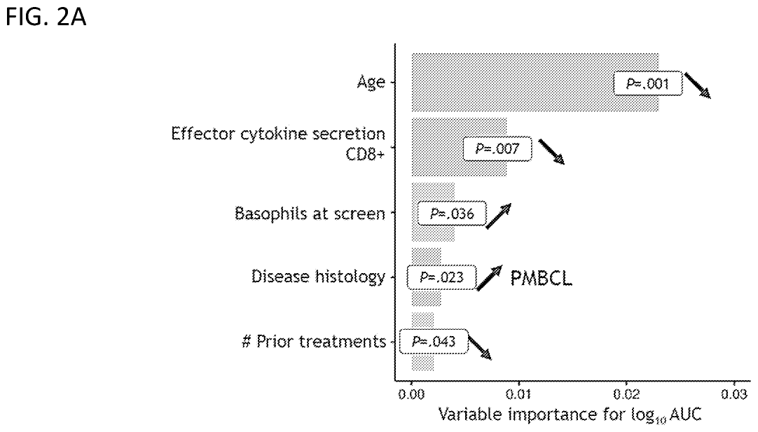

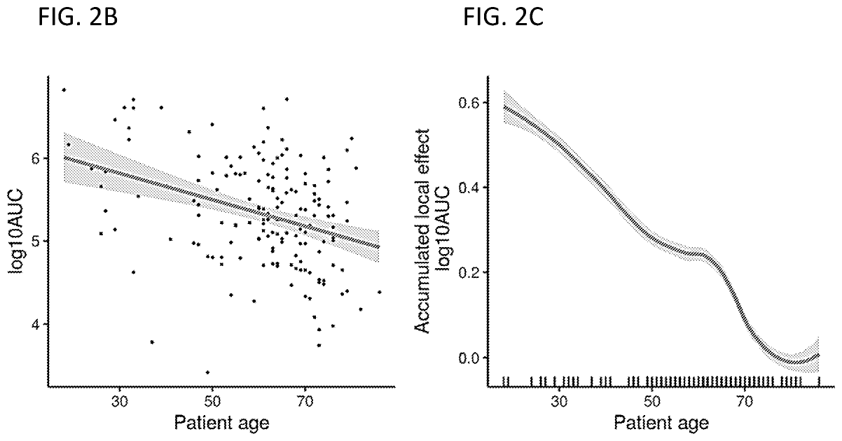

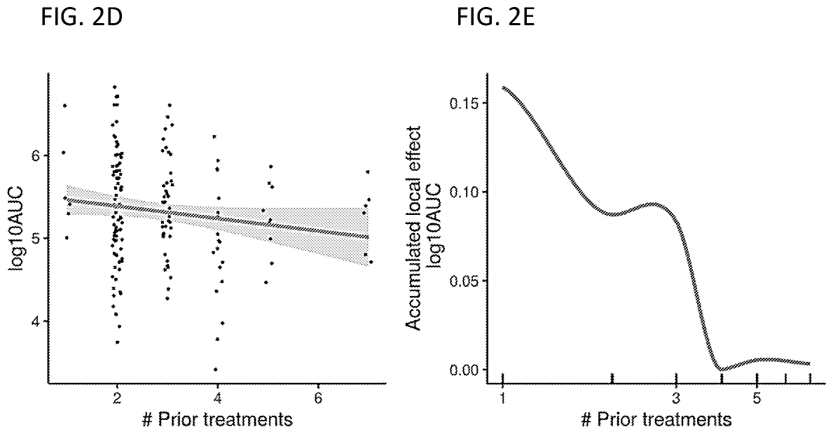

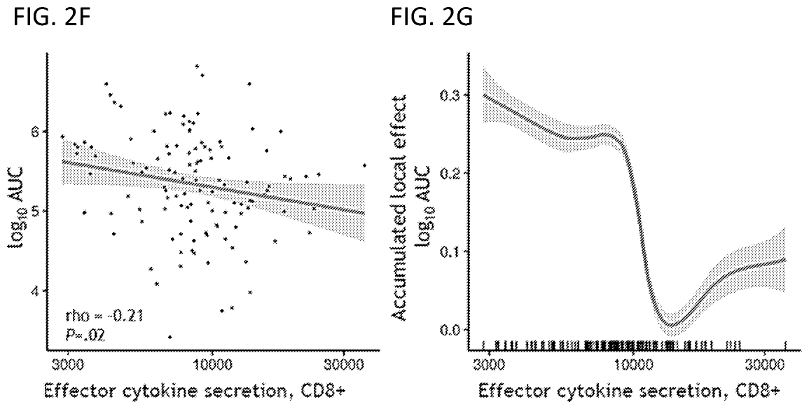

Five exemplary significant feature clusters were identified as important for correlating with log₁₀AUC₀₋₂₈ following treatment with the therapeutic cell composition. The exemplary features included age, effector cytokine secretion of CD8+ T cells in the therapeutic cell composition, basophils during screening, disease histology (PBMCL: primary mediastinal large B-cell lymphoma), and the number of prior treatments the patient received (FIG. 2A).

Younger patients treated with the therapeutic cell composition tended to experience higher CAR T cell expansion. CAR T expansion was measured by quantitative polymerase chain reaction and validated by flow cytometry. Univariately, this was also true (FIG. 2B), and the contribution of age given all the other factors in the multifactorial model was quantified using a technique called accumulative local effect estimation. This technique can quantify, for example, how each 1-year increase in age is associated with log₁₀AUC₀₋₂₈, independent of all other subject, input composition, and therapeutic cell composition features. FIG. 2C shows the accumulated local effect of patient age on log₁₀AUC₀₋₂₈.

Log₁₀AUC₀₋₂₈ was also found to correlate with the number of treatments a patient received prior to treatment with the therapeutic cell composition. Fewer prior therapies were associated with higher CAR T cell expansion (FIGS. 2D-2E).

Lower effector cytokine secretion of CD8+ cells of the therapeutic cell composition, which is reflective of a more naïve phenotype, also correlated with increased cell expansion as measured by AUC₀₋₂₈ (FIGS. 2F-2G).

2. Efficacy: PFS and CR

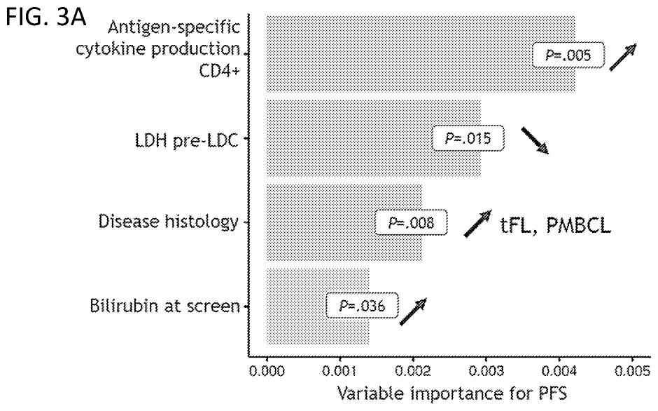

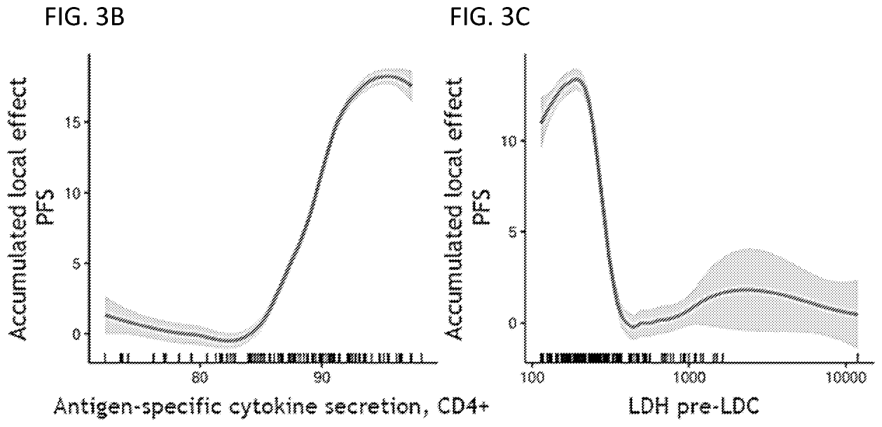

FIGS. 3A-3C show exemplary features associated with PFS. Longer PFS was associated with higher functional cytokine production of the CD4+ cells in the therapeutic cell composition (FIGS. 3A-3B).

Patients with reduced tumor burden (reflected by lower LDH levels prior to lymphodepleting chemotherapy) and patients with LBCL transformed from follicular lymphoma (tFL) or primary mediastinal B-cell lymphoma (PMBCL) also achieved longer PFS (FIGS. 3A and 3C). A weak association was seen between bilirubin and PFS; however, the effect appeared to be driven by subjects with lower than normal levels of bilirubin.

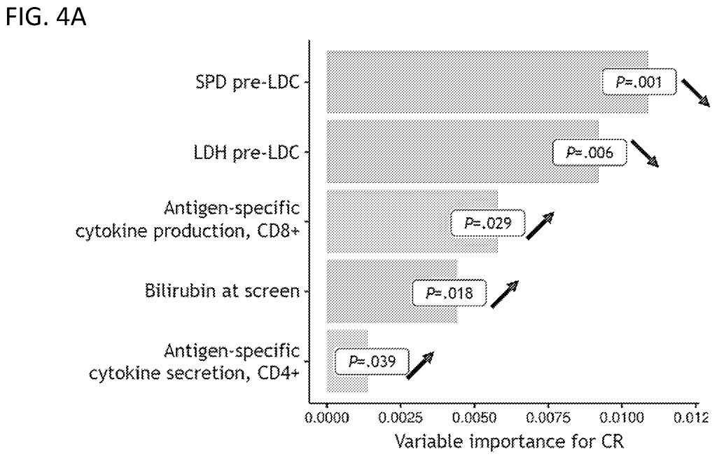

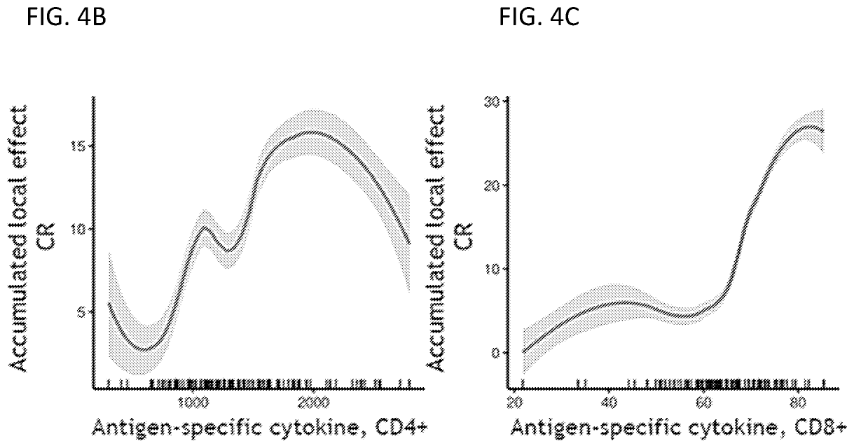

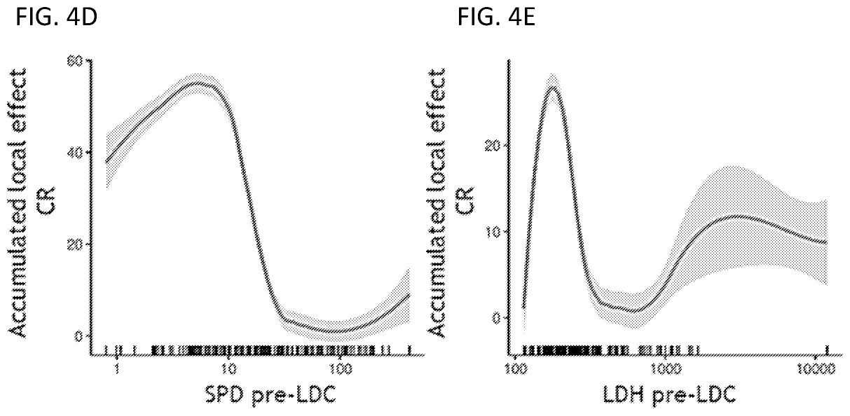

FIGS. 4A-4E show exemplary features associated with complete response (CR). Higher functional cytokine production by the therapeutic cell composition was associated with a higher probability of achieving complete response (CR; FIGS. 4A-4C). Patients with reduced tumor burden were also more likely to achieve complete response (FIGS. 4A and 4D-4E).

3. Safety: NE and CRS

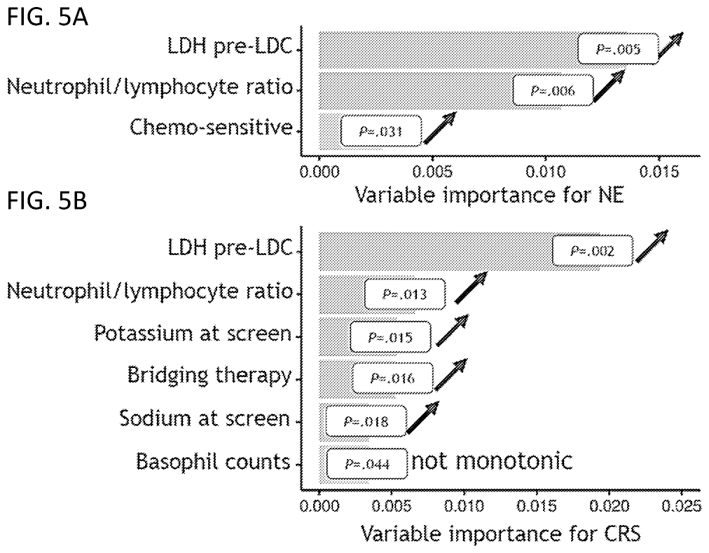

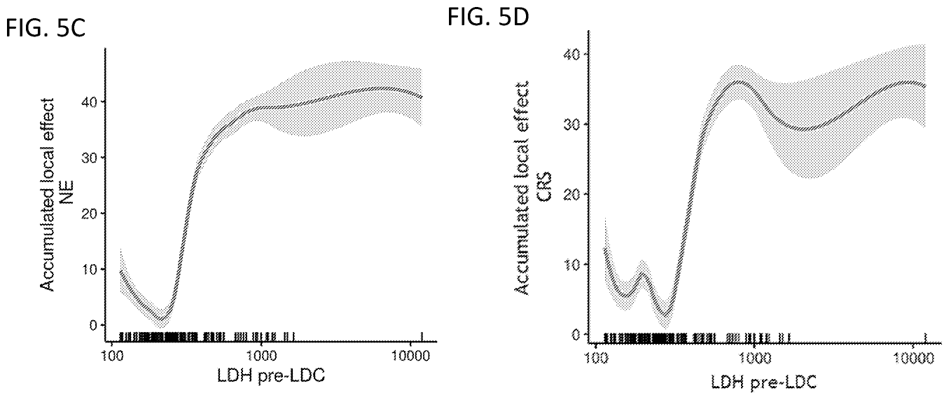

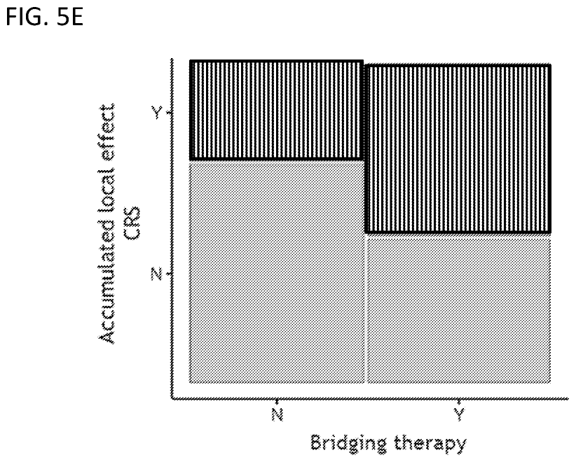

FIGS. 5A-5E show exemplary features associated with safety responses. Patients with greater tumor burden as measured by LDH level prior to receiving lymphodepleting chemotherapy were more likely to experience any-grade neurological event (NE) or cytokine release syndrome (CRS) after receiving the therapeutic cell composition (FIGS. 5A-5D).

Patients who required bridging therapy between apheresis and infusion of the therapeutic cell composition also had a higher risk for experiencing CRS, potentially due to the severity of their condition, which necessitated bridging therapy (FIGS. 5A, 5B, and 5E).

TABLE E6
summarizes exemplary features associated with clinical responses.
Exemplary features correlated with exemplary clinical outcomes.

| | |
|---|---|
| PK | Age ↑: log₁₀ AUC₀₋₂₈↓ |
| | Prior treatment ↑: log₁₀ AUC₀₋₂₈↓ |
| | Effector cytokine secretion ↑: log₁₀ AUC₀₋₂₈↓ |
| EFFICACY | Tumor burden (LDH) ↑: PFS ↓ |
| | Tumor burden (LDH, SPD) ↑: CR ↓ |
| | tFL, PMBCL: PFS ↑ |
| | Antigen-Specific Cytokine ↑: PFS ↑CR ↑ |
| SAFETY | Tumor burden (LDH) ↑: NE ↑CRS ↑ |
| | Bridging Therapy: CRS ↑ |

Tumor burden and antigen specific function of the cells of the therapeutic cell composition were the most important features associated with PK (cell expansion; AUC₀₋₂₈), efficacy (PFS, CR), and safety (CRS, NE) responses to treatment with the therapeutic cell composition.

Higher tumor burden was associated with increased CAR T cell expansion (AUC₀₋₂₈), risk of CRS and neurological events, and reduced efficacy. Independent of tumor burden, increased expression of antigen-specific cytokine levels and less-differentiated CAR T cells in the therapeutic cell composition were associated with increased expansion and efficacy.

These results show that supervised learning approaches to cell therapy based on subject and manufacturing data (e.g., input composition features and therapeutic cell composition features) offer insights into the multifactorial effects on patient clinical outcomes.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | LEGGGEGRGSLLTCGDVEENPGPR | T2A |
| 2 | MLLLVTSLLLCELPHPAFLLIPRKVCNGIGIGEFKDSLSINATNI | tEGFR |
| | KHFKNCTSISGDLHILPVAFRGDSFTHTPPLDPQELDILKTVKEI | |
| | TGFLLIQAWPENRTDLHAFENLEIIRGRTKQHGQFSLAVVSLNI | |
| | TSLGLRSLKEISDGDVIISGNKNLCYANTINWKKLFGTSGQKTK | |
| | IISNRGENSCKATGQVCHALCSPEGCWGPEPRDCVSCRNVSRG | |
| | RECVDKCNLLEGEPREFVENSECIQCHPECLPQAMNITCTGRG | |
| | PDNCIQCAHYIDGPHCVKTCPAGVMGENNTLVWKYADAGHV | |
| | CHLCHPNCTYGCTGPGLEGCPTNGPKIPSIATGMVGALLLLLV | |
| | VALGIGLFM | |
| 3 | RKVCNGIGIGEFKDSLSINATNIKHFKNCTSISGDLHILPVAFRG | tEGFR |
| | DSFTHTPPLDPQELDILKTVKEITGFLLIQAWPENRTDLHAFEN | |
| | LEIIRGRTKQHGQFSLAVVSLNITSLGLRSLKEISDGDVIISGNK | |
| | NLCYANTINWKKLFGTSGQKTKIISNRGENSCKATGQVCHALC | |
| | SPEGCWGPEPRDCVSCRNVSRGRECVDKCNLLEGEPREFVENS | |
| | ECIQCHPECLPQAMNITCTGRGPDNCIQCAHYIDGPHCVKTCP | |
| | AGVMGENNTLVWKYADAGHVCHLCHPNCTYGCTGPGLEGC | |
| | PTNGPKIPSIATGMVGALLLLLVVALGIGLFM | |
| 4 | EGRGSLLTCGDVEENPGP | T2A |
| 5 | GSGATNFSLLKQAGDVEENPGP | P2A |
| 6 | ATNFSLLKQAGDVEENPGP | P2A |
| 7 | QCTNYALLKLAGDVESNPGP | E2A |
| 8 | VKQTLNFDLLKLAGDVESNPGP | F2A |
| 9 | atgcttctcctggtgacaagccttctgctctgtgagttaccacacccagcattcctcctgatccca | GMCSFR alpha chain |
| | | signal sequence |
| 10 | MLLLVTSLLLCELPHPAFLLIP | GMCSFR alpha chain |
| | | signal sequence |
| 11 | MALPVTALLLPLALLLHA | CD8 alpha signal |
| | | peptide |
| 12 | MPLLLLLPLLWAGALA | CD33 signal peptide |
| 13 | QIQLVQSGPELKKPGETVKISCKASGYTFTDYSINWVKRAPGK | Variable heavy (VH) |
| | GLKWMGWINTETREPAYAYDFRGRFAFSLETSASTAYLQINN | Anti-BCMA |
| | LKYEDTATYFCALDYSYAMDYWGQGTSVTVSS | |
| 14 | DIVLTQSPPSLAMSLGKRATISCRASESVTILGSHLIHWYQQKP | Variable light (VL) |
| | GQPPTLLIQLASNVQTGVPARFSGSGSRTDFTLTIDPVEEDDVA | Anti-BCMA |
| | VYYCLQSRTIPRTFGGGTKLEIK | |
| 15 | QIQLVQSGPDLKKPGETVKLSCKASGYTFTNFGMNWVKQAPG | Variable heavy (VH) |
| | KGFKWMAWINTYTGESYFADDFKGRFAFSVETSATTAYLQIN | Anti-BCMA |
| | NLKTEDTATYFCARGEIYYGYDGGFAYWGQGTLVTVSA | |
| 16 | DVVMTQSHRFMSTSVGDRVSITCRASQDVNTAVSWYQQKPG | Variable light (VL) |
| | QSPKLLIFSASYRYTGVPDRFTGSGSGADFTLTISSVQAEDLAV | Anti-BCMA |
| | YYCQQHYSTPWTFGGGTKLDIK | |
| 17 | EVQLVQSGAEVKKPGESLKISCKGSGYSFTSYWIGWVRQMPG | Variable heavy (VH) |
| | KGLEWMGIIYPGDSDTRYSPSFQGHVTISADKSISTAYLQWSSL | Anti-BCMA |
| | KASDTAMYYCARYSGSFDNWGQGTLVTVSS | |
| 18 | SYELTQPPSASGTPGQRVTMSCSGTSSNIGSHSVNWYQQLPGT | Variable light (VL) |
| | APKLLIYTNNQRPSGVPDRFSGSKSGTSASLAISGLQSEDEADY | Anti-BCMA |
| | YCAAWDGSLNGLVFGGGTKLTVLG | |
| 19 | EVQLVQSGAEMKKPGASLKLSCKASGYTFIDYYVYWMRQAP | Variable heavy (VH) |
| | GQGLESMGWINPNSGGTNYAQKFQGRVTMTRDTSISTAYMEL | Anti-BCMA |
| | SRLRSDDTAMYYCARSQRDGYMDYWGQGTLVTVSS | |
| 20 | QSALTQPASVSASPGQSIAISCTGTSSDVGWYQQHPGKAPKLM | Variable light (VL) |
| | IYEDSKRPSGVSNRFSGSKSGNTASLTISGLQAEDEADYYCSSN | Anti-BCMA |
| | TRSSTLVFGGGTKLTVLG | |
| 21 | EVQLVQSGAEVKKPGSSVKVSCKASGGTFSSYAISWVRQAPG | Variable heavy (VH) |
| | QGLEWMGRIIPILGIANYAQKFQGRVTMTEDTSTDTAYMELSS | Anti-BCMA |
| | LRSEDTAVYYCARSGYSKSIVSYMDYWGQGTLVTVSS | |
| 22 | LPVLTQPPSTSGTPGQRVTVSCSGSSSNIGSNVVFWYQQLPGTA | Variable light (VL) |
| | PKLVIYRNNQRPSGVPDRFSVSKSGTSASLAISGLRSEDEADYY | Anti-BCMA |
| | CAAWDDSLSGYVFGTGTKVTVLG | |
| 23 | QVQLVQSGAEVKKPGSSVKVSCKASGGTFSSYAISWVRQAPG | Variable heavy (VH) |
| | QGLEWMGRIIPILGTANYAQKFQGRVTITADESTSTAYMELSS | Anti-BCMA |
| | LRSEDTAVYYCARSGYGSYRWEDSWGQGTLVTVSS | |
| 24 | QAVLTQPPSASGTPGQRVTISCSGSSSNIGSNYVFWYQQLPGTA | Variable light (VL) |
| | PKLLIYSNNQRPSGVPDRFSGSKSGTSASLAISGLRSEDEADYY | Anti-BCMA |
| | CAAWDDSLSASYVFGTGTKVTVLG | |
| 25 | QVQLVQSGAEVKKPGASVKVSCKASGYTFTDYYMHWVRQAP | Variable heavy (VH) |
| | GQRLEWMGWINPNSGGTNYAQKFQDRITVTRDTSSNTGYME | Anti-BCMA |
| | LTRLRSDDTAVYYCARSPYSGVLDKWGQGTLVTVSS | |
| 26 | QSVLTQPPSVSGAPGQRVTISCTGSSSNIGAGFDVHWYQQLPG | Variable light (VL) |
| | TAPKLLIYGNSNRPSGVPDRFSGSKSGTSASLAITGLQAEDEAD | Anti-BCMA |
| | YYCQSYDSSLSGYVFGTGTKVTVLG | |
| 27 | RASQDISKYLN | CDR L1 |
| 28 | SRLHSGV | CDR L2 |
| 29 | GNTLPYTFG | CDR L3 |
| 30 | DYGVS | CDR H1 |
| 31 | VIWGSETTYYNSALKS | CDR H2 |
| 32 | YAMDYWG | CDR H3 |
| 33 | HYYYGGSYAMDY | HC-CDR3 |
| 34 | HTSRLHS | LC-CDR2 |
| 35 | QQGNTLPYT | LC-CDR3 |
| 36 | EVKLQESGPGLVAPSQSLSVTCTVSGVSLPDYGVSWIRQPPRK | VH |
| | GLEWLGVIWGSETTYYNSALKSRLTIIKDNSKSQVFLKMNSLQ | |
| | TDDTAIYYCAKHYYYGGSYAMDYWGQGTSVTVSS | |
| 37 | DIQMTQTTSSLSASLGDRVTISCRASQDISKYLNWYQQKPDGT | VL |
| | VKLLIYHTSRLHSGVPSRFSGSGSGTDYSLTISNLEQEDIATYFC | |
| | QQGNTLPYTFGGGTKLEIT | |
| 38 | GSTSGSGKPGSGEGSTKG | Linker |
| 39 | gacatccagatgacccagaccacctccagcctgagcgccagcctgggcgaccgggtgaccatca | Sequence encoding |
| | gctgccgggccagccaggacatcagcaagtacctgaactggtatcagcagaagcccgacggca | scFv |
| | ccgtcaagctgctgatctaccacaccagccggctgcacagcggcgtgcccagccggtttagcggc | |
| | agcggctccggcaccgactacagcctgaccatctccaacctggaacaggaagatatcgccaccta | |
| | cttttgccagcagggcaacacactgccctacacctttggcggcggaacaaagctggaaatcaccg | |
| | gcagcacctccggcagcggcaagcctggcagcggcgagggcagcaccaagggcgaggtgaa | |
| | gctgcaggaaagcggccctggcctggtggcccccagccagagcctgagcgtgacctgcaccgt | |
| | gagcggcgtgagcctgcccgactacggcgtgagctggatccggcagccccccaggaagggcct | |
| | ggaatggctgggcgtgatctggggcagcgagaccacctactacaacagcgccctgaagagccg | |
| | gctgaccatcatcaaggacaacagcaagagccaggtgttcctgaagatgaacagcctgcagacc | |
| | gacgacaccgccatctactactgcgccaagcactactactacggcggcagctacgccatggacta | |
| | ctggggccagggcaccagcgtgaccgtgagcagc | |
| 40 | DIQMTQTTSSLSASLGDRVTISCRASQDISKYLNWYQQKPDGT | scFv |
| | VKLLIYHTSRLHSGVPSRFSGSGSGTDYSLTISNLEQEDIATYFC | |
| | QQGNTLPYTFGGGTKLEITGSTSGSGKPGSGEGSTKGEVKLQE | |
| | SGPGLVAPSQSLSVTCTVSGVSLPDYGVSWIRQPPRKGLEWLG | |
| | VIWGSETTYYNSALKSRLTIIKDNSKSQVFLKMNSLQTDDTAIY | |
| | YCAKHYYYGGSYAMDYWGQGTSVTVSS | |
| 41 | SYWMN | CDR H1 |
| 42 | QIYPGDGDTNYNGKFKG | CDR H2 |
| 43 | KTISSVVDFYFDY | CDR H3 |
| 44 | KASQNVGTNVA | CDR L1 |
| 45 | SATYRNS | CDR L2 |
| 46 | QQYNRYPYT | CDR L3 |
| 47 | EVKLQQSGAELVRPGSSVKISCKASGYAFSSYWMNWVKQRPG | VH |
| | QGLEWIGQIYPGDGDTNYNGKFKGQATLTADKSSSTAYMQLS | |
| | GLTSEDSAVYFCARKTISSVVDFYFDYWGQGTTVTVSS | |
| 48 | DIELTQSPKFMSTSVGDRVSVTCKASQNVGTNVAWYQQKPGQ | VL |
| | SPKPLIYSATYRNSGVPDRFTGSGSGTDFTLTITNVQSKDLADY | |
| | FCQQYNRYPYTSGGGTKLEIKR | |
| 49 | GGGGSGGGGSGGGGS | Linker |
| 50 | EVKLQQSGAELVRPGSSVKISCKASGYAFSSYWMNWVKQRPG | scFv |
| | QGLEWIGQIYPGDGDTNYNGKFKGQATLTADKSSSTAYMQLS | |
| | GLTSEDSAVYFCARKTISSVVDFYFDYWGQGTTVTVSSGGGG | |
| | SGGGGSGGGGSDIELTQSPKFMSTSVGDRVSVTCKASQNVGT | |
| | NVAWYQQKPGQSPKPLIYSATYRNSGVPDRFTGSGSGTDFTLT | |
| | ITNVQSKDLADYFCQQYNRYPYTSGGGTKLEIKR | |
| 51 | GGGGS | Linker |
| 52 | GGGS | Linker |
| 53 | GGGGSGGGGSGGGGS | Linker |
| 54 | GSTSGSGKPGSGEGSTKG | Linker |
| 55 | SRGGGGSGGGGSGGGGSLEMA | Linker |
| 56 | ESKYGPPCPPCPAPEFLGGPSVFLFPPKPKDTLMISRTPEVTCVV | Hinge-CH2-CH3 |
| | VDVSQEDPEVQFNWYVDGVEVHNAKTKPREEQFNSTYRVVS | spacer Homo sapiens |
| | VLTVLHQDWLNGKEYKCKVSNKGLPSSIEKTISKAKGQPREPQ | |
| | VYTLPPSQEEMTKNQVSLTCLVKGFYPSDIAVEWESNGQPENN | |
| | YKTTPPVLDSDGSFFLYSRLTVDKSRWQEGNVFSCSVMHEAL | |
| | HNHYTQKSLSLSLGK | |
| 57 | ASTKGPSVFPLAPCSRSTSESTAALGCLVKDYFPEPVTVSWNS | Human IgG2 Fc |
| | GALTSGVHTFPAVLQSSGLYSLSSVVTVPSSNFGTQTYTCNVD | (Uniprot P01859) |
| | HKPSNTKVDKTVERKCCVECPPCPAPPVAGPSVFLFPPKPKDT | |
| | LMISRTPEVTCVVVDVSHEDPEVQFNWYVDGVEVHNAKTKPR | |
| | EEQFNSTFRVVSVLTVVHQDWLNGKEYKCKVSNKGLPAPIEK | |
| | TISKTKGQPREPQVYTLPPSREEMTKNQVSLTCLVKGFYPSDIS | |
| | VEWESNGQPENNYKTTPPMLDSDGSFFLYSKLTVDKSRWQQG | |
| | NVFSCSVMHEALHNHYTQKSLSLSPGK | |
| 58 | ESKYGPPCPPCP | spacer (IgG4hinge) |
| | | (aa) Homo sapiens |
| 59 | ESKYGPPCPPCPGQPREPQVYTLPPSQEEMTKNQVSLTCLVKG | Hinge-CH3 spacer |
| | FYPSDIAVEWESNGQPENNYKTTPPVLDSDGSFFLYSRLTVDK | Homo sapiens |
| | SRWQEGNVFSCSVMHEALHNHYTQKSLSLSLGK | |
| 60 | gaatctaagtacggaccgccctgccccccttgccct | spacer (IgG4hinge) |
| | | (nt) Homo sapiens |
| 61 | RWPESPKAQASSVPTAQPQAEGSLAKATTAPATTRNTGRGGE | IgD-hinge-Fc Homo |
| | EKKKEKEKEEQEERETKTPECPSHTQPLGVYLLTPAVQDLWLR | sapiens |
| | DKATFTCFVVGSDLKDAHLTWEVAGKVPTGGVEEGLLERHSN | |
| | GSQSQHSRLTLPRSLWNAGTSVTCTLNHPSLPPQRLMALREPA | |
| | AQAPVKLSLNLLASSDPPEAASWLLCEVSGFSPPNILLMWLED | |
| | QREVNTSGFAPARPPPQPGSTTFWAWSVLRVPAPPSPQPATYT | |
| | CVVSHEDSRTLLNASRSLEVSYVTDH | |
| 62 | FWVLVVVGGVLACYSLLVTVAFIIFWV | CD28 (amino acids |
| | | 153-179 of Accession |
| | | No. P10747) Homo |
| | | sapiens |
| 63 | IEVMYPPPYLDNEKSNGTIIHVKGKHLCPSPLFPGPSKPFWVLV | CD28 (amino acids |
| | VVGGVLACYSLLVTVAFIIFWV | 114-179 of Accession |
| | | No. P10747) Homo |
| | | sapiens |
| 64 | RSKRSRLLHSDYMNMTPRRPGPTRKHYQPYAPPRDFAAYRS | CD28 (amino acids |
| | | 180-220 of P10747) |
| | | Homo sapiens |
| 65 | RSKRSRGGHSDYMNMTPRRPGPTRKHYQPYAPPRDFAAYRS | CD28 (LL to GG) |
| | | Homo sapiens |
| 66 | KRGRKKLLYIFKQPFMRPVQTTQEEDGCSCRFPEEEEGGCEL | 4-1BB (amino acids |
| | | 214-255 of Q07011.1) |
| | | Homo sapiens |
| 67 | RVKFSRSADAPAYQQGQNQLYNELNLGRREEYDVLDKRRGR | CD3 zeta Homo |
| | DPEMGGKPRRKNPQEGLYNELQKDKMAEAYSEIGMKGERRR | sapiens |
| | GKGHDGLYQGLSTATKDTYDALHMQALPPR | |
| 68 | RVKFSRSAEPPAYQQGQNQLYNELNLGRREEYDVLDKRRGRD | CD3 zeta Homo |
| | PEMGGKPRRKNPQEGLYNELQKDKMAEAYSEIGMKGERRRG | sapiens |
| | KGHDGLYQGLSTATKDTYDALHMQALPPR | |
| 69 | RVKFSRSADAPAYKQGQNQLYNELNLGRREEYDVLDKRRGR | CD3 zeta Homo |
| | DPEMGGKPRRKNPQEGLYNELQKDKMAEAYSEIGMKGERRR | sapiens |
| | GKGHDGLYQGLSTATKDTYDALHMQALPPR | |

1. A method of determining a clinical response, the method comprising:
(a) receiving features comprising:
(i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from an input composition, wherein the input composition comprises T cells selected from a sample from the subject, wherein the T cells are used for producing the therapeutic cell composition comprising T cells comprising the chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject; and
(b) applying the features as input to a random forests model trained to determine, based on informative features identified by preprocessing, a clinical response of the subject to treatment with the therapeutic cell composition prior to treating the subject with the therapeutic cell composition, wherein the features applied as input are the same informative features as those used to train the random forests model.

2. A method of determining a clinical response, the method comprising:
(a) receiving features comprising:
(i) subject features determined from a subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from an input composition, wherein the input composition comprises T cells selected from a sample from the subject, wherein the T cells are used for producing the therapeutic cell composition comprising T cells comprising the chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject; and
(b) applying the features as input to a random survival forests model trained to determine, based on informative features identified by preprocessing, a clinical response of the subject to treatment with the therapeutic cell composition prior to treating the subject with the therapeutic cell composition, wherein the features applied as input are the same informative features as those used to train the random survival forests model.

3. The method of claim 1 or claim 2, wherein the clinical response is or comprises a complete response (CR), a partial response (PR), a durable response, progression free survival (PFS), objective response (OR), a pharmacokinetic response that is or is greater than a target pharmacokinetic response, no or a mild toxicity response, a toxicity response, a reduced pharmacokinetics response compared to a target response, or a lack of CR, PR, durable response, or objective response (OR).

4. The method of claim 1, wherein the clinical response is a complete response (CR) or a lack of complete response (CR).

5. The method of claim 1, wherein the clinical response is a partial response (PR) or a lack of partial response (PR).

6. The method of claim 1, wherein the clinical response is an objective response (OR) or a lack of objective response (OR).

7. The method of claim 1, wherein the clinical response is a toxicity response or a lack of a toxicity response.

8. The method of claim 3 or claim 7, wherein the toxicity response is severe cytokine release syndrome (CRS) or severe neurotoxicity.

9. The method of claim 1 or claim 2, wherein the clinical response is a durable response or a lack of durable response.

10. The method of claim 1 or claim 2, wherein the clinical response is the duration of response (DOR).

11. The method of claim 1 or claim 2, wherein the clinical response is a duration of response (DOR) of at least or at least about three months.

12. The method of claim 1 or claim 2, wherein the clinical response is progression free survival (PFS).

13. The method of claim 1 or claim 2, wherein the clinical response is progression free survival (PFS) of at least or at least about three months.

14. The method of claim 1, wherein the clinical response is a pharmacokinetic response that is or is greater than a target pharmacokinetic response.

15. The method of claim 3 or claim 14, wherein the pharmacokinetic response is a measure of:
(i) expansion of CAR T cells of the therapeutic cell composition following treatment of the subject with the therapeutic cell composition;
(ii) maximum CAR T cell concentration in the subject following treatment of the subject with the therapeutic cell composition;
(iii) a timepoint at which CAR T cell concentration is maximal in the subject following treatment of the subject with the therapeutic cell composition; or
(iv) exposure of the subject to CAR T cells of the therapeutic cell composition following treatment of the subject with the therapeutic cell composition.

16. A method of treating a subject, the method comprising:
(a) selecting T cells from a sample from a subject to produce an input composition comprising T cells;
(b) determining features comprising:
(i) subject features determined from the subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from the input composition, wherein the input composition comprises T cells selected from the sample from the subject, wherein the T cells are used for producing the therapeutic cell composition comprising T cells comprising the chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject;
(c) applying the features as input to a random forests model trained to determine, based on informative features identified by preprocessing, a clinical response of the subject to treatment with the therapeutic cell composition prior to treating the subject with the therapeutic cell composition, wherein the features applied as input are the same informative features as those used to train the random forests model; and
(d) administering a treatment to the subject wherein:
(1) if the subject is determined to have a clinical response selected from the group consisting of a complete response (CR), a partial response (PR), a durable response of greater than 3 months, progression free survival (PFS) for more than 3 months, objective response (OR), a desired pharmacokinetic response that is or is greater than a target pharmacokinetic response, and no or a mild toxicity response (optionally wherein the mild toxicity response is grade 2 or less cytokine release syndrome (CRS) or grade 2 or less neurotoxicity), a predetermined treatment regimen comprising the therapeutic cell composition is administered; or
(2) if the subject is determined to have a clinical response selected from the group consisting of a toxicity response (optionally wherein the toxicity response is a severe cytokine release syndrome (CRS) or severe neurotoxicity), a reduced pharmacokinetic response compared to a target pharmacokinetic response, progressive disease (PD), a durable response of less than 3 months, and PFS of less than 3 months, administering to the subject a treatment regimen comprising the therapeutic cell composition that is altered compared to the predetermined treatment regimen comprising the therapeutic cell composition.

17. A method of treating a subject, the method comprising:
(a) selecting T cells from a sample from a subject to produce an input composition comprising T cells;
(b) determining features comprising:
(i) subject features determined from the subject prior to the subject being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from the input composition, wherein the input composition comprises T cells selected from the sample from the subject, wherein the T cells are used for producing the therapeutic cell composition comprising T cells comprising the chimeric antigen receptor (CAR); and
(iii) therapeutic cell composition features determined from the therapeutic cell composition, wherein the therapeutic cell composition is produced from the input composition and expresses the CAR, wherein the therapeutic composition is to be administered to the subject;
(c) applying the features as input to a random survival forests model trained to determine, based on informative features identified by preprocessing, a clinical response in the subject to be treated with the therapeutic cell composition prior to treating the subject with the therapeutic cell composition, wherein the features applied as input are the same informative features as those used to train the random survival forests model; and
(d) administering a treatment to the subject wherein:
(1) if the subject is determined to have a clinical response selected from the group consisting of a complete response (CR), a partial response (PR), a durable response of greater than 3 months, progression free survival (PFS) for more than 3 months, objective response (OR), a desired pharmacokinetic response that is or is greater than a target pharmacokinetic response, and no or a mild toxicity response (optionally wherein the mild toxicity response is grade 2 or less cytokine release syndrome (CRS) or grade 2 or less neurotoxicity), a predetermined treatment regimen comprising the therapeutic cell composition is administered; or
(2) if the subject is determined to have a clinical response selected from the group consisting of a toxicity response (optionally wherein the toxicity response is a severe cytokine release syndrome (CRS) or severe neurotoxicity), a reduced pharmacokinetics response compared to a target pharmacokinetic response, progressive disease (PD), a durable response of less than 3 months, and PFS of less than 3 months, administering to the subject a treatment regimen comprising the therapeutic cell composition that is altered compared to the predetermined treatment regimen comprising the therapeutic cell composition.

18. The method of claim 16, wherein the random forests model is trained to determine if the subject will have a complete response (CR), and wherein:
(1) the subject is administered the predetermined treatment regimen if the subject is determined to have a complete response (CR); or
(2) the subject is administered the altered treatment regimen if the subject is determined to have progressive disease (PD).

19. The method of claim 16, wherein the random forests model is trained to determine if the subject will have a partial response (PR), and wherein:
(1) the subject is administered the predetermined treatment regimen if the subject is determined to have a partial response (PR); or
(2) the subject is administered the altered treatment regimen if the subject is determined to have progressive disease (PD).

20. The method of claim 16, wherein the random forests model is trained to determine if the subject will have a durable response of greater than 3 months, and wherein:
(1) the subject is administered the predetermined treatment regimen if the subject is determined to have a durable response of greater than three months; or
(2) the subject is administered the altered treatment regimen if the subject is determined to have a durable response of less than three months.

21. The method of claim 17, wherein the random survival forests model is trained to determine if the subject will have a durable response of greater than 3 months, and wherein:
(1) the subject is administered the predetermined treatment regimen if the subject is determined to have a durable response of greater than three months; or
(2) the subject is administered the altered treatment regimen if the subject is determined to have a durable response of less than three months.

22. The method of claim 16, wherein the random forests model is trained to determine if the subject will have progression free survival (PFS) for more than 3 months, and wherein:
(1) the subject is administered the predetermined treatment regimen if the subject is determined to have progression free survival (PFS) for more than three months; or
(2) the subject is administered the altered treatment regimen if the subject is determined to have progression free survival (PFS) of less than three months.

23. The method of claim 17, wherein the random survival forests model is trained to determine if the subject will have progression free survival (PFS) for more than 3 months, and wherein:
(1) the subject is administered the predetermined treatment regimen if the subject is determined to have progression free survival (PFS) for more than three months; or
(2) the subject is administered the altered treatment regimen if the subject is determined to have progression free survival (PFS) of less than three months.

24. The method of claim 16, wherein the random forests model is trained to determine if the subject will have an objective response (OR), and wherein:
(1) the subject is administered the predetermined treatment regimen if the subject is determined to have an objective response (OR); or
(2) the subject is administered the altered treatment regimen if the subject is determined to have progressive disease (PD).

25. The method of claim 16, wherein the random forests model is trained to determine a pharmacokinetic response of the subject, and wherein:
(1) the subject is administered the predetermined treatment regimen if the subject is determined to have a desired pharmacokinetic response that is or is greater than a target pharmacokinetic response; or
(2) the subject is administered the altered treatment regimen if the subject is determined to have a reduced pharmacokinetic response compared to the target pharmacokinetic response.

26. The method of claim 25, wherein the pharmacokinetic response is a measure of:
(i) expansion of CAR T cells of the therapeutic cell composition following treatment of the subject with the therapeutic cell composition;
(ii) maximum CAR T cell concentration in the subject following treatment of the subject with the therapeutic cell composition;
(iii) a timepoint at which CAR T cell concentration is maximal in the subject following treatment of the subject with the therapeutic cell composition; or
(iv) exposure of the subject to CAR T cells of the therapeutic cell composition following treatment of the subject with the therapeutic cell composition.

27. The method of claim 16, wherein the random forests model is trained to determine if the subject will have a toxicity response, and wherein:
(1) the subject is administered the predetermined treatment regimen if the subject is determined to have no or a mild toxicity response; or
(2) the subject is administered the altered treatment regimen if the subject is determined to have a toxicity response.

28. The method of claim 27, wherein the toxicity response is severe CRS or severe neurotoxicity.

29. The method of any of claims 16-28, further comprising generating the therapeutic cell composition.

30. The method of any of claims 1, 3-16, 18-20, 22, and 24-29, wherein the random forests model is trained using supervised training, the supervised training comprising:
(a) receiving features comprising:
(i) subject features determined from each of a plurality of subjects prior to the subjects being treated with a therapeutic cell composition comprising T cells comprising the chimeric antigen receptor (CAR) that binds to the antigen associated with the disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising the chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;
(b) preprocessing the features to identify informative features, the informative features comprising a subset of the features comprising one or more subject features, one or more input composition features, and one or more therapeutic cell composition features;
(c) obtaining clinical responses from each of the plurality of subjects following treatment with one of the plurality of therapeutic compositions; and
(d) applying the informative features from a plurality of subjects and the obtained clinical responses as input to train a random forests model.

31. The method of any of claims 2, 3, 8-13, 15, 17, 21, 23, and 29, wherein the random survival forests model is trained using supervised training, the supervised training comprising:
(a) receiving features comprising:
(i) subject features determined from each of a plurality of subjects prior to the subjects being treated with a therapeutic cell composition comprising T cells comprising the chimeric antigen receptor (CAR) that binds to the antigen associated with the disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising a chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;
(b) preprocessing the features to identify informative features, the informative features comprising a subset of the features comprising one or more subject features, one or more input composition features, and one or more therapeutic cell composition features;
(c) obtaining clinical responses over time from each of the plurality of subjects following treatment with one of the plurality of therapeutic compositions; and
(d) applying the informative features and clinical responses from the plurality of subjects as input to train a random survival forests model using supervised learning.

32. A method of developing a random forests model comprising:
(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising the chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;
(b) preprocessing the features to identify informative features, the informative features comprising a subset of the features comprising one or more subject features, one or more input composition features, and one or more therapeutic cell composition features;
(c) obtaining clinical responses from each of the plurality of subjects following treatment with one of the plurality of therapeutic compositions; and
(d) applying the informative features from a plurality of subjects and the obtained clinical responses as input to train a random forests model.

33. A method of developing a random survival forests model comprising:
(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising the chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;
(b) preprocessing the features to identify informative features, the informative features comprising a subset of the features comprising one or more subject features, one or more input composition features, and one or more therapeutic cell composition features;
(c) obtaining clinical responses over time from each of the plurality of subjects following treatment with one of the plurality of therapeutic compositions; and
(d) applying the informative features and clinical responses from the plurality of subjects as input to train a random survival forests model using supervised learning.

34. A method of identifying features associated with a clinical response, the method comprising:
(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising the CAR; and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;
(b) preprocessing the features to identify informative features, the informative features comprising a subset of the features comprising one or more of the subject features, one or more the input composition features, and one or more of the therapeutic cell composition features;
(c) obtaining clinical responses from each of the plurality of subjects following treatment with one of the plurality of therapeutic compositions;
(d) applying the informative features and the obtained clinical responses from the plurality of subjects as input to train a random forests model using supervised learning; and
(e) identifying from the trained random forests model the informative features associated with the clinical responses.

35. A method of identifying features associated with a clinical response, the method comprising:
(a) receiving features comprising:
(i) subject features determined from each of a plurality of subjects prior to the subjects being treated with a therapeutic cell composition comprising T cells comprising a chimeric antigen receptor (CAR) that binds to an antigen associated with a disease or condition, wherein the therapeutic cell composition is for treating the disease or condition;
(ii) input composition features determined from each of a plurality of input compositions, wherein each of the plurality of input compositions comprises T cells selected from a sample from each of the plurality of subjects, wherein the T cells are used for producing the therapeutic cell composition comprising T cells comprising the chimeric antigen receptor (CAR); and
(iii) therapeutic cell composition features determined from each of a plurality of therapeutic cell compositions, wherein each of the plurality of therapeutic cell compositions is produced from one of the plurality of input compositions and expresses the CAR, wherein the therapeutic composition is to be administered to one of the plurality of subjects;
(b) preprocessing the features to identify informative features, the informative features comprising a subset of the features comprising one or more of the subject features, one or more of the input composition features, and one or more of the therapeutic cell composition features;
(c) obtaining clinical responses over time from each of the plurality of subjects following treatment with one of the plurality of therapeutic compositions;
(d) applying the informative features and clinical responses from the plurality of subjects as input to train a random survival forests model using supervised learning; and
(e) identifying from the trained random survival forests model the informative features associated with the clinical responses.

36. The method of any of claims 32-35, wherein the clinical response is or comprises a complete response (CR), a partial response (PR), a durable response, progression free survival (PFS), objective response (OR), a pharmacokinetic response that is or is greater than a target pharmacokinetic response, no or a mild toxicity response, a toxicity response, a reduced pharmacokinetics response compared to a target response, or a lack of CR, PR, durable response, or objective response (OR).

37. The method of any of claims 34-36, wherein the identifying the informative features associated with the clinical responses comprises determining an importance measure for each of the informative features.

38. The method of claim 37, wherein the importance measure comprises a permutation importance measure, a mean minimal depth, and/or a total number of trees from the trained random forests model wherein the informative feature splits a root node.

39. The method of claim 37, wherein the importance measure comprises a permutation importance measure, a mean minimal depth, and/or a total number of trees from the trained random survival forests model wherein the informative feature splits a root node.

40. The method of any of claims 37-39, wherein the informative features associated with the clinical responses are the first 10, 9, 8, 7, 6, 5, 4, 3, 2, or 1 informative features identified by rank ordering values of the importance measure for each of the informative features, wherein the importance measure is the same for each informative feature.

41. The method of any of claims 30-40, wherein each of the plurality of subjects is administered one of the plurality of therapeutic cell compositions, wherein the one therapeutic cell composition administered to the subject is the therapeutic cell composition produced from the input composition of the sample from the subject.

42. The method of any of claims 30-41, wherein the preprocessing to identify informative features comprises one or more of:
a) removing subject features, input composition features, and therapeutic cell composition features having greater than, than about, or 50% of the data missing;
b) removing subject features, input composition features, and therapeutic cell composition features having (i) zero variance, (ii) greater than, greater than about, or equal to 95% of data values equal to a single value, (iii) and/or fewer than 0.1n unique values, wherein n=number of samples;
c) imputing missing data for subject features, input composition features, and therapeutic cell composition features by multivariate imputation by chained equations; and
d) identifying covariate clusters, the covariate clusters comprising sets of subject features, input composition features, therapeutic cell composition features, and combinations thereof with correlation coefficients having absolute values of greater than, about, or equal to 0.5, and iteratively selecting subject features, input composition features, and therapeutic cell composition features from the covariate cluster, wherein the selected subject features, input composition features, and therapeutic cell composition features have the lowest mean absolute correlation with all remaining subject features, input composition features, and therapeutic cell composition features.

43. The method of any of claims 30-42, wherein the preprocessing to identify informative features comprises or is removing subject features, input composition features, and therapeutic cell composition features having greater than, about, or 50% of the data missing.

44. The method of any of claims 30-43, wherein the preprocessing to identify informative features comprises or is removing subject features, input composition features, and therapeutic cell composition features having greater than, about, or 60% of the data missing.

45. The method of any of claims 30-44, wherein the preprocessing to identify informative features comprises or is removing subject features, input composition features, and therapeutic cell composition features having (i) zero variance or (ii) greater than, about, or 95% of data values equal to a single value and fewer than 0.1n unique values, wherein n=number of samples.

46. The method of any of claims 30-45, wherein the preprocessing to identify informative features comprises or is imputing missing data for subject features, input composition features, and therapeutic cell composition features by multivariate imputation by chained equations.

47. The method of any of claims 30-46, wherein the preprocessing to identify informative features comprises or is identifying covariate clusters, the covariate clusters comprising sets of subject features, input composition features, therapeutic cell composition features, and combinations thereof with correlation coefficients having absolute values of greater than, about, or equal to 0.5, and iteratively selecting subject features, input composition features, and therapeutic cell composition features from the covariate cluster, wherein the selected subject features, input composition features, and therapeutic cell composition features have the lowest mean absolute correlation with all remaining subject features, input composition features, and therapeutic cell composition features.

48. The method of any of claims 30-47, wherein the plurality of subjects is or is about 500, 400, 300, 200, 150, 100, 50, 25, 15, or 10 subjects, or is any number between any of the foregoing.

49. The method of any of claims 30-48, wherein the plurality of subjects is, is about, or is greater than 10 subjects and less than 250 subjects.

50. The method of any of claims 30-49, wherein the plurality of subjects is, is about, or is greater than 20 subject and less than 200 subjects.

51. The method of any of claims 30-50, wherein the plurality of subjects is, is about, or is greater than 20 and less than 150 subjects.

52. The method of any of claims 30-51, wherein the plurality of subjects is, is about, or is greater than 20 subjects and less than 100 subjects.

53. The method of any of claims 1-52, wherein the subject features comprise one or more of subject attributes and clinical attributes.

54. The method of claim 53, wherein the subject attributes comprise one or more of age, weight, height, ethnicity, race, sex, and body mass index.

55. The method of claim 53 or claim 54, wherein the clinical attributes comprise one or more of biomarkers, disease diagnosis, disease burden, disease duration, disease grade, and treatment history.

56. The method of any of claims 1-55, wherein the subject features comprise one or more of dosing arm, bridging chemotherapy, bridging chemotherapy and radiotherapy, bridging chemotherapy systemic treatment, cell origin, relapsed or refractory following chemotherapy, type of diagnosis, disease cohort, disease burden, relapsed or refractory disease, disease origin, gender, therapeutic cell composition administration route, fold change in LDH, height, lesion count, oxygen saturation, temperature (° c), longest tumor diameter pre-treatment with therapeutic cell composition, fold change in SPD, SPD value pre-lymphodepleting chemotherapy, BMI, weight, sex, ethnicity, race, age, IPI score, ECOG score, disease stage, disease burden based on pre-lymphodepleting chemotherapy LDH, disease burden based on pre-lymphodepleting chemotherapy SPD, subject having active CNS disease at time of treatment, disease burden based on extranodal disease classification, number of extranodal sites, disease burden based on bulky disease classification, disease histology, number of prior lines of therapy, number of prior lines of systemic therapy, prior allogenic hematopoietic stem cell transplantation (allo-HSCT), prior autologous hematopoietic stem cell transplantation (auto-HSCT), chemorefractory or chemosensitive disease type, bridging anticancer therapy for disease control, days from date of leukapheresis to first infusion, months from diagnosis to treatment with therapeutic cell composition, baseline C Reactive Protein (CRP), pre-leukapheresis lymphocyte count (10\hat{ }9/L), gene double expressor, gene double hit, gene triple hit, gene double or triple hit, gene double or triple hit or double expressor, albumin level, alkaline phosphatase level, basophils count, absolute basophil count, direct bilirubin, total bilirubin, blood urea nitrogen level, calcium level, carbon dioxide level, chloride level, creatinine level, eosinophils count, eosinophils absolute count, glucose level, hematocrit level, hemoglobin level, LDH level, lesion count, lymphocyte count, lymphocyte absolute count, magnesium level, monocyte absolute count, monocyte count, neutrophil absolute count, neutrophil count, phosphate level, platelet count, potassium level, total protein, red blood cell count, aspartate aminotransferase level, alanine aminotransferase level, sodium level, sum of products of diameters, triglycerides, longest tumor diameter, perpendicular tumor diameter, uric acid level, and white blood cell count.

57. The method of any of claims 1-56, wherein the input composition features comprise cell phenotypes.

58. The method of any of claims 1-57, wherein the input composition features comprise one or more of CAS3−/CCR7−/CD27−/CD4+, CAS3−/CCR7−/CD27+/CD4+, CAS3−/CCR7+/CD4+, CAS3−/CCR7+/CD27−/CD4+, CAS3−/CCR7+/CD27+/CD4+, CAS3−/CD27+/CD4+, CAS3−/CD28−/CD27−/CD4+, CAS3−/CD28−/CD27+/CD4+, CAS3−/CD28+/CD4+, CAS3−/CD28+/CD27−/CD4+, CAS3−/CD28+/CD27+/CD4+, CAS3−/CCR7−/CD45RA−/CD4+, CAS3−/CCR7−/CD4+, CD45RA+/CD4+, CAS3−/CCR7+/CD45RA−/CD4+, CAS3−/CCR7+/CD45RA+/CD4+, CAS+/CD4+, CAS+/CD3+/CD4+, CD4+ clonality, CAS3−/CCR7−/CD27−/CD8+, CAS3−/CCR7−/CD27+/CD8+, CAS3−/CCR7+/CD8+, CAS3−/CCR7+/CD27−/CD8+, CAS3−/CCR7+/CD27+/CD8+, CAS3−/CD27+/CD8+, CAS3−/CD28−/CD27−/CD8+, CAS3−/CD28−/CD27+/CD8+, CAS3−/CD28+/CD8+, CAS3−/CD28+/CD27−/CD8+, CAS3−/CD28+/CD27+/CD8+, CAS3−/CCR7−/CD85RA−/CD8+, CAS3−/CCR7−/CD8+, CD85RA+/CD8+, CAS3−/CCR7+/CD85RA−/CD 8+, CAS3−/CCR7+/CD85RA+/CD8+, CAS+/CD8+, CAS+/CD3+/CD8+, and CD8+ clonality.

59. The method of any of claims 1-58, wherein the therapeutic cell composition features comprise one or more of a cell phenotype, a recombinant receptor-dependent activity, and a dose.

60. The method of any of claims 1-59, wherein the therapeutic cell composition features comprise one or more of CAS3−/CCR7−/CD27−/CD8+, CAS3−/CCR7−/CD27+/CD8+, CAS3−/CCR7+/CD8+, CAS3−/CCR7+/CD27−/CD8+,CAS3−/CCR7+/CD27+/CD8+, CAS3−/CD27+/CD8+, CAS3−/CD28+/CD8+, CAS3−/CD28+/CD27−/CD8+, CAS3−/CD28+/CD27+/CD8+, CAS3−/CCR7−/CD45RA−/CD8+, CAS3−/CCR7−/CD45RA+/CD8+, CAS3−/CCR7+/CD45RA−/CD8+, CAS3−/CCR7+/CD45RA+/CD8+, CAS+/CD3+/CAR+/CD8+, CD3+/CAR+/CD8+, CD3+/CD8+, CAR+/CD8+, clonality of CD8+ cells, EGFRt+/CD8+, cytokine−/CD8+, IFNG+/CD8+, IFNg+/IL2/CD8+, IFNg+/IL17+/TNFa+/CD8+, IFNg+/IL2+/IL17+/TNFa+/CD8+, IFNg+/IL2+/TNFa+/CD8+, CAR+/IFNg+/CD8+, IFNg+/TNFa+/CD8+, CAR+/IL2+/CD8+, IL2+/TNFa+/CD8+, cell lysis by CD8+, CAR+/TNFa+/CD8+, viable cell concentration of CD8+ cells, vector copy number of CD8+ cells, EGFRt+ vector copy number of CD8+, viability of CD8+, GMCSF+/CD8+, IFNG+/CD8+, IL10+/CD8+, IL13+/CD8+, IL2+/CD8+, IL4+/CD8+, IL5+/CD8+, IL6+/CD8+, MIP1A+/CD8+, MIP1B+/CD8+, sCD137+/CD8+, TNFa+/CD8+, dose of CD8+ cells, dose level of CD8+ cells, percent viable cells dosed of CD8+ cells, total nonviable cells dosed of CD8+ cells, total viable cells dosed of CD8+ cells, total dose of CD8+ cells, CAS3−/CCR7−/CD27−/CD4+, CAS3−/CCR7−/CD27+/CD4+, CAS3−/CCR7+/CD4+, CAS3−/CCR7+/CD27−/CD4+,CAS3−/CCR7+/CD27+/CD4+, CAS3−/CD27+/CD4+, CAS3−/CD28+/CD4+, CAS3−/CD28+/CD27−/CD4+, CAS3−/CD28+/CD27+/CD4+, CAS3−/CCR7−/CD45RA−/CD4+, CAS3−/CCR7−/CD45RA+/CD4+, CAS3−/CCR7+/CD45RA−/CD4+, CAS3−/CCR7+/CD45RA+/CD4+, CAS+/CD3+/CAR+/CD4+, CD3+/CAR+/CD4+, CD3+/CD4+, CAR+/CD4+, clonality of CD4+ cells, EGFRt+/CD4+, cytokine−/CD4+, IFNG+/CD4+, IFNg+/IL2/CD4+, IFNg+/IL17+/TNFa+/CD4+, IFNg+/IL2+/IL17+/TNFa+/CD4+, IFNg+/IL2+/TNFa+/CD4+, CAR+/IFNg+/CD4+, IFNg+/TNFa+/CD4+, CAR+/IL2+/CD4+, IL2+/TNFa+/CD4+, cell lysis by CD4+, CAR+/TNFa+/CD4+, viable cell concentration of CD4+ cells, vector copy number of CD4+ cells, EGFRt+ vector copy number of CD4+, viability of CD4+, GMCSF+/CD4+, IFNG+/CD4+, IL10+/CD4+, IL13+/CD4+, IL2+/CD4+, IL4+/CD4+, IL5+/CD4+, IL6+/CD4+, MIP1A+/CD4+, MIP1B+/CD4+, sCD137+/CD4+, TNFa+/CD4+, dose of CD4+ cells, dose level of CD4+ cells, percent viable cells dosed of CD4+ cells, total nonviable cells dosed of CD4+ cells, total viable cells dosed of CD4+ cells, and total dose of CD4+ cells.

61. The method of any of claims 1-60, wherein the sample comprises a whole blood sample, a buffy coat sample, a peripheral blood mononuclear cell (PBMC) sample, an unfractionated T cell sample, a lymphocyte sample, a white blood cell sample, an apheresis product, or a leukapheresis product.

62. The method of any of claims 1-61, wherein the sample is an apheresis product or leukapheresis product.

63. The method of claim 62, wherein the apheresis product or leukapheresis product has been previously cryopreserved.

64. The method of any of claims 1-63, wherein the T cells comprise primary cells obtained from the subject.

65. The method of any of claims 1-64, wherein the T cells comprise CD3+, CD4+, and/or CD8+ T cells.

66. The method of any of claims 1-65, wherein the input composition comprises CD4+, CD8+, or CD4+ and CD8+ T cells and the therapeutic cell composition comprises CD4+, CD8+, or CD4+ and CD8+ T cells expressing the CAR and is produced from the input composition, wherein the input composition features comprise input composition features from the CD4+, CD8+, or CD4+ and CD8+ T cell compositions of the input composition, and the therapeutic cell composition features comprise therapeutic cell composition features from the CD4+, CD8+, or CD4+ and CD8+ T cells of the therapeutic composition.

67. The method of any of claims 1-65, wherein the input composition comprises separate compositions of CD4+ and CD8+ T cells and the therapeutic cell composition comprises separate compositions of CD4+ and CD8+ T cells expressing the CAR, and is produced from the respective CD4+ or CD8+ T cell composition of the input composition, wherein the input composition features comprise input composition features from the CD4+ and CD8+ T cell compositions of the input composition, and the therapeutic cell composition features comprise therapeutic cell composition features from the CD4+ and CD8+ T cells of each of the separate compositions of the therapeutic composition.

68. The method of any of claims 1-65, wherein the input composition comprises separate compositions of CD4+ and CD8+ T cells and the therapeutic cell composition comprises a mixed composition of CD4+ and CD8+ T cells expressing the CAR, and is produced from the separate CD4+ and CD8+ T cell compositions of the input composition, wherein the input composition features comprise input composition features from the separate CD4+ and CD8+ T cell compositions of the input composition, and the therapeutic cell composition features comprise therapeutic cell composition features from the mixed composition of CD4+ and CD8+ cells of the therapeutic composition.

69. The method of any of claims 16-31 and 41-68, wherein the predetermined treatment regimen comprises or is a single treatment comprising administering:
a) 25×10⁶ CD8+CAR+ T cells and 25×10⁶ CD4+CAR+ T cells separately to the subject;
b) 50×10⁶ CD8+CAR+ T cells and 50×10⁶ CD4+CAR+ T cells separately to the subject; or
c) 75×10⁶ CD8+CAR+ T cells and 75×10⁶ CD4+CAR+ T cells separately to the subject.

70. The method of any of claims 16-31 and 41-68, wherein the altered treatment regimen comprises or is a single treatment comprising administering:
a) 50×10⁶ CD8+CAR+ T cells and 50×10⁶ CD4+CAR+ T cells separately to the subject when the predetermined treatment regimen comprises or is a single treatment comprising administering 25×10⁶ CD8+CAR+ T cells and 25×10⁶ CD4+CAR+ T cells separately to the subject;
b) 75×10⁶ CD8+CAR+ T cells and 75×10⁶ CD4+CAR+ T cells separately to the subject when the predetermined treatment regimen comprises or is a single treatment comprising administering 50×10⁶ CD8+CAR+ T cells and 50×10⁶ CD4+CAR+ T cells separately to the subject; or
c) 75×10⁶ CD8+CAR+ T cells and 75×10⁶ CD4+CAR+ T cells separately to the subject when the predetermined treatment regimen comprises or is a single treatment comprising administering 25×10⁶ CD8+CAR+ T cells and 25×10⁶ CD4+CAR+ T cells separately to the subject.

71. The method of any of claims 16-31 and 41-68, wherein the altered treatment regimen comprises or is a single treatment comprising administering:
a) 25×10⁶ CD8+CAR+ T cells and 25×10⁶ CD4+CAR+ T cells separately to the subject when the predetermined treatment regimen comprises or is a single treatment comprising administering 50×10⁶ CD8+CAR+ T cells and 50×10⁶ CD4+CAR+ T cells separately to the subject;
b) 50×10⁶ CD8+CAR+ T cells and 50×10⁶ CD4+CAR+ T cells separately to the subject when the predetermined treatment regimen comprises or is a single treatment comprising administering 75×10⁶ CD8+CAR+ T cells and 75×10⁶ CD4+CAR+ T cells separately to the subject; or
c) 25×10⁶ CD8+CAR+ T cells and 25×10⁶ CD4+CAR+ T cells separately to the subject when the predetermined treatment regimen comprises or is a single treatment comprising administering 75×10⁶ CD8+CAR+ T cells and 75×10⁶ CD4+CAR+ T cells separately to the subject.

72. The method of any of claims 16-31 and 41-68, wherein the altered treatment regimen comprises administering the therapeutic cell composition in combination with a second therapeutic agent.


SEQUENCE LISTING (69 sequences)

SEQ ID NO: 1 - protein, 24 aa, Artificial Sequence
  T2A
LEGGGEGRGSLLTCGDVEENPGPR

SEQ ID NO: 2 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 3 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 4 - protein, 18 aa, Artificial Sequence
  T2A
EGRGSLLTCGDVEENPGP

SEQ ID NO: 5 - protein, 22 aa, Artificial Sequence
  P2A
GSGATNFSLLKQAGDVEENPGP

SEQ ID NO: 6 - protein, 19 aa, Artificial Sequence
  P2A
ATNFSLLKQAGDVEENPGP

SEQ ID NO: 7 - protein, 20 aa, Artificial Sequence
  E2A
QCTNYALLKLAGDVESNPGP

SEQ ID NO: 8 - protein, 22 aa, Artificial Sequence
  F2A
VKQTLNFDLLKLAGDVESNPGP

SEQ ID NO: 9 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 10 - protein, 22 aa, Artificial Sequence
  GMCSFR alpha chain signal sequence
MLLLVTSLLLCELPHPAFLLIP

SEQ ID NO: 11 - protein, 18 aa, Artificial Sequence
  CD8 alpha signal peptide
MALPVTALLLPLALLLHA

SEQ ID NO: 12 - protein, 16 aa, Artificial Sequence
  CD33 signal peptide
MPLLLLLPLLWAGALA

SEQ ID NO: 13 - protein, 117 aa, Artificial Sequence
  Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 14 - protein, 111 aa, Artificial Sequence
  Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 15 - protein, 122 aa, Artificial Sequence
  Variable heavy (VH) Anti-BCMA
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 16 - protein, 107 aa, Artificial Sequence
  Variable light (VL) Anti-BCMA
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 17 - protein, 116 aa, Artificial Sequence
  Variable heavy (VH) Anti-BCMA
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 18 - protein, 111 aa, Artificial Sequence
  Variable light (VL) Anti-BCMA
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 19 - protein, 118 aa, Artificial Sequence
  Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 20 - protein, 105 aa, Artificial Sequence
  Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 21 - protein, 122 aa, Artificial Sequence
  Variable heavy (VH) Anti-BCMA
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 22 - protein, 111 aa, Artificial Sequence
  Variable light (VL) Anti-BCMA
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 23 - protein, 120 aa, Artificial Sequence
  Variable heavy (VH) Anti-BCMA
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 24 - protein, 112 aa, Artificial Sequence
  Variable light (VL) Anti-BCMA
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 25 - protein, 118 aa, Artificial Sequence
  Variable heavy (VH) Anti-BCMA
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 26 - protein, 112 aa, Artificial Sequence
  Variable light (VL) Anti-BCMA
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 27 - protein, 11 aa, Artificial Sequence
  CDR L1
RASQDISKYLN

SEQ ID NO: 28 - protein, 7 aa, Artificial Sequence
  CDR L2
SRLHSGV

SEQ ID NO: 29 - protein, 9 aa, Artificial Sequence
  CDR L3
GNTLPYTFG

SEQ ID NO: 30 - protein, 5 aa, Artificial Sequence
  CDR H1
DYGVS

SEQ ID NO: 31 - protein, 16 aa, Artificial Sequence
  CDR H2
VIWGSETTYYNSALKS

SEQ ID NO: 32 - protein, 7 aa, Artificial Sequence
  CDR H3
YAMDYWG

SEQ ID NO: 33 - protein, 12 aa, Artificial Sequence
  HC-CDR3
HYYYGGSYAMDY

SEQ ID NO: 34 - protein, 7 aa, Artificial Sequence
  LC-CDR2
HTSRLHS

SEQ ID NO: 35 - protein, 9 aa, Artificial Sequence
  LC-CDR3
QQGNTLPYT

SEQ ID NO: 36 - protein, 120 aa, Artificial Sequence
  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - protein, 107 aa, Artificial Sequence
  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 38 - protein, 18 aa, Artificial Sequence
  Linker
GSTSGSGKPGSGEGSTKG

SEQ ID NO: 39 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 40 - protein, 245 aa, Artificial Sequence
  scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 41 - protein, 5 aa, Artificial Sequence
  CDR H1
SYWMN

SEQ ID NO: 42 - protein, 17 aa, Artificial Sequence
  CDR H2
QIYPGDGDTNYNGKFKG

SEQ ID NO: 43 - protein, 13 aa, Artificial Sequence
  CDR H3
KTISSVVDFYFDY

SEQ ID NO: 44 - protein, 11 aa, Artificial Sequence
  CDR L1
KASQNVGTNVA

SEQ ID NO: 45 - protein, 7 aa, Artificial Sequence
  CDR L2
SATYRNS

SEQ ID NO: 46 - protein, 9 aa, Artificial Sequence
  CDR L3
QQYNRYPYT

SEQ ID NO: 47 - protein, 122 aa, Artificial Sequence
  VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 48 - protein, 108 aa, Artificial Sequence
  VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 49 - protein, 15 aa, Artificial Sequence
  Linker
GGGGSGGGGSGGGGS

SEQ ID NO: 50 - protein, 245 aa, Artificial Sequence
  scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 51 - protein, 5 aa, Artificial Sequence
  Linker
GGGGS

SEQ ID NO: 52 - protein, 4 aa, Artificial Sequence
  Linker
GGGS

SEQ ID NO: 53 - protein, 15 aa, Artificial Sequence
  Linker
GGGGSGGGGSGGGGS

SEQ ID NO: 54 - protein, 18 aa, Artificial Sequence
  Linker
GSTSGSGKPGSGEGSTKG

SEQ ID NO: 55 - protein, 21 aa, Artificial Sequence
  Linker
SRGGGGSGGGGSGGGGSLEMA

SEQ ID NO: 56 - protein, 229 aa, Homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 326 aa, Homo sapiens
  Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 58 - protein, 12 aa, Homo sapiens
  spacer (IgG4hinge)
ESKYGPPCPPCP

SEQ ID NO: 59 - protein, 119 aa, Homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - DNA, 36 bp, Homo sapiens
  spacer (IgG4hinge)
gaatctaagtacggaccgccctgccccccttgccct

SEQ ID NO: 61 - protein, 282 aa, Homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 62 - protein, 27 aa, Homo sapiens
  CD28 (amino acids 153-179 of Accession No. P10747)
FWVLVVVGGVLACYSLLVTVAFIIFWV

SEQ ID NO: 63 - protein, 66 aa, Homo sapiens
  CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 64 - protein, 41 aa, Homo sapiens
  CD28 (amino acids 180-220 of P10747)
RSKRSRLLHSDYMNMTPRRPGPTRKHYQPYAPPRDFAAYRS

SEQ ID NO: 65 - protein, 41 aa, Homo sapiens
  CD28 (LL to GG)
RSKRSRGGHSDYMNMTPRRPGPTRKHYQPYAPPRDFAAYRS

SEQ ID NO: 66 - protein, 42 aa, Homo sapiens
  4-1BB (amino acids 214-255 of Q07011.1)
KRGRKKLLYIFKQPFMRPVQTTQEEDGCSCRFPEEEEGGCEL

SEQ ID NO: 67 - protein, 112 aa, Homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 112 aa, Homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 112 aa, Homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR